(19) United States
(12) Patent Application Publication
(10) Pub. No.: US 2015/0299804 A1
(43) Pub. Date: Oct. 22, 2015

(54) BIOMARKERS FOR PREDICTING CLINICAL RESPONSE OF CANCER PATIENTS TO TREATMENT WITH IMMUNOTHERAPEUTIC AGENT

(71) Applicant: BRISTOL-MYERS SQUIBB COMPANY, Princeton, NJ (US)

(72) Inventors: Maksym ARTOMOV, Cambridge, MA (US); Scott D. Chasalow, Pennington, NJ (US); Kevin Daniel Fowler, Cambridge, MA (US); Ruiru Ji, Princeton, NJ (US); Vafa Shahabi, Valley Forge, PA (US); Fadi George Towfic, Cambridge, MA (US); Benjamin James Zeskind, Cambridge, MA (US)

(21) Appl. No.: 14/442,749

(22) PCT Filed: Nov. 14, 2013
(86) PCT No.: PCT/US13/69975

§ 371 Date: May 14, 2015

Related U.S. Application Data
(60) Provisional application No. 61/726,953, filed on Nov. 15, 2012.

(51) Int. Cl. C12Q 1/68; G06F 19/00; C07K 16/28

(52) U.S. Cl. CPC C12Q 1/6886; C07K 16/2818; G06F 19/706; C12Q 2600/106; C12Q 2600/158; C07K 2317/76

(57) ABSTRACT

Provided herein are prognostic and diagnostic methods for predicting likelihood of clinical response of a subject having cancer to treatment with an immunotherapeutic agent. Also provided herein are methods for treating a subject having cancer with an immunotherapeutic agent after determining likelihood of clinical response of the subject to such treatment.

### BACKGROUND

The National Cancer Institute has estimated that in the United States alone, 1 in 3 people will be struck with cancer during their lifetime. Moreover, approximately 50% to 60% of people contracting cancer will eventually succumb to the disease. The widespread occurrence of this disease underscores the need for improved anticancer regimens for the treatment of malignancy.

Due to the wide variety of cancers presently observed, numerous anticancer agents have been developed to destroy cancer within the body. These compounds are administered to cancer patients with the objective of destroying or otherwise inhibiting the growth of malignant cells while leaving normal, healthy cells undisturbed. Anticancer agents have been classified based upon their mechanism of action, and are often referred to as chemotherapeutics or immunotherapeutics (agents whose therapeutic effects are mediated by their immuno-modulating properties). The vertebrate immune system requires multiple signals to achieve optimal immune activation; see, e.g., Janeway, Cold Spring Harbor Symp. Quant. Biol., 54:1-14 (1989); and Paul, W. E., ed., Fundamental Immunology, 4th Edition, Raven Press, NY (1998), particularly Chapters 12 and 13, pp. 411-478. Interactions between T lymphocytes (T cells) and antigen presenting cells (APCs) are essential to the immune response. Levels of many cohesive molecules found on T cells and APC's increase during an immune response (Springer et al., Ann. Rev. Immunol., 5:223-252 (1987); Shaw et al., Curr. Opin. Immunol., 1:92-97 (1988); and Hemler, Immunology Today, 9:109-113 (1988)). Increased levels of these molecules may help explain why activated APCs are more effective at stimulating antigen-specific T cell proliferation than are resting APCs (Kaiuchi et al., J. Immunol., 131:109-114 (1983); Kreiger et al., J. Immunol., 135:2937-2945 (1985); McKenzie, J. Immunol., 141:2907-2911 (1988); and Hawrylowicz et al., J. Immunol., 141:4083-4088 (1988)).

T cell immune response is a complex process that involves cell-cell interactions (Springer et al., Ann. Rev. Immunol., 5:223-252 (1987)), particularly between T and accessory cells such as APCs, and production of soluble immune mediators (cytokines or lymphokines) (Dinarello, New Engl. J. Med., 317:940-945 (1987); and Sallusto, J. Exp. Med., 179:1109-1118 (1997)). This response is regulated by several T-cell surface receptors, including the T-cell receptor complex (Weiss, Ann. Rev. Immunol., 4:593-619 (1986)) and other “accessory” surface molecules (Allison, Curr. Opin. Immunol., 6:414-419 (1994); Springer (1987), supra). Many of these accessory molecules are naturally occurring cell surface differentiation (CD) antigens defined by the reactivity of monoclonal antibodies on the surface of cells (McMichael, ed., Leukocyte Typing Iff, Oxford Univ. Press, Oxford, N.Y. (1987)).

Early studies suggested that B lymphocyte activation requires two signals (Bretscher, Science, 169:1042-1049 (1970)) and now it is believed that all lymphocytes require two signals for their optimal activation, an antigen specific or clonal signal, as well as a second, antigen non-specific signal. (Janeway, supra). Freeman (J. Immunol., 143:2714-2722 (1989)) isolated and sequenced a cDNA clone encoding a B cell activation antigen recognized by MAb B7 (Freeman, J. Immunol., 139:3260 (1987)). COS cells transfected with this cDNA have been shown to stain by both labeled MAb B7 and MAb BB-1 (Clark, Human Immunol., 16:100-113 (1986); Yokochi, J. Immunol., 128:823 (1981); Freeman et al. (1989), supra; and Freeman et al. (1987), supra). In addition, expression of this antigen has been detected on cells of other lineages, such as monocytes (Freeman et al., (1989) supra).

T helper cell (Th) antigenic response requires signals provided by APCs. The first signal is initiated by interaction of the T cell receptor complex (Weiss, J. Clin. Invest., 86:1015 (1990)) with antigen presented in the context of major histocompatibility complex (MHC) molecules on the APC (Allen, Immunol. Today, 8:270 (1987)). This antigen-specific signal is not sufficient to generate a full response, and in the absence of a second signal may actually lead to clonal inactivation or anergy (Schwartz, Science, 248:1349 (1990)). The requirement for a second “costimulatory” signal has been demonstrated in a number of experimental systems (Schwartz, supra; Weaver et al., Immunol. Today, 11:49 (1990)).

CD28 antigen, a homodimeric glycoprotein of the immunoglobulin superfamily (Aruffo et al., Proc. Natl. Acad. Sci., 84:8573-8577 (1987)), is an accessory molecule found on most mature human T cells (Damle et al., J. Immunol., 131:2296-2300 (1983)). Current evidence suggests that this molecule functions in an alternative T cell activation pathway distinct from that initiated by the T-cell receptor complex (June et al., Mol. Cell. Biol., 7:4472-4481 (1987)). Some studies have indicated that CD28 is a counter-receptor for the B cell activation antigen, B7/BB-1 (Linsley et al., Proc. Natl. Acad. Sci. USA, 87:5031-5035 (1990)). The B7 ligands are also members of the immunoglobulin superfamily but have, in contrast to CD28, two Ig domains in their extracellular region, an N-terminal variable (V)-like domain followed by a constant (C)-like domain.

Delivery of a non-specific costimulatory signal to the T cell requires at least two homologous B7 family members found on APCs, B7-1 (also called B7, B7. 1, or CD80) and B7-2 (also called B7.2 or CD86), both of which can deliver costimulatory signals to T cells via CD28. Costimulation through CD28 promotes T cell activation.

CD28 has a single extracellular variable region (V)-like domain (Aruffo et al., supra). A homologous molecule, CTLA-4, has been identified by differential screening of a murine cytolytic-T cell cDNA library (Brunet, Nature, 328:267-270 (1987)). CTLA-4 (CD152) is a T cell surface molecule and also a member of the immunoglobulin (Ig) superfamily, comprising a single extracellular Ig domain. Researchers have reported the cloning and mapping of a gene for the human counterpart of CTLA-4 (Dariavach et al., Eur. J. Immunol., 18:1901-1905 (1988)) to the same chromosomal region (2q33-34) as CD28 (Lafage-Pochitaloff et al., Immunogenetics, 31:198-201 (1990)). Sequence comparison between this human CTLA-4 and CD28 proteins reveals significant homology of sequence, with the greatest degree of homology in the juxtamembrane and cytoplasmic regions (Brunet et al. (1988), supra; Dariavach et al. (1988), supra).

The CTLA-4 is inducibly expressed by T cells. It binds to the B7-family of molecules (primarily CD80 and CD86) on APCs (Chambers et al., Ann. Rev. Immunol., 19:565-594 (2001)). When triggered, it inhibits T-cell proliferation and function. Mice genetically deficient in CTLA-4 develop lymphoproliferative disease and autoimmunity (Tivol et al., Immunity, 3:541-547 (1995)). In pre-clinical models, CTLA-4 blockade also augments anti-tumor immunity (Leach et al., Science, 271:1734-1736 (1996); and van Elsas et al., J. Exp. Med., 190:355-366 (1999)). These findings led to the development of antibodies that block CTLA-4 for use in cancer immunotherapy.

Blockade of CTLA-4 by a monoclonal antibody leads to the expansion of all T cell populations, with activated CD4⁺ and CD8⁺ T cells mediating tumor cell destruction (Melero et al., Nat. Rev. Cancer, 7:95-106 (2007); and Wolchok et al., The Oncologist, 13 (Suppl. 4):2-9 (2008)). The antitumor response that results from the administration of anti-CTLA-4 antibodies is believed to be due to an increase in the ratio of effector T cells to regulatory T cells within the tumor microenvironment, rather than simply from changes in T cell populations in the peripheral blood (Quezada et al., J. Clin. Invest., 116:1935-1945 (2006)). One such agent is ipilimumab.

Ipilimumab (previously MDX-010; Medarex Inc., marketed by Bristol-Myers Squibb as YERVOY™) is a fully human anti-human CTLA-4 monoclonal antibody that blocks the binding of CTLA-4 to CD80 and CD86 expressed on antigen presenting cells, thereby, blocking the negative down-regulation of the immune responses elicited by the interaction of these molecules. Initial studies in patients with melanoma showed that ipilimumab could cause objective durable tumor regressions (Phan et al., Proc. Natl. Acad. Sci. USA, 100:8372-8377 (2003)). Also, reductions of serum tumor markers such as CA125 and PSA were seen for some patients with ovarian or prostate cancer, respectively (Hodi et al., Proc. Natl. Acad. Sci. USA, 100:4712-4717 (2003)). Ipilimumab has demonstrated antitumor activity in patients with advanced melanoma (Weber et al., J. Clin. Oncol., 26:5950-5956 (2008); Weber, Cancer Immunol. Immunother., 58:823-830 (2009)). In addition, in a number of phase II and two phase III clinical trials, ipilimumab was shown to increase the overall survival in advanced melanoma patients (Hodi, F. S. et al., “Improved survival with ipilimumab in patients with metastatic melanoma”, New Engl. J. Med., 363:711-723 (2010), and Robert, C. et al., “Ipilimumab plus dacarbazine for previously untreated metastatic melanoma”, New Engl. J. Med., 364:2517-2526 (2011)). Treatment with ipilimumab, however, can result in adverse events in some patients and individual survival outcome may be different.

Provided herein are biomarkers that may be used to predict clinical response of patients to treatment with an immunotherapeutic agent, for example, an anti-CTLA4 antibody such as ipilimumab, prior to receiving the agent, and methods of using the biomarkers for treatment with the immunotherapeutic agent, or for predicting clinical response of a patient treated with the immunotherapeutic agent.

### SUMMARY

Provided herein are methods for treating a subject having cancer with an immunotherapeutic agent, comprising (a) determining expression level of at least one gene in a blood sample obtained from the subject, wherein the at least one gene is selected from a first group of genes as listed in Table 2 and a second group of genes as listed in Table 3; (b) determining likelihood of clinical response of the subject to the treatment based on the expression level of the at least one gene in the blood sample, wherein the expression level of the at least one gene selected from the first group of genes is positively correlated with the likelihood of clinical response, and wherein the expression level of the at least one gene selected from the second group of genes is negatively correlated with the likelihood of clinical response; and (c) administering to the subject a therapeutically effective amount of the immunotherapeutic agent for treating the cancer.

Also provided herein are methods for predicting likelihood of clinical response of a subject having cancer to treatment with an immunotherapeutic agent, comprising (a) obtaining a blood sample from the subject before the treatment, (b) determining expression level of at least one gene in the blood sample, wherein the at least one gene is selected from a first group of genes as listed in Table 2 and a second group of genes as listed in Table 3; (c) determining likelihood of clinical response to the treatment based on the expression level of the at least one gene in the blood sample, wherein the expression level of the at least one gene selected from the first group of genes is positively correlated with the likelihood of clinical response, and wherein the expression level of the at least one gene selected from the second group of genes is negatively correlated with the likelihood of clinical response.

Also provided herein are methods for determining whether to treat a subject having cancer with a immunotherapeutic agent, comprising (a) obtaining a blood sample from the subject, (b) determining expression level of at least one gene in a blood sample obtained from the subject, wherein the at least one gene is selected from a first group of genes as listed in Table 2 and a second group of genes as listed in Table 3; (c) determining likelihood of clinical response to the treatment based on the expression level of the at least one gene in the blood sample, wherein the expression level of the at least one gene selected from the first group of genes is positively correlated with the likelihood of clinical response, and wherein the expression level of the at least one gene selected from the second group of genes is negatively correlated with the likelihood of clinical response; and (d) determining whether to treat the subject having cancer with the immunotherapeutic agent based on the likelihood of clinical response.

Also provided herein are methods for treating a subject having cancer with an immunotherapeutic agent, comprising (a) determining expression levels of a first gene and a second gene in a blood sample obtained from the subject, wherein the first gene is IL2RB and a second gene is selected from ASGR1 and ASGR2; (b) determining likelihood of longer overall survival of the subject following the treatment based on the expression levels of the first gene and the second gene in the blood sample, wherein the expression levels of the first gene and the second gene are used to calculate a score according to formula:

Score=−C₁*X_{first gene}+C₂*X_{second gene},

wherein X_{first gene} and X_{second gene} are normalized mRNA expression levels of the first and the second gene, respectively, and C1 and C2 are each, independently, a number ranging from 0.01 to 3, wherein the score is negatively correlated with the likelihood of longer overall survival; and (c) administering to the subject a therapeutically effective amount of the immunotherapeutic agent for treating the cancer.

Also provided herein are methods for predicting likelihood of longer overall survival of a subject having cancer following treatment with an immunotherapeutic agent, comprising: (a) obtaining a blood sample from the subject before the treatment; (b) determining expression levels of a first gene and a second gene in the blood sample obtained from the subject, wherein the first gene is IL2RB and a second gene is selected from ASGR1 and ASGR2; and (c) determining likelihood of longer overall survival of the subject following the treatment based on the expression levels of the first gene and the second gene in the blood sample, wherein the expression levels of the first gene and the second gene are used to calculate a score according to formula:

Score=−C₁*X_{first gene}+C₂*X_{second gene},

wherein X_{first gene} and X_{second gene} are normalized mRNA expression levels of the first and the second gene, respectively, and C1 and C2 are each, independently, a number ranging from 0.01 to 3, wherein the score is negatively correlated with the likelihood of longer overall survival.

Also provided herein are methods for determining whether to treat a subject having cancer with a immunotherapeutic agent, comprising: (a) obtaining a blood sample from the subject; (b) determining expression levels of a first gene and a second gene in the blood sample obtained from the subject, wherein the first gene is IL2RB and a second gene is selected from ASGR1 and ASGR2; and (c) determining likelihood of longer overall survival of the subject following the treatment based on the expression levels of the first gene and the second gene in the blood sample, wherein the expression levels of the first gene and the second gene are used to calculate a score according to formula:

Score=−C₁*X_{first gene}+C₂*X_{second gene},

wherein X_{first gene} and X_{second gene} are normalized mRNA expression levels of the first and the second gene, respectively, and C1 and C2 are each, independently, a number ranging from 0.01 to 3, wherein the score is negatively correlated with the likelihood of longer overall survival; and (d) determining whether to treat the subject with the immunotherapeutic agent based on the likelihood of longer overall survival.

Also provided herein are kits for use for the methods disclosed herein. The kits may comprise one or more reagents for determining expression level of at least one gene in a blood sample, wherein the at least one gene is selected from a first group of genes as listed in Table 2 and a second group of genes as listed in Table 3.

Also provided herein are kits for use for the methods disclosed herein. The kits may comprise one or more reagents for determining expression levels of a first gene and a second gene in a blood sample, wherein the first gene is IL2RB and a second gene is selected from ASGR1 and ASGR2.

TABLE 2
First group of genes

| | | |
|---|---|---|
| IL2RB | PMS2L11 | CCND3 |
| KLRK1 | ZMYND11 | TRATRD |
| G3BP | TTC17 | ZAP70 |
| PPP1R16B | CLDN15 | ADA |
| CLIC3 | TBX21 | LOC130074 |
| PRF1 | LUC7L2 | GFOD1 |
| SPON2 | CAT | HLA-A/// |
| | | HLA-H/// |
| | | LOC642047 /// |
| | | LOC649853 /// |
| | | LOC649864 |
| HOP | IMP3 | CECR7 |
| GNLY | CD2 | C7ORF24 |
| TMEM161A | GZMA | ZNF364 |
| PRKCH | SPCS2 | ID2 |
| RUNX3 | RPA2 | KLRD1 |
| GZMB | SLC25A5 | SH2D2A |
| CCND2 | CHST12 | MATK |
| NKG7 | MNAB | CDC25B |
| ARL2BP | GPR56 | GIMAP4 |
| CCL4 | TXNIP | EOMES |

TABLE 3
Second group of genes

| | | |
|---|---|---|
| ASGR1 | ING2 | TSPO |
| ASGR2 | HOMER3 | SERTAD3 |
| CENTA2 | RAB31 | SULT1A1 |
| PGLS | ARF5 | S100A6 |
| CEBPA | IL1RN | STX10 |
| ZBP1 | LILRA5 | IFI6 |
| MAPBPIP | PYCARD | C16ORF68 |
| CEACAM3 | HPSE | |

### BRIEF DESCRIPTION OF THE DRAWINGS

FIG. 1. Kaplan-Meier estimates of overall survival (OS) for patients split into 2 groups based on the two-gene signature (IL2RB+ASGR2): training cohort (Panel A), test cohort (Panel B), and both cohorts pooled (Panel C). IL2RB and ASGR2 were identified by applying two different methods to the training cohort: multivariable Cox PH regression with elastic-net penalties, and unregularized univariate Cox PH regression coupled with evaluation of 2- and 3-gene combinations. Once genes were identified, coefficients were estimated using unregularized Cox PH regression on the training cohort, and a classification threshold was selected. Finally, the selected genes, coefficients, and thresholds were applied to the test cohort and to both cohorts pooled.

FIG. 2. Combining the two-gene signature with prognostic factor baseline LDH in the training cohort (Panel A), test cohort (Panel B), both cohorts pooled (Panel C), and both cohorts pooled using two thresholds (Panel D). Coefficients were estimated using Cox PH regression in the training cohort alone. They were then applied to the training cohort, test cohort, and both cohorts pooled to obtain patient scores. The threshold for panels A-C was determined using threshold optimization in the training cohort alone, then applying this threshold to the training cohort, test cohort, and both cohorts pooled. The two thresholds used in panel D were determined using threshold optimization on both cohorts pooled together. Time-dependent ROC curves at 12 months for the training cohort (Panel E), test cohort (Panel F), and both cohorts pooled (Panel G) are presented for both the two-gene signature (red) and the three-factor signature (black), along with the relevant AUCs. The stars indicate the points on the ROC curve corresponding to the selected thresholds.

FIG. 3. Functional and enrichment analysis yields insights into the biological mechanisms underlying the two-gene signature's association with OS in advanced metastatic melanoma patients receiving ipilimumab. Network analysis of genes (red) correlated with IL2RB (Panel A) suggests a role for EOMES in connecting IL2RB with the genes significantly correlated with it, as well as with CTLA-4 itself. For genes found to be associated with OS (Panel B, row headings) the relative expression of each gene across cell types (Panel B, columns) in the DMAP¹⁸ data is shown in a heat map. This analysis suggests roles for NK and T cells (Panel B, upper left) and B cells (Panel B, middle) in genes positively associated with OS, and a role for myeloid cells (Panel B, lower right) in genes negatively associated with OS. The genes and biological mechanisms (Panel C) suggest that the two-gene signature may represent a balance of anti-tumor lymphocyte-driven functions and pro-tumor myeloid-driven functions.

FIG. 4. Time-dependent ROC curves at 12 months comparing the two-gene signature (IL2RB+ASGR2) (black) with the three-gene signatures (red) (IL2RB+ASGR2+ZBP1), (IL2RB+ASGR2+CAT), and (IL2RB+ASGR2+ASGR1).

FIG. 5. Boxplot summarizing the distribution of normalized expression levels for genes ASGR1, ASGR2, and IL2RB in the training and test cohorts pooled. Mean expression of ASGR2 was 1.54-fold higher than ASGR1, and the difference was significant by a paired t-test (P=1.32×10⁻⁶⁹).

FIG. 6. Kaplan-Meier estimates of OS, and log-rank test p-values, for patients split into 2 groups based on the two-gene signature, IL2RB+ASGR1: training cohort (Panel A), test cohort (Panel B), and both cohorts pooled (Panel C). The results are comparable to those achieved by IL2RB and ASGR2 (FIG. 1).

FIG. 7. Estimation of classification threshold(s) using the log-rank test chi-square statistic for (A) two-gene signature (IL2RB+ASGR2) in training cohort, (B) three-factor signature (IL2RB+ASGR2+LDH) in training cohort, and (C) three-factor signature (IL2RB+ASGR2+LDH) in pooled cohort (two thresholds).

FIG. 8. Analysis of EOMES by qPCR yielded a highly significant Kaplan-Meier plot (log-rank p=6.86×10⁻⁸).

The patent or application file contains at least one drawing executed in color. Copies of this patent or patent application publication with color drawing(s) will be provided by the Office upon request and payment of the necessary fee.

### DETAILED DESCRIPTION

The methods described herein are based on certain gene expression signatures. The gene expression signatures may be used as biomarkers, e.g., prognostic, predictive biomarkers for clinical efficacy and/or safety.

Provided herein are methods for treating a subject having cancer with an immunotherapeutic agent, comprising (a) determining expression level of at least one gene in a blood sample obtained from the subject, wherein the at least one gene is selected from a first group of genes as listed in Table 2 and a second group of genes as listed in Table 3; (b) determining likelihood of clinical response of the subject to the treatment based on the expression level of the at least one gene in the blood sample, wherein the expression level of the at least one gene selected from the first group of genes is positively correlated with the likelihood of clinical response, and wherein the expression level of the at least one gene selected from the second group of genes is negatively correlated with the likelihood of clinical response; and (c) administering to the subject a therapeutically effective amount of the immunotherapeutic agent for treating the cancer.

Also provided herein are methods of predicting likelihood of clinical response of a subject having cancer to treatment with an immunotherapeutic agent, comprising (a) obtaining a blood sample from the subject before the treatment, (b) determining expression level of at least one gene in the blood sample, wherein the at least one gene is selected from a first group of genes as listed in Table 2 and a second group of genes as listed in Table 3; (c) determining likelihood of clinical response of the subject to the treatment based on the expression level of the at least one gene in the blood sample, wherein the expression level of the at least one gene selected from the first group of genes is positively correlated with the likelihood of clinical response, and wherein the expression level of the at least one gene selected from the second group of genes is negatively correlated with the likelihood of clinical response.

Also provided herein are methods for determining whether to treat a subject having cancer with a immunotherapeutic agent, comprising (a) obtaining a blood sample from the subject, (b) determining expression level of at least one gene in a blood sample obtained from the subject, wherein the at least one gene is selected from a first group of genes as listed in Table 2 and a second group of genes as listed in Table 3; (c) determining likelihood of clinical response of the subject to the treatment based on the expression level of the at least one gene in the blood sample, wherein the expression level of the at least one gene selected from the first group of genes is positively correlated with the likelihood of clinical response, and wherein the expression level of the at least one gene selected from the second group of genes is negatively correlated with the likelihood of clinical response; and (d) determining whether to treat the subject with the immunotherapeutic agent based on the likelihood of clinical response.

Also provided herein are methods for treating a subject having cancer with an immunotherapeutic agent, comprising (a) determining expression levels of a first gene and a second gene in a blood sample obtained from the subject, wherein the first gene is IL2RB and a second gene is selected from ASGR1 and ASGR2; (b) determining likelihood of longer overall survival of the subject following the treatment based on the expression levels of the first gene and the second gene in the blood sample, wherein the expression levels of the first gene and the second gene are used to calculate a score according to formula:

Score=−C₁*X_{first gene}+C₂*X_{second gene},

wherein X_{first gene} and X_{second gene} are normalized mRNA expression levels of the first and the second gene, respectively, and C1 and C2 are each, independently, a number ranging from 0.01 to 3, wherein the score is negatively correlated with the likelihood of longer overall survival; and (c) administering to the subject a therapeutically effective amount of the immunotherapeutic agent for treating the cancer.

Also provided herein are methods of predicting likelihood of longer overall survival of a subject having cancer following treatment with an immunotherapeutic agent, comprising: (a) obtaining a blood sample from the subject before the treatment; (b) determining expression levels of a first gene and a second gene in the blood sample obtained from the subject, wherein the first gene is IL2RB and a second gene is selected from ASGR1 and ASGR2; and (c) determining likelihood of longer overall survival of the subject following the treatment based on the expression levels of the first gene and the second gene in the blood sample, wherein the expression levels of the first gene and the second gene are used to calculate a score according to formula:

Score=−C₁*X_{first gene}+C₂*X_{second gene},

wherein X_{first gene} and X_{second gene} are normalized mRNA expression levels of the first and the second gene, respectively, and C1 and C2 are each, independently, a number ranging from 0.01 to 3, wherein the score is negatively correlated with the likelihood of longer overall survival.

Also provided herein are methods for determining whether to treat a subject having cancer with a immunotherapeutic agent, comprising: (a) obtaining a blood sample from the subject; (b) determining expression levels of a first gene and a second gene in the blood sample obtained from the subject, wherein the first gene is IL2RB and a second gene is selected from ASGR1 and ASGR2; and (c) determining likelihood of longer overall survival of the subject following the treatment based on the expression levels of the first gene and the second gene in the blood sample, wherein the expression levels of the first gene and the second gene are used to calculate a score according to formula:

Score=−C₁*X_{first gene}+C₂*X_{second gene},

wherein X_{first gene} and X_{second gene} are normalized mRNA expression levels of the first and the second gene, respectively, and C1 and C2 are each, independently, a number ranging from 0.01 to 3, wherein the score is negatively correlated with the likelihood of longer overall survival; and (d) determining whether to treat the subject with the immunotherapeutic agent based on the likelihood of longer overall survival.

Also provided herein are kits for use for the methods disclosed herein. The kits may comprise one or more reagents for determining expression level of at least one gene in a blood sample, wherein the at least one gene is selected from a first group of genes as listed in Table 2 and a second group of genes as listed in Table 3.

Also provided herein are kits for use for the methods disclosed herein. The kits may comprise one or more reagents for determining expression levels of a first gene and a second gene in a blood sample, wherein the first gene is IL2RB and a second gene is selected from ASGR1 and ASGR2.

The term “treating” or “treatment” refers to administering an immunotherapeutic agent described herein to a subject that has cancer, or has a symptom of cancer, or has a predisposition toward cancer, with the purpose to cure, heal, alleviate, relieve, alter, remedy, ameliorate, improve, or affect cancer, the symptoms of cancer, or the predisposition toward cancer.

The terms “patient” or “subject” are used interchangeably and refer to mammals such as human patients and non-human primates, as well as experimental animals such as rabbits, rats, and mice, and other animals. Animals include all vertebrates, e.g., mammals and non-mammals, such as sheep, dogs, cows, chickens, amphibians, and reptiles.

The term “immunotherapeutic agent” means an agent that may enhance or alter immune response to a disease or disorder such as cancer. The term “immune response” refers to the concerted action of immune cells, including lymphocytes, antigen presenting cells, phagocytic cells, and granulocytes, and soluble macromolecules produced by the above cells or the liver (including antibodies, cytokines, and complement), that results in selective damage to, destruction of, or elimination from the human body of invading pathogens, cells or tissues infected with pathogens, or cancerous cells. An immunotherapeutic agent may block immuno-regulatory proteins on immune cells, such as cytotoxic T lymphocyte antigen-4 (CTLA-4), Programmed Death 1 (PD-1), PD-1 ligand 1 (PD-L1), OX40, KIR (Killer-cell Immunoglobulin-Like Receptor), or CD137. The immunotherapeutic agent may be, for example, an anti-CTLA-4 antibody, an anti-PD-1 antibody, an anti-PD-L1 antibody, an anti-KIR antibody, an OX40 agonist, a CD137 agonist, IL21 or other cytokines. In some embodiments, the immunotherapeutic agent may be an anti-CTLA-4 antibody, such as ipilimumab or tremelimumab.

The term “effective amount” refers to an amount of an immunotherapeutic agent described herein effective to “treat” a disease or disorder in a subject. In the case of cancer, the effective amount may cause any of the changes observable or measurable in a subject as described in the definition of “treating” and “treatment” above. For example, the effective amount can reduce the number of cancer or tumor cells; reduce the tumor size; inhibit or stop tumor cell infiltration into peripheral organs including, for example, the spread of tumor into soft tissue and bone; inhibit and stop tumor metastasis; inhibit and stop tumor growth; relieve to some extent one or more of the symptoms associated with the cancer, reduce morbidity and/or mortality; improve quality of life; increase or prolong overall survival; or a combination of such effects. In some embodiments, an effective amount may be an amount sufficient to decrease the symptoms of the cancer, or an amount sufficient to prolong overall survival. Efficacy in vivo can, for example, be measured by assessing the duration of survival (e.g. overall survival), time to disease progression (TTP), the response rates (RR), duration of response, and/or quality of life. Effective amounts may vary, as recognized by those skilled in the art, depending on route of administration, excipient usage, and co-usage with other agents.

The term “clinical response” refers to positive clinical outcome of a patient to the treatment defined above, and may be expressed in terms of various measures of clinical outcome. Positive clinical outcome may be considered as an improvement in any measure of patient status, including those measures ordinarily used in the art, such as tumor regression, a decrease in tumor (or lesion) size or growth, a decrease in tumor (or lesion) burden, an increase in the duration of Recurrence-Free interval (RFI), an increase in the time of Progression Free Survival (PFS), an increase in the time of Overall Survival (OS) (from treatment to death), an increase in the time of Disease-Free Survival (DFS), an increase in the duration of Distant Recurrence-Free Interval (DRFI), and/or an increase in the duration of response, and the like. Clinical response may be a complete or partial response, or stable or controlled disease progression. Clinical response may be measured, for example, at 2-4 weeks, 4-8 weeks, 8-12 weeks, 12-16 weeks, 4-6 months, 6-9 months, 9 months to 1 year, 1-2 years, 2-5 years, 5-10 years or longer, from initiation of treatment. For example, clinical response may be measured at week 8, 12, 16, 20, 24, or 36, survival at one year, 18 months, 2 years, 3 years, 4 years, 5 years, or 10 years, from initiation of treatment.

In some embodiments of the methods described herein, the likelihood of clinical response may be expressed in terms of the likelihood of an increase in the time of survival, such as longer overall survival, as compared to some patients, for example, a control or test patient group; patients who have a higher or lower expression level of a gene than the subject; patients who have a higher or lower score based on a formula and expression level of one or more genes; other patients treated with the immunotherapeutic agent; patients not treated with the immunotherapeutic agent; or patients treated with a different anti-cancer agent or procedure (e.g. surgical procedure). In some embodiments of the methods described herein, clinical response is expressed in terms of longer overall survival as compared to patients receiving the immunotherapeutic agent, e.g., ipilimumab or tremelimumab, who have a higher or lower expression level of a gene than the subject; or patients receiving the immunotherapeutic agent, e.g., ipilimumab or tremelimumab, who have a higher or lower score based on a formula and expression level of one or more genes. In some embodiments the term “longer overall survival” may mean overall survival longer than 6, 8, 9, 10, 12, or 18 months, or longer than 2, 3, 4, 5, 6, 7, 8, 9, 10, 12, 15, or 20 years. In some embodiments, “longer overall survival” may mean overall survival longer than 10, 20, 30, 40, 50, or 60 months.

In some embodiments, “likelihood of clinical response” may mean higher probability of survival at certain time points, for example, at 6, 8, 9, 10, 12, 18, 20, 30, 40, 50, or 60 months, or 1 year, 2 years, 3 years, 4 years, 5 years, 6 years, 10 years, or more than 10 years, from initiation of treatment, as compared to some patients, for example, a control or test patient group; patients who have a higher or lower expression level of a gene than the subject; patients who have a higher or lower score based on a formula and expression level of one or more genes; other patients treated with the immunotherapeutic agent; patients not treated with the immunotherapeutic agent; or patients treated with a different anti-cancer agent or procedure.

In some embodiments, the likelihood of clinical response may be expressed in terms of likelihood of an increase in the time of progression free survival (PSF). In some embodiments, “likelihood of clinical response” may mean the likelihood of an increase in the time of PSF as compared to some patients, for example, a control or test patient group; patients who have a higher or lower expression level of a gene than the subject; patients who have a higher or lower score based on a formula and expression level of one or more genes; a group of other patients treated with the immunotherapeutic agent; patients not treated with the immunotherapeutic agent; or patients treated with a different anti-cancer agent or procedure. In some embodiments, “likelihood of clinical response” may mean higher probability of PSF at certain time points, for example, at 1 year, 18 months, 2 years, 3 years, 5 years, 10 years, or more than 10 years, from initiation of treatment, as compared to some patients, for example, a control or test patient group; patients who have a higher or lower expression level of a gene than the subject; patients who have a higher or lower score based on a formula and expression level of one or more genes; other patients treated with the immunotherapeutic agent; patients not treated with the immunotherapeutic agent; or patients treated with a different anti-cancer agent.

The term “advanced cancer” means cancer that is no longer localized to the primary tumor site, or a cancer that is Stage III or IV according to the American Joint Committee on Cancer (AJCC). In some embodiments, the subject may have advanced cancer, such as advanced melanoma. Advanced melanoma may be, for example, metastatic melanoma, or stage III or IV melanoma, such as unresectable stage III or IV melanoma.

In some embodiments of the methods described herein, a blood sample may be obtained from the subject having cancer, and the expression level of at least one gene in the blood sample may be determined. The at least one gene may be selected from the genes listed in the first group of genes as listed in Table 2, wherein the expression level of the at least one gene is positively correlated with the likelihood of clinical response. For example, the at least one gene may be selected from IL2RB, KLRK1, G3BP, PPP1R16B, CLIC3, PRF1, SPON2, HOP, GNLY, TMEM161A, PRKCH, RUNX3, EOMES, SLC25A5, GZMB, IMP3, and ZAP70. It may be determined that the subject may have a high likelihood of clinical response, for example, longer overall survival, if the expression level of the at least one gene is higher than a predetermined value.

In some embodiments, the at least one gene may be selected from the genes listed in the second group of genes as listed in Table 3, wherein the expression level of the at least one gene is negatively correlated with the likelihood of clinical response. For example, the at least one gene may be selected from ASGR1, ASGR2, CENTA2, PGLS, MAPBPIP, STX10, C16ORF68, and RAB31. It may be determined that the subject may have a high likelihood of clinical response, for example, longer overall survival, if the expression level of the at least one gene is lower than a predetermined value.

In some embodiments, the expression level of at least two genes in the blood sample may be determined, and the likelihood of clinical response may be predicted based on the expression level of the at least two genes in the blood sample. The at least two genes may be selected from the genes listed in Tables 2 and 3. In some embodiments, the first gene of the at least two genes may be selected from the first group of genes as listed in Table 2, and a second gene of the at least two genes may be selected from the second group of genes as listed in Table 3. For example, the first gene may be selected from IL2RB, KLRK1, G3BP, PPP1R16B, CLIC3, PRF1, SPON2, HOP, GNLY, TMEM161A, PRKCH, RUNX3, EOMES, SLC25A5, GZMB, IMP3, and ZAP70. In some embodiments, the first gene may be IL2RB.

In some embodiments, the second gene of the at least two genes may be selected from ASGR1, ASGR2, CENTA2, PGLS, MAPBPIP, STX10, C16ORF68, and RAB31. For example, the second gene may be selected from ASGR1 and ASGR2.

In some embodiments, the at least two genes may be selected from the pairs of genes (two-gene signatures) listed in Tables 7 and 10 (see the Example section). In some embodiments, the first gene may be IL2RB and the second gene may be ASGR2. In some embodiments, the first gene may be IL2RB and the second gene may be ASGR1.

In some embodiments, the expression level of at least three genes in the blood sample may be determined, and the likelihood of clinical response may be predicted based on the expression level of the at least three genes in the blood sample. The at least three genes may be selected from the genes listed in Tables 2 and 3. A first gene of the at least three genes may be selected from the first group of genes as listed in Table 2. A second gene of the at least three genes may be selected from the second group of genes as listed in Table 3. In some embodiments, the at least three genes may be selected from three-gene groups (three-gene signatures) listed in Table 8 (see the Example section).

In some embodiments of the methods described herein, determining the likelihood of clinical response may comprise subjecting the expression level of the at least two genes to a formula to calculate a score, wherein the formula may be pre-determined by statistical analysis of (a) clinical response of a plurality of patients having the cancer to treatment with the immunotherapeutic agent and (b) the expression level of the at least two genes in pre-treatment blood samples from the plurality of patients. For example, coefficients may be calculated for each gene based on the clinical response and the gene expression level in the pre-treatment blood samples. The statistical analysis may be performed with any statistical method that is suitable for analyzing gene expression data, for example, Cox proportional-hazards (PH) regression.

In some embodiments, the formula for calculating the score is

Score=−C₁*X_{first gene}+C₂*X_{second gene},

wherein X_{first gene} and X_{second gene} may be expression level of the first and the second gene, respectively, and C1 and C2 may be, independently, pre-determined values. For example, C1 and C2 may be, independently, pre-determined coefficients of the first and the second gene, respectively, based on gene expression data obtained from pre-treatment blood samples from a patient group. For example, C1 and C2 may be each, independently, a number ranging from 0.01 to 3, wherein the score may be negatively correlated with the likelihood of survival.

In some embodiments, C₁ may range from 0.1 to 2.5, from 0.2 to 1.8, or from 0.3 to 1.4. In some embodiments, C₁ may be about 1.3.

In some embodiments, C₂ may range from 0.1 to 1.2, from 0.1 to 1.0, or from 0.2 to 0.8. In some embodiments, C₂ may be about 0.7 to 0.8.

In some embodiments, X_{first gene} and X_{second gene} may be mRNA expression level of the first and the second gene, respectively. For example, X_{first gene} and X_{second gene} may be mRNA expression level of IL2RB and ASGR2, respectively, or X_{first gene} and X_{second gene} may be mRNA expression level of IL2RB and ASGR1, respectively. The mRNA expression level may be normalized. In some embodiments, where the mRNA expression level is measured by microarray, the mRNA expression level may be normalized using a standard robust multichip average (RMA) approach.

In some embodiments, X_{first gene} and X_{second gene} may be mRNA expression level of IL2RB and ASGR2, respectively, C₁ may be about 1.3, and C₂ may be about 0.7 to 0.8.

The score described above may be compared to a predetermined threshold. A score that is lower than the threshold may be indicative of high likelihood of clinical response, for example, longer overall survival, or higher probability of survival at a time point, while a score that is higher than the threshold may be indicative of low likelihood of clinical response, for example, shorter overall survival, or lower probability of survival at a time point, as compared to a selected or control group of patients, such as, patients treated with the immunotherapeutic agent, patients not treated with the immunotherapeutic agent, or patients treated with a different anti-cancer agent or procedure.

The expression level of the at least one gene may be measured by at least one method selected from microarray, quantitative polymerase chain reaction (qPCR), and flow cytometry. “Microarray” refers to an ordered arrangement of hybridizable array elements, preferably polynucleotide probes, on a substrate.

The immunotherapeutic agent may be an antibody. In some embodiments, the immunotherapeutic agent may be an anti-CTLA4 antibody, such as a human or humanized or chimeric anti-CTLA4 antibody. In some embodiments, the immunotherapeutic agent may be ipilimumab or tremelimumab. In some embodiments, the immunotherapeutic agent may be ipilimumab

In some embodiments, the subject may have cancer selected from melanoma; prostate cancer, prostatic neoplasms, adenocarcinoma of the prostate; lung cancer, e.g., small cell lung cancer and non-small cell lung cancer; ovarian cancer; gastric cancer; adenocarcinoma of the gastric and gastro-esophageal junction; gastrointestinal stromal tumor; glioblastoma; cervical cancer; adenocarcinoma; breast cancer, invasive adenocarcinoma of the breast; pancreatic cancer; duct cell adenocarcinoma of the pancreas; sarcoma, such as chondrosarcoma, clear cell sarcoma of the kidney, endometrial stromal sarcoma, Ewing's sarcoma, osteosarcoma, peripheral primitive neuroectodermal tumor, ovarian sarcoma, soft tissue sarcoma, uterine sarcoma, adult soft tissue sarcoma, and synovial sarcoma; transitional cell carcinoma; urothelial carcinoma; Wilm's tumor and neuroblastoma; lymphoma; leukemia; ocular melanoma, intraocular melanoma, cutaneous melanoma; and kidney cancer. In some embodiments, the subject may have cancer selected from melanoma; prostate cancer, prostatic neoplasms, adenocarcinoma of the prostate; lung cancer, e.g., small cell lung cancer, non-small cell lung cancer; ovarian cancer; gastric cancer; and glioblastoma. In some embodiments, the subject may have advanced melanoma or metastatic melanoma. In some embodiments, the subject may have stage III or IV melanoma, such as unresectable stage III or IV melanoma. In some embodiments, the subject may have prostate cancer. In some embodiments, the subject may have lung cancer, e.g., small cell lung cancer or non-small cell lung cancer.

In some embodiments of the methods described herein, determining the likelihood of clinical response may be based on the gene expression level and at least one additional factor. In some embodiments, the at least one additional factor may be selected from baseline serum LDH level and disease stage (e.g., M category). In some embodiments, the at least one additional factor may be baseline serum LDH level.

In some embodiments, at the time the likelihood of clinical response of the subject is determined, the subject may be not being treated, or may have not been treated, with the immunotherapeutic agent. In some embodiments, the subject may have been treated with the immunotherapeutic agent at the time the likelihood of clinical response of the subject is determined. For example, the expression level of the at least one gene may change over time in the subject. Thus, the likelihood of clinical response may be determined to decide whether to administer (or re-administer) the immunotherapeutic agent to the subject.

Also provided are kits comprising one or more reagents for determining expression level of at least one gene in a blood sample, wherein the at least one gene is selected from a first group of genes as listed in Table 2 and a second group of genes as listed in Table 3. In some embodiments, the one or more reagents may be used to determine mRNA expression level of the at least one gene. For example, the kit may comprise at least one nucleic acid or polynucleotide capable of specifically hybridizing to the at least one gene. For example, the kit may comprise at least one probe set capable of specifically hybridizing to the at least one gene. In some embodiments, the kit may comprise at least one probe set for microarray. In some embodiments, the kit may comprise at least one reagent for performing quantitative polymerase chain reaction (qPCR). In some embodiments, the kit may comprise at least one reagent for flow cytometry.

In some embodiments, the kit may comprise one or more reagents for determining expression level of at least one gene selected from IL2RB, KLRK1, G3BP, PPP1R16B, CLIC3, PRF1, SPON2, HOP, GNLY, TMEM161A, PRKCH, RUNX3, EOMES, SLC25A5, GZMB, IMP3, and ZAP70. In some embodiments, the kit may comprise one or more reagents for determining expression level of at least one gene selected from ASGR1, ASGR2, CENTA2, PGLS, MAPBPIP, STX10, C16ORF68, and RAB31.

In some embodiments, the kit may comprise one or more reagents for determining expression level of at least two genes in the blood sample. The at least two genes may be selected from the genes listed in Tables 2 and 3. In some embodiments, the first gene of the at least two genes may be selected from the first group of genes as listed in Table 2. In some embodiments, a second gene of the at least two genes may be selected from the second group of genes as listed in Table 3. For example, the first gene may be selected from IL2RB, KLRK1, G3BP, PPP1R16B, CLIC3, PRF1, SPON2, HOP, GNLY, TMEM161A, PRKCH, RUNX3, EOMES, SLC25A5, GZMB, IMP3, and ZAP70. For example, the first gene may be IL2RB. In some embodiments, the second gene may be selected from ASGR1, ASGR2, CENTA2, PGLS, MAPBPIP, STX10, C16ORF68, and RAB31. For example, the second gene may be selected from ASGR1 and ASGR2. In some embodiments, the first gene may be IL2RB and the second gene may be ASGR2. In some embodiments, the first gene may be IL2RB and the second gene may be ASGR1. In some embodiments, the at least two genes may be selected from the pairs of genes listed in Tables 7 and 10 (Example section).

In some embodiments, the kit may comprise one or more reagents for determining expression level of at least three genes in the blood sample. The first gene of the at least three genes may be selected from the first group of genes as listed in Table 2. The second gene of the at least three genes may be selected from the second group of genes as listed in Table 3. In some embodiments, the at least three genes may be selected from three-gene groups listed in Table 8 (Example section).

The following Example contains additional information, exemplification and guidance which can be adapted to the practice of this invention in its various embodiments and the equivalents thereof. The example is intended to help illustrate the invention, and is not intended to, nor should it be construed to, limit its scope.

### EXAMPLE

### Gene Signatures in Pre-Treatment Blood of Ipilimumab Treated Patients: Predictive and Prognostic Biomarkers of Response and Survival Introduction

Ipilimumab, a fully human monoclonal antibody against the cytotoxic T-lymphocyte-associated antigen 4 (CTLA-4), promotes antitumor immunity and improves overall survival (OS) in metastatic melanoma patients.^{1,2}

Several markers have been found to associate with OS or tumor response in patients receiving ipilimumab, including tumor expression of immune-related genes,³ changes in absolute lymphocyte count (ALC),⁴ EOMES-positive CD8⁺ T cells,⁵ ICOS^{hi} CD4⁺ T cells,⁶ NY-ESO-1 seropositivity,⁷ polyfunctional NY-ESO-1 specific T cell responses,⁸ and baseline myeloid-derived suppressor cell (MDSC) levels.⁹

Despite these insights, no marker has yet emerged that meets five key criteria: (1) can be measured prior to treatment in a readily-accessible sample (e.g. blood), (2) is significantly associated with OS in patients receiving ipilimumab, (3) has a clear mechanistic explanation rooted in the underlying biology, (4) has been repeated in a test cohort independent from the training cohort on which it was developed, and (5) has an effect of a magnitude sufficient to provide clinically meaningful predictions of OS.

In this study biomarkers that meet those five criteria were identified by analyzing gene expression levels in blood drawn from 88 patients prior to receiving ipilimumab and then testing candidate predictive models in a separate cohort of 69 patients.

### Materials and Methods

1. Study Design

The multicenter, phase II clinical trial CA184-004 enrolled 82 previously-treated and untreated patients with unresectable stage III or IV melanoma, randomized 1:1 into 2 arms to receive up to 4 intravenous infusions of either 3 or 10 mg/kg ipilimumab every 3 weeks (Q3W) in the induction phase. In the phase II CA184-007 trial, treatment-naïve or previously treated patients with unresectable stage III/IV melanoma (N=115) received open-label ipilimumab (10 mg/kg every 3 wks for four doses) and were randomized to receive concomitant blinded prophylactic oral budesonide (9 mg/d with gradual taper through week 16) or placebo. Data for baseline (pre-treatment) serum lactate dehydrogenase (LDH) were available for 154 out of 157 patients in the two studies (67 in CA184004 and 87 in CA184007). Clinical variables including OS and disease stage (M category) were recorded. Patient disease stage (M category) information for each cohort appears in Table 1. Complete study design, patient characteristics and endpoint reports of these trials have been described elsewhere^{10,11}. Both studies were conducted in accordance with the ethical principles originating from the current Declaration of Helsinki and consistent with International Conference on Harmonization Good Clinical Practice and the ethical principles underlying European Union Directive 2001/20/EC and the United States Code of Federal Regulations, Title 21, Part 50 (21 C.F.R. 50). The protocols and patient informed consent forms received appropriate approval by all Institutional Review Boards or Independent Ethics Committees prior to study initiation. All participating patients (or their legally acceptable representatives) gave written informed consent for these biomarker focused studies.

TABLE 1
Disease stage (M Category) of patients in training and test cohorts

| | |
|---|---|
| Training Cohort (CA184-007) | Test Cohort (CA184-004) |

| M Category | N (%) | | M Category | N (%) | |
|---|---|---|---|---|---|
| M0 | 0 | (0%) | M0 | 1 | (1.4%) |
| M1A | 17 | (19%) | M1A | 17 | (24.6%) |
| M1B | 29 | (33%) | M1B | 5 | (7.3%) |
| M1C | 42 | (48%) | M1C | 46 | (66.7%) |
| Total | 88 | (100%) | Total | 69 | (100%) |

2. Affymetrix Gene Expression Analysis

Whole blood was collected prior to treatment. Total RNA was extracted using the Prism 6100 (Applied Biosystems, Foster City, Calif.), purified by RNAClean Kit (Agencourt Bioscience Corporation; Beverly, Mass.), and evaluated on a 2100 Bioanalyzer (Agilent Technologies, Santa Clara, Calif.). Complementary DNA preparation and hybridization on HT-HG-U133A 96-array plates followed manufacturer's protocols (Affymetrix, Santa Clara, Calif.).

3. Computational Analysis

The training cohort consisted of 88 patients from CA184007, and the test cohort comprised 69 patients from CA184004. All raw microarray data for the training and test cohorts were normalized together using a standard robust multichip average (RMA) approach,¹² which combines background adjustment, quantile normalization, and summarization, implemented in the Bioconductor package (v2.10, http://www.bioconductor.org)¹³ of the statistical computing language R (v2.15.1, http://www.r-project.org). For genes with multiple probes, the probe with the greatest mean expression level was selected.¹⁴

Feature Selection

A pathwise algorithm for Cox proportional-hazards (PH) regression, regularized by a lasso or elastic-net penalty, was applied to all probe sets for unique genes in the pre-treatment gene expression data from the training cohort to identify genes predictive of OS. This method has been previously described at length¹⁵ and is implemented as the glmnet package in the statistical computing language R. For much of the work the glmnet default alpha=1 (lasso penalty) was used, but it was also verified that alpha=0.95 yielded comparable results.

As a second method, a univariate Cox regression was applied to the pre-treatment gene expression data from the training cohort to rank the genes that were most significantly associated with OS.

Two-Gene Signature: Coefficient Estimation and Threshold Selection

Cox PH regression was used to estimate the coefficients for selected genes in order to best fit the OS data in the training cohort. Using the resulting coefficients and the gene expression values of the candidate genes, a two-gene score for each patient was calculated. For purposes of illustration, these scores were dichotomized by application of a classification threshold. This threshold was selected by minimizing, over all possible thresholds, the log-rank test p-value for comparing the OS curve in training-cohort patients with scores below the threshold to that in training-cohort patients with scores above the threshold.

Two-Gene Signature: Testing

For each patient in the test cohort, the coefficients previously estimated using the training cohort were used to calculate a score. Then the previously selected threshold was applied to classify patients into 2 groups, the Kaplan-Meier method¹⁶ was used to estimate the survival functions, and a log-rank test was used to compare OS in the 2 groups.

The scores for the training and test cohorts were then pooled, and the previously selected classification threshold was applied. Survival curves for the resulting 2 groups again were estimated by the Kaplan-Meier method and compared using a log-rank test.

Three-Factor Signature

Multivariable Cox PH regression was used to explore the relationship between selected genes and two of the most established prognostic factors in advanced melanoma: baseline serum lactate dehydrogenase (LDH) levels and disease stage (M category).¹⁷

An optimal three-factor signature (combining the previously-identified two-gene signature with LDH) was identified by performing a multivariable Cox regression on the training cohort to determine the best-fitting coefficients. Next, the comprehensive threshold exploration method described above was used to determine a good threshold.

Cell-Type Enrichment Analysis

A statistical method was developed to determine whether genes specific to particular cell types were over-represented in the set of genes positively associated with OS, and whether genes specific to particular cell types were over-represented in the set of genes negatively associated with OS. The publicly available Broad Institute Differentiation Map Portal (DMAP)¹⁸ data set was used. This data set contains a comprehensive collection of genome-wide gene expression profiles for all major human hematopoietic cell types in several replicates. To evaluate a given gene's cell-type specificity, for each gene profiled in the DMAP data an enrichment score was computed based on a published algorithm.¹⁹ Each enrichment score is a measure of how specific the expression of a particular gene is for a particular cell type. Next, for each cell type, cell-type specific gene sets were compiled using an enrichment score cut off of 10 as the criterion for inclusion of the gene into the gene set. Finally, separately for the set of genes positively associated with OS and the set of genes negatively associated with OS, a hypergeometric test was used to evaluate whether each gene set was enriched in genes specific for each of the cell types. The resulting hypergeometric p-values are reported in Tables 15-16, along with the hypergeometric p-values adjusted to control for false discovery rate (FDR) using the Benjamini-Hochberg method.

qPCR Data Analysis

Quantitative polymerase chain reaction (qPCR) was conducted using the TAQMAN® Gene Expression Assay (Life Technologies/Applied Biosystems) with Assay IDs Hs00172872_ml (EOMES) (target sequence RefSeq ID: NM_{—}005442.2) and Hs99999905_ml (GAPDH) (target sequence RefSeq ID: NM_{—}002046.4), respectively, according to methods previously described.³ The qPCR data were normalized using GAPDH as the housekeeping gene. An optimal threshold was identified using methods described above, and then a Kaplan-Meier plot was generated using R. The association with OS was determined by univariate Cox regression. In addition, Spearman's rank correlation was determined between the normalized EOMES expression by qPCR and the expression of selected genes by microarray.

### Results

### Identification of Potential Predictive-Prognostic Gene Signatures in Ipilimumab Treated Patients

Two analytical methods were used to identify genes predictive of OS: elastic-net regularized Cox PH regression, and univariate (unregularized) Cox PH regression.

When the elastic-net regularized regression method was applied to the gene expression profiles for the selected probe sets for 13,341 unique genes from 88 patients in the training cohort (treated in the CA184007 trial), with the regularization parameter, lambda between 0.3713 and 0.2443, it identified a combination of two genes predictive of OS: IL2RB (interleukin-2 receptor beta, also known as CD122; probe 205291_at) and ASGR1 (asialoglycoprotein receptor 1; probe 206743_s_at). Relaxing lambda to a number between 0.2443 and 0.2226 to identify the next gene yielded ASGR2 (asialoglycoprotein receptor 2; probe 206130_s_at). Further, the gene expression profiles of ASGR1 and ASGR2 were found to be highly correlated in the training cohort (Spearman's rank correlation, R=0.562, P=1.22×10⁻¹⁴) (Table 4). The two genes also have a close biological relationship, encoding two proteins that together form the asialoglycoprotein receptor.²⁰

TABLE 4
Genes with expression most highly correlated with that of IL2RB and ASGR2 in
both cohorts pooled, sorted by Spearman's rank correlation coefficient, R.

| IL2RB | | | | ASGR2 | | | |
|---|---|---|---|---|---|---|---|
| Gene | Probe Set | R | P Value | Gene | Probe Set | R | P Value |
| PRF1 | 214617_at | 0.735 | 2.77E−28 | CSPG2 | 221731_x_at | 0.605 | 2.91E−17 |
| RUNX3 | 204197_s_at | 0.729 | 1.24E−27 | FCN1 | 205237_at | 0.588 | 3.71E−16 |
| SPON2 | 218638_s_at | 0.692 | 5.13E−24 | CD14 | 201743_at | 0.588 | 3.75E−16 |
| CLIC3 | 219529_at | 0.692 | 5.44E−24 | GRN | 200678_x_at | 0.569 | 5.32E−15 |
| RFTN1 | 212646_at | 0.682 | 4.26E−23 | ASGR1 | 206743_s_at | 0.562 | 1.22E−14 |
| CD247 | 210031_at | 0.671 | 4.03E−22 | APLP2 | 208248_x_at | 0.551 | 5.26E−14 |
| TXK | 206828_at | 0.665 | 1.11E−21 | IFI30 | 201422_at | 0.538 | 2.52E−13 |
| PRKCH | 218764_at | 0.655 | 7.35E−21 | TSPO | 202096_s_at | 0.537 | 2.96E−13 |
| ZAP70 | 214032_at | 0.644 | 5.51E−20 | DUSP3 | 201536_at | 0.532 | 5.55E−13 |
| LUC7L2 | 220099_s_at | 0.641 | 9.34E−20 | HK3 | 205936_s_at | 0.526 | 1.08E−12 |
| FYN | 210105_s_at | 0.640 | 1.01E−19 | CENTA2 | 219358_s_at | 0.523 | 1.52E−12 |
| SYNE1 | 209447_at | 0.640 | 1.02E−19 | STAB1 | 204150_at | 0.520 | 2.26E−12 |
| TH1L | 220607_x_at | 0.637 | 1.67E−19 | LTA4H | 208771_s_at | 0.501 | 1.75E−11 |
| CHST12 | 218927_s_at | 0.636 | 2.05E−19 | CYFIP1 | 208923_at | 0.498 | 2.31E−11 |
| GZMB | 210164_at | 0.634 | 2.72E−19 | PLXNB2 | 208890_s_at | 0.491 | 5.17E−11 |
| DENND2D | 221081_s_at | 0.633 | 3.54E−19 | GNA15 | 205349_at | 0.489 | 5.94E−11 |
| CBLB | 209682_at | 0.632 | 3.98E−19 | CTSH | 202295_s_at | 0.488 | 6.61E−11 |
| IARS | 204744_s_at | 0.628 | 8.65E−19 | ANXA2P2 | 208816_x_at | 0.488 | 6.84E−11 |
| KLRD1 | 210606_x_at | 0.627 | 9.92E−19 | LILRB4 | 210152_at | 0.471 | 3.82E−10 |
| CCND2 | 200953_s_at | 0.623 | 1.67E−18 | CD33 | 206120_at | 0.457 | 1.34E−09 |
| PTGDR | 215894_at | 0.621 | 2.52E−18 | ANXA2 | 210427_x_at | 0.450 | 2.68E−09 |
| GPR56 | 212070_at | 0.620 | 2.90E−18 | LGALS1 | 201105_at | 0.399 | 1.90E−07 |
| NONO | 200057_s_at | 0.616 | 5.12E−18 | | | | |
| MAPRE2 | 202501_at | 0.615 | 6.48E−18 | | | | |
| HOP | 211597_s_at | 0.605 | 2.83E−17 | | | | |
| STAT4 | 206118_at | 0.605 | 2.88E−17 | | | | |
| NCAM1 | 212843_at | 0.604 | 3.56E−17 | | | | |
| RNPS1 | 200060_s_at | 0.603 | 4.00E−17 | | | | |
| NKG7 | 213915_at | 0.603 | 4.24E−17 | | | | |
| EVL | 217838_s_at | 0.601 | 5.14E−17 | | | | |
| KLRF1 | 220646_s_at | 0.600 | 6.35E−17 | | | | |
| PRKCQ | 210038_at | 0.598 | 8.34E−17 | | | | |
| TGFBR3 | 204731_at | 0.597 | 9.62E−17 | | | | |
| PYHIN1 | 216748_at | 0.597 | 9.66E−17 | | | | |
| CCL4 | 204103_at | 0.594 | 1.46E−16 | | | | |
| RBBP7 | 201092_at | 0.593 | 1.79E−16 | | | | |
| KLRK1 | 205821_at | 0.592 | 1.99E−16 | | | | |
| PVRIG | 219812_at | 0.591 | 2.32E−16 | | | | |
| SLC25A3 | 200030_s_at | 0.591 | 2.55E−16 | | | | |
| ST6GAL1 | 201998_at | 0.590 | 2.70E−16 | | | | |
| TBX21 | 220684_at | 0.589 | 3.29E−16 | | | | |
| GTF3C2 | 212429_s_at | 0.586 | 4.87E−16 | | | | |
| SIDT1 | 219734_at | 0.586 | 5.14E−16 | | | | |
| ARHGEF7 | 202548_s_at | 0.584 | 6.53E−16 | | | | |
| MAGED1 | 209014_at | 0.584 | 6.54E−16 | | | | |
| CD160 | 207840_at | 0.582 | 8.66E−16 | | | | |
| ADA | 204639_at | 0.581 | 9.91E−16 | | | | |
| LPXN | 216250_s_at | 0.579 | 1.31E−15 | | | | |
| CX3CR1 | 205898_at | 0.579 | 1.34E−15 | | | | |
| DNMT1 | 201697_s_at | 0.576 | 1.85E−15 | | | | |
| NFATC3 | 210555_s_at | 0.576 | 2.06E−15 | | | | |
| ATP2B4 | 212135_s_at | 0.575 | 2.29E−15 | | | | |
| PPP1R16B | 212750_at | 0.574 | 2.62E−15 | | | | |
| TRA@//TRD@ | 217143_s_at | 0.574 | 2.63E−15 | | | | |
| SMAD3 | 218284_at | 0.573 | 2.91E−15 | | | | |
| HSP90AB1 | 200064_at | 0.572 | 3.55E−15 | | | | |
| DDX47 | 220890_s_at | 0.571 | 3.73E−15 | | | | |
| CDC25B | 201853_s_at | 0.570 | 4.25E−15 | | | | |
| PLEKHA1 | 219024_at | 0.569 | 4.81E−15 | | | | |
| CS | 208660_at | 0.568 | 6.10E−15 | | | | |
| YPEL1 | 213996_at | 0.566 | 7.16E−15 | | | | |
| IL10RA | 204912_at | 0.566 | 7.54E−15 | | | | |
| ITPR3 | 201189_s_at | 0.566 | 7.73E−15 | | | | |
| TMEM109 | 201361_at | 0.566 | 7.86E−15 | | | | |
| IMP3 | 221688_s_at | 0.566 | 8.03E−15 | | | | |
| NCALD | 211685_s_at | 0.565 | 8.51E−15 | | | | |
| WWP1 | 212638_s_at | 0.564 | 1.02E−14 | | | | |
| SPTBN1 | 212071_s_at | 0.562 | 1.30E−14 | | | | |
| NPIP | 204538_x_at | 0.562 | 1.31E−14 | | | | |
| KIFAP3 | 203333_at | 0.562 | 1.32E−14 | | | | |
| PLEKHF1 | 219566_at | 0.561 | 1.38E−14 | | | | |
| OFD1 | 203569_s_at | 0.561 | 1.43E−14 | | | | |
| CTSW | 214450_at | 0.561 | 1.47E−14 | | | | |
| BLMH | 202179_at | 0.560 | 1.75E−14 | | | | |
| AUTS2 | 212599_at | 0.558 | 2.12E−14 | | | | |
| GNLY | 37145_at | 0.557 | 2.54E−14 | | | | |
| LCK | 204891_s_at | 0.556 | 2.65E−14 | | | | |
| KIR3DL2 | 207314_x_at | 0.555 | 3.32E−14 | | | | |
| LOC339047 | 221501_x_at | 0.554 | 3.39E−14 | | | | |
| ZMYND11 | 202136_at | 0.552 | 4.61E−14 | | | | |
| SLC35E2 | 217122_s_at | 0.549 | 6.37E−14 | | | | |
| CRTC3 | 218648_at | 0.548 | 7.38E−14 | | | | |

Applying the univariate (unregularized) Cox PH regression approach to the pre-treatment blood gene expression data from the 88 patients in the training cohort yielded 73 genes associated with OS with p<0.005 (Table 5), including a subset of 16 genes with p<0.001 (Table 6). IL2RB had the smallest p-value (p=4.62×10⁻⁷) in the training cohort, and higher expression of this gene was positively associated with longer survival (hazard ratio=0.28, 95% CI=0.17 to 0.46). Among the genes for which higher expression was associated with shorter survival (hazard ratio>1), ASGR1 and ASGR2 had the smallest p-values in the training cohort (P=1.18×10⁻⁶ and 1.42×10⁴, respectively).

TABLE 5
Top overall survival-associated genes in training cohort
by univariate Cox PH regression analysis, p < 0.005.

| | | Hazard Ratio | |
|---|---|---|---|
| Gene | Probe Set | (95% CI) | P Value |
| IL2RB | 205291_at | 0.28 (0.17-0.46) | 4.62E−07 |
| ASGR1 | 206743_s_at | 4.00 (2.30-6.94) | 1.18E−06 |
| KLRK1 | 205821_at | 0.40 (0.26-0.62) | 3.51E−05 |
| G3BP | 201503_at | 0.17 (0.07-0.41) | 6.44E−05 |
| PPP1R16B | 212750_at | 0.20 (0.08-0.46) | 1.24E−04 |
| ASGR2 | 206130_s_at | 2.05 (1.41-2.99) | 1.42E−04 |
| CLIC3 | 219529_at | 0.45 (0.29-0.70) | 1.58E−04 |
| PRF1 | 214617_at | 0.49 (0.34-0.70) | 2.60E−04 |
| SPON2 | 218638_s_at | 0.53 (0.38-0.73) | 3.77E−04 |
| HOP | 211597_s_at | 0.50 (0.33-0.76) | 4.76E−04 |
| GNLY | 37145_at | 0.50 (0.34-0.73) | 4.92E−04 |
| TMEM161A | 43977_at | 0.12 (0.04-0.43) | 6.26E−04 |
| CENTA2 | 219358_s_at | 3.99 (1.76-9.05) | 6.43E−04 |
| PRKCH | 218764_at | 0.50 (0.34-0.73) | 6.75E−04 |
| PGLS | 218388_at | 5.03 (1.89-13.37) | 9.13E−04 |
| RUNX3 | 204197_s_at | 0.40 (0.24-0.69) | 9.65E−04 |
| CEBPA | 204039_at | 3.61 (1.65-7.88) | 1.06E−03 |
| GZMB | 210164_at | 0.50 (0.32-0.76) | 1.07E−03 |
| CCND2 | 200953_s_at | 0.42 (0.25-0.70) | 1.11E−03 |
| ZBP1 | 208087_s_at | 3.36 (1.67-6.76) | 1.16E−03 |
| NKG7 | 213915_at | 0.48 (0.31-0.75) | 1.17E−03 |
| ARL2BP | 202092_s_at | 0.30 (0.15-0.62) | 1.19E−03 |
| CCL4 | 204103_at | 0.53 (0.37-0.78) | 1.31E−03 |
| PMS2L11 | 210707_x_at | 0.34 (0.18-0.65) | 1.42E−03 |
| ZMYND11 | 202136_at | 0.49 (0.32-0.76) | 1.72E−03 |
| TTC17 | 218972_at | 0.35 (0.19-0.67) | 1.80E−03 |
| MAPBPIP | 218291_at | 4.34 (1.73-10.91) | 1.87E−03 |
| CLDN15 | 219640_at | 0.22 (0.08-0.58) | 2.00E−03 |
| TBX21 | 220684_at | 0.49 (0.31-0.77) | 2.09E−03 |
| CEACAM3 | 208052_x_at | 3.71 (1.57-8.75) | 2.11E−03 |
| ING2 | 205981_s_at | 3.79 (1.67-8.60) | 2.23E−03 |
| LUC7L2 | 220099_s_at | 0.40 (0.23-0.71) | 2.28E−03 |
| CAT | 201432_at | 0.40 (0.22-0.73) | 2.30E−03 |
| IMP3 | 221688_s_at | 0.37 (0.20-0.70) | 2.31E−03 |
| CD2 | 205831_at | 0.50 (0.33-0.76) | 2.37E−03 |
| GZMA | 205488_at | 0.55 (0.38-0.81) | 2.39E−03 |
| SPCS2 | 201240_s_at | 0.37 (0.21-0.68) | 2.47E−03 |
| HOMER3 | 215489_x_at | 4.22 (1.66-10.69) | 2.57E−03 |
| RPA2 | 201756_at | 0.48 (0.31-0.76) | 2.61E−03 |
| RAB31 | 217763_s_at | 3.31 (1.48-7.41) | 2.63E−03 |
| SLC25A5 | 200657_at | 0.18 (0.07-0.52) | 2.69E−03 |
| ARF5 | 201526_at | 4.80 (1.72-13.42) | 2.70E−03 |
| CHST12 | 218927_s_at | 0.30 (0.13-0.68) | 2.75E−03 |
| MNAB | 220202_s_at | 0.31 (0.14-0.67) | 3.01E−03 |
| IL1RN | 212657_s_at | 2.36 (1.33-4.21) | 3.02E−03 |
| GPR56 | 212070_at | 0.52 (0.34-0.80) | 3.11E−03 |
| TXNIP | 201010_s_at | 0.16 (0.05-0.54) | 3.19E−03 |
| CCND3 | 201700_at | 0.34 (0.17-0.72) | 3.38E−03 |
| TRATRD | 217147_s_at | 0.56 (0.38-0.81) | 3.45E−03 |
| LILRA5 | 215838_at | 1.87 (1.23-2.84) | 3.47E−03 |
| ZAP70 | 214032_at | 0.48 (0.29-0.79) | 3.48E−03 |
| PYCARD | 221666_s_at | 3.67 (1.54-8.74) | 3.49E−03 |
| ADA | 204639_at | 0.37 (0.18-0.75) | 3.69E−03 |
| HPSE | 219403_s_at | 1.89 (1.23-2.92) | 3.71E−03 |
| TSPO | 202096_s_at | 3.96 (1.54-10.21) | 3.71E−03 |
| LOC130074 | 212017_at | 0.33 (0.15-0.69) | 3.82E−03 |
| GFOD1 | 219821_s_at | 0.41 (0.22-0.76) | 4.13E−03 |
| HLA-A /// | 213932_x_at | 0.18 (0.06-0.58) | 4.15E−03 |
| HLA-H /// | | | |
| LOC642047 /// | | | |
| LOC649853 /// | | | |
| LOC649864 | | | |
| CECR7 | 220452_x_at | 0.16 (0.04-0.59) | 4.23E−03 |
| SERTAD3 | 219382_at | 3.96 (1.51-10.38) | 4.25E−03 |
| C7ORF24 | 215380_s_at | 0.24 (0.09-0.65) | 4.31E−03 |
| ZNF364 | 212742_at | 0.20 (0.06-0.62) | 4.34E−03 |
| SULT1A1 | 215299_x_at | 2.16 (1.26-3.71) | 4.38E−03 |
| S100A6 | 217728_at | 3.69 (1.49-9.17) | 4.41E−03 |
| ID2 | 201565_s_at | 0.33 (0.16-0.70) | 4.42E−03 |
| STX10 | 212625_at | 3.51 (1.44-8.55) | 4.47E−03 |
| KLRD1 | 210606_x_at | 0.55 (0.36-0.85) | 4.57E−03 |
| SH2D2A | 207351_s_at | 0.33 (0.15-0.73) | 4.58E−03 |
| MATK | 206267_s_at | 0.41 (0.23-0.75) | 4.60E−03 |
| IFI6 | 204415_at | 1.49 (1.15-1.94) | 4.88E−03 |
| CDC25B | 201853_s_at | 0.54 (0.35-0.82) | 4.92E−03 |
| C16ORF68 | 218945_at | 2.40 (1.33-4.35) | 4.94E−03 |
| GIMAP4 | 219243_at | 0.25 (0.09-0.66) | 4.97E−03 |

TABLE 6
Top overall survival-associated genes in training cohort
by univariate Cox PH regression analysis, with p < 0.001

| | | Hazard Ratio | |
|---|---|---|---|
| Gene | Probe Set | (95% CI) | P Value |
| IL2RB | 205291_at | 0.28 (0.17-0.46) | 4.62E−07 |
| ASGR1 | 206743_s_at | 4.00 (2.30-6.94) | 1.18E−06 |
| KLRK1 | 205821_at | 0.40 (0.26-0.62) | 3.51E−05 |
| G3BP | 201503_at | 0.17 (0.07-0.41) | 6.44E−05 |
| PPP1R16B | 212750_at | 0.20 (0.08-0.46) | 1.24E−04 |
| ASGR2 | 206130_s_at | 2.05 (1.41-2.99) | 1.42E−04 |
| CLIC3 | 219529_at | 0.45 (0.29-0.70) | 1.58E−04 |
| PRF1 | 214617_at | 0.49 (0.34-0.70) | 2.60E−04 |
| SPON2 | 218638_s_at | 0.53 (0.38-0.73) | 3.77E−04 |
| HOP | 211597_s_at | 0.50 (0.33-0.76) | 4.76E−04 |
| GNLY | 37145_at | 0.50 (0.34-0.73) | 4.92E−04 |
| TMEM161A | 43977_at | 0.12 (0.04-0.43) | 6.26E−04 |
| CENTA2 | 219358_s_at | 3.99 (1.76-9.05) | 6.43E−04 |
| PRKCH | 218764_at | 0.50 (0.34-0.73) | 6.75E−04 |
| PGLS | 218388_at | 5.03 (1.89-13.37) | 9.13E−04 |
| RUNX3 | 204197_s_at | 0.40 (0.24-0.69) | 9.65E−04 |

Next, the 73 genes identified above were analyzed in all 2,628 possible two-gene and all 62,196 possible three-gene combinations. For each such combination, an unregularized Cox PH model to predict OS as an additive function of the two or three expression values was fit to the training-cohort data. A likelihood-ratio test was used to compare each model to a null (constant) model. Among the top 10 two-gene signatures in the training cohort (Table 7) by p-value (where p-value is used solely for ranking), two stood out as being the highest ranked: IL2RB+ASGR1 (p=1.56×10⁻¹⁰) and IL2RB+ASGR2 (p=2.79×10⁻¹⁰).

TABLE 7
Top two-gene signatures in training
cohort by Cox PH regression analysis.

| | | Training Cohort | Test Cohort | Both Cohorts |
|---|---|---|---|---|
| Gene 1 | Gene 2 | P Value | P Value | P Value |
| IL2RB | ASGR1 | 1.56E−10 | 2.21E−03 | 2.21E−13 |
| IL2RB | ASGR2 | 2.79E−10 | 5.00E−04 | 1.32E−13 |
| IL2RB | PGLS | 1.25E−09 | 4.05E−02 | 3.00E−09 |
| IL2RB | CENTA2 | 2.31E−09 | 1.38E−02 | 1.47E−10 |
| ASGR1 | PRF1 | 3.23E−09 | 1.66E−02 | 3.15E−11 |
| ASGR1 | SLC25A5 | 3.80E−09 | 3.45E−03 | 6.23E−12 |
| ASGR1 | SPON2 | 6.36E−09 | 1.17E−02 | 3.95E−11 |
| ASGR1 | GNLY | 9.32E−09 | 5.06E−02 | 3.78E−10 |
| IL2RB | MAPBPIP | 1.06E−08 | 6.61E−03 | 3.07E−10 |
| ASGR1 | GZMB | 1.11E−08 | 4.91E−02 | 2.02E−09 |

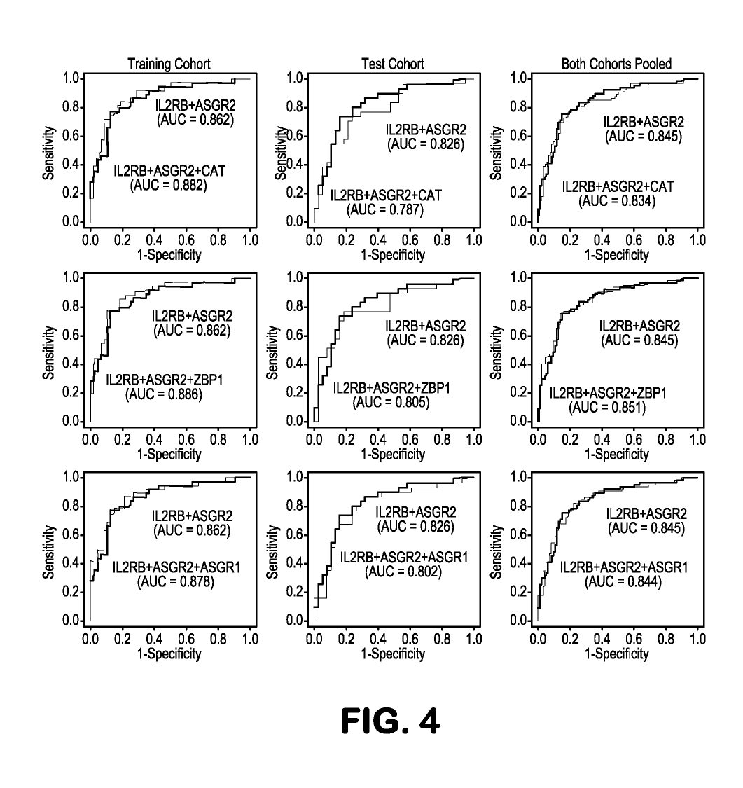

The three-gene signature with the smallest p-value in the training cohort was comprised of the combination of IL2RB, ASGR2, and CAT (catalase, probe 201432_at), p=2.41×10⁴¹. However, the p-value of this signature in the test cohort (as determined by applying the training model coefficients and threshold to the test cohort and calculating the log-rank p-value) was p=6.40×10⁻³, not below the p<0.001 threshold. To further explore the potential value of adding a third gene, possible three-gene signatures with a p<0.001 in the test cohort were examined. Among these, the three-gene signature with the smallest p-value in the training cohort (p=1.94×10⁻¹⁰) was IL2RB+ASGR2+ZBP1 (Z-DNA binding protein 1, probe 208087_s_at), with a significant p value also in the test cohort (p=9.53×10⁻⁴). For the training cohort, adding a third gene decreased the p-value for association with OS by at most one order of magnitude over the best two-gene signature (IL2RB+ASGR2). Furthermore, time-dependent Receiver Operating Characteristic (ROC) curves at 12 months²¹ show that the majority of the predictive power comes from IL2RB+ASGR2 (FIG. 4). In addition, among the top ten three-gene signatures in the training cohort (Table 8), six contained IL2RB and six contained either ASGR1 or ASGR2.

TABLE 8
Top three-gene signatures in training
cohort by Cox PH regression analysis.

| | | | Training | Test | Both |
|---|---|---|---|---|---|
| | | | Cohort | Cohort | Cohorts |
| Gene 1 | Gene 2 | Gene 3 | P Value | P Value | P Value |
| IL2RB | ASGR2 | CAT | 2.41E−11 | 6.40E−03 | 3.56E−13 |
| IL2RB | ASGR2 | PGLS | 3.13E−11 | 1.22E−02 | 2.28E−12 |
| SPON2 | PGLS | SLC25A5 | 3.26E−11 | 1.64E−01 | 4.50E−08 |
| IL2RB | ASGR1 | CAT | 4.02E−11 | 1.57E−02 | 8.19E−13 |
| IL2RB | ASGR2 | ASGR1 | 6.38E−11 | 2.99E−03 | 5.45E−14 |
| SPON2 | MAPBPIP | SLC25A5 | 6.71E−11 | 1.48E−02 | 2.90E−11 |
| IL2RB | PGLS | SLC25A5 | 6.97E−11 | 8.00E−02 | 2.60E−09 |
| IL2RB | ASGR1 | SLC25A5 | 8.16E−11 | 3.57E−03 | 1.07E−13 |
| PRF1 | PGLS | SLC25A5 | 8.42E−11 | 1.92E−01 | 1.07E−07 |
| PRF1 | ASGR1 | SLC25A5 | 1.01E−10 | 5.45E−03 | 2.36E−13 |

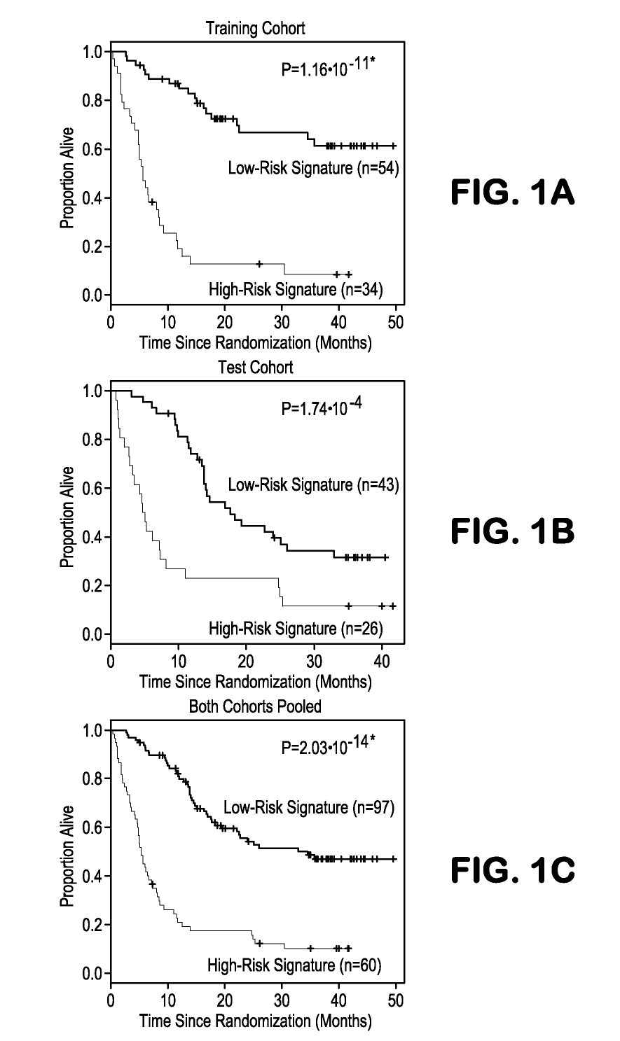

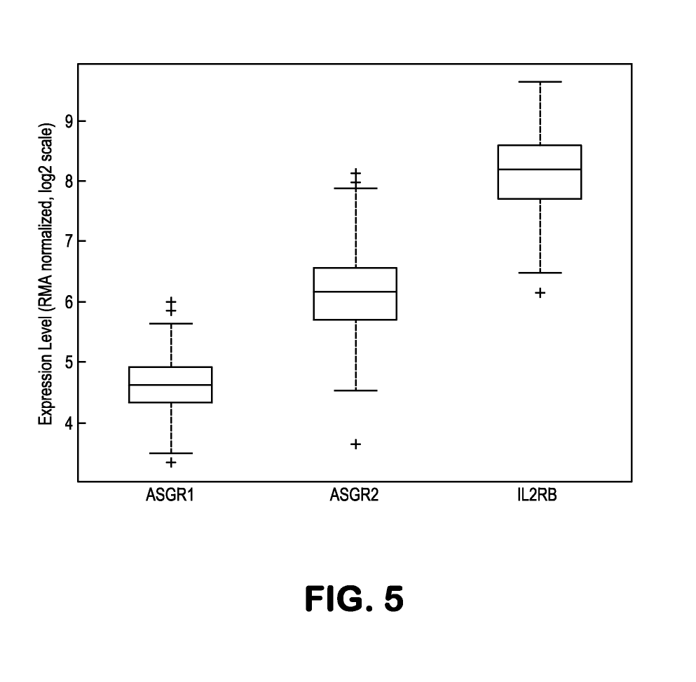

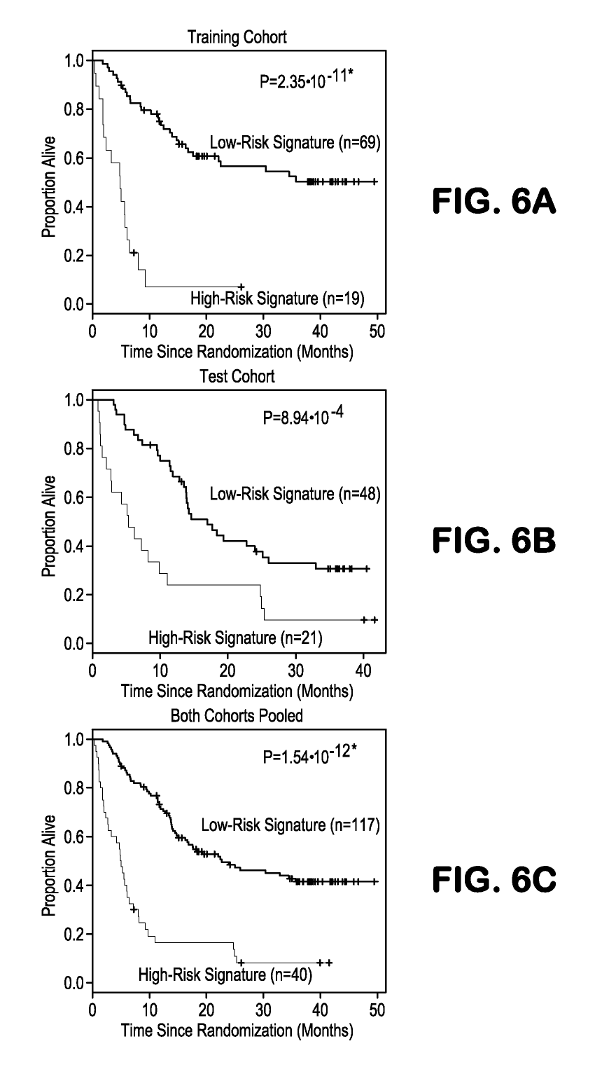

In summary, two different methods converged on two signatures associated with OS in metastatic melanoma patients receiving ipilimumab: IL2RB+ASGR1 and IL2RB+ASGR2. Both signatures yielded comparable log-rank p-values and Kaplan-Meier plots in the training, test, and pooled cohorts (IL2RB+ASGR2, FIG. 1; IL2RB+ASGR1, FIG. 6). However, ASGR2 had a significantly higher mean expression level than ASGR1 (1.54-fold higher, P=1.32×10⁻⁶⁹ by paired t-test, FIG. 5), and therefore is likely to confer more consistency, less inter-assay variability and higher clinical robustness to a predictive signature. For this reason, the combination of IL2RB+ASGR2 was chosen as the primary two-gene signature for the analyses that follow.

The two coefficients for combining IL2RB and ASGR2 in a two-gene signature to predict OS were estimated using unregularized Cox PH regression in the training cohort. The estimated coefficients were −1.312 for IL2RB and 0.748 for ASGR2 (Table 9). The two-gene score for each patient could thus be calculated from the following equation: −1.312*X_{IL2RB}+0.748*X_{ASGR2}, where Xⱼ gives the log 2-scale RMA-normalized expression level for gene j. The signs of the coefficients indicate that higher expression of IL2RB was associated with longer survival (lesser hazard) whereas higher expression of ASGR2 was associated with shorter survival (greater hazard).

TABLE 9
Coefficients based on the training and test cohorts and the two cohorts pooled
together, as well as coefficients based on regularized Cox regression.

| | | Training Cohort | | Test Cohort | | Both Cohorts Pooled | |
|---|---|---|---|---|---|---|---|
| | | Lambda | | Lambda | | Lambda | |
| Gene | Model | (by CV) | Coefficient | (by CV) | Coefficient | (by CV) | Coefficient |
| IL2RB | alpha = 1 | 0.02895 | −1.20684 | 0.115523 | −0.36107 | 0.0417252 | −0.804715 |
| | alpha = 0.95 | 0.023 | −1.2291964 | 0.0696 | −0.458378 | 0.0482 | −0.791582 |
| | Unregularized | 0 | −1.3123 | 0 | −0.5861 | 0 | −0.9063 |
| ASGR2 | alpha = 1 | 0.02895 | 0.66974 | 0.115523 | 0.28155 | 0.0417252 | 0.5239357 |
| | alpha = 0.95 | 0.023 | 0.686752 | 0.0696 | 0.350097 | 0.0482 | 0.5149287 |
| | Unregularized | 0 | 0.7475 | 0 | 0.4419 | 0 | 0.59948 |

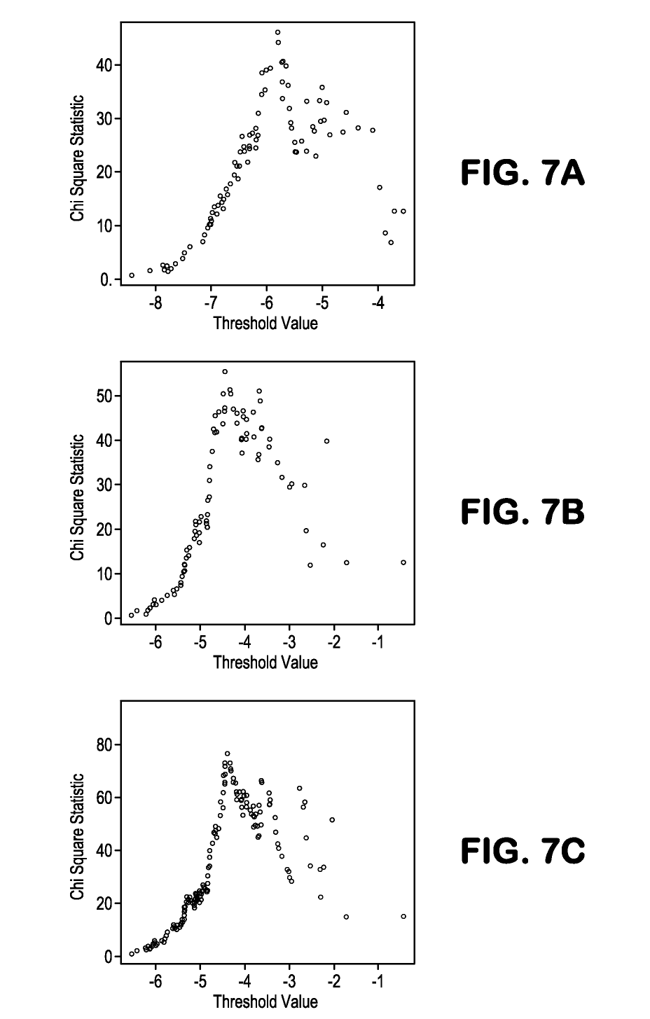

In order to generate Kaplan-Meier plots evaluating the association of the two-gene score with OS, it was necessary to select a threshold separating scores for high risk patients (shorter survival) from those with low risk (longer survival). Thus, each possible threshold was applied to classify the training cohort into two risk groups, and a log-rank test was used to compare OS in the two groups (FIG. 7A). The threshold yielding the largest chi-square statistic was −5.80, with longer survivors having smaller score values and shorter survivors having greater values (FIG. 1A).

In order to test our findings from the training cohort, the same coefficients and threshold were applied to the gene expression data from patients in the test cohort (CA184004 trial). The two-gene signature maintained a highly significant association with OS in the test cohort (log-rank p=1.74×10⁻⁴) with a clear separation of the survival curve estimates (FIG. 1B).

Finally, for illustration purposes, training- and test-cohort scores were pooled for the same two-gene signature, using the coefficients and threshold estimated from the training-cohort data alone, and again estimated OS curves for the two resulting risk groups (FIG. 1C).

While the two-gene signatures comprised of IL2RB+ASGR2 and IL2RB+ASGR1 were optimal with regard to our model-selection criteria in the training cohort, and were significant and had good predictive accuracy in the test cohort, for completeness this study sought to identify additional pairs of genes that were strongly associated with OS in both the training and test cohorts. For the 2,628 possible two-gene signatures derived from the 73 best genes in the training cohort, Cox PH regression was used to estimate the coefficients and p-values in the training cohort, then the coefficients from the training cohort was applied to the test cohort and the resulting p-values determined. All signatures that had p<0.001 in both the training cohort and the test cohort were retained (Table 10). Then the same procedure was used in reverse: all genes with a univariate Cox regression p<0.005 in the test cohort were selected, then all two-gene combinations formed from those genes were evaluated and the ones with p<0.001 in both the test and training cohorts were retained. More than 88% of the resulting signatures included IL2RB or ASGR2 (Table 11).

TABLE 10
Two-gene signatures with p < 0.001 by Cox PH regression
in both cohorts, sorted by training-cohort P value.

| | | Training Cohort | Test Cohort | Both Cohorts |
|---|---|---|---|---|
| Gene 1 | Gene 2 | P Value | P Value | P Value |
| IL2RB | ASGR2 | 2.79E−10 | 5.00E−04 | 1.32E−13 |
| IL2RB | STX10 | 1.87E−07 | 7.97E−04 | 6.82E−10 |
| IL2RB | C16ORF68 | 4.55E−07 | 4.10E−04 | 4.59E−10 |
| ASGR2 | RUNX3 | 5.55E−07 | 3.99E−04 | 8.43E−10 |
| ASGR2 | IMP3 | 2.19E−06 | 8.47E−04 | 4.58E−09 |
| ASGR2 | SLC25A5 | 2.61E−06 | 4.72E−04 | 4.93E−10 |
| ASGR2 | C16ORF68 | 3.44E−05 | 3.05E−04 | 4.60E−09 |
| ZAP70 | STX10 | 2.30E−04 | 5.71E−04 | 5.39E−07 |
| RAB31 | C16ORF68 | 2.39E−04 | 5.14E−05 | 4.74E−07 |
| STX10 | C16ORF68 | 3.50E−04 | 2.11E−04 | 1.30E−06 |
| RUNX3 | STX10 | 3.88E−04 | 3.72E−04 | 1.58E−05 |
| SLC25A5 | STX10 | 5.25E−04 | 3.26E−04 | 2.74E−06 |
| PRKCH | C16ORF68 | 6.27E−04 | 3.80E−04 | 8.95E−06 |
| RUNX3 | C16ORF68 | 6.38E−04 | 9.48E−05 | 9.07E−06 |

TABLE 11
Additional two-gene signatures with p < 0.001 by Cox
PH regression in both cohorts, determined by training on
original test cohort and testing on original training cohort,
and sorted by P Value in original training cohort.

| | | Original | Original | |
|---|---|---|---|---|
| | | Test | Training | Both |
| | | Cohort | Cohort | Cohorts |
| Gene 1 | Gene 2 | P Value | P Value | P Value |
| IL2RB | ASGR2 | 4.81E−04 | 5.05E−10 | 1.66E−13 |
| ASGR2 | RUNX3 | 3.95E−04 | 5.29E−07 | 9.20E−10 |
| IL2RB | MT1M | 3.04E−05 | 3.95E−06 | 2.37E−11 |
| IL2RB | C16ORF68 | 3.51E−05 | 4.66E−06 | 1.19E−09 |
| ASGR2 | WBP11 | 2.03E−04 | 5.98E−06 | 5.01E−09 |
| ASGR2 | EIF4B | 8.09E−04 | 6.39E−06 | 2.25E−09 |
| IL2RB | HIST2H2AA /// | 3.52E−04 | 6.82E−06 | 2.16E−09 |
| | LOC653610 /// | | | |
| | H2AR | | | |
| ASGR2 | RFTN1 | 9.59E−05 | 1.08E−05 | 4.71E−08 |
| IL2RB | IFI27 | 2.41E−06 | 1.09E−05 | 1.24E−10 |
| IL2RB | AMFR | 4.60E−04 | 1.13E−05 | 9.65E−10 |
| IL2RB | FOLR3 | 1.80E−05 | 1.42E−05 | 5.33E−10 |
| ASGR2 | AMFR | 1.51E−04 | 1.83E−05 | 3.04E−10 |
| IL2RB | C4A /// C4B | 1.94E−04 | 1.88E−05 | 3.51E−08 |
| IL2RB | VPREB3 | 3.07E−04 | 1.89E−05 | 3.98E−08 |
| ASGR2 | C4A /// C4B | 1.67E−05 | 2.16E−05 | 6.90E−10 |
| RBBP7 | ASGR2 | 3.96E−04 | 2.36E−05 | 1.54E−08 |
| IL2RB | FTHP1 | 6.47E−04 | 2.74E−05 | 1.24E−06 |
| IL2RB | HK3 | 5.36E−04 | 2.99E−05 | 7.44E−08 |
| ASGR2 | ZAP70 | 9.49E−04 | 3.16E−05 | 1.02E−08 |
| IL2RB | KIAA1026 | 2.66E−04 | 3.26E−05 | 3.08E−08 |
| IL2RB | ACTA2 | 3.13E−05 | 3.82E−05 | 7.67E−10 |
| IL2RB | FTH1 | 8.81E−05 | 4.41E−05 | 8.58E−06 |
| IL2RB | SLC7A1 | 1.85E−07 | 5.45E−05 | 1.43E−10 |
| ASGR2 | C16ORF68 | 1.78E−04 | 5.46E−05 | 9.66E−09 |
| ASGR2 | HSPA8 | 4.44E−04 | 7.11E−05 | 1.68E−08 |
| IL2RB | SUMO2 | 6.35E−04 | 7.21E−05 | 2.41E−07 |
| ASGR2 | HNRPH1 | 5.96E−04 | 7.25E−05 | 2.15E−08 |
| IL2RB | HP /// HPR | 8.00E−05 | 7.64E−05 | 8.25E−09 |
| IL2RB | GTF3A | 4.37E−04 | 7.71E−05 | 8.72E−07 |
| IL2RB | LOC171220 | 3.96E−05 | 8.25E−05 | 3.81E−05 |
| FOXO3A | IL2RB | 5.36E−04 | 8.34E−05 | 2.48E−06 |
| IL2RB | TCF3 | 6.30E−06 | 8.75E−05 | 7.56E−08 |
| ASGR2 | CD247 | 7.57E−04 | 9.46E−05 | 6.59E−08 |
| ASGR2 | MAGED1 | 5.19E−04 | 1.01E−04 | 6.89E−07 |
| ASGR2 | CAMP | 2.97E−06 | 1.06E−04 | 1.40E−09 |
| ASGR2 | XBP1 | 5.16E−04 | 1.12E−04 | 2.61E−08 |
| ASGR2 | IFI27 | 1.70E−05 | 1.14E−04 | 9.88E−10 |
| IL2RB | CA4 | 3.67E−04 | 1.30E−04 | 2.12E−07 |
| ASGR2 | LOC171220 | 2.29E−04 | 1.44E−04 | 4.79E−05 |
| IL2RB | NCF1 /// | 2.28E−04 | 1.57E−04 | 1.51E−08 |
| | LOC653361 /// | | | |
| | LOC653840 | | | |
| ASGR2 | MTMR1 | 2.37E−05 | 1.63E−04 | 1.57E−08 |
| IL2RB | HSPA6 /// | 4.48E−04 | 1.66E−04 | 5.98E−07 |
| | LOC652878 | | | |
| C4A /// C4B | RAB31 | 6.00E−05 | 1.66E−04 | 5.94E−07 |
| IL2RB | ACTN1 | 7.68E−04 | 1.66E−04 | 1.98E−07 |
| ASGR2 | IL10RA | 2.36E−04 | 1.69E−04 | 3.04E−07 |
| ASGR2 | SUMO2 | 2.71E−04 | 1.82E−04 | 5.19E−08 |
| ASGR2 | HP /// HPR | 4.21E−04 | 1.91E−04 | 1.65E−08 |
| IL2RB | PQLC1 | 1.79E−04 | 1.92E−04 | 1.52E−07 |
| ASGR2 | TCF3 | 1.19E−05 | 1.95E−04 | 3.51E−08 |
| IL2RB | HNRPH1 | 4.93E−04 | 1.95E−04 | 2.83E−07 |
| IL2RB | MAG | 2.75E−05 | 1.98E−04 | 1.39E−08 |
| IL2RB | WNK1 | 9.21E−05 | 2.01E−04 | 5.41E−07 |
| IL2RB | HIST1H2BD | 2.83E−04 | 2.14E−04 | 4.80E−08 |
| ASGR2 | EVL | 4.26E−04 | 2.30E−04 | 1.15E−07 |
| RAB31 | C16ORF68 | 3.82E−05 | 2.37E−04 | 3.45E−07 |
| ASGR2 | FTH1 | 4.46E−04 | 2.46E−04 | 1.33E−05 |
| ASGR2 | FAM102A | 3.02E−04 | 2.50E−04 | 1.41E−07 |
| ASGR2 | NPM1 | 5.13E−04 | 2.57E−04 | 1.80E−07 |
| IL2RB | HSPA6 | 2.72E−04 | 2.59E−04 | 3.81E−07 |
| ASGR2 | FOLR3 | 3.22E−06 | 2.68E−04 | 7.96E−10 |
| IL2RB | FAM102A | 1.95E−04 | 2.86E−04 | 4.65E−07 |
| IL2RB | HLADQB1 /// | 1.25E−05 | 3.10E−04 | 1.28E−08 |
| | LOC650557 | | | |
| IL2RB | RALBP1 | 2.13E−04 | 3.22E−04 | 7.06E−08 |
| IL2RB | ECGF1 | 4.85E−04 | 3.26E−04 | 1.54E−06 |
| ASGR2 | MAP3K4 | 5.38E−04 | 3.46E−04 | 2.54E−06 |
| IL2RB | PPP1R10 | 4.73E−06 | 3.61E−04 | 1.03E−09 |
| ASGR2 | PDCD4 | 4.33E−04 | 3.64E−04 | 1.24E−06 |
| RUNX3 | KIAA0690 | 7.23E−04 | 3.75E−04 | 1.10E−06 |
| IL2RB | MTMR1 | 3.55E−04 | 3.94E−04 | 1.13E−06 |
| IL2RB | CKAP4 | 3.83E−05 | 4.14E−04 | 8.02E−08 |
| RFTN1 | RAB31 | 5.89E−04 | 4.17E−04 | 6.86E−05 |
| ASGR2 | KIAA1026 | 1.24E−04 | 4.18E−04 | 4.68E−08 |
| IL2RB | P2RX5 | 9.59E−05 | 4.21E−04 | 3.48E−07 |
| IL2RB | ZAP70 | 7.15E−04 | 4.27E−04 | 7.38E−07 |
| IFI27 | RAB31 | 7.89E−06 | 4.32E−04 | 6.29E−08 |
| ASGR2 | KIAA0746 | 5.33E−04 | 4.36E−04 | 1.83E−07 |
| IL2RB | UBE2M | 9.69E−06 | 4.55E−04 | 6.23E−06 |
| IL2RB | PGCP | 2.49E−04 | 4.70E−04 | 4.14E−07 |
| IL2RB | NAGK | 2.73E−04 | 4.91E−04 | 6.93E−07 |
| IL2RB | MARK3 | 1.56E−04 | 4.92E−04 | 1.17E−05 |
| IL2RB | ENDOD1 | 9.02E−06 | 4.97E−04 | 1.08E−07 |
| IL2RB | CD6 | 1.51E−04 | 5.14E−04 | 5.39E−07 |
| IL2RB | MRPL46 | 2.26E−04 | 5.34E−04 | 1.51E−04 |
| C4A /// C4B | KIAA0690 | 3.40E−05 | 5.50E−04 | 7.24E−08 |
| IL2RB | HDAC5 | 1.25E−05 | 5.66E−04 | 1.27E−07 |
| ASGR2 | NOL7 | 8.39E−04 | 5.81E−04 | 4.07E−06 |
| ASGR2 | LCN2 | 1.12E−09 | 5.84E−04 | 6.77E−11 |
| RUNX3 | MT1M | 1.24E−04 | 6.51E−04 | 7.87E−08 |
| IL2RB | HPCAL1 | 1.70E−04 | 6.53E−04 | 1.87E−06 |
| MTF1 | C4A /// C4B | 9.36E−05 | 6.55E−04 | 3.71E−08 |
| IL2RB | SMO | 3.17E−04 | 6.73E−04 | 9.23E−07 |
| ASGR2 | MARK3 | 9.66E−05 | 6.87E−04 | 1.19E−06 |
| ASGR2 | RALBP1 | 4.75E−05 | 6.88E−04 | 4.14E−08 |
| IL2RB | TALDO1 | 5.83E−04 | 6.91E−04 | 6.20E−06 |
| AMFR | RAB31 | 3.22E−04 | 6.97E−04 | 7.85E−08 |
| ASGR2 | CIRBP | 6.12E−04 | 7.00E−04 | 4.53E−07 |
| IL2RB | HLADQA1 | 1.08E−05 | 7.16E−04 | 1.02E−08 |
| IL2RB | UBE2G2 | 7.87E−04 | 7.19E−04 | 3.22E−06 |
| ASGR2 | GOLGA8G /// | 2.18E−04 | 7.21E−04 | 1.08E−07 |
| | GOLGA8D /// | | | |
| | LOC388189 /// | | | |
| | GOLGA8E /// | | | |
| | GOLGA8C /// | | | |
| | GOLGA8F | | | |
| IL2RB | HIP1R | 2.50E−04 | 7.41E−04 | 6.31E−06 |
| ASGR2 | TCN1 | 1.09E−05 | 7.52E−04 | 1.17E−08 |
| IL2RB | C2ORF17 | 2.00E−05 | 7.56E−04 | 1.59E−08 |
| IL2RB | DHX34 | 5.38E−05 | 7.76E−04 | 7.76E−07 |
| RUNX3 | C16ORF68 | 3.81E−05 | 8.04E−04 | 4.76E−06 |
| ZAP70 | KIAA0690 | 3.81E−04 | 8.28E−04 | 2.65E−07 |
| HNRPH1 | DHX34 | 1.19E−04 | 8.47E−04 | 7.65E−07 |
| ASGR2 | PQLC1 | 6.91E−05 | 8.62E−04 | 8.70E−08 |
| IL2RB | BLR1 | 4.49E−06 | 8.90E−04 | 1.13E−07 |
| IL2RB | TSTA3 | 3.80E−04 | 8.99E−04 | 4.62E−06 |
| IL2RB | VTI1B | 5.48E−05 | 9.10E−04 | 1.46E−06 |
| TCF3 | RAB31 | 5.63E−07 | 9.45E−04 | 2.27E−06 |
| MTF1 | RFTN1 | 3.28E−04 | 9.49E−04 | 2.84E−06 |
| ZAP70 | HIST2H2AA /// | 2.90E−04 | 9.59E−04 | 2.96E−07 |
| | LOC653610 /// | | | |
| | H2AR | | | |
| ASGR2 | GTF3A | 1.32E−04 | 9.76E−04 | 9.90E−07 |

### The Three-Factor Signature and Overall Survival

To determine whether the two-gene signature, IL2RB+ASGR2, was an independent predictor of OS given established prognostic factors in metastatic melanoma, we performed a multivariable Cox PH regression analysis including the expression levels of each of the genes or that of the two-gene signature as well as baseline serum LDH levels or disease stage (M category). The results suggest that the two-gene signature was an independent predictor of OS in this context in the training, test, and pooled cohorts (Table 12). Each p-value is for a likelihood-ratio test comparing the full model to a model that excludes the corresponding variable. Similarly, expression of each of the individual genes that comprise the two-gene signature (Table 13) also was an independent predictor of OS given baseline serum LDH levels or disease stage (M Category) in the training, test, and pooled cohorts. The two-gene signature was also an independent predictor of OS when absolute lymphocyte count (ALC) at baseline or prior to the third ipilimumab dose was added to the multivariable Cox PH model (Table 14).

TABLE 12
Marginal tests of significance from
multivariable Cox PH regression

| | Coefficient | |
|---|---|---|
| Variable | Estimate | P Value |
| Training Cohort | | |
| LDH | 0.0012 | 0.042 |
| 2-Gene Signature | 0.82 | 1.3 × 10⁻⁶ |
| M1B vs M1A | −0.72 | 0.14 |
| M1C vs M1A | 0.26 | 0.55 |
| Test Cohort | | |
| LDH | 0.0025 | 1.9 × 10⁻⁴ |
| 2-Gene Signature | 0.54 | 5.5 × 10⁻⁴ |
| M1B vs M1A | 0.70 | 0.31 |
| M1C vs M1A | 0.95 | 0.011 |
| Both Cohorts Pooled | | |
| LDH | 0.0017 | 4.6 × 10⁻⁵ |
| 2-Gene Signature | 0.62 | 7.6 × 10⁻⁹ |
| M1B vs M1A | −0.23 | 0.55 |
| M1C vs M1A | 0.69 | 0.013 |

TABLE 13
Multivariable Cox PH regression showing that each key gene individually was an
independent predictor of OS, given both baseline LDH and M Category.

| IL2RB | | | ASGR2 | | | ASGR1 | | |
|---|---|---|---|---|---|---|---|---|
| | Coefficient | | | Coefficient | | | Coefficient | |
| Variable | Estimate | P Value | Variable | Estimate | P Value | Variable | Estimate | P Value |
| Training Cohort | | | Training Cohort | | | Training Cohort | | |
| LDH | 0.0019 | 8.8 × 10⁻⁴ | LDH | 0.0018 | 2.6 × 10⁻³ | LDH | 0.0016 | 1.2 × 10⁻² |
| IL2RB | −1.04 | 9 × 10⁻⁵ | ASGR2 | 0.45 | 1.7 × 10⁻² | ASGR1 | 0.81 | 1.1 × 10⁻² |
| M1B vs M1A | −0.64 | 0.19 | M1B vs M1A | −0.49 | 0.32 | M1B vs M1A | −0.47 | 0.33 |
| M1C vs M1A | 0.30 | 0.49 | M1C vs M1A | 0.49 | 0.25 | M1C vs M1A | 0.34 | 0.44 |
| Test Cohort | | | Test Cohort | | | Test Cohort | | |
| LDH | 0.0026 | 6.4 × 10⁻⁵ | LDH | 0.0025 | 1.2 × 10⁻⁴ | LDH | 0.0026 | 8.5 × 10⁻⁵ |
| IL2RB | −0.66 | 1.6 × 10⁻² | ASGR2 | 0.72 | 6.8 × 10⁻⁴ | ASGR1 | 0.61 | 5.2 × 10⁻² |
| M1B vs M1A | 0.48 | 0.48 | M1B vs M1A | 0.42 | 0.53 | M1B vs M1A | 0.33 | 0.62 |
| M1C vs M1A | 0.76 | 3.7 × 10⁻² | M1C vs M1A | 0.98 | 7.9 × 10⁻³ | M1C vs M1A | 0.86 | 1.8 × 10⁻² |
| Both Cohorts | | | Both Cohorts | | | Both Cohorts | | |
| Pooled | | | Pooled | | | Pooled | | |
| LDH | 0.0022 | 1.6 × 10⁻⁷ | LDH | 0.0019 | 3.5 × 10⁻⁶ | LDH | 0.00020 | 1.8 × 10⁻⁶ |
| IL2RB | −0.81 | 1.2 × 10⁻⁵ | ASGR2 | 0.55 | 5.7 × 10⁻⁵ | ASGR1 | 0.68 | 1.0 × 1.0⁻³ |
| M1B vs M1A | −0.36 | 0.33 | M1B vs M1A | −0.26 | 0.50 | M1B vs M1A | −0.16 | 0.68 |
| M1C vs M1A | 0.55 | 4.2 × 10⁻² | M1C vs M1A | 0.71 | 9.8 × 10⁻³ | M1C vs M1A | 0.62 | 2.2 × 10⁻² |

TABLE 14
Multivariable Cox PH regression showing that the two-gene
signature was an independent predictor of OS, given ALC
(at baseline or prior to dose 3), LDH, and M category.

| | | |
|---|---|---|
| | Coefficient | |
| Variable | Estimate | P Value |

Baseline ALC (ALC1)

| | | |
|---|---|---|
| Training Cohort | | |
| 2-Gene Signature | 0.846 | 3.2 × 10⁻⁶ |
| LDH | 0.0011 | 0.08 |
| ALC1 | 0.110 | 0.67 |
| M1B vs M1A | −0.701 | 0.16 |
| M1C vs M1A | 0.249 | 0.57 |
| Test Cohort | | |
| 2-Gene Signature | 0.522 | 0.0092 |
| LDH | 0.00288 | 0.033 |
| ALC1 | 0.209 | 0.34 |
| M1B vs M1A | 0.38 | 0.64 |
| M1C vs M1A | 1.06 | 0.019 |
| Both Cohorts Pooled | | |
| 2-Gene Signature | 0.65 | 1.4 × 10⁻⁷ |
| LDH | 0.00164 | 2.2 × 10⁻³ |
| ALC1 | 0.154 | 0.32 |
| M1B vs M1A | −0.152 | 0.71 |
| M1C vs M1A | 0.799 | 9.0 × 10⁻³ |

ALC Prior to Dose 3 (ALC3)

| | | |
|---|---|---|
| Training Cohort | | |
| 2-Gene Signature | 0.792 | 1.1 × 10⁻⁵ |
| LDH | 0.00112 | 0.075 |
| ALC3 | −0.127 | 0.56 |
| M1B vs M1A | −0.756 | 0.13 |
| M1C vs M1A | 0.204 | 0.64 |
| Test Cohort | | |
| 2-Gene Signature | 0.403 | 0.023 |
| LDH | 0.00249 | 0.069 |
| ALC3 | −0.385 | 0.065 |
| M1B vs M1A | 0.488 | 0.550 |
| M1C vs M1A | 0.852 | 0.046 |
| Both Cohorts Pooled | | |
| 2-Gene Signature | 0.572 | 9.9 × 10⁻⁷ |
| LDH | 0.00155 | 4.4 × 10⁻³ |
| ALC3 | −0.267 | 0.071 |
| M1B vs M1A | −0.338 | 0.39 |
| M1C vs M1A | 0.662 | 0.027 |

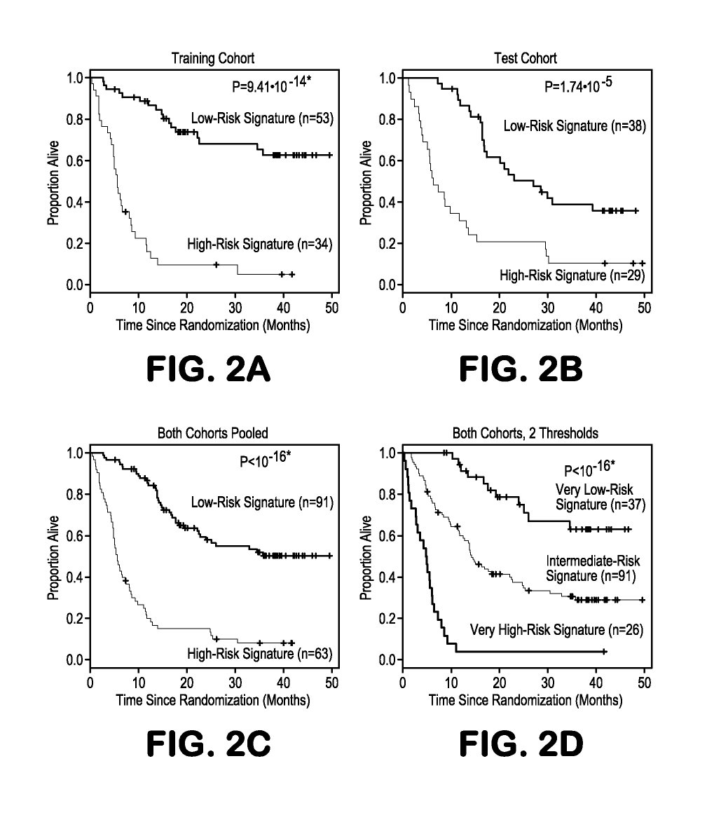

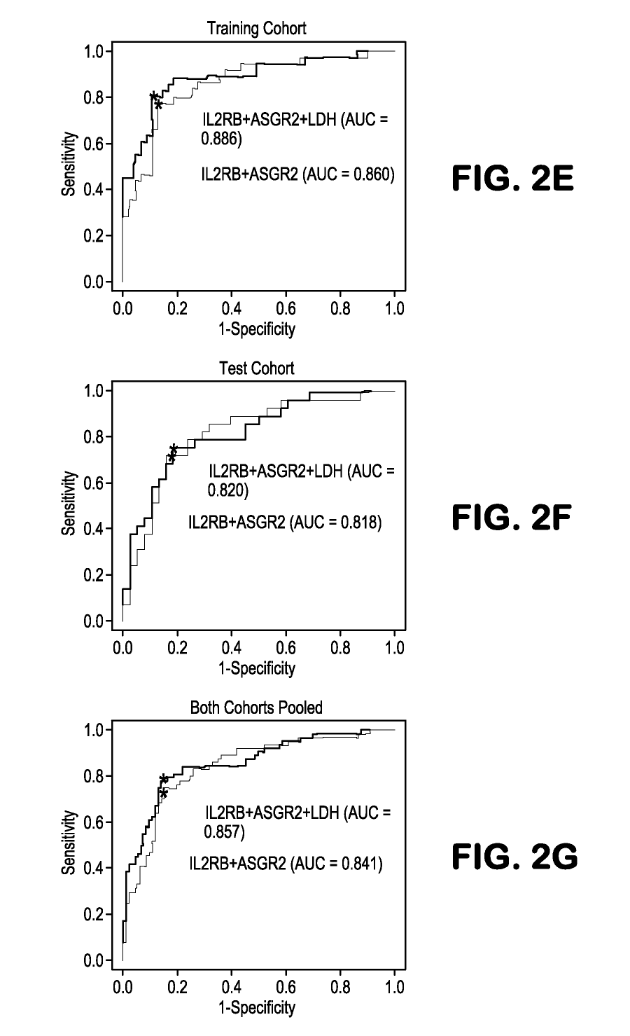

As it was established that LDH and the two-gene signature, IL2RB+ASGR2, were independent predictors of OS, it was next determined whether the two-gene signature could be improved by combining it with LDH to create a three-factor signature. Coefficients were estimated using Cox PH regression on the training cohort (0.00158 for LDH and 0.816 for the two-gene signature). The three-factor score for each patient could thus be calculated from the following equation: 0.00158*Y_{LDH}+0.816*(−1.312*X_{IL2RB}+0.748*X_{ASGR2}), where Yⱼ gives the concentration of factor j. Next the log-rank p-value was calculated for all possible thresholds. The threshold with the smallest p-value was −4.437 (FIG. 7B). The Kaplan-Meier curves were plotted for the training cohort (FIG. 2A), then the same coefficients and threshold were applied to the test cohort (FIG. 2B), yielding a log-rank p-value of p=1.74×10⁻⁵. The Kaplan-Meier plot for both cohorts pooled together appears in FIG. 2C.

It was next determined whether using two thresholds instead of one could provide better separation among survival curves. Using the three-factor signature described above with coefficients from the training cohort, two-threshold exploration was performed on the pooled cohort. Using thresholds at both −5.29 and −3.62 (FIG. 7C), three groups of patients were identified that corresponded to high, intermediate and low risk (FIG. 2D).

Time dependent ROC curves at 12 months were then plotted for both the two-gene signature (IL2RB+ASGR2) and the three-factor signature (IL2RB+ASGR2+LDH) in the training cohort (FIG. 2E), test cohort (FIG. 2F), and both cohorts pooled (FIG. 2G). These curves show that at best, baseline LDH only slightly improves predictive performance when added to the two-gene signature.

### Functional and Gene Set Enrichment Analysis

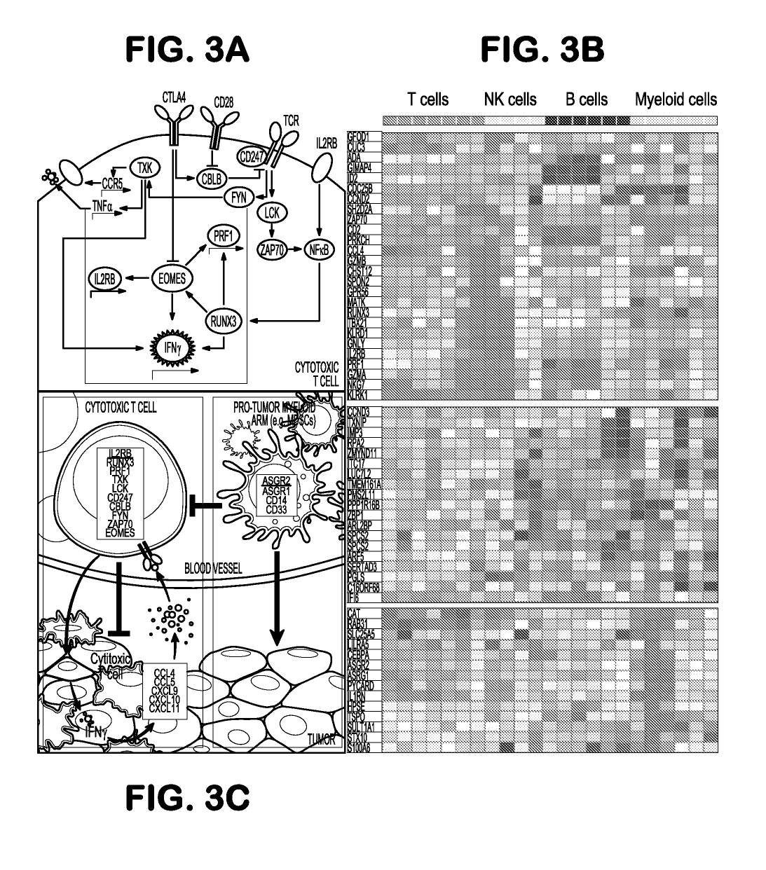

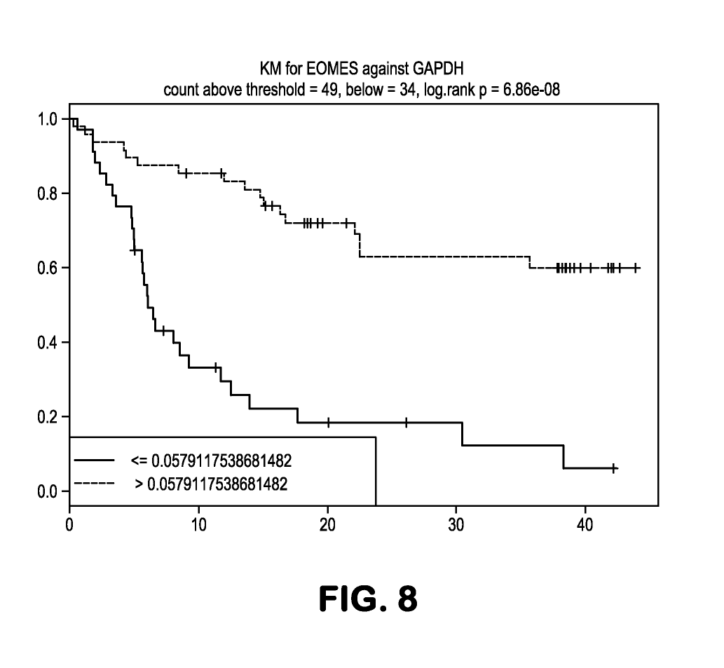

This study also sought to determine whether the various gene sets emerging in the above analyses were characteristic of particular blood cell types. Among the genes most highly correlated with IL2RB across the pooled training and test cohorts, the top two were PRF1 (perforin 1, probe 214617_at) (Spearman R=0.735, p=2.77×10⁻²⁸) and RUNX3 (runt-related transcription factor 3, probe 204197_s_at) (Spearman R=0.729, p=1.24×10⁻²⁷) (Table 5), genes that are highly interrelated, established to be associated with T-cells,^{22,23} and point clearly to underlying biological mechanisms (see Discussion). Also present among the 100 genes most correlated with IL2RB are a number of other genes established to be associated with T-cells including CD247,²⁴ LCK,²⁵ FYN,²⁵ ZAP70,²⁶ CBLB,²⁷ and TXK.²⁸ RUNX3, PRF1, and ZAP70 are also present on the list of genes associated with OS by univariate Cox regression with p<0.005. RUNX3 has been reported to induce transcription of PRF1 and EOMES (eomesodermin),²² which has been implicated in the regulation of IL2RB expression.²⁹ These analyses pointed to a role for EOMES as a central regulator of the expression of various genes in our model (FIG. 3A). Since there were no probes on the HT-HG-U133A 96-array for testing the expression of this gene, the expression of EOMES was tested separately by qPCR. There was significant association between the expression of EOMES and overall survival by both log-rank test (p=6.86×10⁻⁸) (FIG. 8) and univariate Cox regression (p=1.808×10⁻³). In addition, expression of key genes as determined by microarray were all highly correlated with EOMES expression (by qPCR) as determined by Spearman's rank correlation, including IL2RB (R=0.474, p=1.50×10⁻⁵), PRF1 (R=0.585, p=2.90×10⁻⁸), and RUNX3 (R=0.594, p=1.57×10⁻⁸).

Among the genes most highly correlated with ASGR2 are ASGR1, CD14 (cluster of differentiation 14, probe 201743_at) (Spearman R=0.588, p=3.75×10⁻¹⁶), and CD33 (cluster of differentiation 33, probe 206120_at) (Spearman R=0.457, p=1.34×10⁻⁹) (Table 5). CD14 expression is a characteristic of myeloid-derived suppressor cells (MDSCs) in melanoma patients,⁹ and CD33 expression is a characteristic of myeloid cells more generally.³⁰ Our cell type enrichment analysis found that among the 73 genes associated with OS by univariate Cox PH regression (p<0.005), the set of genes negatively associated with OS was most enriched in genes specific for CD14+ monocytes (P=2.17×10⁻⁷) (P values by hyper-geometric test as described in Methods), and also highly enriched in genes specific for CD33+ monocytes (P=2.62×10⁻⁴) as well as two types of granulocytes (Table 15). This is illustrated graphically (FIG. 3B, lower right) in a heat map of the DMAP¹⁸ expression data by cell type (columns) for the set of genes negatively associated with OS (rows).

TABLE 15
Enrichment of genes specific for particular cell types in the list of genes
negatively associated with OS, including adjusted hypergeometric P values.

| Cell Type | Score | P-value | Adjusted P-value |
|---|---|---|---|
| MONO2\|CD14+\|CD45dim | 36.64 | 1.11E−08 | 2.17E−07 |
| GRAN2\|CD34−\|SSChi\|CD45+\|CD11b+\|CD16− | 25.54 | 4.64E−07 | 6.04E−06 |
| MONO1\|CD34−\|CD33+\|CD13+ | 13.91 | 2.69E−05 | 2.62E−04 |
| GRAN3\|CD16+\|CD11b+ | 10.18 | 3.74E−03 | 2.91E−02 |

The set of genes positively associated with OS was most enriched in genes specific for two types of NK cells (CD56⁺CD16⁺CD3⁻, P=2.50×10⁻¹⁸ and CD56⁻CD16⁻CD3⁻, P=7.95×10⁻¹²) and two types of T cells (CD8⁺CD62L⁻CD45RA⁺, P=3.41×10⁻¹⁷ and CD8⁺CD62L⁻CD45RA⁻, P=8.05×10⁻¹⁴) (Table 16) (P values by hyper-geometric test as described in Methods). This is illustrated graphically (FIG. 3B, top and middle) in a heat map of the DMAP expression data¹⁸ by cell type (columns) for the set of genes positively associated with OS (rows).

TABLE 16
Enrichment of genes specific for particular cell types in the list of genes
positively associated with OS, including adjusted hypergeometric P values.

| Cell Type | Score | P-value | Adjusted P-value |
|---|---|---|---|
| NKA2\|CD56+\|CD16+\|CD3− | 53.89 | 1.28E−19 | 2.50E−18 |
| TCELLA1\|CD8+\|CD62L−\|CD45RA+ | 42.86 | 2.62E−18 | 3.41E−17 |
| TCELLA3\|CD8+\|CD62L−\|CD45RA− | 34.51 | 8.26E−15 | 8.05E−14 |
| NKA3\|CD56−\|CD16−\|CD3− | 41.42 | 1.02E−12 | 7.95E−12 |
| GRAN3\|CD16+\|CD11b+ | 32.67 | 3.37E−09 | 2.19E−08 |
| TCELLA4\|CD8+\|CD62L+\|CD45RA− | 15.54 | 8.98E−09 | 5.00E−08 |
| NKA4\|CD14−\|CD19−\|CD3+\|CD1d+ | 2.95 | 7.20E−06 | 3.51E−05 |
| MEGA2\|CD34−\|CD41+\|CD61+\|CD45− | 2.86 | 8.97E−04 | 3.89E−03 |
| GRAN1\|CD34−\|SSChi\|CD45+\|CD11b−\|CD16− | 9.78 | 1.65E−03 | 6.44E−03 |
| TCELLA2\|CD8+\|CD62L+\|CD45RA+ | 9.80 | 1.98E−03 | 7.01E−03 |
| TCELLA7\|CD4+\|CD62L−\|CD45RA− | 5.12 | 2.38E−03 | 7.73E−03 |

Taken together, these analyses suggest that greater expression of genes more highly expressed in natural killer (NK) and T-cells (such as IL2RB) was associated with longer survival, while greater expression of genes expressed in CD14⁺ cells and other myeloid lineage cells (such as ASGR1 and ASGR2) was associated with shorter survival (FIG. 3C).

### Discussion

Ongoing research aims to discover biomarkers that could select patients with an enhanced benefit/risk profile. Whereas ipilimumab has shown significant survival benefit in a subset of metastatic melanoma patients, in some patients the treatment can result in adverse events. Thus, identification of biomarkers that can predict a patient's response and are easily measured in peripheral blood is important. In the present study, a novel approach was used to identify blood gene-signatures that may predict OS in metastatic melanoma patients receiving ipilimumab.

When using microarray data to develop predictive gene-signatures there is a high likelihood of developing a signature that may be strongly associated with OS in a training cohort, but not significantly associated with OS in a test cohort, due to over-fitting in the training cohort. Signatures consisting of large numbers of genes are more likely to suffer from over-fitting and are less practical in the clinical context.

Using gene expression microarray data from a training cohort of 88 patients, two independent methods were applied to evaluate association of gene expression with OS. Results from both methods pointed to a lead two-gene signature of IL2RB+ASGR2 that was highly associated with OS in the training cohort. Using these two genes, a signature was calculated that included two coefficients and a threshold in the training cohort, and it was determined that the same signature was also significantly associated with OS in an independent test cohort of 69 patients (p<0.001). The signature also had strong predictive performance in the independent test cohort (AUC=0.818 for a time-dependent ROC curve at 12 months).

The size of the signature is noteworthy. While signatures comprised of many genes carry risk of over-fitting, a two-gene signature significantly mitigates this risk. Adding additional genes improved the signature incrementally, but in this study, the majority of the predictive power came from the combination of two top genes, IL2RB and ASGR2.

Mechanistic investigation of the two genes with expression most highly correlated with that of IL2RB (RUNX3 and PRF1) yielded insights into its underlying biology. RUNX3 has been reported to induce transcription of PRF1 and EOMES (eomesodermin),²² which has been implicated in the regulation of IL2RB expression.²⁹ Based on the high correlation between IL2RB, RUNX3, and PRF1 expression and the mechanistic linkage between EOMES, RUNX3 and IL2RB, it may be hypothesized that EOMES is a core transcription factor that underlies the observed coexpression of IL2RB, RUNX3 and PRF1 in the data. Further analyses of EOMES by qPCR supported this notion, as we found strong correlation of the expression levels of EOMES and other genes in our model. Greater baseline expression levels of this gene were also associated with longer survival in the data set. Moreover, a direct relationship between EOMES and CTLA-4 has been established,³¹ as well as interactions between EOMES and IFNγ,²² the factor underlying many of the tumor chemokine changes linked with ipilimumab response (FIG. 3A).³

Mechanistic investigation of ASGR2 linked it to myeloid cells and particularly MDSCs, as its expression was highly correlated with the MDSC surface markers CD14 and CD33.^{9,30} MDSCs have the capacity to suppress both the cytotoxic activities of natural killer (NK) and natural killer T (NKT) cells, and the adaptive immune response mediated by CD4⁺ and CD8⁺ T cells. MDSCs act through multiple pathways including upregulation of nitric oxide synthase 2 (NOS2) and production of arginase 1 (ARG1). ARG1 and NOS2 metabolize L-arginine and either together, or separately, block translation of the T cell CD3 zeta chain, inhibit T cell proliferation, and promote T cell apoptosis.³² Additionally, MDSCs are believed to secrete immunosuppressive cytokines such as TGFβ and induce regulatory T cell development.³⁰ High frequency of MDSCs have been reported in the peripheral blood of patients affected by breast, lung, renal and head and neck carcinomas³³ and in melanoma.³⁴

While in this study gene expression was mainly measured via microarray, it may also be assayed via quantitative polymerase chain reaction (qPCR). Moreover, IL2RB and ASGR2 are both cell surface markers and therefore may be detected via flow cytometry. The magnitude of the two-gene signature may change over time in a given patient (either inherently or in response to additional therapies such as a CD137-agonist), and may be monitored to determine the best times to administer or re-administer ipilimumab.

### REFERENCES

1. Hodi F S, O'Day S J, McDermott D F, et al. Improved survival with ipilimumab in patients with metastatic melanoma. N Engl J Med 2010; 363:711-723.
2. Robert C, Thomas L, Bondarenko I, et al. Ipilimumab plus Dacarbazine for Previously Untreated Metastatic Melanoma. N Engl J Med 2011; 364:2517-2526.
3. Ji R-R, Chasalow S D, Wang L, et al. An immune-active tumor microenvironment favors clinical response to ipilimumab. Cancer Immunol Immunother 2011.
4. Berman D M, Wolchok J, Weber J, et al. Association of peripheral blood absolute lymphocyte count (ALC) and clinical activity in patients (pts) with advanced melanoma treated with ipilimumab. ASCO Annual Meeting 2009; Abs 3020.
5. Weber J S, Yu B, Hall D, et al. Pharmacodynamic and predictive markers of ipilimumab on melanoma patients' T-cells. ASCO Annual Meeting 2011; Abs 2503.
6. Chen H, Liakou C I, Kamat A, et al. Anti-CTLA-4 therapy results in higher CD4⁺ICOS^{hi} T cell frequency and IFN-γ levels in both nonmalignant and malignant prostate tissues. Proc Natl Acad Sci USA 2009; 106:2729-2734.
7. Yuan J, Gnjatic S, Li H, et al. CTLA-4 blockade enhances polyfunctional NY-ESO-1 specific T cell responses in metastatic melanoma patients with clinical benefit. Proc Natl Acad Sci USA 2008; 105:20410-20415.
8. Yuan J, Adamow M, Ginsberg B A, et al. Integrated NY-ESO-1 antibody and CD8⁺ T-cell responses correlate with clinical benefit in advanced melanoma patients treated with ipilimumab. Proc Natl Acad Sci USA 2011.
9. Kitano S, Postow M A, Cortez C, et al. Myeloid-derived supressor cell quantity prior to treatment with ipilimumab at 10 mg/kg to predict for overall survival in patients with metastatic melanoma. ASCO Annual Meeting 2012; Abs 2518.
10. Weber J, Thompson J A, Hamid O, et al.: A randomized, double-blind, placebo-controlled, phase II study comparing the tolerability and efficacy of ipilimumab administered with or without prophylactic budesonide in patients with unresectable stage III or IV melanoma. Clin Cancer Res. 2009, 15:5591-5598.
11. Hamid O, Schmidt H, Nissan A, et al.: A prospective phase II trial exploring the association between tumor microenvironment biomarkers and clinical activity of ipilimumab in advanced melanoma. J. Translational Medicine. 2011; 9:204.
12. Irizarry R A, Bolstad B M, Collin F, et al Summaries of Affymetrix GeneChip probe level data. Nucleic Acids Research, 2003; 31 (4):e15.
13. Gentleman R C, Carey V J, Bates D M, et al. Bioconductor: open software development for computational biology and bioinformatics. Genome Biology. 2005, 5:R80.
14. Miller J A, Cai C, Langfelder P, et al. Strategies for aggregating gene expression data: The collapseRows R function. BMC Bioinformatics, 2011; 12:322.
15. Friedman J, Hastie T, Tibshirani R. Regularization paths for generalized linear models via coordinate descent. J Stat Soft 2010; 33.
16. Kaplan E L, Meier P. Nonparametric estimation from incomplete observations. J. Amer. Statist. Assn. 1958; 53:457-481.
17. Balch C M, Gershenwald J E, Soong S, et al. Final version of 2009 AJCC melanoma staging and classification. J Clin Oncol 2009; 27:6199-6206.
18. Novershtern N, Subramanian A, Lawton L N, et al. Densely interconnected transcriptional circuits control cell states in human hematopoiesis. Cell 2011; 144:296-309.
19. Benita Y, Cao Z, Giallourakis C, et al. Gene enrichment profiles reveal T-cell development, differentiation, and lineage-specific transcription factors including ZBTB25 as a novel NF-AT repressor. Blood 2010; 115:5376-5384.
20. Harris R L, van den Berg C W, and Bowen D J. ASGR1 and ASGR2, the Genes that Encode the Asialoglycoprotein Receptor (Ashwell Receptor), Are Expressed in Peripheral Blood Monocytes and Show Interindividual Differences in Transcript Profile. Molecular Biology International 2012; 283974.
21. Heagerty P J, Lumley T, Pepe M S. Time-Dependent ROC Curves for Censored Survival Data and a Diagnostic Marker. Biometrics 2000; 56:337-344.
22. Cruz-Guilloty F, Pipkin M E, Djuretic I M, et al. Runx3 and T-box proteins cooperate to establish the transcriptional program of effector CTLs. J Exp Med 2009; 206:51-59.
23. Voskoboinik I, Smyth M J, Trapani J A. Perforin-mediated target-cell death and immune homeostasis. Nat Rev Immunol. 2006; 6 (12):940-52.
24. Lundholm M, Mayans S, Motta V, et al. Variation in the Cd3 zeta (Cd247) gene correlates with altered T cell activation and is associated with autoimmune diabetes. J Immunol 2010; 184 (10):5537-44.
25. Filipp D, Ballek O, Manning J. Lck, Membrane Microdomains, and TCR Triggering Machinery: Defining the New Rules of Engagement. Front Immunol. 2012; 3:155.
26. Yokosuka T, Sakata-Sogawa K, Kobayashi W, et al. Newly generated T cell receptor microclusters initiate and sustain T cell activation by recruitment of Zap70 and SLP-76. Nat Immunol. 2005 December; 6 (12):1253-62.
27. Teh C E, Daley S R, Enders A, et al. T-cell regulation by casitas B-lineage lymphoma (Cblb) is a critical failsafe against autoimmune disease due to autoimmune regulator (Aire) deficiency. Proc Natl Acad Sci USA. 2010; 107(33):14709-14.
28. Sahu N, Venegas A M, Jankovic D, et al. Selective expression rather than specific function of Txk and Itk regulate Th1 and Th2 responses. J Immunol. 2008 Nov. 1; 181 (9):6125-31.
29. Intlekofer A M, Takemoto N, Wherry E J, et al. Effector and memory CD8⁺ T cell fate coupled by T-bet and eomesodermin. Nat Immunol 2005; 6:1236-1244.
30. Lechner, M G, Liebertz D J, Epstein A L. Characterization of Cytokine-Induced Myeloid-Derived Suppressor Cells from Normal Human Peripheral Blood Mononuclear Cells. J Immunol 2010; 185:2273-2284.
31. Hegel J K, Knieke K, Kolar P, et al. CD152 (CTLA-4) regulates effector functions of CD8⁺ T lymphocytes by repressing Eomesodermin. Eur J Immunol 2009; 39:883-893.
32. Bronte V, Serafini P, Mazzoni A, et al. L-arginine metabolism in myeloid cells controls T-lymphocyte functions. Trends Immunol. 2003; 24 (6):302-306.
33. Talmadge J E. Pathways mediating the expansion and immunosuppressive activity of myeloid-derived suppressor cells and their relevance to cancer therapy. Clin Cancer Res. 2007; 13 (18 Pt 1):5243-8.
34. Filipazzi P, Valenti R, Huber V, et al. Identification of a new subset of myeloid suppressor cells in peripheral blood of melanoma patients with modulation by a granulocyte-macrophage colony-stimulation factor-based antitumor vaccine. J Clin Oncol. 2007; 25 (18):2546-53.

TABLE 17
Probe Sets

| Target Name | Probe Set ID | SEQ ID NO. | Probe Sequences | Target Sequence | Target Genbank ID |
|---|---|---|---|---|---|
| SLC25A3 | 200030_s_at | 1 | TCATCATGATTGGTACCCTGACTGC | acaccatgatgaagttcgcctgctttgaacgtactgttgaagcactgtacaag | NM_002635.1 |
| | | | | tttgtggttcctaagccccgcagtgaatgttcaaagccagagcagctggttgt | |
| | | | | aacatttgtagcaggttacatagctggagtcttttgtgcaattgtttctcacc | |
| | | | | ctgctgattctgtggtatctgtgttgaataaagaaaaaggtagcagtgcttct | |
| | | | | ctggtcctcaagagacttggatttaaaggtgtatggaagggactgtttgcccg | |
| | | | | tatcatcatgattggtaccctgactgcactacagtggtttatctatgactccgtgaag | |
| | | | | gtctacttcagacttcctc | |
| | | 2 | GTACCCTGACTGCACTACAGTGGTT | | |
| | | 3 | GTGGTTTATCTATGACTCCGTGAAG | | |
| | | 4 | GTGAAGGTCTACTTCAGACTTCCTC | | |
| | | 5 | ACACCATGATGAAGTTCGCCTGCTT | | |
| | | 6 | TCGCCTGCTTTGAACGTACTGTTGA | | |
| | | 7 | TGTACAAGTTTGTGGTTCCTAAGCC | | |
| | | 8 | TCCTAAGCCCCGCAGTGAATGTTCA | | |
| | | 9 | ACCCTGCTGATTCTGTGGTATCTGT | | |
| | | 10 | AAAGGTAGCAGTGCTTCTCTGGTCC | | |
| | | 11 | GTGCTTCTCTGGTCCTCAAGAGACT | | |
| NONO | 200057_s_at | 12 | GCCCCAGAGAAACTGCCACATACAC | gccccagagaaactgccacatacaccacaaaaaccaaacatgccccaatgacc | NM_007363.2 |
| | | | | ttagccccattgctccattcactcccaggtgagaattcaggcaaacgtccaca | |
| | | | | aaggtcacaggcagcgtacatacggttctgttataccccatatattacccctt | |
| | | | | catgtcctaaagaagacattttctcttagagattttcattttagtgtatcttt | |
| | | | | aaaaaaaaaatcttgtgttaacttgcctccatctttttcttggggtgagggac | |
| | | | | accagggaatgacccttttgtgtctatgatgttgctgttcacagcttttcttg | |
| | | | | ataggcctagtacaatcttgggaacagggttactgtatactgaaggtctgaca | |
| | | | | gtagctcttagactcgcctatcttaggtagtcatgctgtgcattttttttttcattggt | |
| | | | | gtactgtgtttgatttgtctca | |
| | | 13 | GCTCCATTCACTCCCAGGTGAGAAT | | |
| | | 14 | GGCAAACGTCCACAAAGGTCACAGG | | |
| | | 15 | AGGTCACAGGCAGCGTACATACGGT | | |
| | | 16 | CATACGGTTCTGTTATACCCCATAT | | |
| | | 17 | TATTACCCCTTCATGTCCTAAAGAA | | |
| | | 18 | AAATCTTGTGTTAACTTGCCTCCAT | | |
| | | 19 | GGAATGACCCTTTTGTGTCTATGAT | | |
| | | 20 | CACAGCTTTTCTTGATAGGCCTAGT | | |
| | | 21 | TGACAGTAGCTCTTAGACTCGCCTA | | |
| | | 22 | GGTGTACTGTGTTTGATTTGTCTCA | | |
| RNPS1 | 200060_s_at | 23 | CAGGGAAAAGTGAGGCTCTTGGGGG | cagggaaaagtgaggctcttgggggtggtttgaccctgcttacctgggagcac | BC001659.1 |
| | | | | acttttcccttccccgatgacctgggatggtggccaggccgtgcccttgctgt | |
| | | | | tgctgggcagtgtccttttggaaagggagctgccccaggctttagtgcagctg | |
| | | | | ccaaccctgttaggcctggcctctcgaggcctcttctgatctcaagggtcaca | |
| | | | | ccccctcaaagatcctctcacccatggtagttgctgctcgtggttctgtctgt | |
| | | | | ccgtgcaccgatgcacacaccgcaccccaccactgtactctgaaattggcgag | |
| | | | | tgagtggagagccagctctgcggagtcatcacgcagccatggttgtgcctgcc | |
| | | | | gttcatggtggtctttcaggttatcttggcaacatgtacattgcttttatttt | |
| | | | | ttttcttttttgctttcattgtacagtcagtactataaaatttctcttttgagtttta | |
| | | | | tacctttgtagcattttagatgacattgtgtttgtactttgttg | |
| | | 24 | TTACCTGGGAGCACACTTTTCCCTT | | |
| | | 25 | CCTTCCCCGATGACCTGGGATGGTG | | |
| | | 26 | CCCCGATGACCTGGGATGGTGGCCA | | |
| | | 27 | CCCACCACTGTACTCTGAAATTGGC | | |
| | | 28 | CACTGTACTCTGAAATTGGCGAGTG | | |
| | | 29 | CCGTTCATGGTGGTCTTTCAGGTTA | | |
| | | 30 | GGTGGTCTTTCAGGTTATCTTGGCA | | |
| | | 31 | GGTCTTTCAGGTTATCTTGGCAACA | | |
| | | 32 | TCAGGTTATCTTGGCAACATGTACA | | |
| | | 33 | ATGACATTGTGTTTGTACTTTGTTG | | |
| HSP90AB1 | 200064_at | 34 | AATAGACTTGTGTCTTCACCTTGCT | aatagacttgtgtcttcaccttgctgcattgtgaccagcacctacggctggac | AF275719.1 |
| | | | | agccaatatggagcggatcatgaaagcccaggcacttcgggacaactccacca | |
| | | | | tgggctatatgatggccaaaaagcacctggagatcaaccctgaccaccccatt | |
| | | | | gtggagacgctgcggcagaaggctgaggccgacaagaatgataaggcagttaa | |
| | | | | ggacctggtggtgctgctgtttgaaaccgccctgctatcttctggcttttccc | |
| | | | | ttgaggatccccagacccactccaaccgcatctatcgcatgatcaagctaggt | |
| | | | | ctaggtattgatgaagatgaagtggcagcagaggaacccaatgctgcagttcc | |
| | | | | tgatgagatcccccctctcgagggcgatgaggatgcgtctcgcatggaagaagtcgat | |
| | | | | taggttaggagttcatagttggaaaacttgtgcccttgtatagtgtccc | |
| | | 35 | GTCTTCACCTTGCTGCATTGTGACC | | |
| | | 36 | GTGACCAGCACCTACGGCTGGACAG | | |
| | | 37 | GAGCGGATCATGAAAGCCCAGGCAC | | |
| | | 38 | AAAAGCACCTGGAGATCAACCCTGA | | |
| | | 39 | TGGTGGTGCTGCTGTTTGAAACCGC | | |
| | | 40 | CAACCGCATCTATCGCATGATCAAG | | |
| | | 41 | GCAGAGGAACCCAATGCTGCAGTTC | | |
| | | 42 | TCCCCCCTCTCGAGGGCGATGAGGA | | |
| | | 43 | GGGCGATGAGGATGCGTCTCGCATG | | |
| | | 44 | AACTTGTGCCCTTGTATAGTGTCCC | | |
| SLC25A5 | 200657_at | 45 | TAACACAATCTTGAGCATTCTTGAC | cctacttcggtatctatgacactgcaaagggaatgcttccggatcccaagaac | NM_001152.1 |
| | | | | actcacatcgtcatcagctggatgatcgcacagactgtcactgctgttgccgg | |
| | | | | gttgacttcctatccatttgacaccgttcgccgccgcatgatgatgcagtcag | |
| | | | | ggcgcaaaggaactgacatcatgtacacaggcacgcttgactgctggcggaag | |
| | | | | attgctcgtgatgaaggaggcaaagcttttttcaagggtgcatggtccaatgt | |
| | | | | tctcagaggcatgggtggtgcttttgtgcttgtcttgtatgatgaaatcaaga | |
| | | | | agtacacataagttatttcctaggatttttccccctgtgaacaggcatgttgt | |
| | | | | attctataacacaatcttgagcattcttgacagactcctggctgtcagtttctcagtg | |
| | | | | gcaac | |
| | | 46 | CATTCTTGACAGACTCCTGGCTGTC | | |
| | | 47 | TGGCTGTCAGTTTCTCAGTGGCAAC | | |
| | | 48 | CCTACTTCGGTATCTATGACACTGC | | |
| | | 49 | GGGAATGCTTCCGGATCCCAAGAAC | | |
| | | 50 | CAAGAACACTCACATCGTCATCAGC | | |
| | | 51 | ATGATCGCACAGACTGTCACTGCTG | | |
| | | 52 | GCTGGCGGAAGATTGCTCGTGATGA | | |
| | | 53 | GGGTGCATGGTCCAATGTTCTCAGA | | |
| | | 54 | GAGGCATGGGTGGTGCTTTTGTGCT | | |
| | | 55 | TGCTTTTGTGCTTGTCTTGTATGAT | | |
| GRN | 200678_x_at | 56 | CGTAGCCCTCACGTGGGTGTGAAGG | cgtagccctcacgtgggtgtgaaggacgtggagtgtggggaaggacacttctg | NM_002087.1 |
| | | | | ccatgataaccagacctgctgccgagacaaccgacagggctgggcctgctgtc | |
| | | | | cctaccgccagggcgtctgttgtgctgatcggcgccactgctgtcctgctggc | |
| | | | | ttccgctgcgcagccaggggtaccaagtgtttgcgcagggaggccccgcgctg | |
| | | | | ggacgcccctttgagggacccagccttgagacagctgctgtgagggacagtac | |
| | | | | tgaagactctgcagccctcgggaccccactcggagggtgccctctgctcaggc | |
| | | | | ctccctagcacctccccctaaccaaattctccctggaccccattctgagctcc | |
| | | | | ccatcaccatgggaggtggggcctcaatctaaggccttccctgtcagaagggg | |
| | | | | gttgtggcaaaagccacattacaagctgccatcccctccccgtttcagtggac | |
| | | | | cctgtggccaggtgcttttccctatccacaggggtgtttgtgtgtgtgcgcgtgtgc | |
| | | | | gtttcaata | |
| | | 57 | GAAGGACACTTCTGCCATGATAACC | | |
| | | 58 | TGCCATGATAACCAGACCTGCTGCC | | |
| | | 59 | GCCGAGACAACCGACAGGGCTGGGC | | |
| | | 60 | GCCAGGGGTACCAAGTGTTTGCGCA | | |
| | | 61 | GACCCAGCCTTGAGACAGCTGCTGT | | |
| | | 62 | CAGTACTGAAGACTCTGCAGCCCTC | | |
| | | 63 | TGAGCTCCCCATCACCATGGGAGGT | | |
| | | 64 | TGGGGCCTCAATCTAAGGCCTTCCC | | |
| | | 65 | AAAGCCACATTACAAGCTGCCATCC | | |
| | | 66 | GTGTGTGCGCGTGTGCGTTTCAATA | | |
| CCND2 | 200953_s_at | 67 | GCCATTACAGTATCCAATGTCTTTT | gccattacagtatccaatgtcttttgacaggtgcctgtccttgaaaaacaaag | NM_001759.1 |
| | | | | tttctatttttatttttaattggtttagttcttaactgctggccaactcttac | |
| | | | | atccccagcaaatcatcgggccattggattttttccattatgttcatcaccct | |
| | | | | tatatcatgtacctcagatctctctctctctcctctctctcagttatatagtt | |
| | | | | tcttgtcttggactttttttttcttttctttttctttttttttttgctttaaa | |
| | | | | acaagtgtgatgccatatcaagtccatgttattctctcacagtgtactctata | |
| | | | | agaggtgtgggtgtctgtttggtcaggatgttagaaagtgctgataagtagca | |
| | | | | tgatcagtgtatgcgaaaaggtttttaggaagtatggcaaaaatgttgtattg | |
| | | | | gctatgatggtgacatgatatagtcagctgccttttaagaggtcttatctgttcagtg | |
| | | | | tt | |
| | | 68 | GTTTAGTTCTTAACTGCTGGCCAAC | | |
| | | 69 | CTTACATCCCCAGCAAATCATCGGG | | |
| | | 70 | TATGTTCATCACCCTTATATCATGT | | |
| | | 71 | TTATATCATGTACCTCAGATCTCTC | | |
| | | 72 | TCTCCTCTCTCTCAGTTATATAGTT | | |
| | | 73 | GTGTGATGCCATATCAAGTCCATGT | | |
| | | 74 | AGTCCATGTTATTCTCTCACAGTGT | | |
| | | 75 | GGTGTGGGTGTCTGTTTGGTCAGGA | | |
| | | 76 | ATGTTGTATTGGCTATGATGGTGAC | | |
| | | 77 | TAAGAGGTCTTATCTGTTCAGTGTT | | |
| TXNIP | 201010_s_at | 78 | GTGTTCTCCTACTGCAAATATTTTC | gtgttctcctactgcaaatattttcatatgggaggatggttttctcttcatgt | NM_006472.1 |
| | | | | aagtccttggaattgattctaaggtgatgttcttagcactttaattcctgtca | |
| | | | | aattttttgttctccccttctgccatcttaaatgtaagctgaaactggtctac | |
| | | | | tgtgtctctagggttaagccaaaagacaaaaaaaattttactacttttgagat | |
| | | | | tgccccaatgtacagaattatataattctaacgcttaaatcatgtgaaagggt | |
| | | | | tgctgctgtcagccttgcccactgtgacttcaaacccaaggaggaactcttga | |
| | | | | tcaagatgcccaaccctgtgatcagaacctccaaatactgccatgagaaacta | |
| | | | | gagggcaggtgttcataaaagccctttgaacccccttcctgccctgtgttagg | |
| | | | | agatagggatattggcccctcactgcagctgccagcacttggtcagtcactct | |
| | | | | cagccatagcactttgttcactgtcctgtgtcagagcactgagctccacccttttctg | |
| | | | | agagttat | |
| | | 79 | GGTTTTCTCTTCATGTAAGTCCTTG | | |
| | | 80 | TGTTCTTAGCACTTTAATTCCTGTC | | |
| | | 81 | GCTGAAACTGGTCTACTGTGTCTCT | | |
| | | 82 | GAAAGGGTTGCTGCTGTCAGCCTTG | | |
| | | 83 | CAACCCTGTGATCAGAACCTCCAAA | | |
| | | 84 | AGATAGGGATATTGGCCCCTCACTG | | |
| | | 85 | CACTCTCAGCCATAGCACTTTGTTC | | |
| | | 86 | ACTTTGTTCACTGTCCTGTGTCAGA | | |
| | | 87 | TGTGTCAGAGCACTGAGCTCCACCC | | |
| | | 88 | AGCTCCACCCTTTTCTGAGAGTTAT | | |
| RBBP7 | 201092_at | 89 | GCAGAAGATGGGCCTCCAGAACTCC | gcagaagatgggcctccagaactcctgtttattcatggaggacacactgctaa | NM_002893.2 |
| | | | | gatttcagattttagctggaaccccaatgagccttgggtcatttgctcagtgt | |
| | | | | ctgaggataacatcatgcagatatggcaaatggctgaaaatatttacaatgat | |
| | | | | gaagagtcagatgtcacgacatccgaactggagggacaaggatcttaaaccca | |
| | | | | aagtacgagaaatgtttctgttgaatgtaatgctacatgaatgcttgatttat | |
| | | | | caagcgccaaaaaggcattgtatagtaggaaatgtaagtggggtggcttatgg | |
| | | | | cttctttatcctctgattctagcactttcaagtgagctgttgcgtactgtatc | |
| | | | | atattgtagctattagggaagagaagaatgttgcttaagaaagaacatcacca | |
| | | | | ttgattttaaatacaagtagcagggtattgcctttgattcaactgttttaagtcctca | |
| | | | | ttttctcaaactaagtgcttgctgtt | |
| | | 90 | TATTCATGGAGGACACACTGCTAAG | | |
| | | 91 | TTAGCTGGAACCCCAATGAGCCTTG | | |
| | | 92 | AGCCTTGGGTCATTTGCTCAGTGTC | | |
| | | 93 | GTCACGACATCCGAACTGGAGGGAC | | |
| | | 94 | TGGGGTGGCTTATGGCTTCTTTATC | | |
| | | 95 | TTATCCTCTGATTCTAGCACTTTCA | | |
| | | 96 | GTGAGCTGTTGCGTACTGTATCATA | | |
| | | 97 | GTAGCAGGGTATTGCCTTTGATTCA | | |
| | | 98 | CAACTGTTTTAAGTCCTCATTTTCT | | |
| | | 99 | TTTCTCAAACTAAGTGCTTGCTGTT | | |
| LGALS1 | 201105_at | 100 | AAACCTGGAGAGTGCCTTCGAGTGC | ctcctggactcaatcatggcttgtggtctggtcgccagcaacctgaatctcaa | NM_002305.2 |
| | | | | acctggagagtgccttcgagtgcgaggcgaggtggctcctgacgctaagagct | |
| | | | | tcgtgctgaacctgggcaaagacagcaacaacctgtgcctgcacttcaaccct | |
| | | | | cgcttcaacgcccacggcgacgccaacaccatcgtgtgcaacagcaaggacgg | |
| | | | | cggggcctgggggaccgagcagcgggaggctgtctttcccttccagcctggaa | |
| | | | | gtgttgcagaggtgtgcatcaccttcgaccaggccaacctgaccgtcaagctg | |
| | | | | ccagatggatacgaattcaagttccccaaccgcctcaacctggaggccatcaactaca | |
| | | | | tggcagctgacggtgacttcaa | |
| | | 101 | GTGCCTTCGAGTGCGAGGCGAGGTG | | |
| | | 102 | CTCCTGACGCTAAGAGCTTCGTGCT | | |
| | | 103 | GCTTCGTGCTGAACCTGGGCAAAGA | | |
| | | 104 | TGTGCAACAGCAAGGACGGCGGGGC | | |
| | | 105 | ACCGAGCAGCGGGAGGCTGTCTTTC | | |
| | | 106 | GACCGTCAAGCTGCCAGATGGATAC | | |
| | | 107 | ACATGGCAGCTGACGGTGACTTCAA | | |
| | | 108 | CTCCTGGACTCAATCATGGCTTGTG | | |
| | | 109 | ATCATGGCTTGTGGTCTGGTCGCCA | | |
| | | 110 | GTCGCCAGCAACCTGAATCTCAAAC | | |
| ITPR3 | 201189_s_at | 111 | ACAGTCCTGCTTAGAGCCCTTAAAA | acagtcctgcttagagcccttaaaaagacttgaaagttcactgggactcagtt | NM_002224.1 |
| | | | | taccttaatgccttagcagaagataaatcctacctagagacctttgttcctta | |
| | | | | aagcaataactgacaactctttgtagtcctccttgtgggtagttaagagtggg | |
| | | | | gtcacccctttaactccaagcactacattttggcggctgcggcctctggggga | |
| | | | | ggtggcagttatgctgttactagtgattttagggctttgttatttaacttatt | |
| | | | | tcaagggtgctgtgctcagccctgcccatggctgtgcagctccctccgtgcct | |
| | | | | cagatctgctgtagccagtgcagacctcactgtcgtgtccatgccacccccgg | |
| | | | | catggctccaggtggcctggtgactccatgatggacgatcttgctcccaggac | |
| | | | | ctgcctcttcccaggcttcctggggaagagttgtacgcccaggcaacaagggctgag | |
| | | | | ctgcgcttgcgtggctgtttcatgaccgc | |
| | | 112 | GGACTCAGTTTACCTTAATGCCTTA | | |
| | | 113 | TAAATCCTACCTAGAGACCTTTGTT | | |
| | | 114 | AACTGACAACTCTTTGTAGTCCTCC | | |
| | | 115 | GGGAGGTGGCAGTTATGCTGTTACT | | |
| | | 116 | TGCCTCAGATCTGCTGTAGCCAGTG | | |
| | | 117 | GCTGTAGCCAGTGCAGACCTCACTG | | |
| | | 118 | CTCCAGGTGGCCTGGTGACTCCATG | | |
| | | 119 | CCATGATGGACGATCTTGCTCCCAG | | |
| | | 120 | GGGGAAGAGTTGTACGCCCAGGCAA | | |
| | | 121 | GCTTGCGTGGCTGTTTCATGACCGC | | |
| SPCS2 | 201240_s_at | 122 | GTATAGCTTTGGGCCATGTAGCATT | gagaagttgtagctctgatgtctagctgtagtctccttgatctgctgattgca | NM_014752.1 |
| | | | | ttattttaatttgcttttctgggaaagcagttttgctaaaagctgtacagact | |
| | | | | ttttcttttgtacctagcagtactttatatagtatagctttgggccatgtagc | |
| | | | | attttaagactcaattttaaaaaattattaatctgttgctgactcttaattcc | |
| | | | | tatttcaatatgtgtttccttgaagaattcaggatacaacttcttgtgtatga | |
| | | | | cagctttccttcacacactatttttgtgggtgtgtatatatctgatttgggaa | |
| | | | | gaatttaaaaaacacatagctttttaatttgtttgaaacagactttctgcctg | |
| | | | | ttacatttttgcttttaaccaattaaagaagccaatggcattttagttttatattgt | |
| | | | | gttttccactagtatatccctgttgatttgtttgtgccttt | |
| | | 123 | AAATTATTAATCTGTTGCTGACTCT | | |
| | | 124 | GTTGCTGACTCTTAATTCCTATTTC | | |
| | | 125 | GTGTATGACAGCTTTCCTTCACACA | | |
| | | 126 | TCCTTCACACACTATTTTTGTGGGT | | |
| | | 127 | GACTTTCTGCCTGTTACATTTTTGC | | |
| | | 128 | GTGTTTTCCACTAGTATATCCCTGT | | |
| | | 129 | TCCCTGTTGATTTGTTTGTGCCTTT | | |
| | | 130 | GAGAAGTTGTAGCTCTGATGTCTAG | | |
| | | 131 | GATGTCTAGCTGTAGTCTCCTTGAT | | |
| | | 132 | GTCTCCTTGATCTGCTGATTGCATT | | |
| TMEM109 | 201361_at | 133 | GAGCAGTCACTCTCAGAATCTTGAT | gagcagtcactctcagaatcttgattccccatcagccaaagcaaaagatggct | NM_024092.1 |
| | | | | gctgctttgtaggcatgtgcctgcaagtgggaccttgctgggcattatatgcc | |
| | | | | ctgtgggggtttcagagaccctgaaagaggagggaggacccgcctccttgtct | |
| | | | | gcacaactgcatgcacttctctccccatcgctccacaacctgaaaccgagaag | |
| | | | | gagttgctgaccagtgcccaccccggcagcccgggaggaacacaggcagctcc | |
| | | | | tttcccttcacgtggtctgcagagagcagggtgagctgccagctgcccctctc | |
| | | | | caccagggtaccctgtcttggtggttaggggccacttttcctttgaggctcta | |
| | | | | gtggaggtggatgtccttctctgccaggcttggcacatgatgtgaagaataaatgcc | |
| | | | | caattcttactgttcaggt | |
| | | 134 | TCAGAATCTTGATTCCCCATCAGCC | | |
| | | 135 | AAGTGGGACCTTGCTGGGCATTATA | | |
| | | 136 | ATGCCCTGTGGGGGTTTCAGAGACC | | |
| | | 137 | TCTGCACAACTGCATGCACTTCTCT | | |
| | | 138 | TCGCTCCACAACCTGAAACCGAGAA | | |
| | | 139 | AGAAGGAGTTGCTGACCAGTGCCCA | | |
| | | 140 | AGCCCGGGAGGAACACAGGCAGCTC | | |
| | | 141 | CTCCACCAGGGTACCCTGTCTTGGT | | |
| | | 142 | TAGTGGAGGTGGATGTCCTTCTCTG | | |
| | | 143 | AATGCCCAATTCTTACTGTTCAGGT | | |
| IFI30 | 201422_at | 144 | TGGAGGCCTGCGTGTTGGATGAACT | tggaggcctgcgtgttggatgaacttgacatggagctagccttcctgaccatg | NM_006332.1 |
| | | | | tctggcatggcatggaagagtttgaggacatggagagaagtctgccactatgc | |
| | | | | ctgcagctctacgccccagggctgtcgccagaactatcatggagtgtgcaatg | |
| | | | | ggggaccgcggcatgcagctcatgcacgccaacgcccagcggacagatgctct | |
| | | | | ccagccaccgcacgagtatgtgccctgggtcaccgtcaatgggaaacccttgg | |
| | | | | aagatcagacccagctccttacccttgtctgccagttgtaccagggcaagaag | |
| | | | | ccggatgtctgcccttcctcaaccagctccctccggagtgtttgcttcgagtg | |
| | | | | ttggccggtgggctgcggagagctcatggaaggcgagtgggaactcggctgcc | |
| | | | | tgcctttttttctgatccagaccctcggcacctgctacttaccaactggaaaa | |
| | | | | ttttatgcatcccatgaagcccagatacacaaaattccacccctagatcaagaatcct | |
| | | | | gctccacta | |
| | | 145 | TTGACATGGAGCTAGCCTTCCTGAC | | |
| | | 146 | CAGGGCTGTCGCCAGAACTATCATG | | |
| | | 147 | TGGAGTGTGCAATGGGGGACCGCGG | | |
| | | 148 | TCCAGCCACCGCACGAGTATGTGCC | | |
| | | 149 | TGCCCTGGGTCACCGTCAATGGGAA | | |
| | | 150 | CCTTGTCTGCCAGTTGTACCAGGGC | | |
| | | 151 | GGCAAGAAGCCGGATGTCTGCCCTT | | |
| | | 152 | GGAGTGTTTGCTTCGAGTGTTGGCC | | |
| | | 153 | ATGCATCCCATGAAGCCCAGATACA | | |
| | | 154 | CTAGATCAAGAATCCTGCTCCACTA | | |
| CAT | 201432_at | 155 | TTAGCGTTCATCCGTGTAACCCGCT | ttagcgttcatccgtgtaacccgctcatcactggatgaagattctcctgtgct | NM_001752.1 |
| | | | | agatgtgcaaatgcaagctagtggcttcaaaatagagaatcccactttctata | |
| | | | | gcagattgtgtaacaattttaatgctatttccccaggggaaaatgaaggttag | |
| | | | | gatttaacagtcatttaaaaaaaaaatttgttttgacggatgattggattatt | |
| | | | | catttaaaatgattagaaggcaagtttctagctagaaatatgattttatttga | |
| | | | | caaaatttgttgaaattatgtatgtttacatatcacctcatggcctattatat | |
| | | | | taaaatatggctataaatatataaaaagaaaagataaagatgatctactcaga | |
| | | | | aatttttatttttctaaggttctcataggaaaagtacatttaatacagcagtgtcatc | |
| | | | | agaagataacttgagcaccgtcatggcttaatgtttatt | |
| | | 156 | GTAACCCGCTCATCACTGGATGAAG | | |
| | | 157 | GATGAAGATTCTCCTGTGCTAGATG | | |
| | | 158 | GATTCTCCTGTGCTAGATGTGCAAA | | |
| | | 159 | GTGCAAATGCAAGCTAGTGGCTTCA | | |
| | | 160 | GAGAATCCCACTTTCTATAGCAGAT | | |
| | | 161 | CAATTTTAATGCTATTTCCCCAGGG | | |
| | | 162 | GTATGTTTACATATCACCTCATGGC | | |
| | | 163 | TATCACCTCATGGCCTATTATATTA | | |
| | | 164 | GATAACTTGAGCACCGTCATGGCTT | | |
| | | 165 | GCACCGTCATGGCTTAATGTTTATT | | |
| G3BP | 201503_at | 166 | AAAACCCAGATAACAACCAGAGCAA | aaaacccagataacaaccagagcaaaactgttgtgccttctatttatctttga | BG500067 |
| | | | | tttcagtcttggcaattgtttaaaaaaaaaatctagatttgttttattaggtt | |
| | | | | cagagtatgtggggaattatagaatccctctttcatcactttgtgtatgtctt | |
| | | | | ttgttaacatatttgttatgccttattctaaaattgagtctcaaactggaatg | |
| | | | | cctttgaagacagatgcttctatagaggttctttgacctaaatagttcagcat | |
| | | | | ttgtatttttattctggtatctaatcagattcctaatcatagcccgtaagaag | |
| | | | | gaatgttactttaatattggactttgctcatgtgctcgtgtccgcattttttt | |
| | | | | ttttncttaaaatcatagccatatggtaaattttctattttgttatggttctctttta | |
| | | | | ttgatgggcatgcagtgggtgttacttgga | |
| | | 167 | GCAAAACTGTTGTGCCTTCTATTTA | | |
| | | 168 | TTATCTTTGATTTCAGTCTTGGCAA | | |
| | | 169 | CATATTTGTTATGCCTTATTCTAAA | | |
| | | 170 | TTGAGTCTCAAACTGGAATGCCTTT | | |
| | | 171 | GACAGATGCTTCTATAGAGGTTCTT | | |
| | | 172 | GGTTCTTTGACCTAAATAGTTCAGC | | |
| | | 173 | CAGATTCCTAATCATAGCCCGTAAG | | |
| | | 174 | TGCTCGTGTCCGCATTTTTTTTTTT | | |
| | | 175 | GGTTCTCTTTTATTGATGGGCATGC | | |
| | | 176 | GGGCATGCAGTGGGTGTTACTTGGA | | |
| ARF5 | 201526_at | 177 | GCAGTGCTGCTGGTATTTGCCAACA | gcagtgctgctggtatttgccaacaagcaggacatgcccaacgccatgcccgt | NM_001662.2 |
| | | | | gagcgagctgactgacaagctggggctacagcacttacgcagccgcacgtggt | |
| | | | | atgtccaggccacctgtgccacccaaggcacaggtctgtacgatggtctggac | |
| | | | | tggctgtcccacgagctgtcaaagcgctaaccagccaggggcaggcccctgat | |
| | | | | gcccggaagctcctgcgtgcatccccgggatgaccagactcccggactcctca | |
| | | | | ggcagtgccctttcctcccacttttcctcccccatagccacaggcctctgctc | |
| | | | | ctgctcctgcctgcatgttctctctgttgttggagcctggagccttgctctct | |
| | | | | gggcacagaggggtccactctcctgcctgctgggacctatggaaggggcttcc | |
| | | | | tggccaaggccccctcttccagaggaggagcagggatctgggtttcctttttttttt | |
| | | | | ctgttttgggtgtactctaggggccaggttggga | |
| | | 178 | TGCCCGTGAGCGAGCTGACTGACAA | | |
| | | 179 | TGCCACCCAAGGCACAGGTCTGTAC | | |
| | | 180 | GTACGATGGTCTGGACTGGCTGTCC | | |
| | | 181 | TCCCACGAGCTGTCAAAGCGCTAAC | | |
| | | 182 | CTGCGTGCATCCCCGGGATGACCAG | | |
| | | 183 | TCTCTGTTGTTGGAGCCTGGAGCCT | | |
| | | 184 | GCCTTGCTCTCTGGGCACAGAGGGG | | |
| | | 185 | GCCTGCTGGGACCTATGGAAGGGGC | | |
| | | 186 | GCCCCCTCTTCCAGAGGAGGAGCAG | | |
| | | 187 | GTGTACTCTAGGGGCCAGGTTGGGA | | |
| DUSP3 | 201536_at | 188 | GATTTAGCTCTTAGTTCTTCAAGTA | gatttagctcttagttcttcaagtaaaattaaagtctcttgtgtaagagccaa | AL048503 |
| | | | | cacatgcccagctgcggatgggagctgttcctggacagccttctactgcctgg | |
| | | | | gaagtgatggaacaggaactcagggtgcccttaccccctccccagacctgttc | |
| | | | | cctttctttgactgacagagcaccatccaggcaaaattagagcgccaaatggt | |
| | | | | tttcttctcaatcttaaagcagtatacctttccacaggctcgtctgtgtccct | |
| | | | | gccactctgagttatccagaaaccaccacctacaaatgaggggactcatctag | |
| | | | | aagacctctaaggtccccttttggctctgaggggtctctaataatccccactt | |
| | | | | ggaattcagcaccgcaaggaaattatgggtatgtgagccataatatgatggcc | |
| | | | | agcaggtngcgctgccttccacccatggtgatggatggtttggaaagggaatgttggt | |
| | | | | gccttttgtgccaca | |
| | | 189 | GAACAGGAACTCAGGGTGCCCTTAC | | |
| | | 190 | TGACTGACAGAGCACCATCCAGGCA | | |
| | | 191 | AAAGCAGTATACCTTTCCACAGGCT | | |
| | | 192 | TCCCTGCCACTCTGAGTTATCCAGA | | |
| | | 193 | GAAACCACCACCTACAAATGAGGGG | | |
| | | 194 | AGGGGACTCATCTAGAAGACCTCTA | | |
| | | 195 | CCTTTTGGCTCTGAGGGGTCTCTAA | | |
| | | 196 | GGGTCTCTAATAATCCCCACTTGGA | | |
| | | 197 | CCCACTTGGAATTCAGCACCGCAAG | | |
| | | 198 | GAATGTTGGTGCCTTTTGTGCCACA | | |
| ID2 | 201565_s_at | 199 | GAAAAACAGCCTGTCGGACCACAGC | gaaaaacagcctgtcggaccacagcctgggcatctcccggagcaaaacccctg | NM_002166.1 |
| | | | | tggacgacccgatgagcctgctatacaacatgaacgactgctactccaagctc | |
| | | | | aaggagctggtgcccagcatcccccagaacaagaaggtgagcaagatggaaat | |
| | | | | cctgcagcacctcatcgactacatcttggacctgcagatcgccctggactcgc | |
| | | | | atcccactattgtcagcctgcatcaccagagacccgggcagaaccagcgctcc | |
| | | | | aggacgccgctgaccaccctcaacacggatatcagcatcctgtccttgcaggc | |
| | | | | ttctgaattcccttctgagttaatgtcaaatgacagcaaagcactgtgtggct | |
| | | | | gaataagcggtgttcatgatttcttttattctttgcacaacaacaacaacaacaaattc | |
| | | | | acggaatcttttaagtgctgaac | |
| | | 200 | GACCCGATGAGCCTGCTATACAACA | | |
| | | 201 | CCCGATGAGCCTGCTATACAACATG | | |
| | | 202 | GAGCCTGCTATACAACATGAACGAC | | |
| | | 203 | TATACAACATGAACGACTGCTACTC | | |
| | | 204 | GTGTGGCTGAATAAGCGGTGTTCAT | | |
| | | 205 | GAATAAGCGGTGTTCATGATTTCTT | | |
| | | 206 | AGCGGTGTTCATGATTTCTTTTATT | | |
| | | 207 | GGTGTTCATGATTTCTTTTATTCTT | | |
| | | 208 | CAACAACAAATTCACGGAATCTTTT | | |
| | | 209 | TCACGGAATCTTTTAAGTGCTGAAC | | |
| DNMT1 | 201697_s_at | 210 | ACCCAGAGCAGCACCGTGTGGTGAG | acccagagcagcaccgtgtggtgagcgtgcgggagtgtgcccgctcccagggc | NM_001379.1 |
| | | | | ttccctgacacctaccggctcttcggcaacatcctggacaagcaccggcaggt | |
| | | | | gggcaatgccgtgccaccgcccctggccaaagccattggcttggagatcaagc | |
| | | | | tttgtatgttggccaaagcccgagagagtgcctcagctaaaataaaggaggag | |
| | | | | gaagctgctaaggactagttctgccctcccgtcacccctgtttctggcaccag | |
| | | | | gaatccccaacatgcactgatgttgtgtttttaacatgtcaatctgtccgttc | |
| | | | | acatgtgtggtacatggtgtttgtggccttggctgacatgaagctgttgtgtg | |
| | | | | aggttcgcttatcaactaatgatttagtgatcaaattgtgcagtactttgtgc | |
| | | | | attctggattttaaaagttttttattatgcattatatcaaatctaccactgtatgagt | |
| | | 211 | ACATCCTGGACAAGCACCGGCAGGT | | |
| | | 212 | CGGCAGGTGGGCAATGCCGTGCCAC | | |
| | | 213 | CCCCTGGCCAAAGCCATTGGCTTGG | | |
| | | 214 | GAGATCAAGCTTTGTATGTTGGCCA | | |
| | | 215 | AGCTGCTAAGGACTAGTTCTGCCCT | | |
| | | 216 | CAATCTGTCCGTTCACATGTGTGGT | | |
| | | 217 | GGCTGACATGAAGCTGTTGTGTGAG | | |
| | | 218 | GTGTGAGGTTCGCTTATCAACTAAT | | |
| | | 219 | GCAGTACTTTGTGCATTCTGGATTT | | |
| | | 220 | ATATCAAATCTACCACTGTATGAGT | | |
| CCND3 | 201700_at | 221 | TTGCATTTGGATTGGGGTCCCTCTA | ttgcatttggattggggtccctctaaaatttaatgcatgatagacacatatga | NM_001760.1 |
| | | | | gggggaatagtctagatggctcctctcagtactttggaggcccctatgtagtc | |
| | | | | cgtgctgacagctgctcctagagggaggggcctaggcctcagccagagaagct | |
| | | | | ataaattcctctttgctttgctttctgctcagcttctcctgtgtgattgacag | |
| | | | | ctttgctgctgaaggctcattttaatttattaattgctttgagcacaacttta | |
| | | | | agaggacataatgggggcctggccatccacaagtggtggtaaccctggtggtt | |
| | | | | gctgttttcctcccttctgctactggcaaaaggatctttgtggccaaggagct | |
| | | | | gctatagcctggggtggggtcatgccctcctctcccattgtccctctgcccca | |
| | | | | tcctccagcagggaaaatgcagcagggatgccctggaggtggctgagcccctg | |
| | | | | tctagagagggaggcaagccctgttgacacaggtctttcctaaggctgcaaggtttag | |
| | | | | gctggtggccc | |
| | | 222 | GGGAATAGTCTAGATGGCTCCTCTC | | |
| | | 223 | GGCTCCTCTCAGTACTTTGGAGGCC | | |
| | | 224 | CTATGTAGTCCGTGCTGACAGCTGC | | |
| | | 225 | GCTCAGCTTCTCCTGTGTGATTGAC | | |
| | | 226 | GCTTTGCTGCTGAAGGCTCATTTTA | | |
| | | 227 | TAACCCTGGTGGTTGCTGTTTTCCT | | |
| | | 228 | TGGCCAAGGAGCTGCTATAGCCTGG | | |
| | | 229 | GGCTGAGCCCCTGTCTAGAGAGGGA | | |
| | | 230 | GACACAGGTCTTTCCTAAGGCTGCA | | |
| | | 231 | GCTGCAAGGTTTAGGCTGGTGGCCC | | |
| CD14 | 201743_at | 232 | GTGCCTAAAGGACTGCCAGCCAAGC | ccatccagaatctagcgctgcgcaacacaggaatggagacgcccacaggcgtg | NM_000591.1 |
| | | | | tgcgccgcactggcggcggcaggtgtgcagccccacagcctagacctcagcca | |
| | | | | caactcgctgcgcgccaccgtaaaccctagcgctccgagatgcatgtggtcca | |
| | | | | gcgccctgaactccctcaatctgtcgttcgctgggctggaacaggtgcctaaa | |
| | | | | ggactgccagccaagctcagagtgctcgatctcagctgcaacagactgaacag | |
| | | | | ggcgccgcagcctgacgagctgcccgaggtggataacctgacactggacggga | |
| | | | | atcccttcctggtccctggaactgccctcccccacgagggctcaatgaactcc | |
| | | | | ggcgtggtcccagcctgtgcacgttcgaccctgtcggtgggggtgtcgggaac | |
| | | | | cctggtgctgctccaaggggcccggggctttgcctaagatccaagacagaata | |
| | | | | atgaatggactcaaactgccttggcttcaggggagtcccgtcaggacgttgaggact | |
| | | | | tttcgaccaattcaacc | |
| | | 233 | GCCAAGCTCAGAGTGCTCGATCTCA | | |
| | | 234 | GCAACAGACTGAACAGGGCGCCGCA | | |
| | | 235 | TGACGAGCTGCCCGAGGTGGATAAC | | |
| | | 236 | CTGACACTGGACGGGAATCCCTTCC | | |
| | | 237 | ACGAGGGCTCAATGAACTCCGGCGT | | |
| | | 238 | CCCGGGGCTTTGCCTAAGATCCAAG | | |
| | | 239 | GGGAGTCCCGTCAGGACGTTGAGGA | | |
| | | 240 | TGAGGACTTTTCGACCAATTCAACC | | |
| | | 241 | CCATCCAGAATCTAGCGCTGCGCAA | | |
| | | 242 | CCCTAGCGCTCCGAGATGCATGTGG | | |
| RPA2 | 201756_at | 243 | GGTTTCATCTATCAAATGTCTCCTC | gatattttacagctggacctagtttcacaatctgttgtctccagctctgcata | NM_002946.1 |
| | | | | tgtctggccagggggcttctaggaagtaggtttcatctatcaaatgtctcctc | |
| | | | | tgacttccttttgaaacttactgctcttctgttttattttgttttgtttgaag | |
| | | | | ctcagagggagatgggcaattgacagggatgcaatccagggtgggatttcttg | |
| | | | | aggaagttacaaataagcttgttacaacatcaagatagatggaattggaagga | |
| | | | | tgctaccaggagagtacttacatagtgctcaggagtttctcttcttaaaatgt | |
| | | | | ttactgctgaaagatgagcaggaccagggcgttataggcagagccctagccag | |
| | | | | aaacctgctggcctctgcctgttttcatttcccactttggttgtgtggcatta | |
| | | | | ctttcagaattgcactttcctgcttgtcatgactttttgacacacttgccatgac | |
| | | 244 | TCCTCTGACTTCCTTTTGAAACTTA | | |
| | | 245 | ACTTACTGCTCTTCTGTTTTATTTT | | |
| | | 246 | GACAGGGATGCAATCCAGGGTGGGA | | |
| | | 247 | TAGCCAGAAACCTGCTGGCCTCTGC | | |
| | | 248 | TGTTTTCATTTCCCACTTTGGTTGT | | |
| | | 249 | ACTTTCCTGCTTGTCATGACTTTTT | | |
| | | 250 | GACTTTTTGACACACTTGCCATGAC | | |
| | | 251 | GATATTTTACAGCTGGACCTAGTTT | | |
| | | 252 | GCTGGACCTAGTTTCACAATCTGTT | | |
| | | 253 | CTCCAGCTCTGCATATGTCTGGCCA | | |
| CDC25B | 201853_s_at | 254 | GCTTGGTCTGTTTGACTTTACGCCC | gcttggtctgtttgactttacgcccatctcaggacacttccgtagactgttta | NM_021873.1 |
| | | | | ggttcccctgtcaaatatcagttacccactcggtcccagttttgttgccccag | |
| | | | | aaagggatgttattatccttgggggctcccagggcaagggttaaggcctgaat | |
| | | | | catgagcctgctggaagcccagcccctactgctgtgaaccctggggcctgact | |
| | | | | gctcagaacttgctgctgtcttgttgcggatggatggaaggttggatggatgg | |
| | | | | gtggatggccgtggatggccgtggatgcgcagtgccttgcatacccaaaccag | |
| | | | | gtgggagcgttttgttgagcatgacacctgcagcaggaatatatgtgtgccta | |
| | | | | tttgtgtggacaaaaatatttacacttagggtttggagctattcaagaggaaa | |
| | | | | tgtcacagaagcagctaaaccaaggactgagcaccctctggattctgaatctc | |
| | | | | aagatgggggcagggctgtgcttgaaggccctgctgagtcatctgttagggccttgg | |
| | | | | ttc | |
| | | 255 | CCATCTCAGGACACTTCCGTAGACT | | |
| | | 256 | GTTTAGGTTCCCCTGTCAAATATCA | | |
| | | 257 | CAAATATCAGTTACCCACTCGGTCC | | |
| | | 258 | TGAATCATGAGCCTGCTGGAAGCCC | | |
| | | 259 | CCCCTACTGCTGTGAACCCTGGGGC | | |
| | | 260 | TTGCTGCTGTCTTGTTGCGGATGGA | | |
| | | 261 | GATGGCCGTGGATGGCCGTGGATGC | | |
| | | 262 | GTGGGAGCGTTTTGTTGAGCATGAC | | |
| | | 263 | GCACCCTCTGGATTCTGAATCTCAA | | |
| | | 264 | GAGTCATCTGTTAGGGCCTTGGTTC | | |
| ST6GAL1 | 201998_at | 265 | GGCTGCTTAACTGCTGTATAGGACA | ggctgcttaactgctgtataggacaagccccttacccctctctgggcccatga | AI743792 |
| | | | | attcctggcttggtttatgttctgatttgacacactgattttaatcttcgaat | |
| | | | | catgacactgagtgcagaggaggtggcattccgacagcaggacatacatgttg | |
| | | | | gtgtgaagactgggacgacactgggtagaatctagtttttaattattattaat | |
| | | | | ataaaggatcaaattaatttaaatatgattctgaagtctacagaacttttagt | |
| | | | | tctgtgctgtctatgtggacactttggtaaaatgcaaattatgatatggacgt | |
| | | | | tatcattggtctggtgagatgtttcatatttgtgacagttaatttaaaaatta | |
| | | | | tganttaatgctgcctgtgtctatggggttctgtcttctttgatagccatctattcat | |
| | | | | ctggatcatgggaccctctctaa | |
| | | 266 | TGCTGTATAGGACAAGCCCCTTACC | | |
| | | 267 | GCCCATGAATTCCTGGCTTGGTTTA | | |
| | | 268 | GGCTTGGTTTATGTTCTGATTTGAC | | |
| | | 269 | GGTGGCATTCCGACAGCAGGACATA | | |
| | | 270 | GAAGACTGGGACGACACTGGGTAGA | | |
| | | 271 | AGTTCTGTGCTGTCTATGTGGACAC | | |
| | | 272 | AATGCTGCCTGTGTCTATGGGGTTC | | |
| | | 273 | TATGGGGTTCTGTCTTCTTTGATAG | | |
| | | 274 | GATAGCCATCTATTCATCTGGATCA | | |
| | | 275 | ATCTGGATCATGGGACCCTCTCTAA | | |
| ARL2BP | 202092_s_at | 276 | GGGCCACAGTTTCAGTACTTCAGCC | ccctcctggacctatttatcctgaaacaccttcttgtattcattaaccatagt | NM_012106.1 |
| | | | | actcctccccacctcaagtagacacctctctcaggagcttctgagtcagacgc | |
| | | | | ctctggagcgagccctatgtcaggcactccacctggggggcccttccccagca | |
| | | | | tacctgctggtgtgtaagtgtggactaacccgccgccaccaccctctgttcca | |
| | | | | gcaggctctgcatgaatctttgtgcacttgcacctctttttcacatgggccac | |
| | | | | agtttcagtacttcagcctcagtggggttcctgatgtttatctagggtgttac | |
| | | | | tcaagcccagtttgagattttggagtctcctgtgatcacatcttgtctcggct | |
| | | | | gtaggaatcaacagaaggagacgtcctctacataaaagctccatgtgaaaagc | |
| | | | | tactcctagtcttaacatttgcagtccttgtgtcactgtcttctggtcctgatgtag | |
| | | | | tccc | |
| | | 277 | CTTCAGCCTCAGTGGGGTTCCTGAT | | |
| | | 278 | TTTGGAGTCTCCTGTGATCACATCT | | |
| | | 279 | TCACATCTTGTCTCGGCTGTAGGAA | | |
| | | 280 | GACGTCCTCTACATAAAAGCTCCAT | | |
| | | 281 | GCTACTCCTAGTCTTAACATTTGCA | | |
| | | 282 | TGTCTTCTGGTCCTGATGTAGTCCC | | |
| | | 283 | CCCTCCTGGACCTATTTATCCTGAA | | |
| | | 284 | ATTCATTAACCATAGTACTCCTCCC | | |
| | | 285 | GACACCTCTCTCAGGAGCTTCTGAG | | |
| | | 286 | CTCTGGAGCGAGCCCTATGTCAGGC | | |
| TSPO | 202096_s_at | 287 | GGCTCCTACCTGGTCTGGAAAGAGC | ggctcctacctggtctggaaagagctgggaggcttcacagagaaggctgtggt | NM_000714.2 |
| | | | | tcccctgggcctctacactgggcagctggccctgaactgggcatggcccccca | |
| | | | | tcttctttggtgcccgacaaatgggctgggccttggtggatctcctgctggtc | |
| | | | | agtggggcggcggcngccactaccgtggcctggtaccaggtgagcccgctggc | |
| | | | | cgcccgcctgctctacccctacctggcctggctggccttcgcgaccacactca | |
| | | | | actactgcgtatggcgggacaaccatggctggcatgggggacggcggctgcca | |
| | | | | gagtgagtgcccggcccaccagggactgcagctgcaccagcaggtgccatcac | |
| | | | | gcttgtgatgtggtggccgtcacgctttcatgaccactgggcctgctagtctg | |
| | | | | tcagggccttggcccaggggtcagcagagcttcagaggttgccccacctgagc | |
| | | | | ccccacccgggagcagtgtcctgtgctttctgcatgcttagagcatg | |
| | | 288 | GGAAAGAGCTGGGAGGCTTCACAGA | | |
| | | 289 | CATCTTCTTTGGTGCCCGACAAATG | | |
| | | 290 | CCGACAAATGGGCTGGGCCTTGGTG | | |
| | | 291 | CGTGGCCTGGTACCAGGTGAGCCCG | | |
| | | 292 | GACCACACTCAACTACTGCGTATGG | | |
| | | 293 | AACTACTGCGTATGGCGGGACAACC | | |
| | | 294 | ATGGCGGGACAACCATGGCTGGCAT | | |
| | | 295 | TGCACCAGCAGGTGCCATCACGCTT | | |
| | | 296 | TCACGCTTGTGATGTGGTGGCCGTC | | |
| | | 297 | GTGCTTTCTGCATGCTTAGAGCATG | | |
| ZMYND11 | 202136_at | 298 | AGGTTTGTCAGGGTCACTCTAAAGA | aggtttgtcagggtcactctaaagataaaaatgtaactaagtcttctgtgaaa | BE250417 |
| | | | | tatcatccatctaatcttgatgctgttgcagatggtggtgacacaagttaatt | |
| | | | | gacaaactactgccaaatggtgcacaatattttgtaaaaagtacccagtagcc | |
| | | | | ccatttcatacaatgtacctaaattatgcagtaacttggcatcatcgttccct | |
| | | | | ccttgttgctgtgtaattagtcagtgttgccacagtgtgtggcgctgatggag | |
| | | | | atgtcagaaccgagaacacttaaccttctttgattgtttttcaagttttaaga | |
| | | | | cttcgatccacccctatgagagcaagtaattgtggaaatatttttggtgtaaa | |
| | | | | atcattccagagtatgtaatatttaactgatagctgcatgaaagtgagattcg | |
| | | | | tgttactttggcttttctgtctctgttgacacggttgcacatttccaagtta | |
| | | 299 | GTGAAATATCATCCATCTAATCTTG | | |
| | | 300 | TGTAAAAAGTACCCAGTAGCCCCAT | | |
| | | 301 | GTAGCCCCATTTCATACAATGTACC | | |
| | | 302 | GCAGTAACTTGGCATCATCGTTCCC | | |
| | | 303 | AGTGTGTGGCGCTGATGGAGATGTC | | |
| | | 304 | GTCAGAACCGAGAACACTTAACCTT | | |
| | | 305 | GATCCACCCCTATGAGAGCAAGTAA | | |
| | | 306 | GAGATTCGTGTTACTTTGGCTTTTC | | |
| | | 307 | GCTTTTCTGTCTCTGTTGACACGGT | | |
| | | 308 | GACACGGTTGCACATTTCCAAGTTA | | |
| BLMH | 202179_at | 309 | GCATGTCCCTGAAGAGGTGCTAGCT | gcatgtccctgaagaggtgctagctgtgttagagcaggaacccattatcctgc | NM_000386.1 |
| | | | | cagcatgggaccccatgggagctttggctgagtgatactgccctccagctctt | |
| | | | | tcctccttccatggaacctgacgtagctgcaaaggacagatccagggactgaa | |
| | | | | gccaaagttatgcaagggactgtgtgttgccacaggacacagtcagatttcca | |
| | | | | gtctccaccaggaacctcttcagaaagtgtgctttatgctgaaacagaatact | |
| | | | | gttaaaggaaaaaaaagaggggggaagatcaggtcatactatctactctcctc | |
| | | | | atctctaacagctcaggatctcttagcattttaattagatgtaattgtttgtc | |
| | | | | tttaactgtcaaaaagtttggttctgtgtctgtgttttaataagacgagagga | |
| | | | | cgagcgattgaggtgtatggagagaaaacagacctaatgctccttgttcctag | |
| | | | | agtagagtggagggagggtggcctaagagttgagctctcggaactgcatgctgc | |
| | | 310 | GAACCCATTATCCTGCCAGCATGGG | | |
| | | 311 | ACCCCATGGGAGCTTTGGCTGAGTG | | |
| | | 312 | TTTGGCTGAGTGATACTGCCCTCCA | | |
| | | 313 | TCCTCCTTCCATGGAACCTGACGTA | | |
| | | 314 | AGGAACCTCTTCAGAAAGTGTGCTT | | |
| | | 315 | TCTCCTCATCTCTAACAGCTCAGGA | | |
| | | 316 | AACAGCTCAGGATCTCTTAGCATTT | | |
| | | 317 | AAACAGACCTAATGCTCCTTGTTCC | | |
| | | 318 | GAGGGTGGCCTAAGAGTTGAGCTCT | | |
| | | 319 | TTGAGCTCTCGGAACTGCATGCTGC | | |
| CTSH | 202295_s_at | 320 | AGCCGCAGCGCAGACTGGCGGAGAA | tagaacgggcatctactccagtacttcctgccataaaactccagataaagtaa | NM_004390.1 |
| | | | | accatgcagtactggctgttgggtatggagaaaaaaatgggatcccttactgg | |
| | | | | atcgtgaaaaactcttggggtccccagtggggaatgaacgggtacttcctcat | |
| | | | | cgagcgcggaaagaacatgtgtggcctggctgcctgcgcctcctaccccatcc | |
| | | | | ctctggtgtgagccgtggcagccgcagcgcagactggcggagaaggagaggaa | |
| | | | | cgggcagcctgggcctgggtggaaatcctgccctggaggaagttgtggggaga | |
| | | | | tccactgggacccccaacattctgccctcacctctgtgcccagcctggaaacc | |
| | | | | tacagacaaggaggagttccaccatgagctcacccgtgtctatgacgcaaaga | |
| | | | | tcaccagccatgtgccttagtgtccttcttaacagactcaaaccacatggacc | |
| | | | | acgaatattctttctgtccagaagggctactttccacatatagagctccagggactgt | |
| | | | | ctttt | |
| | | 321 | TGTGGGGAGATCCACTGGGACCCCC | | |
| | | 322 | AAGGAGGAGTTCCACCATGAGCTCA | | |
| | | 323 | CTCACCCGTGTCTATGACGCAAAGA | | |
| | | 324 | AAAGATCACCAGCCATGTGCCTTAG | | |
| | | 325 | GCCTTAGTGTCCTTCTTAACAGACT | | |
| | | 326 | GGACCACGAATATTCTTTCTGTCCA | | |
| | | 327 | TGTCCAGAAGGGCTACTTTCCACAT | | |
| | | 328 | TATAGAGCTCCAGGGACTGTCTTTT | | |
| | | 329 | TAGAACGGGCATCTACTCCAGTACT | | |
| | | 330 | CAGTACTTCCTGCCATAAAACTCCA | | |
| MAPRE2 | 202501_at | 331 | CAGCCACAAAACTGTCATTCACTCT | cagccacaaaactgtcattcactctaggggacccctactaaagggtaacttca | NM_014268.1 |
| | | | | ggtgtgcagccctgagctccaaggctctgcaccatgccacacacttgctgtaa | |
| | | | | ggctagaagtgaagaccttattaataggagcataattgcgagggagaatcatg | |
| | | | | gttctgcagtctggtgtagacactggaataacagcacagaaaaatctatgact | |
| | | | | cccaatatcttctagaataaagaattttccctctttaacacaagggccctcct | |
| | | | | tgtcattgaccttagctaaaccatggcaattcataaatagaggaaacattaat | |
| | | | | gaattaaaagcattccttattttttaactaatatttgtacattttcttagtct | |
| | | | | ctttccaagtctttgcctcttttttttctttatttttattttttcctttgacagatg | |
| | | | | gtatcccttcctggatcattcatttcaccttggtt | |
| | | 332 | TCATTCACTCTAGGGGACCCCTACT | | |
| | | 333 | ACTTCAGGTGTGCAGCCCTGAGCTC | | |
| | | 334 | CATGCCACACACTTGCTGTAAGGCT | | |
| | | 335 | GAATCATGGTTCTGCAGTCTGGTGT | | |
| | | 336 | AATCTATGACTCCCAATATCTTCTA | | |
| | | 337 | AACACAAGGGCCCTCCTTGTCATTG | | |
| | | 338 | CCCTCCTTGTCATTGACCTTAGCTA | | |
| | | 339 | GACCTTAGCTAAACCATGGCAATTC | | |
| | | 340 | TTGACAGATGGTATCCCTTCCTGGA | | |
| | | 341 | CTGGATCATTCATTTCACCTTGGTT | | |
| ARHGEF7 | 202548_s_at | 342 | GTTACGGCATTGCCTTTTCTTTCTG | gttacggcattgccttttctttctgtggatccagtatcttcctcggcttttta | NM_003899.1 |
| | | | | gggagcaggaaaaatgcgtctgagagcaactctttttaaaaacctgccctgtt | |
| | | | | gtatataactgtgtctgtttcaccgtgtgacctcccaagggggtgggaacttg | |
| | | | | atataaacgtttaaaggggccacgatttgcccgagggttactcctttgctctc | |
| | | | | accttgtatggatgaggagatgaagccatttcttatcctgtagatgtgaagca | |
| | | | | ctttcagttttcagcgatgttggaatgtagcatcagaagctcgttccttcaca | |
| | | | | ctcagtggcgtctgtgcttgtccacatgcactgggcgtctgggaccttgaatg | |
| | | | | cctgccctggttgtgtggactccttaatgccaatcatttcttcacttctctgggaca | |
| | | | | cccagggcgcctgttgacaagtg | |
| | | 343 | TTCTGTGGATCCAGTATCTTCCTCG | | |
| | | 344 | ATCTTCCTCGGCTTTTTAGGGAGCA | | |
| | | 345 | AAACCTGCCCTGTTGTATATAACTG | | |
| | | 346 | AACTGTGTCTGTTTCACCGTGTGAC | | |
| | | 347 | GCCACGATTTGCCCGAGGGTTACTC | | |
| | | 348 | CCTTTGCTCTCACCTTGTATGGATG | | |
| | | 349 | GATGAAGCCATTTCTTATCCTGTAG | | |
| | | 350 | GTAGCATCAGAAGCTCGTTCCTTCA | | |
| | | 351 | CACACTCAGTGGCGTCTGTGCTTGT | | |
| | | 352 | CACCCAGGGCGCCTGTTGACAAGTG | | |
| KIFAP3 | 203333_at | 353 | CCACCAAGCCACAAGAGACGTCATA | ccaccaagccacaagagacgtcataatcaaggaaacacaggctccagcatatc | NM_014970.1 |
| | | | | tcatagacctaatgcatgataagaataatgaaatccgaaaggtctgtgataat | |
| | | | | acattagatattatagcggaatatgatgaagaatgggctaagaaaattcagag | |
| | | | | tgaaaagtttcgctggcataactctcagtggctggagatggtagagagtcgtc | |
| | | | | agatggatgagagtgagcagtacttgtatggtgatgatcgaattgagccatac | |
| | | | | attcatgaaggagatattctcgaaagacctgaccttttctacaactcagatgg | |
| | | | | attaattgcctctgaaggagccataagtcccgatttcttcaatgattaccacc | |
| | | | | ttcaaaatggagatgttgttgggcagcattcatttcctggcagccttggaatg | |
| | | | | gatggctttggccaaccagttggcattcttggacgccctgccacagcatatgg | |
| | | | | attccgccctgatgaaccttactactatggctatggatcttgataaagtatctgtttc | |
| | | | | catgtgtaatctca | |
| | | 354 | AACACAGGCTCCAGCATATCTCATA | | |
| | | 355 | GTTTCGCTGGCATAACTCTCAGTGG | | |
| | | 356 | CTCGAAAGACCTGACCTTTTCTACA | | |
| | | 357 | GGAGCCATAAGTCCCGATTTCTTCA | | |
| | | 358 | GATTTCTTCAATGATTACCACCTTC | | |
| | | 359 | GGATGGCTTTGGCCAACCAGTTGGC | | |
| | | 360 | CCCTGCCACAGCATATGGATTCCGC | | |
| | | 361 | GCATATGGATTCCGCCCTGATGAAC | | |
| | | 362 | GAACCTTACTACTATGGCTATGGAT | | |
| | | 363 | GTATCTGTTTCCATGTGTAATCTCA | | |
| OFD1 | 203569_s_at | 364 | AGCAGGAGCAAGACCAGGAGTCGGC | agcaggagcaagaccaggagtcggcagataagagctcaaaaaagatggtccaa | NM_003611.1 |
| | | | | gaaggctccctagtggacacgctgcaatctagtgacaaagtcgaaagtttaac | |
| | | | | aggcttttctcatgaagaactagacgactcttggtaaccatgtttgctgccca | |
| | | | | gcttctaacttacataccgtgagaagttacgtaacatttactcctttgtaaat | |
| | | | | gtttccctatcatcagacaaaactcaataaaaatgtgtgtaatccaatgtggg | |
| | | | | tttttttttccataattaattttgataccatagtgtgtgaaccaagaataatctagtc | |
| | | | | acgtgaaacctcttctccagtcatagtatt | |
| | | 365 | CAAGAAGGCTCCCTAGTGGACACGC | | |
| | | 366 | GTGGACACGCTGCAATCTAGTGACA | | |
| | | 367 | AAGTTTAACAGGCTTTTCTCATGAA | | |
| | | 368 | GAACTAGACGACTCTTGGTAACCAT | | |
| | | 369 | CTTGGTAACCATGTTTGCTGCCCAG | | |
| | | 370 | TGTTTGCTGCCCAGCTTCTAACTTA | | |
| | | 371 | CCAGCTTCTAACTTACATACCGTGA | | |
| | | 372 | AAATGTTTCCCTATCATCAGACAAA | | |
| | | 373 | GATACCATAGTGTGTGAACCAAGAA | | |
| | | 374 | AAACCTCTTCTCCAGTCATAGTATT | | |
| CEBPA | 204039_at | 375 | AAGCTAGGTCGTGGGTCAGCTCTGA | aagctaggtcgtgggtcagctctgaggatgtatacccctggtgggagagggag | NM_004364.1 |
| | | | | acctagagatctggctgtggggcgggcatggggggtgaagggccactgggacc | |
| | | | | ctcagccttgtttgtactgtatgccttcagcattgcctaggaacacgaagcac | |
| | | | | gatcagtccatccagagggaccggagttatgacaagcttcccaaatattttgc | |
| | | | | tttatcagccgatatcaacacttgtatctggcctctgtgcccagcagtgcctt | |
| | | | | gtgcaatgtgaatgtaccgtctctgctaaaccaccattttatttggttttgtt | |
| | | | | ttgtttggttttctcggatacttgccaaaatgagactctccgtcggcagctgg | |
| | | | | gggaagggtctgagactctctttccttttggttttgggattacttttgatcct | |
| | | | | gggggaccaatgaggtgaggggggttctcctttgccctcagctttcccagccc | |
| | | | | tccggcctgggctgcccacaaggcttctcccccagaggccctggctcctggtcgggaa | |
| | | | | gggag | |
| | | 376 | AGCTCTGAGGATGTATACCCCTGGT | | |
| | | 377 | GAGGGAGACCTAGAGATCTGGCTGT | | |
| | | 378 | AGCCTTGTTTGTACTGTATGCCTTC | | |
| | | 379 | ATGCCTTCAGCATTGCCTAGGAACA | | |
| | | 380 | GAACACGAAGCACGATCAGTCCATC | | |
| | | 381 | TCAACACTTGTATCTGGCCTCTGTG | | |
| | | 382 | TGTGAATGTACCGTCTCTGCTAAAC | | |
| | | 383 | TGTTTGGTTTTCTCGGATACTTGCC | | |
| | | 384 | GCCAAAATGAGACTCTCCGTCGGCA | | |
| | | 385 | CCCTGGCTCCTGGTCGGGAAGGGAG | | |
| CCL4 | 204103_at | 386 | TACCATGAAGCTCTGCGTGACTGTC | taccatgaagctctgcgtgactgtcctgtctctcctcatgctagtagctgcct | NM_002984.1 |
| | | | | tctgctctccagcgctctcagcaccaatgggctcagaccctcccaccgcctgc | |
| | | | | tgcttttcttacaccgcgaggaagcttcctcgcaactttgtggtagattacta | |
| | | | | tgagaccagcagcctctgctcccagccagctgtggtattccaaaccaaaagaa | |
| | | | | gcaagcaagtctgtgctgatcccagtgaatcctgggtccaggagtacgtgtat | |
| | | | | gacctggaactgaactgagctgctcagagacaggaagtcttcagggaaggtca | |
| | | | | cctgagcccggatgcttctccatgagacacatctcctccatactcaggactcc | |
| | | | | tctccgcagttcctgtcccttctcttaatttaatcttttttatgtgccgtgtt | |
| | | | | attgtattaggtgtcatttccattatttatattagtttagccaaaggataagtgtcc | |
| | | | | tatggggatggtccactgtcactg | |
| | | 387 | CTCATGCTAGTAGCTGCCTTCTGCT | | |
| | | 388 | GCTCTCAGCACCAATGGGCTCAGAC | | |
| | | 389 | TTTCTTACACCGCGAGGAAGCTTCC | | |
| | | 390 | GCTTCCTCGCAACTTTGTGGTAGAT | | |
| | | 391 | AGTCTGTGCTGATCCCAGTGAATCC | | |
| | | 392 | GACCTGGAACTGAACTGAGCTGCTC | | |
| | | 393 | TCAGGGAAGGTCACCTGAGCCCGGA | | |
| | | 394 | TCCATGAGACACATCTCCTCCATAC | | |
| | | 395 | ATCTTTTTTATGTGCCGTGTTATTG | | |
| | | 396 | CTATGGGGATGGTCCACTGTCACTG | | |
| STAB1 | 204150_at | 397 | GTGACGCAGGCCCTGACAACAGTTC | gtgacgcaggccctgacaacagttcctgggcccctgtggccccagggacagtt | NM_015136.1 |
| | | | | gtggttagccgtatcattgtgtgggacatcatggccttcaatggcatcatcca | |
| | | | | tgctctggccagccccctcctggcacccccacagccccaggcagtgctggcgc | |
| | | | | ctgaagccccacctgtggcggcaggcgtgggggctgtgcttgccgctggagca | |
| | | | | ctgcttggcttggtggccggagctctctacctccgtgcccgaggcaagcccac | |
| | | | | gggctttggcttctctgccttccaggcggaagatgatgctgacgacgacttct | |
| | | | | caccgtggcaagaagggaccaaccccaccctggtctctgtccccaaccctgtc | |
| | | | | tttggcagcgacaccttttgtgaacccttcgatgactcactgctggaggagga | |
| | | | | cttccctgacacccagaggatcctcacagtcaagtgacgaggctggggctgaa | |
| | | | | agcagaagcatgcacagggaggagaccacttttattgcttgtctgggtggat | |
| | | 398 | GCCCCAGGGACAGTTGTGGTTAGCC | | |
| | | 399 | GGTTAGCCGTATCATTGTGTGGGAC | | |
| | | 400 | TGTGTGGGACATCATGGCCTTCAAT | | |
| | | 401 | TCAATGGCATCATCCATGCTCTGGC | | |
| | | 402 | CGAGGCAAGCCCACGGGCTTTGGCT | | |
| | | 403 | AGATGATGCTGACGACGACTTCTCA | | |
| | | 404 | TGGCAGCGACACCTTTTGTGAACCC | | |
| | | 405 | CACTGCTGGAGGAGGACTTCCCTGA | | |
| | | 406 | CCTGACACCCAGAGGATCCTCACAG | | |
| | | 407 | ACTTTTATTGCTTGTCTGGGTGGAT | | |
| RUNX3 | 204197_s_at | 408 | ATCCATTGTCCTTGTAGTTTCTTCC | atccattgtccttgtagtttcttccctcctgttctctggttatagctggtccc | NM_004350.1 |
| | | | | aggtcagcgtgggaggcacctttgggttcccagtgcccagcactttgtagtct | |
| | | | | catcccagattactaacccttcctgatcctggagaggcagggatagtaaataa | |
| | | | | attgctcttcctaccccatcccccatcccctgacaaaaagtgacggcagccgt | |
| | | | | actgagtctgtaaggcccaaagtgggtacagacagcctgggctggtaaaagta | |
| | | | | ggtccttatttacaaggctgcgttaaagttgtactaggcaaacacactgatgt | |
| | | | | aggaagcacgaggaaaggaagacgttttgatatagtgttactgtgagcctgtc | |
| | | | | agtagtgggtaccaatcttttgtgacatattgtcatgctgaggtgtgacacct | |
| | | | | gctgcactcatctgatgtaaaaccatcccagagctggcgagaggatggagctgggtg | |
| | | | | gaaactgctttgcactatcgtttgctt | |
| | | 409 | CTGTTCTCTGGTTATAGCTGGTCCC | | |
| | | 410 | GGTCCCAGGTCAGCGTGGGAGGCAC | | |
| | | 411 | CACTTTGTAGTCTCATCCCAGATTA | | |
| | | 412 | ATCCCAGATTACTAACCCTTCCTGA | | |
| | | 413 | CAGCCGTACTGAGTCTGTAAGGCCC | | |
| | | 414 | AAGTGGGTACAGACAGCCTGGGCTG | | |
| | | 415 | TGAGCCTGTCAGTAGTGGGTACCAA | | |
| | | 416 | ACCTGCTGCACTCATCTGATGTAAA | | |
| | | 417 | TGTAAAACCATCCCAGAGCTGGCGA | | |
| | | 418 | AACTGCTTTGCACTATCGTTTGCTT | | |
| IFI6 | 204415_at | 419 | TGACCTTCATGGCCGTCGGAGGAGG | tgaccttcatggccgtcggaggaggactcgcagtcgccgggctgcccgcgctg | NM_022873.1 |
| | | | | ggcttcaccggcgccggcatcgcggccaactcggtggctgcctcgctgatgag | |
| | | | | ctggtctgcgatcctgaatgggggcggcgtgcccgccggggggctagtggcca | |
| | | | | cgctgcagagcctcggggctggtggcagcagcgtcgtcataggtaatattggt | |
| | | | | gccctgatgggctacgccacccacaagtatctcgatagtgaggaggatgagga | |
| | | | | gtagccagcagctcccagaacctcttcttccttcttggcctaactcttccagt | |
| | | | | taggatctagaactttgcctttttttttttttttttttttttttgagatgggt | |
| | | | | tctcactatattgtccaggctagagtgcagtggctattcacagatgcgaacat | |
| | | | | agtacactgcagcctccaactcctagcctcaagtgatcctcctgtctcaacct | |
| | | | | cccaagtaggattacaagcatgcgccgacgatgcccagaatccagaacttt | |
| | | 420 | TGGCAGCAGCGTCGTCATAGGTAAT | | |
| | | 421 | GTCGTCATAGGTAATATTGGTGCCC | | |
| | | 422 | ATTGGTGCCCTGATGGGCTACGCCA | | |
| | | 423 | GCCACCCACAAGTATCTCGATAGTG | | |
| | | 424 | GGATGAGGAGTAGCCAGCAGCTCCC | | |
| | | 425 | TTCTTGGCCTAACTCTTCCAGTTAG | | |
| | | 426 | AACTCTTCCAGTTAGGATCTAGAAC | | |
| | | 427 | GATGCGAACATAGTACACTGCAGCC | | |
| | | 428 | ATTACAAGCATGCGCCGACGATGCC | | |
| | | 429 | GACGATGCCCAGAATCCAGAACTTT | | |
| NPIP | 204538_x_at | 430 | CCTTCCACCCTCAGCGGATGATAAT | cagatgcaaaatcaccccttctgcaagaaagcctctttgcaaccgggtcagaa | NM_006985.1 |
| | | | | tggcggcagtggagcatcgtcattcttcaggattgccctactggccctacctc | |
| | | | | acagctgaaactttaaaaaacaggatgggccaccagccacctcctccaactca | |
| | | | | acaacattctataattgataactccctgagcctcaagacaccttccgagtgtc | |
| | | | | tgctcactccccttccaccctcagctctaccctcagcggatgataatctcaag | |
| | | | | acacctgcggagtgtctgctctatccccttccaccctcagcggatgataatct | |
| | | | | caagacacctcccgagtgtctgctcactccccttccaccctcagctccaccct | |
| | | | | cagcggatgataatctcaagacacctcccgagtgtgtctgctcactccccttccaccc | |
| | | | | tcagcggatgataat | |
| | | 431 | CAGATGCAAAATCACCCCTTCTGCA | | |
| | | 432 | GCCTCTTTGCAACCGGGTCAGAATG | | |
| | | 433 | GAATGGCGGCAGTGGAGCATCGTCA | | |
| | | 434 | CATCGTCATTCTTCAGGATTGCCCT | | |
| | | 435 | TGATAACTCCCTGAGCCTCAAGACA | | |
| | | 436 | AGCTCTACCCTCAGCGGATGATAAT | | |
| | | 437 | GACACCTGCGGAGTGTCTGCTCTAT | | |
| | | 438 | TGATAATCTCAAGACACCTCCCGAG | | |
| | | 439 | GCTCCACCCTCAGCGGATGATAATC | | |
| | | 440 | AAGACACCTCCCGAGTGTGTCTGCT | | |
| ADA | 204639_at | 441 | GTGGGGCTGAGCAACATTTTTACAT | gtggggctgagcaacatttttacatttattccttccaagaagaccatgatctc | NM_000022.1 |
| | | | | aatagtcagttactgatgctcctgaaccctatgtgtccatttctgcacacacg | |
| | | | | tatacctcggcatggccgcgtcacttctctgattatgtgccctggcagggacc | |
| | | | | agcgcccttgcacatgggcatggttgaatctgaaaccctccttctgtggcaacttgta | |
| | | | | ctga | |
| | | 442 | TTTTACATTTATTCCTTCCAAGAAG | | |
| | | 443 | GACCATGATCTCAATAGTCAGTTAC | | |
| | | 444 | GTCAGTTACTGATGCTCCTGAACCC | | |
| | | 445 | TGAACCCTATGTGTCCATTTCTGCA | | |
| | | 446 | ATGTGTCCATTTCTGCACACACGTA | | |
| | | 447 | GCGTCACTTCTCTGATTATGTGCCC | | |
| | | 448 | GATTATGTGCCCTGGCAGGGACCAG | | |
| | | 449 | CAGCGCCCTTGCACATGGGCATGGT | | |
| | | 450 | TGGTTGAATCTGAAACCCTCCTTCT | | |
| | | 451 | CTCCTTCTGTGGCAACTTGTACTGA | | |
| TGFBR3 | 204731_at | 452 | TGTATTTCTTACAGGCCTACAGAAA | tgtatttcttacaggcctacagaaattgaaaatgaccaaaatcaggaaccaca | NM_003243.1 |
| | | | | gatttgtgcccattcctaatattttgttctgcaaattaatgtataatttgagg | |
| | | | | tgaaattcagttataaagtcaaggacgaatttgcacagtgatatatttctatg | |
| | | | | tgtatgcaagtacaagtatataatatgtcacctggcacattcattttctcagt | |
| | | | | tgaagaagagaaaatttgaaaatgtccttatgcttttagagttgcaacttaag | |
| | | | | tatatttggtagggtgagtgtttccactcaaaatatgtcaacttaaaaaaaaa | |
| | | | | taggccctttcataaaaaccaaactgtagcaagatgcaaatgcatggcaaatc | |
| | | | | tgtcggtctccagttggttatctgaatagtgtcaccaattccaccaagacagtgctga | |
| | | | | gat | |
| | | 453 | GATTTGTGCCCATTCCTAATATTTT | | |
| | | 454 | GTCAAGGACGAATTTGCACAGTGAT | | |
| | | 455 | AAGTATATAATATGTCACCTGGCAC | | |
| | | 456 | CACCTGGCACATTCATTTTCTCAGT | | |
| | | 457 | AATGTCCTTATGCTTTTAGAGTTGC | | |
| | | 458 | TTTGGTAGGGTGAGTGTTTCCACTC | | |
| | | 459 | ATGCATGGCAAATCTGTCGGTCTCC | | |
| | | 460 | GTCGGTCTCCAGTTGGTTATCTGAA | | |
| | | 461 | GAATAGTGTCACCAATTCCACCAAG | | |
| | | 462 | AATTCCACCAAGACAGTGCTGAGAT | | |
| IARS | 204744_s_at | 463 | TTGGCCTTCGGAGCAGGAAGCTAAA | ttggccttcggagcaggaagctaaagctgtttctgaatgagacccaaacgcag | NM_013417.1 |
| | | | | gaaattacagaagacatccccgtgaagactttgaatatgaagactgtgtatgt | |
| | | | | ttctgtgttaccaacaacagcagacttctagcatgtacttatcaatgttgttc | |
| | | | | ggtcagcccttccctaattacacctatcccctacacatacatgcacatagaca | |
| | | | | cacacatgaacacactgaagatatttccttcaggtgtgtgtaaaatatgctgc | |
| | | | | ttggattgaaattcaaatgggattgattagtcaagtaacttgagacctcacag | |
| | | | | taatcttcacacttaaccttagacacctatgcagtcatgttgggagcaggtta | |
| | | | | caatgttacttcagcccacagtttatttctattcttgagttcttaagtacaga | |
| | | | | agatagaagtgatttaaatggcatagtatatatatcattttctggccttttaa | |
| | | | | aatttatttgagacctcttgatgaaatggacatattatatatttctgccacctggatt | |
| | | | | ttcctggata | |
| | | 464 | GAAGACATCCCCGTGAAGACTTTGA | | |
| | | 465 | GTTACCAACAACAGCAGACTTCTAG | | |
| | | 466 | GCAGACTTCTAGCATGTACTTATCA | | |
| | | 467 | TGAGACCTCACAGTAATCTTCACAC | | |
| | | 468 | CTTCACACTTAACCTTAGACACCTA | | |
| | | 469 | GACACCTATGCAGTCATGTTGGGAG | | |
| | | 470 | AGGTTACAATGTTACTTCAGCCCAC | | |
| | | 471 | TGTTACTTCAGCCCACAGTTTATTT | | |
| | | 472 | CATATTATATATTTCTGCCACCTGG | | |
| | | 473 | TCTGCCACCTGGATTTTCCTGGATA | | |
| LCK | 204891_s_at | 474 | GACTTGGGGAGATGGAGTTCTTGTG | gacttggggagatggagttcttgtgccatagtcacatggcctatgcacatatg | NM_005356.1 |
| | | | | gactctgcacatgaatcccacccacatgtgacacatatgcaccttgtgtctgt | |
| | | | | acacgtgtcctgtagttgcgtggactctgcacatgtcttgtgcatgtgtagcc | |
| | | | | tgtgcatgtatgtcttggacactgtacaaggtacccctttctggctctcccat | |
| | | | | ttcctgagaccaccagagagaggggagaagcctgggattgacagaagcttctg | |
| | | | | cccacctacttttctttcctcagatcatccagaagttcctgaagggccaggactttat | |
| | | | | ctaatacctctgtgtgctc | |
| | | 475 | TGGAGTTCTTGTGCCATAGTCACAT | | |
| | | 476 | TATGGACTCTGCACATGAATCCCAC | | |
| | | 477 | AATCCCACCCACATGTGACACATAT | | |
| | | 478 | ACATATGCACCTTGTGTCTGTACAC | | |
| | | 479 | TGTGTAGCCTGTGCATGTATGTCTT | | |
| | | 480 | GCATGTATGTCTTGGACACTGTACA | | |
| | | 481 | CACTGTACAAGGTACCCCTTTCTGG | | |
| | | 482 | GCCTGGGATTGACAGAAGCTTCTGC | | |
| | | 483 | GGGCCAGGACTTTATCTAATACCTC | | |
| | | 484 | CTTTATCTAATACCTCTGTGTGCTC | | |
| IL10RA | 204912_at | 485 | TAGGCCATTTGGACTCTGCCTTCAA | taggccatttggactctgccttcaaacaaaggcagttcagtccacaggcatgg | NM_001558.1 |
| | | | | aagctgtgaggggacaggcctgtgcgtgccatccagagtcatctcagccctgc | |
| | | | | ctttctctggagcattctgaaaacagatattctggcccagggaatccagccat | |
| | | | | gacccccacccctctgccaaagtactcttaggtgccagtctggtaactgaact | |
| | | | | ccctctggaggcaggcttgagggaggattcctcagggttcccttgaaagcttt | |
| | | | | atttatttattttgttcatttatttattggagaggcagcattgcacagtgaaa | |
| | | | | gaattctggatatctcaggagccccgaaattctagctctgactttgctgtttc | |
| | | | | cagtggtatgaccttggagaagtcacttatcctcttggagcctcagtttcctc | |
| | | | | atctgcagaataatgactgacttgtctaattcatagggatgtgaggttctgctgagg | |
| | | 486 | GGCAGTTCAGTCCACAGGCATGGAA | | |
| | | 487 | CTGGCCCAGGGAATCCAGCCATGAC | | |
| | | 488 | AGTACTCTTAGGTGCCAGTCTGGTA | | |
| | | 489 | GTAACTGAACTCCCTCTGGAGGCAG | | |
| | | 490 | TCAGGGTTCCCTTGAAAGCTTTATT | | |
| | | 491 | ATTCTGGATATCTCAGGAGCCCCGA | | |
| | | 492 | GGAGCCCCGAAATTCTAGCTCTGAC | | |
| | | 493 | GCTGTTTCCAGTGGTATGACCTTGG | | |
| | | 494 | AGAAGTCACTTATCCTCTTGGAGCC | | |
| | | 495 | ATAGGGATGTGAGGTTCTGCTGAGG | | |
| FCN1 | 205237_at | 496 | GGTATCAACTGGAGTGCGGCGAAGG | gagggcaaccaccagtttgctaagtacaaatcattcaaggtggctgacgaggc | NM_002003.2 |
| | | | | agagaagtacaagctggtactgggagcctttgtcgggggcagtgcgggtaat | |
| | | | | ctctaacgggccacaacaacaacttcttctccaccaaagaccaagacaatgat | |
| | | | | gtgagttcttcgaattgtgctgagaagttccagggagcctggtggtacgccga | |
| | | | | ctgtcatgcttcaaacctcaatggtctctacctcatgggaccccatgagagct | |
| | | | | atgccaatggtatcaactggagtgcggcgaaggggtacaaatatagctacaag | |
| | | | | gtgtcagagatgaaggtgcggcccgcctagacgggccaggacccctccacatg | |
| | | | | cacctgctagtggggaggccacacccacaagcgctgcgtcgtggaag | |
| | | 497 | CCTCCACATGCACCTGCTAGTGGGG | | |
| | | 498 | ACCCACAAGCGCTGCGTCGTGGAAG | | |
| | | 499 | GAGGGCAACCACCAGTTTGCTAAGT | | |
| | | 500 | GGGCAGTGCGGGTAATTCTCTAACG | | |
| | | 501 | TTCTCTAACGGGCCACAACAACAAC | | |
| | | 502 | GTGAGTTCTTCGAATTGTGCTGAGA | | |
| | | 503 | TCCAGGGAGCCTGGTGGTACGCCGA | | |
| | | 504 | GACTGTCATGCTTCAAACCTCAATG | | |
| | | 505 | CTCAATGGTCTCTACCTCATGGGAC | | |
| | | 506 | ATGGGACCCCATGAGAGCTATGCCA | | |
| IL2RB | 205291_at | 507 | GACAAGCGTTGAGCCACTAAGCAGA | gacaagcgttgagccactaagcagaggaccttgggttcccaatacaaaaatac | NM_000878.1 |
| | | | | ctactgctgagagggctgctgaccatttggtcaggattcctgttgcctttata | |
| | | | | tccaaaataaactcccctttcttgaggttgtctgagtcttgggtctatgcctt | |
| | | | | gaaaaaagctgaattattggacagtctcacctcctgccatagggtcctgaatg | |
| | | | | tttcagaccacaaggggctccacacctttgctgtgtgttctggggcaacctac | |
| | | | | taatcctctctgcaagtcggtctccttatccccccaaatggaaattgtatttg | |
| | | | | ccttctccactttgggaggctcccacttcttgggagggttacattttttaagt | |
| | | | | cttaatcatttgtgacatatgtatctatacatccgtatcttttaatgatccgtgtgta | |
| | | | | ccatctttgtgat | |
| | | 508 | TAAGCAGAGGACCTTGGGTTCCCAA | | |
| | | 509 | TGAGAGGGCTGCTGACCATTTGGTC | | |
| | | 510 | GATTCCTGTTGCCTTTATATCCAAA | | |
| | | 511 | CTTTCTTGAGGTTGTCTGAGTCTTG | | |
| | | 512 | CTGAGTCTTGGGTCTATGCCTTGAA | | |
| | | 513 | AATTATTGGACAGTCTCACCTCCTG | | |
| | | 514 | CCTCCTGCCATAGGGTCCTGAATGT | | |
| | | 515 | TTTGCTGTGTGTTCTGGGGCAACCT | | |
| | | 516 | GGAAATTGTATTTGCCTTCTCCACT | | |
| | | 517 | GATCCGTGTGTACCATCTTTGTGAT | | |
| GNA15 | 205349_at | 518 | AACGGCCATTTGGGATGCCAGGGTG | aacggccatttgggatgccagggtggatgaaaaggtgaagaaatcaggggatt | NM_002068.1 |
| | | | | gagacttgggtgggtgggcatctctcaggagccccatctccgggcgtgtcacc | |
| | | | | tcctgggcagggttctgggaccctctgtgggtgacgcacaccctgggatgggg | |
| | | | | ctagtagagccttcaggcgccttcgggcgtggactctggcgcactctagtgga | |
| | | | | caggagaaggaacgccttccaggaacctgtggactaggggtgcagggacttcc | |
| | | | | ctttgcaaggggtaacagaccgctggaaaacactgtcactttcagagctcggt | |
| | | | | ggctcacagcgtgtcctgccccggtttgcggacgagagaaatcgcggcccaca | |
| | | | | agcatcccccatcccttgcaggctgggggctgggcatgctgcatcttaaccttttgta | |
| | | | | tttat | |
| | | 519 | GAGACTTGGGTGGGTGGGCATCTCT | | |
| | | 520 | TGACGCACACCCTGGGATGGGGCTA | | |
| | | 521 | GGGCTAGTAGAGCCTTCAGGCGCCT | | |
| | | 522 | ACTCTGGCGCACTCTAGTGGACAGG | | |
| | | 523 | GAACGCCTTCCAGGAACCTGTGGAC | | |
| | | 524 | CTAGGGGTGCAGGGACTTCCCTTTG | | |
| | | 525 | CAGACCGCTGGAAAACACTGTCACT | | |
| | | 526 | AACACTGTCACTTTCAGAGCTCGGT | | |
| | | 527 | GCGGACGAGAGAAATCGCGGCCCAC | | |
| | | 528 | CTGCATCTTAACCTTTTGTATTTAT | | |
| GZMA | 205488_at | 529 | CAGCCACACGCGAAGGTGACCTTAA | cagccacacgcgaaggtgaccttaaacttttacagctgacggaaaaagcaaaa | NM_006144.2 |
| | | | | attaacaaatatgtgactatccttcatctacctaaaaagggggatgatgtgaa | |
| | | | | accaggaaccatgtgccaagttgcagggtgggggaggactcacaatagtgcat | |
| | | | | cttggtccgatactctgagagaagtcaatatcaccatcatagacagaaaagtc | |
| | | | | tgcaatgatcgaaatcactataattttaaccctgtgattggaatgaatatggt | |
| | | | | ttgtgctggaagcctccgaggtggaagagactcgtgcaatggagattctggaa | |
| | | | | gccctttgttgtgcgagggtgttttccgaggggtcacttcctttggccttgaa | |
| | | | | aataaatgcggagaccctcgtgggcctggtgtctatattcttctctcaaagaaacacc | |
| | | | | tcaactgga | |
| | | 530 | GACCTTAAACTTTTACAGCTGACGG | | |
| | | 531 | TATGTGACTATCCTTCATCTACCTA | | |
| | | 532 | GGAACCATGTGCCAAGTTGCAGGGT | | |
| | | 533 | GACTCACAATAGTGCATCTTGGTCC | | |
| | | 534 | GCATCTTGGTCCGATACTCTGAGAG | | |
| | | 535 | TGCTGGAAGCCTCCGAGGTGGAAGA | | |
| | | 536 | TGTTGTGCGAGGGTGTTTTCCGAGG | | |
| | | 537 | AATAAATGCGGAGACCCTCGTGGGC | | |
| | | 538 | CTGGTGTCTATATTCTTCTCTCAAA | | |
| | | 539 | CTCTCAAAGAAACACCTCAACTGGA | | |
| KLRK1 | 205821_at | 540 | AGGCAATTCAGATATCCCCAAGGCT | aggcaattcagatatccccaaggctgcctctcccaccacaagcccagagtgga | NM_007360.1 |
| | | | | tgggctgggggaggggtgctgttttaatttctaaaggtaggaccaacacccag | |
| | | | | gggatcagtgaaggaagagaaggccagcagatcagtgagagtgcaaccccacc | |
| | | | | ctccacaggaaattgcctcatgggcagggccacagcagagagacacagcatgg | |
| | | | | gcagtgccttccctgcctgtgggggtcatgctgccacttttaatgggtcctcc | |
| | | | | acccaacggggtcagggaggtggtgctgccccagtgggccatgattatcttaa | |
| | | | | aggcattattctccagccttaagatcttaggacgtttcctttgctatgatttg | |
| | | | | tacttgcttgagtcccatgactgtttctcttcctctctttcttccttttggaa | |
| | | | | tagtaatatccatcctatgtttgtcccactattgta | |
| | | 541 | GTAGGACCAACACCCAGGGGATCAG | | |
| | | 542 | AGATCAGTGAGAGTGCAACCCCACC | | |
| | | 543 | CACCCTCCACAGGAAATTGCCTCAT | | |
| | | 544 | AATTGCCTCATGGGCAGGGCCACAG | | |
| | | 545 | CTGCCACTTTTAATGGGTCCTCCAC | | |
| | | 546 | GGCATTATTCTCCAGCCTTAAGATC | | |
| | | 547 | GATCTTAGGACGTTTCCTTTGCTAT | | |
| | | 548 | GATTTGTACTTGCTTGAGTCCCATG | | |
| | | 549 | TTGAGTCCCATGACTGTTTCTCTTC | | |
| | | 550 | ATCCTATGTTTGTCCCACTATTGTA | | |
| CD2 | 205831_at | 551 | AGACCTCGAGTTCAGCCAAAACCTC | agacctcgagttcagccaaaacctccccatggggcagcagaaaactcattgtc | NM_001767.1 |
| | | | | cccttcctctaattaaaaaagatagaaactgtctttttcaataaaaagcactg | |
| | | | | tggatttctgccctcctgatgtgcatatccgtacttccatgaggtgttttctg | |
| | | | | tgtgcagaacattgtcacctcctgaggctgtgggccacagccacctctgcatc | |
| | | | | ttcgaactcagccatgtggtcaacatctggagtttttggtctcctcagagagc | |
| | | | | tccatcacaccagtaaggagaagcaatataagtgtgattgcaagaatggtaga | |
| | | | | ggaccgagcacagaaatcttagagatttcttgtcccctctcaggtcatgtgta | |
| | | | | gatgcgataaatcaagtgattggtgtgcctgggtctcactacaagcagcctatctgc | |
| | | 552 | GGCAGCAGAAAACTCATTGTCCCCT | | |
| | | 553 | AAAAGCACTGTGGATTTCTGCCCTC | | |
| | | 554 | CTGATGTGCATATCCGTACTTCCAT | | |
| | | 555 | GTACTTCCATGAGGTGTTTTCTGTG | | |
| | | 556 | TGTGCAGAACATTGTCACCTCCTGA | | |
| | | 557 | GAGTTTTTGGTCTCCTCAGAGAGCT | | |
| | | 558 | AGAGCTCCATCACACCAGTAAGGAG | | |
| | | 559 | AATCTTAGAGATTTCTTGTCCCCTC | | |
| | | 560 | TCCCCTCTCAGGTCATGTGTAGATG | | |
| | | 561 | GTCTCACTACAAGCAGCCTATCTGC | | |
| CX3CR1 | 205898_at | 562 | AGCCCCTGCCCATCTGGGAAAATAC | agcccctgcccatctgggaaaataccccatcattcatgctactgccaacctgg | U20350.1 |
| | | | | ggagccagggctatgggagcagcttttttttcccccctagaaacgtttggaac | |
| | | | | aatgtaaaactttaaagctcgaaaacaattgtaataatgctaaagaaaaagtc | |
| | | | | atccaatctaaccacatcaatattgtcattcctgtattcacccgtccagacct | |
| | | | | tgttcacactctcacatgtttagagttgcaatcgtaatgtacagatggtttta | |
| | | | | taatctgatttgttttcctcttaacgttagaccacaaatagtgctcgctttct | |
| | | | | atgtagtttggtaattatcattttagaagactctaccagactgtgtattcatt | |
| | | | | gaagtcagatgtggtaactgttaaattgctgtgtatctgatagctctttggca | |
| | | | | gtctatatgtttgtataatgaatgagagaataagtcatgttccttcaagatcatgtac | |
| | | | | cccaatttacttgccattact | |
| | | 563 | GAAAATACCCCATCATTCATGCTAC | | |
| | | 564 | GGCTATGGGAGCAGCTTTTTTTTCC | | |
| | | 565 | GTCATCCAATCTAACCACATCAATA | | |
| | | 566 | CTTGTTCACACTCTCACATGTTTAG | | |
| | | 567 | TTATAATCTGATTTGTTTTCCTCTT | | |
| | | 568 | GACCACAAATAGTGCTCGCTTTCTA | | |
| | | 569 | GTGCTCGCTTTCTATGTAGTTTGGT | | |
| | | 570 | GAAGACTCTACCAGACTGTGTATTC | | |
| | | 571 | TGTTCCTTCAAGATCATGTACCCCA | | |
| | | 572 | GTACCCCAATTTACTTGCCATTACT | | |
| HK3 | 205936_s_at | 573 | AGGTCCGAGCCATCCTAGAGGATCT | aggtccgagccatcctagaggatctggggctacccctgacctcagatgacgcc | NM_002115.1 |
| | | | | ctgatggtgctagaggtgtgccaggctgtgtcccagagggctgcccagctctg | |
| | | | | tggggcgggtgtagctgccgtggtggagaagatccgggggaaccggggcctgg | |
| | | | | aagagctggcagtgtctgtgggggtggatggaacgctctacaagctgcacccg | |
| | | | | cgcttctccagcctggtggcggccacagtgcgggagctggcccctcgctgtgt | |
| | | | | ggtcacgttcctgcagtcagaggatgggtccggcaaaggtgcggccctggtca | |
| | | | | ccgctgttgcctgccgccttgcgcagttgactcgtgtctgaggaaacctccag | |
| | | | | gctgaggaggtctccgccgcagccttgctggagccgggtcggggtctgcctgt | |
| | | | | ttcccagccaggcccagccacccaggactcctgggacatcccatgtgtgaccc | |
| | | | | ctctgcggccatttggccttgctccctggctttccctgagagaagtagcactcaggtt | |
| | | | | agcaatat | |
| | | 574 | CTAGAGGATCTGGGGCTACCCCTGA | | |
| | | 575 | TGACCTCAGATGACGCCCTGATGGT | | |
| | | 576 | GACGCCCTGATGGTGCTAGAGGTGT | | |
| | | 577 | GGTGTAGCTGCCGTGGTGGAGAAGA | | |
| | | 578 | GTCTGTGGGGGTGGATGGAACGCTC | | |
| | | 579 | GATGGAACGCTCTACAAGCTGCACC | | |
| | | 580 | GGATGGGTCCGGCAAAGGTGCGGCC | | |
| | | 581 | TGACTCGTGTCTGAGGAAACCTCCA | | |
| | | 582 | GACTCCTGGGACATCCCATGTGTGA | | |
| | | 583 | GAAGTAGCACTCAGGTTAGCAATAT | | |
| ING2 | 205981_s_at | 584 | GATGGATTCCAGCCAACCAGAAAGA | gatggattccagccaaccagaaagatcttcaagaagaccccgcaggcagcgga | NM_001564.1 |
| | | | | ccagtgaaagccgtgatttatgtcacatggcaaatgggattgaagactgtgat | |
| | | | | gatcagccacctaaagaaaagaaatccaagtcagcaaagaaaaagaaacgctc | |
| | | | | caaggccaagcaggaaagggaagcttcacctgttgagtttgcaatagatccta | |
| | | | | atgaacctacatactgcttatgcaaccaagtgtcttatggggagatgatagga | |
| | | | | tgtgacaatgaacagtgtccaattgaatggtttcacttttcatgtgtttcact | |
| | | | | tacctataaaccaaaggggaaatggtattgcccaaagtgcaggggagataatg | |
| | | | | agaaaacaatggacaaaagtactgaaaagacaaaaaaggatagaagatcgaggtagta | |
| | | | | aaggccatccacattt | |
| | | 585 | GGCAGCGGACCAGTGAAAGCCGTGA | | |
| | | 586 | AAAGCCGTGATTTATGTCACATGGC | | |
| | | 587 | AAGACTGTGATGATCAGCCACCTAA | | |
| | | 588 | GAAAAAGAAACGCTCCAAGGCCAAG | | |
| | | 589 | GGGAAGCTTCACCTGTTGAGTTTGC | | |
| | | 590 | GATCCTAATGAACCTACATACTGCT | | |
| | | 591 | TACTGCTTATGCAACCAAGTGTCTT | | |
| | | 592 | TTTCATGTGTTTCACTTACCTATAA | | |
| | | 593 | GGGGAAATGGTATTGCCCAAAGTGC | | |
| | | 594 | GAGGTAGTAAAGGCCATCCACATTT | | |
| STAT4 | 206118_at | 595 | GCTGACATCCTGCGAGACTACAAAG | gctgacatcctgcgagactacaaagttattatggctgaaaacattcctgaaaa | NM_003151.1 |
| | | | | ccctctgaagtacctatatcctgacattcccaaagacaaagccttcggtaaac | |
| | | | | actacagctctcagccttgcgaagtttcaagaccaacagaaaggggtgacaaa | |
| | | | | ggttatgttccttctgtttttatccccatctcaacaatccgaagtgattcaac | |
| | | | | agagccacattctccatcagaccttcttcccatgtctccaagtgtgtatgcgg | |
| | | | | tgttgagagaaaacctgagtcccacaacaattgaaactgcaatgaagtctcct | |
| | | | | tattctgctgaatgacaggataaactctgacgcaccaagaaaggaagcaaatg | |
| | | | | aaaaagtttaaagactgttctttgcccaataaccacattttatttcttcagct | |
| | | | | ttgtaaataccaggttctaggaaatgtttgacatctgaagctctcttcacactcccgt | |
| | | | | ggcactcctcaattgggag | |
| | | 596 | TCCTGAAAACCCTCTGAAGTACCTA | | |
| | | 597 | GAAGTACCTATATCCTGACATTCCC | | |
| | | 598 | CAAAGCCTTCGGTAAACACTACAGC | | |
| | | 599 | GCTCTCAGCCTTGCGAAGTTTCAAG | | |
| | | 600 | TCCCCATCTCAACAATCCGAAGTGA | | |
| | | 601 | AAACCTGAGTCCCACAACAATTGAA | | |
| | | 602 | TGCAATGAAGTCTCCTTATTCTGCT | | |
| | | 603 | AGACTGTTCTTTGCCCAATAACCAC | | |
| | | 604 | GACATCTGAAGCTCTCTTCACACTC | | |
| | | 605 | TCCCGTGGCACTCCTCAATTGGGAG | | |
| CD33 | 206120_at | 606 | GAGGAGCTGCATTATGCTTCCCTCA | agtgggcagcaatgacacccaccctaccacagggtcagcctccccgaaacacc | NM_001772.1 |
| | | | | agaagaactccaagttacatggccccactgaaacctcaagctgttcaggtgcc | |
| | | | | gcccctactgtggagatggatgaggagctgcattatgcttccctcaactttca | |
| | | | | tgggatgaatccttccaaggacacctccaccgaatactcagaggtcaggaccc | |
| | | | | agtgaggaaccctcaagagcatcaggctcagctagaagatccacatcctctac | |
| | | | | aggtcggggaccaaaggctgattcttggagatttaactccccacaggcaatgg | |
| | | | | gtttatagacattatgtgagtttcctgctatattaacatcatcttgagacttt | |
| | | | | gcaagcagagagtcgtggaatcaaatctgtgctctttcatt | |
| | | 607 | ATGCTTCCCTCAACTTTCATGGGAT | | |
| | | 608 | ATGAATCCTTCCAAGGACACCTCCA | | |
| | | 609 | GACACCTCCACCGAATACTCAGAGG | | |
| | | 610 | AGGAACCCTCAAGAGCATCAGGCTC | | |
| | | 611 | TAGAAGATCCACATCCTCTACAGGT | | |
| | | 612 | AGGTCGGGGACCAAAGGCTGATTCT | | |
| | | 613 | GGAATCAAATCTGTGCTCTTTCATT | | |
| | | 614 | AGTGGGCAGCAATGACACCCACCCT | | |
| | | 615 | AGAACTCCAAGTTACATGGCCCCAC | | |
| | | 616 | AAACCTCAAGCTGTTCAGGTGCCGC | | |
| ASGR2 | 206130_s_at | 617 | TGCAGGTGTACCGCTGGGTGTGTGA | ggagaacgcacacctggtggtcatcaactcctgggaggagcagaaattcattg | NM_001181.1 |
| | | | | tacaacacacgaaccccttcaatacctggataggtctcacggacagtgatggc | |
| | | | | tcttggaaatgggtggatggcacagactataggcacaactacaagaactgggc | |
| | | | | tgtcactcagccagataattggcacgggcacgagctgggtggaagtgaagact | |
| | | | | gtgttgaagtccagccggatggccgctggaacgatgacttctgcctgcaggtg | |
| | | | | taccgctgggtgtgtgagaaaaggcggaatgccaccggcgaggtggcctgacc | |
| | | | | ccagcacacctctggctaacccataccccacacctgcccagctctggcttctc | |
| | | | | tgttgaggattttgaggaaaggaagaaacactgagacaggggtatggggaaga | |
| | | | | gctgagcaaagagagaaaggaggtagtttaagagtccctgaccctggaggact | |
| | | | | gagatcccacctccttctgtaattcattgtaattattataatcgtcagcctcttcaa | |
| | | 618 | ATGCCACCGGCGAGGTGGCCTGACC | | |
| | | 619 | GGTAGTTTAAGAGTCCCTGACCCTG | | |
| | | 620 | CCCTGGAGGACTGAGATCCCACCTC | | |
| | | 621 | TTATTATAATCGTCAGCCTCTTCAA | | |
| | | 622 | GGAGAACGCACACCTGGTGGTCATC | | |
| | | 623 | TCATTGTACAACACACGAACCCCTT | | |
| | | 624 | GAACCCCTTCAATACCTGGATAGGT | | |
| | | 625 | TAATTGGCACGGGCACGAGCTGGGT | | |
| | | 626 | TGTTGAAGTCCAGCCGGATGGCCGC | | |
| | | 627 | GGCCGCTGGAACGATGACTTCTGCC | | |
| MATK | 206267_s_at | 628 | GCCGAGCGGAAGGGGCTAGACTCAA | gccgagcggaaggggctagactcaagccggctgcccgtcaagtggacggcgcc | NM_002378.1 |
| | | | | cgaggctctcaaacacgggttcaccagcaagtcggatgtctggagttttgggg | |
| | | | | tgctgctctgggaggtcttctcatatggacgggctccgtaccctaaaatgtca | |
| | | | | ctgaaagaggtgtcggaggccgtggagaaggggtaccgcatggaaccccccga | |
| | | | | gggctgtccaggccccgtgcacgtcctcatgagcagctgctgggaggcagagc | |
| | | | | cgcccgccggccacccttccgcaaactggccgagaagctggcccgggagctac | |
| | | | | gcagtgcaggtgccccagcctccgtctcagggcaggacgccgacggtccacct | |
| | | | | cgccccgaagccaggagccctgaccccacccggtggcccttggccccagaggaccgag | |
| | | | | agagtggagagtgcggcgtgggggcac | |
| | | 629 | GAAGGGGCTAGACTCAAGCCGGCTG | | |
| | | 630 | GTTCACCAGCAAGTCGGATGTCTGG | | |
| | | 631 | GCAAGTCGGATGTCTGGAGTTTTGG | | |
| | | 632 | CTCTGGGAGGTCTTCTCATATGGAC | | |
| | | 633 | TCTTCTCATATGGACGGGCTCCGTA | | |
| | | 634 | GCTCCGTACCCTAAAATGTCACTGA | | |
| | | 635 | CTGAAAGAGGTGTCGGAGGCCGTGG | | |
| | | 636 | AGGCCGTGGAGAAGGGGTACCGCAT | | |
| | | 637 | CCGTCTCAGGGCAGGACGCCGACGG | | |
| | | 638 | AGTGGAGAGTGCGGCGTGGGGGCAC | | |
| ASGR1 | 206743_s_at | 639 | CTACCGCTGGGTCTGCGAGACAGAG | aggagcagaaatttgtccagcaccacataggccctgtgaacacctggatgggc | NM_001671.1 |
| | | | | ctccacgaccaaaacgggccctggaagtgggtggacgggacggactacgagac | |
| | | | | gggcttcaagaactggaggccggagcagccggacgactggtacggccacgggc | |
| | | | | tcggaggaggcgaggactgtgcccacttcaccgacgacggccgctggaacgac | |
| | | | | gacgtctgccagaggccctaccgctgggtctgcgagacagagctggacaaggc | |
| | | | | cagccaggagccacctctcctttaatttatttcttcaatgcctcgacctgccg | |
| | | | | caggggtccgggattgggaatccgcccatctggggcctcttctgctttctcgg | |
| | | | | gaattttcatctaggattttaagggaaggggaaggatagggtgatgttccgaaggtga | |
| | | | | ggagcttgaaacccgtggcg | |
| | | 640 | GGGTCTGCGAGACAGAGCTGGACAA | | |
| | | 641 | TGCCGCAGGGGTCCGGGATTGGGAA | | |
| | | 642 | TCTTCTGCTTTCTCGGGAATTTTCA | | |
| | | 643 | CTCGGGAATTTTCATCTAGGATTTT | | |
| | | 644 | GATAGGGTGATGTTCCGAAGGTGAG | | |
| | | 645 | GGTGAGGAGCTTGAAACCCGTGGCG | | |
| | | 646 | AGGAGCAGAAATTTGTCCAGCACCA | | |
| | | 647 | GAAATTTGTCCAGCACCACATAGGC | | |
| | | 648 | CCACATAGGCCCTGTGAACACCTGG | | |
| | | 649 | GAGCAGCCGGACGACTGGTACGGCC | | |
| TXK | 206828_at | 650 | TAGCCCCAGGAACCCTTGAGGTTCT | tagccccaggaacccttgaggttcttcttcacaaggctgagagtgcttccttc | NM_003328.1 |
| | | | | ttgaagacgagtgtcattcatcacttcagtgatccatgcatagaatatgaaaa | |
| | | | | taaattcttccaactcatgggataaaggggactcccttgaagaatttcatgtt | |
| | | | | tttgggctgtatagctctttacagaaaatgcacctttataaatcacatgaatg | |
| | | | | ttagtattctggaaatgtcttttgttaatataatcttcccatgttatttaaca | |
| | | | | aattgtttttgcacatatctgattatattgaaagcagtttttttgcattcgag | |
| | | | | ttttaaacactgttataaaatgtagccaaagctcacctttgaacagatcccgg | |
| | | | | tgacattctatttccaggaaaatccggaacctgattttagttctgtgatttta | |
| | | | | cactttttacatgtgagattggacagtttcagaggccttattttgtcatactaagtg | |
| | | | | tctcctgtaatt | |
| | | 651 | TTGAGGTTCTTCTTCACAAGGCTGA | | |
| | | 652 | GACGAGTGTCATTCATCACTTCAGT | | |
| | | 653 | TCACTTCAGTGATCCATGCATAGAA | | |
| | | 654 | GGGACTCCCTTGAAGAATTTCATGT | | |
| | | 655 | GTTTTTGGGCTGTATAGCTCTTTAC | | |
| | | 656 | AGCTCACCTTTGAACAGATCCCGGT | | |
| | | 657 | TCCCGGTGACATTCTATTTCCAGGA | | |
| | | 658 | GAGATTGGACAGTTTCAGAGGCCTT | | |
| | | 659 | TCAGAGGCCTTATTTTGTCATACTA | | |
| | | 660 | GTCATACTAAGTGTCTCCTGTAATT | | |
| KIR3DL2 | 207314_x_at | 661 | GGAACTTCCAAATGCTGAGCCCAGA | ggaacttccaaatgctgagcccagatccaaagttgtctcctgcccacgagcac | NM_006737.1 |
| | | | | cacagtcaggtcttgagggggttttctagggagacaacagccctgtctcaaaa | |
| | | | | ccaggttgccagatccaatgaaccagcagctggaatctgaaggcatcagtctg | |
| | | | | catcttaggggatcgctcttcctcacaccacgaatctgaacatgcctctctct | |
| | | | | tgcttacaaatgcctaaggtcgccactgcctgctgcagagaaaacacactcct | |
| | | | | ttgcttagcccacaaggtatctatttcacttgacccctgcccacctctccaac | |
| | | | | ctaactggcttacttcctagtcctacttgaggctgcaatcacactgaggaact | |
| | | | | cacaattccaaacatacaagaggctccctcttaacacggcacttacacacttg | |
| | | | | ctgttccaccttccctcatgctgttccacctcccctcagactatctttcagcc | |
| | | | | ttctgtcatcagtaaaatttataaattttttttataacttcagtgtagctctctcct | |
| | | 662 | AGCCCAGATCCAAAGTTGTCTCCTG | | |
| | | 663 | CCACGAGCACCACAGTCAGGTCTTG | | |
| | | 664 | CAGTCTGCATCTTAGGGGATCGCTC | | |
| | | 665 | ACACCACGAATCTGAACATGCCTCT | | |
| | | 666 | AAGGTATCTATTTCACTTGACCCCT | | |
| | | 667 | TCTCCAACCTAACTGGCTTACTTCC | | |
| | | 668 | CTTCCTAGTCCTACTTGAGGCTGCA | | |
| | | 669 | AACACGGCACTTACACACTTGCTGT | | |
| | | 670 | TATCTTTCAGCCTTCTGTCATCAGT | | |
| | | 671 | TATAACTTCAGTGTAGCTCTCTCCT | | |
| SH2D2A | 207351_s_at | 672 | CACCCTGTCCTACGGAAGAGCTGGT | caccctgtcctacggaagagctggtccaggcctgtcccaggaggccagaatac | NM_003975.1 |
| | | | | aggtggctcccagctgcattctgagaactctgtgattgggcaaggccctcccc | |
| | | | | tgccccaccagcccccacccgcctggagacacaccctcccccacaatctttct | |
| | | | | agacaggtgcttcaggacagaggacaggcatggcttccccttgggcctcctca | |
| | | | | gtaggcggtctggcctgacccccaacaaagaagcctggaggtcagagaagcaa | |
| | | | | atgcggagcctgctccctcctaagaagatcccaagaatccaatggctcagtcc | |
| | | | | ttggtgatctaagacagcaaagaagtgtgcaaggagggccctgttagctccca | |
| | | | | ctgtcctggtttctcctcctggagtctaatttccttggccctctgagcctttt | |
| | | | | gagtctgggccctggtccaatgctgctgttgtctgaggaatggtttggtgaga | |
| | | | | acagatgttagaacttgtttgttgattcttgtctggctaat | |
| | | 673 | CTCCCAGCTGCATTCTGAGAACTCT | | |
| | | 674 | GAACTCTGTGATTGGGCAAGGCCCT | | |
| | | 675 | TCCCCCACAATCTTTCTAGACAGGT | | |
| | | 676 | AAGCAAATGCGGAGCCTGCTCCCTC | | |
| | | 677 | CTCCCTCCTAAGAAGATCCCAAGAA | | |
| | | 678 | CAATGGCTCAGTCCTTGGTGATCTA | | |
| | | 679 | GTGCAAGGAGGGCCCTGTTAGCTCC | | |
| | | 680 | TCCTGGAGTCTAATTTCCTTGGCCC | | |
| | | 681 | CTCTGAGCCTTTTGAGTCTGGGCCC | | |
| | | 682 | GTTTGTTGATTCTTGTCTGGCTAAT | | |
| CD160 | 207840_at | 683 | AACAGAACAGCTTTCACCAAAGTGG | tcagtgtaatccttgactttgctcctcaccatcagggcaaacttgccttcttc | NM_007053.1 |
| | | | | cctcctaagctccagtaaataaacagaacagctttcaccaaagtgggtagtat | |
| | | | | agtcctcaaatatcggataaatatatgcgtttttgtaccccagaaaaactttt | |
| | | | | cctccctcttcatcaacatagtaaaataagtcaaacaaaatgagaacaccaaa | |
| | | | | ttttgggggaataaatttttatttaacactgcaaaggaaagagagagaaaaca | |
| | | | | agcaaagataggtaggacagaaaggaagacagccagatccagtgattgacttg | |
| | | | | gcatgaaaatgagaaaatgcagacagacctcaacattcaacattcaacaacat | |
| | | | | ccatacagcactgctggaggaagaggaagatttgtgcagaccaagagcaccac | |
| | | | | agactacaactgcccagcttcatctaaatacttgttaacctctttggtcat | |
| | | 684 | GTGGGTAGTATAGTCCTCAAATATC | | |
| | | 685 | ATATATGCGTTTTTGTACCCCAGAA | | |
| | | 686 | GACAGCCAGATCCAGTGATTGACTT | | |
| | | 687 | CAACAACATCCATACAGCACTGCTG | | |
| | | 688 | AAGAGCACCACAGACTACAACTGCC | | |
| | | 689 | TACAACTGCCCAGCTTCATCTAAAT | | |
| | | 690 | GCCCAGCTTCATCTAAATACTTGTT | | |
| | | 691 | AATACTTGTTAACCTCTTTGGTCAT | | |
| | | 692 | TCAGTGTAATCCTTGACTTTGCTCC | | |
| | | 693 | TTCCCTCCTAAGCTCCAGTAAATAA | | |
| CEACAM3 | 208052_x_at | 694 | ATACCAAGAAAATGCCCCAGGCCTT | ataccaagaaaatgccccaggccttcctgtgggggccgtcgccggcatcgtga | NM_001815.1 |
| | | | | ccggggtcctggtcggagtggcgctggtggccgcgctggtgtgtttcctgctc | |
| | | | | cttgccaaaactggaaggccgtggtccctcccacagctctgccttctcgatgt | |
| | | | | cccctctctccactgcctaggcccccctacccaaccccaggacagcagcttcc | |
| | | | | atctatgagaagtggcttcttagcttcctccaggagctgctcctgtgggttga | |
| | | | | tggagagtccccaaggcccccagccctggggatggggaaggacatgaagcctg | |
| | | | | agccagagaaccagctataagtcctgagaagacactggtgtctggggacaggg | |
| | | | | agggatggggtccctgatgaatatctggagacctcgacagcctgccctaggcc | |
| | | | | ctgggtgggtcaggacaaaggcctctcatcaccgcagaaagcgggggcttgcagggaa | |
| | | | | agtgaatgggcctgtggcccacctg | |
| | | 695 | TTCCTGCTCCTTGCCAAAACTGGAA | | |
| | | 696 | CCAGGACAGCAGCTTCCATCTATGA | | |
| | | 697 | TTCTTAGCTTCCTCCAGGAGCTGCT | | |
| | | 698 | GGGTTGATGGAGAGTCCCCAAGGCC | | |
| | | 699 | GAAGCCTGAGCCAGAGAACCAGCTA | | |
| | | 700 | GGATGGGGTCCCTGATGAATATCTG | | |
| | | 701 | AATATCTGGAGACCTCGACAGCCTG | | |
| | | 702 | GGGTGGGTCAGGACAAAGGCCTCTC | | |
| | | 703 | GCCTCTCATCACCGCAGAAAGCGGG | | |
| | | 704 | AGTGAATGGGCCTGTGGCCCACCTG | | |
| ZBP1 | 208087_s_at | 705 | GGGTTCAGGCCAGGTCTTTTGATGG | gggttcaggccaggtcttttgatggccaggagtagatgacagggagttgcctt | NM_030776.1 |
| | | | | ggggaacctttggtgtgccaagaggaggtgggtagatgggagtggggctcggt | |
| | | | | cccccaggcccaggggactctctccactctttcctgggctcggggcatctgcc | |
| | | | | tggagttaccttccatcatggctacctgctgtggtttgaatgtttgagtccca | |
| | | | | acaaaattcatatcaaaacataatcccaactgggtgcagtggctcacgcctgt | |
| | | | | aatcccagcactttgggaggccgaggcgggcggatcaataggtcaggaaatccagac | |
| | | | | cgtcct | |
| | | 706 | CCAGGTCTTTTGATGGCCAGGAGTA | | |
| | | 707 | GGCCAGGAGTAGATGACAGGGAGTT | | |
| | | 708 | TGCCTTGGGGAACCTTTGGTGTGCC | | |
| | | 709 | TGGGGAACCTTTGGTGTGCCAAGAG | | |
| | | 710 | GGTAGATGGGAGTGGGGCTCGGTCC | | |
| | | 711 | TAGATGGGAGTGGGGCTCGGTCCCC | | |
| | | 712 | GTGGTTTGAATGTTTGAGTCCCAAC | | |
| | | 713 | GAGTCCCAACAAAATTCATATCAAA | | |
| | | 714 | ACATAATCCCAACTGGGTGCAGTGG | | |
| | | 715 | TAGGTCAGGAAATCCAGACCGTCCT | | |
| APLP2 | 208248_x_at | 716 | CCCTTCCAACTATGTCCAGATGTGC | cccttccaactatgtccagatgtgcaggctcctcctctctggactttctccaa | NM_001642.1 |
| | | | | aggcactgaccctcggcctctactttgtcccctcacctccaccccctcctgtc | |
| | | | | accggccttgtgacattcactcagagaagaccacaccaaggaggggccgcggc | |
| | | | | tggcccaggagagaacacggggaggtttgtttgtgtgaaaggaaagtagtcca | |
| | | | | ggctgtccctgaaactgagtctgtggacactgtggaaagctttgaacaattgt | |
| | | | | gttttcgtcacaggagtctttgtaatgcttgtacagttgatgtcgatgctcac | |
| | | | | tgcttctgctttttctttctttttattttaaaaaatctgaaggttctggtaac | |
| | | | | ctgtggtgtatttttattttcctgtgactgtttttgttttgtttttttccttt | |
| | | | | ttcctcccctttagccctattcatgtctctacccactatgcacagattaaacttcac | |
| | | | | ctacaaactcct | |
| | | 717 | TCCTCTCTGGACTTTCTCCAAAGGC | | |
| | | 718 | CCGGCCTTGTGACATTCACTCAGAG | | |
| | | 719 | AGAAGACCACACCAAGGAGGGGCCG | | |
| | | 720 | AGGAAAGTAGTCCAGGCTGTCCCTG | | |
| | | 721 | GCTGTCCCTGAAACTGAGTCTGTGG | | |
| | | 722 | GTGTTTTCGTCACAGGAGTCTTTGT | | |
| | | 723 | CAGTTGATGTCGATGCTCACTGCTT | | |
| | | 724 | GGTGTATTTTTATTTTCCTGTGACT | | |
| | | 725 | TCTCTACCCACTATGCACAGATTAA | | |
| | | 726 | GATTAAACTTCACCTACAAACTCCT | | |
| CS | 208660_at | 727 | AGAATACAAGCCACTACCTTCTGAC | agaatacaagccactaccttctgacctccccaccccccaccaacccccatctt | BC000105.1 |
| | | | | ttaatatgctgtggggcatagaactccggaatgaccagcatgatattttcaga | |
| | | | | gtcttgtccccggggtattagcacctctttttgaacagggaattgattcaaga | |
| | | | | ttggacatggtctcctctgattatcaggtactggggctgagggcattaaaaat | |
| | | | | agtaagcctccctcctcgtcccctgcctcaagaaattgcctccttatttatca | |
| | | | | acatctttttcctccctttccctgagagctcacagtacaatgtttcagaagcc | |
| | | | | ccatttgcacaggttttcagcaactcagaatgctctacttctttttctttgag | |
| | | | | aaaggattaagatacactcctgctgtgcccccatctttcctccaaactcctgc | |
| | | | | ctgtgtttgtgtggatacccagtcccagaaccacactgttgagttggacacactgtaa | |
| | | | | acccct | |
| | | 728 | TGCTGTGGGGCATAGAACTCCGGAA | | |
| | | 729 | TGATATTTTCAGAGTCTTGTCCCCG | | |
| | | 730 | TCTTGTCCCCGGGGTATTAGCACCT | | |
| | | 731 | GGTATTAGCACCTCTTTTTGAACAG | | |
| | | 732 | TGGACATGGTCTCCTCTGATTATCA | | |
| | | 733 | TGCCTCCTTATTTATCAACATCTTT | | |
| | | 734 | GCCCCATTTGCACAGGTTTTCAGCA | | |
| | | 735 | GCAACTCAGAATGCTCTACTTCTTT | | |
| | | 736 | TTGTGTGGATACCCAGTCCCAGAAC | | |
| | | 737 | TGAGTTGGACACACTGTAAACCCCT | | |
| LTA4H | 208771_s_at | 738 | GATTGGAATGCCTGGCTCTACTCTC | gattggaatgcctggctctactctcctggactgcctcccataaagcccaatta | J02959.1 |
| | | | | tgatatgactctgacaaatgcttgtattgccttaagtcaaagatggattactg | |
| | | | | ccaaagaagatgatttaaattcattcaatgccacagacctgaaggatctctct | |
| | | | | tctcatcaattgaatgagtttttagcacagacgctccagagggcacctcttcc | |
| | | | | attggggcacataaagcgaatgcaagaggtgtacaacttcaatgccattaaca | |
| | | | | attctgaaatacgattcagatggctgcggctctgcattcaatccaagtgggag | |
| | | | | gacgcaattcctttggcgctaaagatggcaactgaacaaggaagaatgaagtt | |
| | | | | tacccggcccttattcaaggatcttgctgcctttgacaaatcccatgatcaag | |
| | | | | ctgtccgaacctaccaagagcacaaagcaagcatgcatcccgtgactgcaatgctggt | |
| | | | | gg | |
| | | 739 | GCCACAGACCTGAAGGATCTCTCTT | | |
| | | 740 | GGATCTCTCTTCTCATCAATTGAAT | | |
| | | 741 | GAGTTTTTAGCACAGACGCTCCAGA | | |
| | | 742 | TGGGAGGACGCAATTCCTTTGGCGC | | |
| | | 743 | GGCCCTTATTCAAGGATCTTGCTGC | | |
| | | 744 | TTGCTGCCTTTGACAAATCCCATGA | | |
| | | 745 | AAATCCCATGATCAAGCTGTCCGAA | | |
| | | 746 | GTCCGAACCTACCAAGAGCACAAAG | | |
| | | 747 | CAAAGCAAGCATGCATCCCGTGACT | | |
| | | 748 | CATCCCGTGACTGCAATGCTGGTGG | | |
| ANXA2P2 | 208816_x_at | 749 | CAGAAAGCGCTGCTGTACCTGTGTG | tgccccacctccagaaagtatttgataggtacaagagttacagcccttatgac | M62898.1 |
| | | | | atgttggaaagcatcaggaaagaggttaaaggagacctggaaaatgctttcct | |
| | | | | gaacctggtccagcgcattcagaacaagcccttgtattttgctgatcagctgt | |
| | | | | acgactccatgaagggcaaggggacgcgagataaggtcctgatcagaatcatg | |
| | | | | gtctcccgcagtgaagtggacatgttgaaaattaggtctgaattcaagagaaa | |
| | | | | gtacggcaagtccctgtactactatatccagcaagacactaagggcgactacc | |
| | | | | agaaagcgctgctgtacctgtgtggtggagctgactgaagcccgacacagcct | |
| | | | | gagcgtccagaaatggtgctcaccatgcttccagctaacaggtctactaaaca | |
| | | | | tacaaaagtttagccgggcgtggtggcgctcgcctgtagtcccagctagtccggagc | |
| | | | | tgag | |
| | | 750 | TGGTGGAGCTGACTGAAGCCCGACA | | |
| | | 751 | GACACAGCCTGAGCGTCCAGAAATG | | |
| | | 752 | CTCACCATGCTTCCAGCTAACAGGT | | |
| | | 753 | TAGTCCCAGCTAGTCCGGAGCTGAG | | |
| | | 754 | TGCCCCACCTCCAGAAAGTATTTGA | | |
| | | 755 | CTGAACCTGGTCCAGCGCATTCAGA | | |
| | | 756 | AAGCCCTTGTATTTTGCTGATCAGC | | |
| | | 757 | CTGATCAGCTGTACGACTCCATGAA | | |
| | | 758 | CTGATCAGAATCATGGTCTCCCGCA | | |
| | | 759 | GGCAAGTCCCTGTACTACTATATCC | | |
| PLXNB2 | 208890_s_at | 760 | CGCCCAGCGTCTAGACTGTAGCATC | cgcccagcgtctagactgtagcatcttcctctgagcaataccgccgggcaccg | BC004542.1 |
| | | | | caccagcaccagccccagccccagctccctccggccgcagaaccagcatcggg | |
| | | | | tgttcactgtcgagtctcgagtgatttgaaaatgtgccttacgctgccacgct | |
| | | | | gggggcagctggcctccgcctccgcccacgcaccagcagccgcctccatgccc | |
| | | | | taggttgggcccctgggggatctgagggcctgtggcccccagggcaagttccc | |
| | | | | agatcctatgtctgtctgtccaccacgagatgggaggaggagaaaaagcggta | |
| | | | | cgatgccttcctgacctcaccggcctccccaagggtgccggcactctgggtgg | |
| | | | | actcacggctgctgggccccacgtcaaaggtcaagtgagacgtaggtcaagtc | |
| | | | | ctacgtcggggcccagacatcctggggtcctggtctgtcagacaggctgccct | |
| | | | | agagccccacccagtccggggggactgggagcagttccaagaccaccc | |
| | | 761 | GAACCAGCATCGGGTGTTCACTGTC | | |
| | | 762 | GTGTTCACTGTCGAGTCTCGAGTGA | | |
| | | 763 | TACGCTGCCACGCTGGGGGCAGCTG | | |
| | | 764 | CCAGGGCAAGTTCCCAGATCCTATG | | |
| | | 765 | TCCCAGATCCTATGTCTGTCTGTCC | | |
| | | 766 | AGAAAAAGCGGTACGATGCCTTCCT | | |
| | | 767 | GGCCCCACGTCAAAGGTCAAGTGAG | | |
| | | 768 | TCTGTCAGACAGGCTGCCCTAGAGC | | |
| | | 769 | TCCGGGGGGACTGGGAGCAGTTCCA | | |
| | | 770 | ACTGGGAGCAGTTCCAAGACCACCC | | |
| CYFIP1 | 208923_at | 771 | GCACTCCGTAACTCAACATGGCATG | gcactccgtaactcaacatggcatgcctttctctccgtaaactatttagtgag | BC005097.1 |
| | | | | atttttagggactatttttcagtatctctgtacctgttaaagggggtgctttt | |
| | | | | cgatctaaaaacttaattttataaaattgacttatttttctagactaaaattg | |
| | | | | tatatgcttttggtaattaggaactcttgagaatattggctgctgattgttgc | |
| | | | | catcacgttcctacaaaattgtttttctatgggatgttctggcagctgtgtca | |
| | | | | taaaatgctgctgggttcattcattcattccataagaaacttaataccagcaa | |
| | | | | atgcattaaatcccttgccagttaccattaactataactatttagcttttgtt | |
| | | | | tagggatctttctgatggtcttttatgagcaatcttagttctaagtcattgtt | |
| | | | | cccatcccttttttgtgtgtttcagaaaatagtgaacttgattcccctgcttccacta | |
| | | | | aatccagttgtga | |
| | | 772 | GCCTTTCTCTCCGTAAACTATTTAG | | |
| | | 773 | TTTTCAGTATCTCTGTACCTGTTAA | | |
| | | 774 | GAGAATATTGGCTGCTGATTGTTGC | | |
| | | 775 | TTGTTGCCATCACGTTCCTACAAAA | | |
| | | 776 | TGGGATGTTCTGGCAGCTGTGTCAT | | |
| | | 777 | ATGCTGCTGGGTTCATTCATTCATT | | |
| | | 778 | GTTTAGGGATCTTTCTGATGGTCTT | | |
| | | 779 | CTTAGTTCTAAGTCATTGTTCCCAT | | |
| | | 780 | AATAGTGAACTTGATTCCCCTGCTT | | |
| | | 781 | CTGCTTCCACTAAATCCAGTTGTGA | | |
| MAGED1 | 209014_at | 782 | GGACTGCACAGTTCATGGAGGCTGC | ggactgcacagttcatggaggctgcagatgaggccttggatgctctggatgct | AF217963.1 |
| | | | | gctgcagctgaggccgaagcccgggctgaagcaagaacccgcatgggaattgg | |
| | | | | agatgaggctgtgtctgggccctggagctgggatgacattgagtttgagctgc | |
| | | | | tgacctgggatgaggaaggagattttggagatccctggtccagaattccattt | |
| | | | | accttctgggccagataccaccagaatgcccgctccagattccctcagacctt | |
| | | | | tgccggtcccattattggtcctggtggtacagccagtgccaacttcgctgcca | |
| | | | | actttggtgccattggtttcttctgggttgagtgagatgttggatattgctat | |
| | | | | caatcgcagtagtctttcccctgtgtgagctgaagcctcagattccttctaaa | |
| | | | | cacagctatctagagagccacatcctgttgactgaaagtggcatgcaagataa | |
| | | | | atttatttgctgttccttgtctactgctttttttccccttgtgtgctgtcaagt | |
| | | 783 | ATGAGGCCTTGGATGCTCTGGATGC | | |
| | | 784 | TGGAGATCCCTGGTCCAGAATTCCA | | |
| | | 785 | TTCCATTTACCTTCTGGGCCAGATA | | |
| | | 786 | GGTCCCATTATTGGTCCTGGTGGTA | | |
| | | 787 | CCAACTTCGCTGCCAACTTTGGTGC | | |
| | | 788 | GTGCCATTGGTTTCTTCTGGGTTGA | | |
| | | 789 | TCCCCTGTGTGAGCTGAAGCCTCAG | | |
| | | 790 | CTATCTAGAGAGCCACATCCTGTTG | | |
| | | 791 | ATTTATTTGCTGTTCCTTGTCTACT | | |
| | | 792 | TTTTTCCCCTTGTGTGCTGTCAAGT | | |
| SYNE1 | 209447_at | 793 | GAGGACCTTGATCTTGGCGAAAGCC | gaggaccttgatcttggcgaaagccatcggtgtggcagctttagccctcctcc | AF043290.1 |
| | | | | agatcacatgtgtgcaaattatggcttcagagggtggaagataaacagtgacg | |
| | | | | ggggaacaaacagacaacaagaaggtttggaagaaatctggtttgagactctg | |
| | | | | aaccttagcactaaggagattgagtaaggacctccaaagttccccggactcat | |
| | | | | gaattctgggcccttggcattcgtgtgcacagccaaggacttcagtagaccat | |
| | | | | ctgggcagctttcccatggtgctgctccaaccatcagataaatgaccctcccc | |
| | | | | aagcaccatgtcagtgtcgtacaatctaccaaccaaccagtgctgaagagatt | |
| | | | | ttagaaccttgtaacatacaatttttaagagcttatatggcagcttcctttt | |
| | | 794 | GCCATCGGTGTGGCAGCTTTAGCCC | | |
| | | 795 | TTGAGACTCTGAACCTTAGCACTAA | | |
| | | 796 | AAAGTTCCCCGGACTCATGAATTCT | | |
| | | 797 | CCCTTGGCATTCGTGTGCACAGCCA | | |
| | | 798 | GCACAGCCAAGGACTTCAGTAGACC | | |
| | | 799 | TCAGTAGACCATCTGGGCAGCTTTC | | |
| | | 800 | AACCATCAGATAAATGACCCTCCCC | | |
| | | 801 | GCACCATGTCAGTGTCGTACAATCT | | |
| | | 802 | GTGTCGTACAATCTACCAACCAACC | | |
| | | 803 | AGAGCTTATATGGCAGCTTCCTTTT | | |
| CBLB | 209682_at | 804 | GGAGACCGATGCTTGCTCAGGATGT | ggagaccgatgcttgctcaggatgtcgacagctgtggcttccttgtttttgct | U26710.1 |
| | | | | agccatatttttaaatcagggttgaactgacaaaaataatttaaagacgttta | |
| | | | | cttcccttgaactttgaacctgtgaaatgctttaccttgtttacaatttggca | |
| | | | | aagttgcagtttgttcttgtttttagtttagttttgttttggtgttttgatac | |
| | | | | ctgtactgtgttcttcacagaccctttgtagcgtggtcaggtctgctgtaaca | |
| | | | | tttcccaccaactctcttgctgtccacatcaacagctaaatcatttattcata | |
| | | | | tggatctctaccatccccatgccttgcccaggtccagttccatttctctcatt | |
| | | | | cacaagatgctttgaaggttctgattttcaactgatcaaactaatgcaaaaaa | |
| | | | | aaaaagtatgtattcttcactactgagtttcttctttggaaaccatcactatt | |
| | | 805 | CCTTGTTTTTGCTAGCCATATTTTT | | |
| | | 806 | GAACCTGTGAAATGCTTTACCTTGT | | |
| | | 807 | GGCAAAGTTGCAGTTTGTTCTTGTT | | |
| | | 808 | GTACTGTGTTCTTCACAGACCCTTT | | |
| | | 809 | CTTCACAGACCCTTTGTAGCGTGGT | | |
| | | 810 | GTAGCGTGGTCAGGTCTGCTGTAAC | | |
| | | 811 | GTTCCATTTCTCTCATTCACAAGAT | | |
| | | 812 | GAAGGTTCTGATTTTCAACTGATCA | | |
| | | 813 | TTCTTCACTACTGAGTTTCTTCTTT | | |
| | | 814 | TTCTTCTTTGGAAACCATCACTATT | | |
| CD247 | 210031_at | 815 | ACTGTACTGGGCCATGTTGTGCCTC | aagcgcagatgctagcacatgccctaatgtctgtatcactctgtgtctgagtg | J04132.1 |
| | | | | gcttcactcctgctgtaaatttggcttctgttgtcaccttcacctcctttcaa | |
| | | | | ggtaactgtactgggccatgttgtgcctccctggtgagagggccgggcagagg | |
| | | | | ggcagatggaaaggagcctaggccaggtgcaaccagggagctgcaggggcatg | |
| | | | | ggaaggtgggcgggcaggggagggtcagccagggcctgcgagggcagcgggag | |
| | | | | cctccctgcctcaggcctctgtgccgcaccattgaactgtaccatgtgctaca | |
| | | | | ggggccagaagatgaacagactgaccttgatgagctgtgcacaaagtggcata | |
| | | | | aaaaacagtgtggttacacagtgtgaataaagtgctgcggagcaagaggaggc | |
| | | | | cgttgattcacttcacgctttcagcgaatgacaaaatcatctttgtgaaggcctcgca | |
| | | | | ggaagacgcaacacatgggacctat | |
| | | 816 | AAAGGAGCCTAGGCCAGGTGCAACC | | |
| | | 817 | TGCCGCACCATTGAACTGTACCATG | | |
| | | 818 | GACTGACCTTGATGAGCTGTGCACA | | |
| | | 819 | TGATTCACTTCACGCTTTCAGCGAA | | |
| | | 820 | ATCATCTTTGTGAAGGCCTCGCAGG | | |
| | | 821 | GGAAGACGCAACACATGGGACCTAT | | |
| | | 822 | AAGCGCAGATGCTAGCACATGCCCT | | |
| | | 823 | AATGTCTGTATCACTCTGTGTCTGA | | |
| | | 824 | GGCTTCACTCCTGCTGTAAATTTGG | | |
| | | 825 | AAATTTGGCTTCTGTTGTCACCTTC | | |
| PRKCQ | 210038_at | 826 | AATCCATTCATCCTGATTGGGCATG | aatccattcatcctgattgggcatgaaatccatggtcaagaggacaagtggaa | AL137145 |
| | | | | agtgagagggaaggtttgctagacaccttcgcttgttatcttgtcaagataga | |
| | | | | aaagatagtatcatttcacccttgccagtaaaaacctttccatccacccattc | |
| | | | | tcagcagactccagtattggcacagtcactcactgccattctcacactataac | |
| | | | | aagaaaagaaatgaagtgcataagtctcctgggaaaagaaccttaaccccttc | |
| | | | | tcgtgccatgactggtgatttcatgactcataagcccctccgtaggcatcattcaaga | |
| | | | | tcaatggcccatgcatgctgtttgcagca | |
| | | 827 | GACACCTTCGCTTGTTATCTTGTCA | | |
| | | 828 | ATCATTTCACCCTTGCCAGTAAAAA | | |
| | | 829 | CCATTCTCAGCAGACTCCAGTATTG | | |
| | | 830 | CCAGTATTGGCACAGTCACTCACTG | | |
| | | 831 | ACTGCCATTCTCACACTATAACAAG | | |
| | | 832 | GAAGTGCATAAGTCTCCTGGGAAAA | | |
| | | 833 | CCTTCTCGTGCCATGACTGGTGATT | | |
| | | 834 | TGATTTCATGACTCATAAGCCCCTC | | |
| | | 835 | CCCCTCCGTAGGCATCATTCAAGAT | | |
| | | 836 | TGGCCCATGCATGCTGTTTGCAGCA | | |
| FYN | 210105_s_at | 837 | GGCCCGGGTCTGCGGAGAGAGGCCT | ggcccgggtctgcggagagaggccttgtcccagaggctgccccacccctcccc | M14333.1 |
| | | | | attagctttcaattccgtagccagctgctccccagcagcggaaccgcccagga | |
| | | | | tcagattgcatgtgactctgaagctgacgaacttccatggccctcattaatga | |
| | | | | cacttgtccccaaatccgaacctcctctgtgaagcattcgagacagaaccttg | |
| | | | | ttatttctcagactttggaaaatgcattgtatcgatgttatgtaaaaggccaa | |
| | | | | acctctgttcagtgtaaatagttactccagtgccaacaatcctagtgctttcc | |
| | | | | ttttttaaaaatgcaaatcctatgtgattttaactctgtcttcacctgattca | |
| | | | | actaaaaaaaaaaagtattattttccaaaagtggcctctttgtctaa | |
| | | 838 | AGCTTTCAATTCCGTAGCCAGCTGC | | |
| | | 839 | AACCGCCCAGGATCAGATTGCATGT | | |
| | | 840 | GATTGCATGTGACTCTGAAGCTGAC | | |
| | | 841 | CTTCCATGGCCCTCATTAATGACAC | | |
| | | 842 | TAATGACACTTGTCCCCAAATCCGA | | |
| | | 843 | GACAGAACCTTGTTATTTCTCAGAC | | |
| | | 844 | AAAGGCCAAACCTCTGTTCAGTGTA | | |
| | | 845 | TCCAGTGCCAACAATCCTAGTGCTT | | |
| | | 846 | CCTATGTGATTTTAACTCTGTCTTC | | |
| | | 847 | TTCCAAAAGTGGCCTCTTTGTCTAA | | |
| LILRB4 | 210152_at | 848 | AGGACGGGGTGGAAATGGACACTCG | ccaacactggcgtcagggaaaacacaggacattggcccagagacaggctgatt | U82979.1 |
| | | | | tccaacgtcctccaggggctgccgagccagagcccaaggacgggggcctacag | |
| | | | | aggaggtccagcccagctgctgacgtccagggagaaaacttctgtgctgccgt | |
| | | | | gaagaacacacagcctgaggacggggtggaaatggacactcggagcccacacg | |
| | | | | atgaagacccccaggcagtgacgtatgccaaggtgaaacactccagacctagg | |
| | | | | agagaaatggcctctcctccctccccactgtctggggaattcctggacacaaa | |
| | | | | ggacagacaggcagaagaggacagacagatggacactgaggctgctgcatctg | |
| | | | | aagccccccaggatgtgacctacgcccagctgcacagctttaccctcagacagaagg | |
| | | | | caactg | |
| | | 849 | CACAGCTTTACCCTCAGACAGAAGG | | |
| | | 850 | TTTACCCTCAGACAGAAGGCAACTG | | |
| | | 851 | CCAACACTGGCGTCAGGGAAAACAC | | |
| | | 852 | GGAAAACACAGGACATTGGCCCAGA | | |
| | | 853 | ATTGGCCCAGAGACAGGCTGATTTC | | |
| | | 854 | GAGACAGGCTGATTTCCAACGTCCT | | |
| | | 855 | GAGCCCAAGGACGGGGGCCTACAGA | | |
| | | 856 | GCTGACGTCCAGGGAGAAAACTTCT | | |
| | | 857 | AAACTTCTGTGCTGCCGTGAAGAAC | | |
| | | 858 | TCTGTGCTGCCGTGAAGAACACACA | | |
| GZMB | 210164_at | 859 | GCCAAGCGGACCAGAGCTGTGCAGC | gccaagcggaccagagctgtgcagcccctcaggctacctagcaacaaggccca | J03189.1 |
| | | | | ggtgaagccagggcagacatgcagtgtggccggctgggggcagacggcccccc | |
| | | | | tgggaaaacattcacacacactacaagaggtgaagatgacagtgcaggaagat | |
| | | | | cgaaagtgcgaatctgacttacgccattattacgacagtaccattgagttgtg | |
| | | | | cgtgggggacccagagattaaaaagacttcctttaagggggactctggaggcc | |
| | | | | ctcttgtgtgtaacaaggtggcccagggcattgtctcctatggacgaaacaat | |
| | | | | ggcatgcctccacgagcctgcaccaaagtctcaagctttgtacactggataaa | |
| | | | | gaaaaccatgaaacgctactaactacaggaagcaaactaagcccccgctgtaatgaa | |
| | | | | acaccttctctggagcca | |
| | | 860 | TGCGAATCTGACTTACGCCATTATT | | |
| | | 861 | GACTTACGCCATTATTACGACAGTA | | |
| | | 862 | ACGACAGTACCATTGAGTTGTGCGT | | |
| | | 863 | TTGAGTTGTGCGTGGGGGACCCAGA | | |
| | | 864 | ACTCTGGAGGCCCTCTTGTGTGTAA | | |
| | | 865 | GCATTGTCTCCTATGGACGAAACAA | | |
| | | 866 | AAGTCTCAAGCTTTGTACACTGGAT | | |
| | | 867 | TACAGGAAGCAAACTAAGCCCCCGC | | |
| | | 868 | CTAAGCCCCCGCTGTAATGAAACAC | | |
| | | 869 | TAATGAAACACCTTCTCTGGAGCCA | | |
| ANXA2 | 210427_x_at | 870 | CTGATCAGAATCATGGTCTCCCGCA | gaaaatgctttcctgaacctggttcagtgcattcagaacaagcccctgtattt | BC001388.1 |
| | | | | tgctgatcggctgtatgactccatgaagggcaaggggacgcgagataaggtcc | |
| | | | | tgatcagaatcatggtctcccgcagtgaagtggacatgttgaaaattaggtct | |
| | | | | gaattcaagagaaagtacggcaagtccctgtactattatatccagcaagacac | |
| | | | | taagggcgactaccagaaagcgctgctgtacctgtgtggtggagatgactgaa | |
| | | | | gcccgacacggcctgagcgtccagaaatggtgctcaccatgcttccagctaac | |
| | | | | aggtctagaaaaccagcttgcgaataacagtccccgtggccatccctgtgagg | |
| | | | | gtgacgttagcattacccccaacctcattttagttgcctaagcattgcctggc | |
| | | | | cttcctgtctagtctctcctgtaagccaaagaaatgaacattccaaggagttg | |
| | | | | gaagtgaagtctatgatgtgaaacactttgcctcctgtgtactgtgtcataaa | |
| | | 871 | CAGAAAGCGCTGCTGTACCTGTGTG | | |
| | | 872 | CTCACCATGCTTCCAGCTAACAGGT | | |
| | | 873 | ACCAGCTTGCGAATAACAGTCCCCG | | |
| | | 874 | CGTGGCCATCCCTGTGAGGGTGACG | | |
| | | 875 | GAGGGTGACGTTAGCATTACCCCCA | | |
| | | 876 | AGTTGCCTAAGCATTGCCTGGCCTT | | |
| | | 877 | TGCCTCCTGTGTACTGTGTCATAAA | | |
| | | 878 | GAAAATGCTTTCCTGAACCTGGTTC | | |
| | | 879 | CAAGCCCCTGTATTTTGCTGATCGG | | |
| | | 880 | GCTGATCGGCTGTATGACTCCATGA | | |
| NFATC3 | 210555_s_at | 881 | TCTGCACCTTCATCCTTAATATGTC | tctgcaccttcatccttaatatgtcacagtttgtgtgatccagcgtcatttcc | U85430.1 |
| | | | | acctgatggggcaactgtgagcattaaacctgaaccagaagatcgagagccta | |
| | | | | actttgcaaccattggtctgcaggacatcactttagat | |
| | | 882 | CATCCTTAATATGTCACAGTTTGTG | | |
| | | 883 | ACAGTTTGTGTGATCCAGCGTCATT | | |
| | | 884 | TTGTGTGATCCAGCGTCATTTCCAC | | |
| | | 885 | GTCATTTCCACCTGATGGGGCAACT | | |
| | | 886 | GGGGCAACTGTGAGCATTAAACCTG | | |
| | | 887 | ACCTGAACCAGAAGATCGAGAGCCT | | |
| | | 888 | GATCGAGAGCCTAACTTTGCAACCA | | |
| | | 889 | GAGCCTAACTTTGCAACCATTGGTC | | |
| | | 890 | CAACCATTGGTCTGCAGGACATCAC | | |
| | | 891 | TGGTCTGCAGGACATCACTTTAGAT | | |
| KLRD1 | 210606_x_at | 892 | GAAAGACTCTGACTGCTGTTCTTGC | gaaagactctgactgctgttcttgccaagaaaaatgggttgggtaccggtgca | U30610.1 |
| | | | | actgttacttcatttccagtgaacagaaaacttggaacgaaagtcggcatctc | |
| | | | | tgtgcttctcagaaatccagcctgcttcagcttcaaaacacagatgaactgga | |
| | | | | ttttatgagctccagtcaacaattttactggattggactctcttacagtgagg | |
| | | | | agcacaccgcctggttgtgggagaatggctctgcactctcccagtatctattt | |
| | | | | ccatcatttgaaacttttaatacaaagaactgcatagcgtataatccaaatgg | |
| | | | | aaatgctttagatgaatcctgtgaagataaaaatcgttatatctgtaagcaac | |
| | | | | agctcatttaaatgtttcttggggcagagaaggtggagagtaaagacccaaca | |
| | | | | ttactaacaatgatacagttgcatgttatattattactaattgtctacttctggagt | |
| | | | | cta | |
| | | 893 | GTACCGGTGCAACTGTTACTTCATT | | |
| | | 894 | ACGAAAGTCGGCATCTCTGTGCTTC | | |
| | | 895 | CTGTGCTTCTCAGAAATCCAGCCTG | | |
| | | 896 | CAGCCTGCTTCAGCTTCAAAACACA | | |
| | | 897 | TTTTACTGGATTGGACTCTCTTACA | | |
| | | 898 | CTTACAGTGAGGAGCACACCGCCTG | | |
| | | 899 | GCACACCGCCTGGTTGTGGGAGAAT | | |
| | | 900 | GTGGGAGAATGGCTCTGCACTCTCC | | |
| | | 901 | TCCCAGTATCTATTTCCATCATTTG | | |
| | | 902 | ACTAATTGTCTACTTCTGGAGTCTA | | |
| PMS2L11 | 210707_x_at | 903 | GAAGTCAGTCCATCAGATTTGCTCT | ctggaccctatcgtacagaacctgctaaggccatcaaacctattgatcggaag | U38980.1 |
| | | | | tcagtccatcagatttgctctgggccagtggtactgagtctaagcactgcagt | |
| | | | | gaaggagttagtagaaaacagtctggatgctggtgccactaatattgatctaa | |
| | | | | agcttaaggactatggaatggatctcattgaagtttcaggcaatggatgtggg | |
| | | | | gtagaagaagaaaacttcgaaggcttaatgatgtcaccatttctacctgccac | |
| | | | | gtctcggcgaaggttgggactcgactggtgtttgatcacgatgggaaaatcat | |
| | | | | ccagaagaccccctacccccaccccagagggaccacagtcagcgtgaagcagt | |
| | | | | tattttctacgctacctgtgcgccataaggaatttcaaaggaatattaagaagaaac | |
| | | | | atgctgcttccccttc | |
| | | 904 | GCTCTGGGCCAGTGGTACTGAGTCT | | |
| | | 905 | AACAGTCTGGATGCTGGTGCCACTA | | |
| | | 906 | TAATGATGTCACCATTTCTACCTGC | | |
| | | 907 | GCCACGTCTCGGCGAAGGTTGGGAC | | |
| | | 908 | GTTGGGACTCGACTGGTGTTTGATC | | |
| | | 909 | GAGGGACCACAGTCAGCGTGAAGCA | | |
| | | 910 | CTACGCTACCTGTGCGCCATAAGGA | | |
| | | 911 | AGAAGAAACATGCTGCTTCCCCTTC | | |
| | | 912 | CTGGACCCTATCGTACAGAACCTGC | | |
| | | 913 | GAACCTGCTAAGGCCATCAAACCTA | | |
| HOP | 211597_s_at | 914 | AAGCTATGTGTATCTTCTGTGTAAA | aagctatgtgtatcttctgtgtaaagcagtggcttcactggaaaaatggtgtg | AB059408.1 |
| | | | | gctagcatttccctttgagtcatgatgacagatggtgtgaaaaccatctaagt | |
| | | | | ttgcttttgaccatcacctcccagtagcaatttgctttcataatccatttagc | |
| | | | | aatccaggcctctgttgaaaagataatatgagggagaagggaacacatttcct | |
| | | | | tctgaacttacttccctaagtcactttccttatgtatcatctaatacaatgat | |
| | | | | ggttgagtgaaaatacagaaggggtgtttgagtattcagatttcataaaacac | |
| | | | | ttccttggaatatagctgcattaacttggaaagaagcctgttgggccagaagacaga | |
| | | 915 | AATGGTGTGGCTAGCATTTCCCTTT | | |
| | | 916 | TAAGTTTGCTTTTGACCATCACCTC | | |
| | | 917 | TCACCTCCCAGTAGCAATTTGCTTT | | |
| | | 918 | TAATCCATTTAGCAATCCAGGCCTC | | |
| | | 919 | GCAATCCAGGCCTCTGTTGAAAAGA | | |
| | | 920 | GAAGGGAACACATTTCCTTCTGAAC | | |
| | | 921 | CTTCCCTAAGTCACTTTCCTTATGT | | |
| | | 922 | AGTCACTTTCCTTATGTATCATCTA | | |
| | | 923 | ACTTCCTTGGAATATAGCTGCATTA | | |
| | | 924 | GAAGCCTGTTGGGCCAGAAGACAGA | | |
| NCALD | 211685_s_at | 925 | TGGGTGAGGAGACCTAGCATGCCCT | tgggtgaggagacctagcatgccctattggcagtgctcaggagctgcatccca | AF251061.1 |
| | | | | cttttccctgctctgaatcgaagtcctagttccttcctttgattctcctttgg | |
| | | | | taggtggaatcagttaatgttttgagaaacctgcctgggctctgcccttagtc | |
| | | | | atgacatctcgctgagccagacccactctgttccttggaacctagagctggag | |
| | | | | tgaggagtagaggtctccggctattccagaaagaaaagtgagccacatgcagg | |
| | | | | ctgatgaatgccgacacttccagaatgtatagaaatagtccctgtcctggcct | |
| | | | | gccactgaccctgtctgtattttctcggaggttgtttttctccttctccttcc | |
| | | | | caggaaggtctttgtatgtcgaatccagtgcactcaagtttggccaagggact | |
| | | | | ccacagcacccagaagactgcatgcctcaaggtttatgtcactcctctgctgggctg | |
| | | | | ttcattgtcattgc | |
| | | 926 | AGCATGCCCTATTGGCAGTGCTCAG | | |
| | | 927 | TCCCTGCTCTGAATCGAAGTCCTAG | | |
| | | 928 | TTAGTCATGACATCTCGCTGAGCCA | | |
| | | 929 | GGAGTAGAGGTCTCCGGCTATTCCA | | |
| | | 930 | GAATGCCGACACTTCCAGAATGTAT | | |
| | | 931 | ACCCTGTCTGTATTTTCTCGGAGGT | | |
| | | 932 | TCTCGGAGGTTGTTTTTCTCCTTCT | | |
| | | 933 | GTATGTCGAATCCAGTGCACTCAAG | | |
| | | 934 | GCCTCAAGGTTTATGTCACTCCTCT | | |
| | | 935 | CTGCTGGGCTGTTCATTGTCATTGC | | |
| LOC130074 | 212017_at | 936 | GGATGAGCGGCGTCTGTGTAGGGAC | ggatgagcggcgtctgtgtagggacccccccccgggcctgcagaagggtggtg | BF677404 |
| | | | | tgctcccaggactggcatgacaggtgtctcctcctcaccacaggctgtgccca | |
| | | | | tgngtccctgtgcagaccagtgggcaaggcagctgggccagatctcaggccag | |
| | | | | ccgtttgtgctcctagcagggttgctgtgctggccacacggagaggccctaga | |
| | | | | gagcctcatggattgtaactaaagaagaaacggttcctttttgntttttttaa | |
| | | | | aaatgatttttaaataccgttttttacaccgttctctcggtactttttttaag | |
| | | | | ctaagtcagcattgtcttccagtgttaaaggcatccctcacctctgcattgaa | |
| | | | | cttacgtatccatgccaaggaatggaatttccatcctgagccagttcagttaggtgt | |
| | | | | caatt | |
| | | 937 | TGCAGAAGGGTGGTGTGCTCCCAGG | | |
| | | 938 | GGACTGGCATGACAGGTGTCTCCTC | | |
| | | 939 | TCCCTGTGCAGACCAGTGGGCAAGG | | |
| | | 940 | TGTGCTCCTAGCAGGGTTGCTGTGC | | |
| | | 941 | CACACGGAGAGGCCCTAGAGAGCCT | | |
| | | 942 | GAGAGCCTCATGGATTGTAACTAAA | | |
| | | 943 | CCAGTGTTAAAGGCATCCCTCACCT | | |
| | | 944 | CTCACCTCTGCATTGAACTTACGTA | | |
| | | 945 | GAACTTACGTATCCATGCCAAGGAA | | |
| | | 946 | GAGCCAGTTCAGTTAGGTGTCAATT | | |
| GPR56 | 212070_at | 947 | TCCAAGGACTGAGACTGACCTCCTC | tccaaggactgagactgacctcctctggtgacactggcctagngcctgacact | AL554008 |
| | | | | ctcctaagaggttctctccaagcccccaaatagctccaggcgccctcggccgc | |
| | | | | ccatcatggttaattctgtccaacaaacacacacgggtagattgctggcctgt | |
| | | | | tgtaggtggtagggacacagatgaccgacctggtcactcctcctgccaacatt | |
| | | | | cagtctggtatgtgaggcgtgcgtgaagcaagaactcctggagctacagggac | |
| | | | | agggagccatcattcctgcctgggaatcctggaagacttcctgcaggagtcag | |
| | | | | cgttcaatcttgaccttgaagatgggaaggatgttctttttacgtaccaattct | |
| | | 948 | ACACTCTCCTAAGAGGTTCTCTCCA | | |
| | | 949 | GGCCGCCCATCATGGTTAATTCTGT | | |
| | | 950 | CACACGGGTAGATTGCTGGCCTGTT | | |
| | | 951 | TAGGGACACAGATGACCGACCTGGT | | |
| | | 952 | CAGTCTGGTATGTGAGGCGTGCGTG | | |
| | | 953 | AGGCGTGCGTGAAGCAAGAACTCCT | | |
| | | 954 | AGGGACAGGGAGCCATCATTCCTGC | | |
| | | 955 | ACTTCCTGCAGGAGTCAGCGTTCAA | | |
| | | 956 | GTCAGCGTTCAATCTTGACCTTGAA | | |
| | | 957 | GATGTTCTTTTTACGTACCAATTCT | | |
| SPTBN1 | 212071_s_at | 958 | AAACCATTTGTATCTGGCATCACTT | aaaccatttgtatctggcatcacttactaacacacgacatgcggcttttctgc | BE968833 |
| | | | | atcaactgctatgacggttaagaatgtcagtatacaagaaggaatagaaaact | |
| | | | | gatactgttttaaataatctgtaatttcaatttttttttttttttngctgaaa | |
| | | | | tacattatattgtacgtttgagataattctagntacaaagtataataaaacta | |
| | | | | gatngtataataaaccctttaaatcattggtaagtgtacaagtggtggnaact | |
| | | | | gaagcatttactggnacaaagtaatgttnactctaatggttacttgctcgtgc | |
| | | | | gttgnnccacactgtgttataatttgcttcatttccttgctatttgatacata | |
| | | | | gtgtgcatttctctgtcactgtaactattgtaatgacaaattttcatcttact | |
| | | | | gcacaatcaaaatgacattgataggaatgaactccagaggctgggcctgaaca | |
| | | | | gggaggtggtcgctcaggcctggtgctcagtcgtacgacctgtacct | |
| | | 959 | TCTGGCATCACTTACTAACACACGA | | |
| | | 960 | TAACACACGACATGCGGCTTTTCTG | | |
| | | 961 | ACATGCGGCTTTTCTGCATCAACTG | | |
| | | 962 | TCTAATGGTTACTTGCTCGTGCGTT | | |
| | | 963 | TAATTTGCTTCATTTCCTTGCTATT | | |
| | | 964 | TGCATTTCTCTGTCACTGTAACTAT | | |
| | | 965 | AATTTTCATCTTACTGCACAATCAA | | |
| | | 966 | TAGGAATGAACTCCAGAGGCTGGGC | | |
| | | 967 | GAGGCTGGGCCTGAACAGGGAGGTG | | |
| | | 968 | GTGCTCAGTCGTACGACCTGTACCT | | |
| ATP2B4 | 212135_s_at | 969 | GTGGAAAAGCCTCTAAATGCATCCC | gtggaaaagcctctaaatgcatcccttcctttctttcctgcttcctttgcctt | AW517686 |
| | | | | acaattgaagcagcccgtggtaccatcacagtatgcagagacttcctcacctt | |
| | | | | tcatatctagggaccacccccgatgcattggtgagggtgggcacttataaatg | |
| | | | | cctgctattgttaagccattccagcctcttcctctgaatagaccagacgccctttca | |
| | | | | cttagttcagtgcca | |
| | | 970 | TGCTTCCTTTGCCTTACAATTGAAG | | |
| | | 971 | CAATTGAAGCAGCCCGTGGTACCAT | | |
| | | 972 | CAGTATGCAGAGACTTCCTCACCTT | | |
| | | 973 | CACCTTTCATATCTAGGGACCACCC | | |
| | | 974 | ACCACCCCCGATGCATTGGTGAGGG | | |
| | | 975 | GGTGGGCACTTATAAATGCCTGCTA | | |
| | | 976 | GCCTGCTATTGTTAAGCCATTCCAG | | |
| | | 977 | CAGCCTCTTCCTCTGAATAGACCAG | | |
| | | 978 | TCTGAATAGACCAGACGCCCTTTCA | | |
| | | 979 | CGCCCTTTCACTTAGTTCAGTGCCA | | |
| GTF3C2 | 212429_s_at | 980 | GTATCTGCATGAAGGCTCCTGTCTG | gtatctgcatgaaggctcctgtctgactattccaggatccaatattactgcct | AW194657 |
| | | | | tctgaaacttcctctttagggtaaccatcatgtatgcccacgagggtgatagt | |
| | | | | aattcgtgagactgaagttgcttagagtacttctttgaccaaggaataccaca | |
| | | | | gacaccctaccgatagaacagtggctcagatcttacttgctcctgcttacgaa | |
| | | | | gtattcccaatcactggtcatctgaccctacttgaacactcctgaacagtcat | |
| | | | | gttttttaaaatcttcctttatatcaagtcagagagtatacttctataaattt | |
| | | | | cactcatggatgttaggaaatctagtcatcttccctgtgattgccctgttaagtattt | |
| | | | | aaccatagctatcatgtgtttccca | |
| | | 981 | GGCTCCTGTCTGACTATTCCAGGAT | | |
| | | 982 | TATTACTGCCTTCTGAAACTTCCTC | | |
| | | 983 | TAACCATCATGTATGCCCACGAGGG | | |
| | | 984 | CAAGGAATACCACAGACACCCTACC | | |
| | | 985 | CCCTACCGATAGAACAGTGGCTCAG | | |
| | | 986 | TGCTCCTGCTTACGAAGTATTCCCA | | |
| | | 987 | TTCCCAATCACTGGTCATCTGACCC | | |
| | | 988 | GGAAATCTAGTCATCTTCCCTGTGA | | |
| | | 989 | TCCCTGTGATTGCCCTGTTAAGTAT | | |
| | | 990 | AACCATAGCTATCATGTGTTTCCCA | | |
| AUTS2 | 212599_at | 991 | TCAGACACACACAGGTCGCCAGTGA | tcagacacacacaggtcgccagtgacttcacacacacctcatgtgagaaccat | AK025298.1 |
| | | | | gccttttttagtgtgtcctatttcatacctgtacacacttcctcgttttgtaa | |
| | | | | tgagatttacttacacccaaacagatcctgaaagaaagcttcaagttttctca | |
| | | | | gatgatggatatgttttcactgtattcaataactgacggatgtaaggtgcacg | |
| | | | | tttcctgatgnntgacgcactgtattccagctggtgatcaagtctgggaacag | |
| | | | | ccgtaacaggtcaaccttgtggagccatcgcgagttagagggtgaaagatggc | |
| | | | | agaaaaaaaagtcttgtgtgtgagtgtgttttttgagtttgcatcaatcttaatgtct | |
| | | | | cttcataatacttttataatacattaagcctcttgtctacat | |
| | | 992 | TAGTGTGTCCTATTTCATACCTGTA | | |
| | | 993 | TGTACACACTTCCTCGTTTTGTAAT | | |
| | | 994 | TACTTACACCCAAACAGATCCTGAA | | |
| | | 995 | GGATGTAAGGTGCACGTTTCCTGAT | | |
| | | 996 | GACGCACTGTATTCCAGCTGGTGAT | | |
| | | 997 | GGTGATCAAGTCTGGGAACAGCCGT | | |
| | | 998 | GAACAGCCGTAACAGGTCAACCTTG | | |
| | | 999 | CAACCTTGTGGAGCCATCGCGAGTT | | |
| | | 1000 | GCATCAATCTTAATGTCTCTTCATA | | |
| | | 1001 | AATACATTAAGCCTCTTGTCTACAT | | |
| STX10 | 212625_at | 1002 | AGCTGGAGAGTAGAGGGTCCCGCCT | ccaggttctgaagcacatgtccggccgcgttggagaagagctggacgagcagg | NM_003765.1 |
| | | | | gcatcatgctggatgccttcgcccaagagatggaccacacccagtcccgcatg | |
| | | | | gacggggtcctcaggaagttggccaaagtatcccacatgacgagtgaccgccg | |
| | | | | acagtggtgtgccatcgccgtgctagtgggggtgcttctcctcgttctcatct | |
| | | | | tactattctctctctgaccccagccctccctggcaggctggtcccttaagcct | |
| | | | | ggggagccaccaagcactttggagctggcctcgccccctaggaggagagggtc | |
| | | | | cctcctgggtagctggagagtagagggtcccgcctggggagctgtccccatgg | |
| | | | | ctctcccctagagccagtgggacccttcaggaccctgggctggaaccaccacc | |
| | | | | actggtcctgtctcaagtgcacttagggggtggtggaggcagggacacctgagacac | |
| | | | | acctgtctccat | |
| | | 1003 | TGGTCCTGTCTCAAGTGCACTTAGG | | |
| | | 1004 | ACACCTGAGACACACCTGTCTCCAT | | |
| | | 1005 | CCAGGTTCTGAAGCACATGTCCGGC | | |
| | | 1006 | CCGGCCGCGTTGGAGAAGAGCTGGA | | |
| | | 1007 | GGCATCATGCTGGATGCCTTCGCCC | | |
| | | 1008 | TGCCTTCGCCCAAGAGATGGACCAC | | |
| | | 1009 | TCCCGCATGGACGGGGTCCTCAGGA | | |
| | | 1010 | GTATCCCACATGACGAGTGACCGCC | | |
| | | 1011 | TGGTCCCTTAAGCCTGGGGAGCCAC | | |
| | | 1012 | GGAGCCACCAAGCACTTTGGAGCTG | | |
| WWP1 | 212638_s_at | 1013 | GGATCTACCACCATATAAGAGTTAT | ggatctaccaccatataagagttatgaacaactaaaggaaaaacttctttttg | BF131791 |
| | | | | caatagaagagacagagggattnggacaagaatgaatgtggcttcttatttng | |
| | | | | gaggagctcttgcatttaaataccccagccaagaaaaattgcacagatagtgt | |
| | | | | atataagctgttcattctgtacagtgaattttccgaacctctcaaagtatgtt | |
| | | | | ttccgttcttccacagaaatatgcaaaacagttcatccttttctactttattt | |
| | | | | attgttcccttgaaatgactgaccaggaaaaagatcatccttaaattttgaag | |
| | | | | caagtgagagactttattaaaaatacatatatatctatataaacatatatgat | |
| | | | | agtggctctagttttatagagctccaagtgtattaaacatgacagccattcattcata | |
| | | | | aagatctggatttgctttaccttgttaa | |
| | | 1014 | GGAGCTCTTGCATTTAAATACCCCA | | |
| | | 1015 | TGCATTTAAATACCCCAGCCAAGAA | | |
| | | 1016 | AAGCTGTTCATTCTGTACAGTGAAT | | |
| | | 1017 | TACAGTGAATTTTCCGAACCTCTCA | | |
| | | 1018 | GAACCTCTCAAAGTATGTTTTCCGT | | |
| | | 1019 | ATGTTTTCCGTTCTTCCACAGAAAT | | |
| | | 1020 | GCAAAACAGTTCATCCTTTTCTACT | | |
| | | 1021 | ATTGTTCCCTTGAAATGACTGACCA | | |
| | | 1022 | TATGATAGTGGCTCTAGTTTTATAG | | |
| | | 1023 | ATCTGGATTTGCTTTACCTTGTTAA | | |
| RFTN1 | 212646_at | 1024 | TGCTGTTCATCCCACATCGTGTGGG | tgctgttcatcccacatcgtgtggggcagtgtccatcccctgcagctacttgg | D42043.1 |
| | | | | tgacttaacaactccaggagccctgtcagctgccctcctccanctaaanccct | |
| | | | | tcgactcttctgctttgacaaagaaaatgacattgggganggggaggtgctcc | |
| | | | | gcctcccagcttttctcaaaatagtcctatagatactggtaatctggaaatga | |
| | | | | agaagtaattctgtctctgcacctacttttgcagaatgttcaaggaagtattc | |
| | | | | tgtgttagtattaatgccaaaaagttgtttttaaaggttttgtactcagcaca | |
| | | | | tcatacaaaccacattacttctgtcacttcagggcatcgggactggctggcgc | |
| | | | | ccttgttatgtgctattttaatcagtgtaacattggtcaagttgttacccatg | |
| | | | | tatgctgtgtttatcatgtgtatatcgtccagaaagtattaaggctttaggta | |
| | | | | gatgcaactggcgaaccttggagagggaatgctgattgtcttgaccaaacccaca | |
| | | 1025 | CCTGCAGCTACTTGGTGACTTAACA | | |
| | | 1026 | TAACAACTCCAGGAGCCCTGTCAGC | | |
| | | 1027 | GTCTCTGCACCTACTTTTGCAGAAT | | |
| | | 1028 | TACAAACCACATTACTTCTGTCACT | | |
| | | 1029 | CTGTCACTTCAGGGCATCGGGACTG | | |
| | | 1030 | GCGCCCTTGTTATGTGCTATTTTAA | | |
| | | 1031 | GTTACCCATGTATGCTGTGTTTATC | | |
| | | 1032 | GTTTATCATGTGTATATCGTCCAGA | | |
| | | 1033 | GATGCAACTGGCGAACCTTGGAGAG | | |
| | | 1034 | GCTGATTGTCTTGACCAAACCCACA | | |
| IL1RN | 212657_s_at | 1035 | GGTACTATGTTAGCCCCATAATTTT | ggtactatgttagccccataattttttttttccttttaaaacacttccataat | AW083357 |
| | | | | ctggactcctctgtccaggcactgctgcccagcctccaagctccatctccact | |
| | | | | ccagattttttacagctgcctgcagtactttacctcctatcagaagtttctca | |
| | | | | gctcccaaggctctgagcaaatgtggctcctgggggttctttcttcctctgct | |
| | | | | gaaggaataaattgctccttgacattgtagagcttctggcacttggagacttg | |
| | | | | tatgaaagatggctgtgcctctgcctgtctcccccaccnggctgggagctctg | |
| | | | | cagagcaggaaacatgactcgtatatgtctcaggtccctgcagggccaagcac | |
| | | | | ctagcctcgctcttggcaggtactcagcgaatgaatgctgtatatgttgggtgcaaag | |
| | | | | ttccctacttcctgtgacttcagctctgtttta | |
| | | 1036 | ACACTTCCATAATCTGGACTCCTCT | | |
| | | 1037 | GATTTTTTACAGCTGCCTGCAGTAC | | |
| | | 1038 | CAGTACTTTACCTCCTATCAGAAGT | | |
| | | 1039 | AGCTCCCAAGGCTCTGAGCAAATGT | | |
| | | 1040 | GAGCAAATGTGGCTCCTGGGGGTTC | | |
| | | 1041 | ATAAATTGCTCCTTGACATTGTAGA | | |
| | | 1042 | TGACTCGTATATGTCTCAGGTCCCT | | |
| | | 1043 | CTCTTGGCAGGTACTCAGCGAATGA | | |
| | | 1044 | TGTTGGGTGCAAAGTTCCCTACTTC | | |
| | | 1045 | TTCCTGTGACTTCAGCTCTGTTTTA | | |
| ZNF364 | 212742_at | 1046 | TGAGGACTCTACTCGGCAAAGCCAG | ttaccttgcaatcacttctttcacagcagttgtattgtgccgtggctagaact | AL530462 |
| | | | | gcatgacacatgtcctgtatgtaggaagagcttaaatggtgaggactctactc | |
| | | | | ggcaaagccagagcactgaggcctctgcaagcaacagatttagcaatgacagt | |
| | | | | cagctacatgaccgatggactttctgaagctaaagaccacacctgaatcaggg | |
| | | | | ctgtggtaatcatcttaccatagctgtaaattgtatcaaaacaaaaaattagt | |
| | | | | agatggatttaggaatatgtaagaaactcaacacataatataaatgcaatgaa | |
| | | | | tgtttttcttctttaaatttaaagttagtatctacagatggaattgtatctac | |
| | | | | aaccaaatgcctcttatccctgaattcagagtgataattttataagtgtgaaa | |
| | | | | cttaattatgtagggctccccccgtctgaatagaattaattccttaaagtcta | |
| | | | | gttagggtcctgctgtctgtcatgttgccttgtaacggatgtttccacctccttctcc | |
| | | | | aacctctaccccaccattagtgtatttt | |
| | | 1047 | CACTGAGGCCTCTGCAAGCAACAGA | | |
| | | 1048 | CATGACCGATGGACTTTCTGAAGCT | | |
| | | 1049 | GAATCAGGGCTGTGGTAATCATCTT | | |
| | | 1050 | ATCTACAACCAAATGCCTCTTATCC | | |
| | | 1051 | TCTAGTTAGGGTCCTGCTGTCTGTC | | |
| | | 1052 | CTGTCTGTCATGTTGCCTTGTAACG | | |
| | | 1053 | CTCTACCCCACCATTAGTGTATTTT | | |
| | | 1054 | TTACCTTGCAATCACTTCTTTCACA | | |
| | | 1055 | ACAGCAGTTGTATTGTGCCGTGGCT | | |
| | | 1056 | GTGCCGTGGCTAGAACTGCATGACA | | |
| PPP1R16B | 212750_at | 1057 | TAACTTGGGGATGGTCTCCCCTGCC | taacttggggatggtctcccctgccccagggcacataagagcaaaggctccaa | AB020630.1 |
| | | | | tggtcagtggatgactctgcaaaagtgaccccctgtgccagaagctatagccc | |
| | | | | tctccccaacaggtctctcttgttggccagagggcctgcttcccatgggcatt | |
| | | | | gcaagtgccaccgtgcggggcctggctctgcacacccaggaaaagtctgcaga | |
| | | | | cccccagccctccgcaataattcaccagaccagaagccactggtgtacagaga | |
| | | | | acacttaaaaaaatgtattttatgtgaaaaaaaattaaaactctgtatactgt | |
| | | | | atcagcagctttgtgtaaaaatggcaatcaagagagtctaatatatttaaaac | |
| | | | | ttttttaaaaaaaatcttcgcagatctttgatatcgtactgaggtaacttcca | |
| | | | | cgtagccccttgccacgcggcaccggtgggccttgggtccaaaactgtggctc | |
| | | | | agccacatcccaaagggggcacatgtccctggagttgcttccagctgccaaggcctgt | |
| | | | | gacagaattcgctgtt | |
| | | 1058 | CTGCCCCAGGGCACATAAGAGCAAA | | |
| | | 1059 | GGATGACTCTGCAAAAGTGACCCCC | | |
| | | 1060 | CTCCCCAACAGGTCTCTCTTGTTGG | | |
| | | 1061 | CCTGCTTCCCATGGGCATTGCAAGT | | |
| | | 1062 | ATGGGCATTGCAAGTGCCACCGTGC | | |
| | | 1063 | CTCCGCAATAATTCACCAGACCAGA | | |
| | | 1064 | GTATACTGTATCAGCAGCTTTGTGT | | |
| | | 1065 | AAAATCTTCGCAGATCTTTGATATC | | |
| | | 1066 | TACTGAGGTAACTTCCACGTAGCCC | | |
| | | 1067 | AAGGCCTGTGACAGAATTCGCTGTT | | |
| NCAM1 | 212843_at | 1068 | GAATGTGAGAGCCTGGGTGTCTGAG | gaatgtgagagcctgggtgtctgagaccgggagggcccagcagtgaggggcag | AA126505 |
| | | | | gctcttctggtcaccaggctgttcagtggactcagttcttcatcttgtaatgt | |
| | | | | cgatggctttgccacaccaggccaagcccatgccataccttgtcaagactgtc | |
| | | | | aaagtggttgtggttaggtcaaactggttttggttctgatggttaggaagaaa | |
| | | | | caggtcagccctcagatcacctggcccgggacagctgaccccctagaaccctg | |
| | | | | gctctgccattagctaggacctaagactctgcccacattttggtctgttctctcccat | |
| | | | | tacacataggtttgtctcagcatgcaagagt | |
| | | 1069 | GCCTGGGTGTCTGAGACCGGGAGGG | | |
| | | 1070 | CTCTTCTGGTCACCAGGCTGTTCAG | | |
| | | 1071 | GGCTGTTCAGTGGACTCAGTTCTTC | | |
| | | 1072 | GGACTCAGTTCTTCATCTTGTAATG | | |
| | | 1073 | CTTGTAATGTCGATGGCTTTGCCAC | | |
| | | 1074 | CATGCCATACCTTGTCAAGACTGTC | | |
| | | 1075 | GAAGAAACAGGTCAGCCCTCAGATC | | |
| | | 1076 | GCTCTGCCATTAGCTAGGACCTAAG | | |
| | | 1077 | TTAGCTAGGACCTAAGACTCTGCCC | | |
| | | 1078 | TAGGTTTGTCTCAGCATGCAAGAGT | | |
| NKG7 | 213915_at | 1079 | ATTTCTGGTTTGAGGCTGTGGGTCC | atttctggtttgaggctgtgggtcccacccactcagctcactcgggcctctgg | NM_005601.1 |
| | | | | ccaacagggcatggngacatcatatcaggctacatccacgtgacgcagacctt | |
| | | | | cagcattatggctgttctgtgggccctggtgtccgtgagcttcctggtcctgt | |
| | | | | cctgcttcccctcactgttccccccaggccacggcccgcttgtctcaaccacc | |
| | | | | gcagcctttgctgcagccatctccatggtggtggccatggcggtgtacaccag | |
| | | | | cgagcggtgggaccagcctccacacccccagatccagaccttcttctcctggt | |
| | | | | ccttctacctgggctgggtctcagctatcctcttgctctgtacaggtgccctg | |
| | | | | agcctgggtgctcactgtggcggtccccgtcctggctatgaaaccttgtgagcagaa | |
| | | | | ggcaagagcggcaagatgagttttgagcgttgtattcca | |
| | | 1080 | GACATCATATCAGGCTACATCCACG | | |
| | | 1081 | TCATATCAGGCTACATCCACGTGAC | | |
| | | 1082 | ACGCAGACCTTCAGCATTATGGCTG | | |
| | | 1083 | CATTATGGCTGTTCTGTGGGCCCTG | | |
| | | 1084 | TGGCCATGGCGGTGTACACCAGCGA | | |
| | | 1085 | TACACCAGCGAGCGGTGGGACCAGC | | |
| | | 1086 | CTATCCTCTTGCTCTGTACAGGTGC | | |
| | | 1087 | CGTCCTGGCTATGAAACCTTGTGAG | | |
| | | 1088 | GTGAGCAGAAGGCAAGAGCGGCAAG | | |
| | | 1089 | GATGAGTTTTGAGCGTTGTATTCCA | | |
| HLA-A | 213932_x_at | 1090 | GAAGAACCCTGACTTTGTTTCTGCA | gacagacctcaggagggctattggtccaggacccacacctgctttcttcatgt | AI923492 |
| /// HLA- | | | | ttcctgatcccgccctgggtctgcagtcacacatttctggaaacttctctggg | |
| H /// | | | | gtccaagactaggaggttcnnctnggaccttanggccntggntcntttctggt | |
| LOC642047 | | | | atctcacanggacattnncttctcacagatagaaaaggagggagttacactca | |
| /// | | | | ggctgcanncagtgacagtgcccaggctctgatgtgtcnctcacagcttgtaa | |
| LOC649853 | | | | agtgtgagacagctgccttgtgtgggactgagaggcaagagttgttcctgccc | |
| /// | | | | ttccctttgtgacttgaagaaccctgactttgtttctgcaaaggcacctgcat | |
| LOC649864 | | | | gtgtctgtgttcgtgtaggcntaatgtgaggaggtggggagaccaccccaccc | |
| | | | | cnatgtccaccatgaccctcttcccacgctgacctgtgctccctccccaatca | |
| | | | | tctttcctgttccagagaggtggggctgaggtgtctccatctctgtctcaacttcat | |
| | | | | ggtgcactgagctgtaacttcttc | |
| | | 1091 | AAGGCACCTGCATGTGTCTGTGTTC | | |
| | | 1092 | CAATCATCTTTCCTGTTCCAGAGAG | | |
| | | 1093 | CATCTCTGTCTCAACTTCATGGTGC | | |
| | | 1094 | TGGTGCACTGAGCTGTAACTTCTTC | | |
| | | 1095 | GACAGACCTCAGGAGGGCTATTGGT | | |
| | | 1096 | GGCTATTGGTCCAGGACCCACACCT | | |
| | | 1097 | GGTCTGCAGTCACACATTTCTGGAA | | |
| | | 1098 | GGAAACTTCTCTGGGGTCCAAGACT | | |
| | | 1099 | AGACAGCTGCCTTGTGTGGGACTGA | | |
| | | 1100 | TGCCCTTCCCTTTGTGACTTGAAGA | | |
| YPEL1 | 213996_at | 1101 | GCCGAACTGTCACCGAACGTACAGC | gccgaactgtcaccgaacgtacagctgtatccactgcagagcacacctggcca | NM_013313.1 |
| | | | | atcatgacgagctcatctccaagtcctttcaggggagccagggacgcgcctac | |
| | | | | ctcttcaattccgtggtgaacgtgggctgcggccctgcagaggagagggtcct | |
| | | | | tctcaccgggctgcatgcggttgccgacatctactgcgagaactgcaagacca | |
| | | | | cgctcgggtggaaatacgagcatgcctttgagagcagtcagaaatataaggaa | |
| | | | | ggaaaattcatcattgagcttgctcatatgatcaaagacaatggctgggagta | |
| | | | | atgtgcgaactttcccttctccttngaatgctgttttgtgaaagaaactgtga | |
| | | | | atgtaatggaaacgtaggagcatctggtgacagcctttcttgccctctgacct | |
| | | | | caaaggctagctgcgcatagctcttgacactcncggccatctctgtgggtaaggtgt | |
| | | | | ccctcggatctgtcctcttcgtgtacacagttgtt | |
| | | 1102 | CGTACAGCTGTATCCACTGCAGAGC | | |
| | | 1103 | ACACCTGGCCAATCATGACGAGCTC | | |
| | | 1104 | TCCAAGTCCTTTCAGGGGAGCCAGG | | |
| | | 1105 | TGCATGCGGTTGCCGACATCTACTG | | |
| | | 1106 | GAGTAATGTGCGAACTTTCCCTTCT | | |
| | | 1107 | TAGGAGCATCTGGTGACAGCCTTTC | | |
| | | 1108 | GCCCTCTGACCTCAAAGGCTAGCTG | | |
| | | 1109 | TAGCTGCGCATAGCTCTTGACACTC | | |
| | | 1110 | TGTGGGTAAGGTGTCCCTCGGATCT | | |
| | | 1111 | TGTCCTCTTCGTGTACACAGTTGTT | | |
| ZAP70 | 214032_at | 1112 | AAGGGCCGGAGGTCATGGCCTTCAT | aagggccggaggtcatggccttcatcgagcagggcaagcggatggantgccca | AI817942 |
| | | | | ccagagtgtnccacccgaactgtacgcactcatgagtgactgctggatctaca | |
| | | | | agtgggaggatcgccccgacttcctgaccgtggagcagcgcatgcgagcctgt | |
| | | | | tactacagcctggccagcaaggtggaagggcccccaggcagcacacagaaggc | |
| | | | | tgaggctgcctgtgcctgagctcccgctgcccaggggagccctccacnccggc | |
| | | | | tcttccccaccctcagccccaccccaggtcctgcagtctggctgagccctgct | |
| | | | | tggttgtctccacacacagctgggctgtggtagggggtgtctcaggccacacc | |
| | | | | ggccttgcattgcctgcctggccccctgtcctctctggctggggagcagggag | |
| | | | | gtccgggagggtgcggctgtgcagcctgtcctgggctggtggctcccggaggg | |
| | | | | ccctgagctgagggcattgcttacacggatgccttcccctgggccctgacatt | |
| | | | | ggagcctgggcatcctcaggtggtcaggcgtagatcaccagaataaacccagcttccc | |
| | | 1113 | CCCGAACTGTACGCACTCATGAGTG | | |
| | | 1114 | TCATGAGTGACTGCTGGATCTACAA | | |
| | | 1115 | CATGCGAGCCTGTTACTACAGCCTG | | |
| | | 1116 | CACAGCTGGGCTGTGGTAGGGGGTG | | |
| | | 1117 | AGCAGGGAGGTCCGGGAGGGTGCGG | | |
| | | 1118 | GGCATTGCTTACACGGATGCCTTCC | | |
| | | 1119 | TGGGCCCTGACATTGGAGCCTGGGC | | |
| | | 1120 | TGACATTGGAGCCTGGGCATCCTCA | | |
| | | 1121 | GGTGGTCAGGCGTAGATCACCAGAA | | |
| | | 1122 | ATCACCAGAATAAACCCAGCTTCCC | | |
| CTSW | 214450_at | 1123 | GGAGAGAAGGGCTATTTCCGGCTGC | caggacttcatcatgctgcagaacaacgagcacagaattgcgcagtacctggc | NM_001335.1 |
| | | | | cacttatggccccatcaccgtgaccatcaacatgaagccccttcagctatacc | |
| | | | | ggaaaggtgtgatcaaggccacacccaccacctgtgacccccagcttgtggac | |
| | | | | cactctgtcctgctggtgggttttggcagcgtcaagtcagaggaggggatatg | |
| | | | | ggcagagacagtctcatcgcagtctcagcctcagcctccacaccccaccccat | |
| | | | | actggatcctgaagaactcctggggggcccaatggggagagaagggctatttc | |
| | | | | cggctgcaccgagggagcaatacctgtggcatcaccaagttcccgctcactgcccgtg | |
| | | | | tgcagaaaccggatatgaagccccgagtctc | |
| | | 1124 | CGGCTGCACCGAGGGAGCAATACCT | | |
| | | 1125 | ATACCTGTGGCATCACCAAGTTCCC | | |
| | | 1126 | CTCACTGCCCGTGTGCAGAAACCGG | | |
| | | 1127 | AACCGGATATGAAGCCCCGAGTCTC | | |
| | | 1128 | CAGGACTTCATCATGCTGCAGAACA | | |
| | | 1129 | GCGCAGTACCTGGCCACTTATGGCC | | |
| | | 1130 | AGCCCCTTCAGCTATACCGGAAAGG | | |
| | | 1131 | GGGTTTTGGCAGCGTCAAGTCAGAG | | |
| | | 1132 | GAGACAGTCTCATCGCAGTCTCAGC | | |
| | | 1133 | CCACCCCATACTGGATCCTGAAGAA | | |
| PRF1 | 214617_at | 1134 | CCAACGCAAATTCGCAAACTTTCTT | ccaacgcaaattcgcaaactttcttaaaacattatgagttncnntttgctatt | AI445650 |
| | | | | tttttttttttttttagctcatcggctatcgttagtgctagtggattttacat | |
| | | | | gtggcccnnnannnnnnnncnnncaacgtggcccagagaagccaaaagattgg | |
| | | | | atacgcatcagacagatggaaaagggagattcagactgtttttcagggaggtg | |
| | | | | gctgggtttacacgctaatcccgattcaccctgtccaaactgcctaagccctc | |
| | | | | cgccattntcaagccctgcagtcacagctacacagatcacagcttcagccagg | |
| | | | | agctgggcagaaggccaanaggctgttcccaccaggctgctcagggntggtct | |
| | | | | tttaggacccttcccttgagccctntatggtgtggcaaagccttcattgcctt | |
| | | | | aactggagccccatcagctccagctgctctgtnttntttgcccncaatgcttt | |
| | | | | gcccctgagacaaatggaggcctgtcctgacctgtctcaccatgtacatagctt | |
| | | 1135 | GCTCATCGGCTATCGTTAGTGCTAG | | |
| | | 1136 | TGCTAGTGGATTTTACATGTGGCCC | | |
| | | 1137 | AGATTGGATACGCATCAGACAGATG | | |
| | | 1138 | GAGGTGGCTGGGTTTACACGCTAAT | | |
| | | 1139 | GGGTTTACACGCTAATCCCGATTCA | | |
| | | 1140 | GCAGTCACAGCTACACAGATCACAG | | |
| | | 1141 | GCCTTCATTGCCTTAACTGGAGCCC | | |
| | | 1142 | CAATGCTTTGCCCCTGAGACAAATG | | |
| | | 1143 | GACAAATGGAGGCCTGTCCTGACCT | | |
| | | 1144 | ACCTGTCTCACCATGTACATAGCTT | | |
| SULT1A1 | 215299_x_at | 1145 | AAGATCCTGGAGTTTGTGGGGCGCT | aagatcctggagtttgtggggcgctccctnccagaggagacngtggacntcat | U37025 |
| | | | | ggttnagcacacgtcgttcaaggagatgaagaagaaccctatgaccaactaca | |
| | | | | ccaccgtccnccnggagttcatggaccacagcatctcccccttcatgaggaaa | |
| | | | | ggcatggctggggacnngngnngnccacnttcaccgtggcgcagaatgagcgc | |
| | | | | ttcgatgcggacntatgcggagaagatggcaggncngcagcctcangcttccg | |
| | | | | ctntgagcngtgagaggggnnncntggagtcacngcagagggagtgtgcgaat | |
| | | | | caaacctgaccaagcggntcaagaataaaatatgaattgagggccngggacgg | |
| | | | | taggtcatgtctgtaatcccagcaatttggaggctgaggtgggaggatcattt | |
| | | | | gagcccaggagttcgagaccaacctgggcaacatagtgagattctgttaaaaa | |
| | | | | aataaaataaaataaaaccaatttttaaaaagagaataaaatatgattgtgggccagg | |
| | | | | cagagtggctcatgc | |
| | | 1146 | AGCACACGTCGTTCAAGGAGATGAA | | |
| | | 1147 | AGAAGAACCCTATGACCAACTACAC | | |
| | | 1148 | GGAGTTCATGGACCACAGCATCTCC | | |
| | | 1149 | CATCTCCCCCTTCATGAGGAAAGGC | | |
| | | 1150 | TTCACCGTGGCGCAGAATGAGCGCT | | |
| | | 1151 | GCAGAATGAGCGCTTCGATGCGGAC | | |
| | | 1152 | GGGAGTGTGCGAATCAAACCTGACC | | |
| | | 1153 | GTGCGAATCAAACCTGACCAAGCGG | | |
| | | 1154 | GGACGGTAGGTCATGTCTGTAATCC | | |
| | | 1155 | GTGGGCCAGGCAGAGTGGCTCATGC | | |
| C7ORF24 | 215380_s_at | 1156 | GAAAATGGTTTGCCGCTGGAGTATC | gaaaatggtttgccgctggagtatcaagagaagttaaaagcaatagaaccaaa | AK021779.1 |
| | | | | tgactatacaggaaaggtctcagaagaaattgaagacatcatcaaaaaggggg | |
| | | | | aaacacaaactctttagaacataacagaatatatctaagggtattctatgtgc | |
| | | | | taatataaaatatttttaacacttgagaacagggatctgggggatctccacgt | |
| | | | | ttgatccattttcagcagtgctctgaaggagtatcttacttgggtgattcctt | |
| | | | | gtttttagactataaaaagaaactgggataggagttagacaatttaaaagggg | |
| | | | | tgtatgagggcctgaaatatgtgacaaatgaatgtgagtaccccttctatgaa | |
| | | | | cactgaaagctattctcttgaattgatcttaagtgtctccttgctctggtaaa | |
| | | | | agatagatttgtagctcacttgatgatggtgctggtgaattgctctgctctgtctgag | |
| | | | | att | |
| | | 1157 | ATCTAAGGGTATTCTATGTGCTAAT | | |
| | | 1158 | ATCTGGGGGATCTCCACGTTTGATC | | |
| | | 1159 | CAGCAGTGCTCTGAAGGAGTATCTT | | |
| | | 1160 | GGAGTATCTTACTTGGGTGATTCCT | | |
| | | 1161 | GGGTGATTCCTTGTTTTTAGACTAT | | |
| | | 1162 | ACAAATGAATGTGAGTACCCCTTCT | | |
| | | 1163 | ATTGATCTTAAGTGTCTCCTTGCTC | | |
| | | 1164 | GTGTCTCCTTGCTCTGGTAAAAGAT | | |
| | | 1165 | AGATAGATTTGTAGCTCACTTGATG | | |
| | | 1166 | GAATTGCTCTGCTCTGTCTGAGATT | | |
| HOMER3 | 215489_x_at | 1167 | CAATGTCCACAGCCAGGGAGCAGCC | gagggacactcatagtccctcctctctccctaggggccaaaccagtgctcctg | AI871287 |
| | | | | ccacctctctggctgccccctagagcctgcccatcccagcctgaccaatgtcc | |
| | | | | acagccagggagcagccaatcttcagcacacgggcgcacgtgttccaaattga | |
| | | | | cccagccaccaagcgaaactggatcccagcgggcaagcacgcactcactgtct | |
| | | | | cctatttctacgatgccacccgcaatgtgtaccgcatcatcagcatcggaggc | |
| | | | | gccaaggccatcatcaacagcactgtcactcccaacatgaccttcaccaaaac | |
| | | | | ttcccagaagttcgggcagtgggccgacagtcgcgccaacacagtctatggcc | |
| | | | | tgggctttgcctctgaacagcatctgacacagtttgccgagaagttccaggaa | |
| | | | | gtgaaggaagcagccaggctggccagggagaaatctcaggatggctggggtgg | |
| | | | | gccccagtcggctctggttgttggcagctttggggctgtttttgagcttctcatt | |
| | | 1168 | GGGCGCACGTGTTCCAAATTGACCC | | |
| | | 1169 | GTACCGCATCATCAGCATCGGAGGC | | |
| | | 1170 | GGCCATCATCAACAGCACTGTCACT | | |
| | | 1171 | AAGTTCGGGCAGTGGGCCGACAGTC | | |
| | | 1172 | AGTCGCGCCAACACAGTCTATGGCC | | |
| | | 1173 | GCTTTGCCTCTGAACAGCATCTGAC | | |
| | | 1174 | GACACAGTTTGCCGAGAAGTTCCAG | | |
| | | 1175 | CCCAGTCGGCTCTGGTTGTTGGCAG | | |
| | | 1176 | TGGGGCTGTTTTTGAGCTTCTCATT | | |
| | | 1177 | GAGGGACACTCATAGTCCCTCCTCT | | |
| LILRA5 | 215838_at | 1178 | TCCTGCAGGTATGGTCAGAACCCAG | tcctgcaggtatggtcagaacccagtgacctcctggagattccggtctcagga | AF212842.1 |
| | | | | gcagctgataacctcagtccgtcacanaacaagtctgactctgggactgcctc | |
| | | | | acaccttcaggattacgcagtagagaatctcatccgcatgggcatggccggct | |
| | | | | tgatcctggtggtccttgggattctgatatttcaggattggcacagccagaga | |
| | | | | agcccccaagctgcagctggaaggtgaacagaagagagaacaatgcaccattg | |
| | | | | aatgctggagccttggaagcgaatctgatggtcctaggaggttcgggaagaccatctg | |
| | | | | aggcctatgccatctggactgtctgctggcaatttcttt | |
| | | 1179 | TGGAGATTCCGGTCTCAGGAGCAGC | | |
| | | 1180 | GAGCAGCTGATAACCTCAGTCCGTC | | |
| | | 1181 | TGCCTCACACCTTCAGGATTACGCA | | |
| | | 1182 | GGTCCTTGGGATTCTGATATTTCAG | | |
| | | 1183 | AATGCACCATTGAATGCTGGAGCCT | | |
| | | 1184 | GCTGGAGCCTTGGAAGCGAATCTGA | | |
| | | 1185 | GCGAATCTGATGGTCCTAGGAGGTT | | |
| | | 1186 | GGTTCGGGAAGACCATCTGAGGCCT | | |
| | | 1187 | TGAGGCCTATGCCATCTGGACTGTC | | |
| | | 1188 | TGGACTGTCTGCTGGCAATTTCTTT | | |
| PTGDR | 215894_at | 1189 | CGCGCGCGGACGGGAGGGAAGCGTC | gccatgcgcaacctctatgcgatgcaccggcggctgcagcggcacccgcgctc | U31099.1 |
| | | | | ctgcaccagggactgtgccgagccgcgcgcggacgggagggaagcgtcccctc | |
| | | | | agcccctggaggagctggatcacctcctgctgctggcgctgatgaccgtgctc | |
| | | | | ttcactatgtgttctctgcccgtaatttatcgcgcttactatggagcatttaa | |
| | | | | ggatgtcaaggagaaaaacaggacctctgaagaagcagaagacctccgagcct | |
| | | | | tgcgatttctatctgtgatttcaattgtggacccttggatttttatcattttc | |
| | | | | agatctccagtatttcggatattttttcacaagattttcattagacctcttag | |
| | | | | gtacaggagccggtgcagcaattccactaacatggaatccagtctgtgacagtgttt | |
| | | | | ttcactc | |
| | | 1190 | TGCCCGTAATTTATCGCGCTTACTA | | |
| | | 1191 | GAAGAAGCAGAAGACCTCCGAGCCT | | |
| | | 1192 | AGCCTTGCGATTTCTATCTGTGATT | | |
| | | 1193 | GCCATGCGCAACCTCTATGCGATGC | | |
| | | 1194 | GATTTCAATTGTGGACCCTTGGATT | | |
| | | 1195 | TTTTCAGATCTCCAGTATTTCGGAT | | |
| | | 1196 | TTTCACAAGATTTTCATTAGACCTC | | |
| | | 1197 | AGACCTCTTAGGTACAGGAGCCGGT | | |
| | | 1198 | AACATGGAATCCAGTCTGTGACAGT | | |
| | | 1199 | CAGTCTGTGACAGTGTTTTTCACTC | | |
| LPXN | 216250_s_at | 1200 | GCTTTCTGCCTGACACAGTTGTCGA | gctttctgcctgacacagttgtcgaagggcattttcagggagcagaatgacaa | X77598.1 |
| | | | | gacctattgtcaaccttgcttcaataagctcttcccactgtaatgccaactga | |
| | | | | tccatagcctcttcagattccttataaaatttaaaccaagagaggagaggaaa | |
| | | | | gggtaaattttctgttactgaccttctgcttaatagtcttatagaaaaaggaa | |
| | | | | aggtgatgagcaaataaaggaacttctagactttacatgactaggctgataat | |
| | | | | cttattttttaggcttctatacagttaattctataaattctctttctccctct | |
| | | | | cttctccaatcaagcacttggagttagatctaggtccttctatctcgtccctc | |
| | | | | tacagatgtattttccacttgcataattcatgccaacactggttttcttaggt | |
| | | | | ttctccattttcacctctagtgatggccctactcatatcttctctaatttggt | |
| | | | | cctgatacttgtttcttttcacgttttcccatttccctgtggctcactgtcttacaa | |
| | | | | tcactg | |
| | | 1201 | AAGACCTATTGTCAACCTTGCTTCA | | |
| | | 1202 | TCTTCCCACTGTAATGCCAACTGAT | | |
| | | 1203 | CCAACTGATCCATAGCCTCTTCAGA | | |
| | | 1204 | CTGTTACTGACCTTCTGCTTAATAG | | |
| | | 1205 | GTTAGATCTAGGTCCTTCTATCTCG | | |
| | | 1206 | TATCTCGTCCCTCTACAGATGTATT | | |
| | | 1207 | TTTCACCTCTAGTGATGGCCCTACT | | |
| | | 1208 | ACTCATATCTTCTCTAATTTGGTCC | | |
| | | 1209 | GGTCCTGATACTTGTTTCTTTTCAC | | |
| | | 1210 | GTGGCTCACTGTCTTACAATCACTG | | |
| PYHIN1 | 216748_at | 1211 | CCACCCTCTGGATCCCAATATTGAG | ccaccctctggatcccaatattgagatcttatcctcagggaatcctcacttag | AK024890.1 |
| | | | | acccctgtaacaggttaaatcttcatggtgttctgtttcctaggaacttcttt | |
| | | | | cttttctactgtttatgacaactgaagttaataagtgtttatctttcccacct | |
| | | | | actcaaagtagttccaagattagggctagtttgtaattctgtggaccactgta | |
| | | | | aacgagggcctagttcagtgtctgcctcatgggaagcttccaataaatacctttg | |
| | | 1212 | TTATCCTCAGGGAATCCTCACTTAG | | |
| | | 1213 | CCTCACTTAGACCCCTGTAACAGGT | | |
| | | 1214 | AAATCTTCATGGTGTTCTGTTTCCT | | |
| | | 1215 | GTGTTCTGTTTCCTAGGAACTTCTT | | |
| | | 1216 | TAATAAGTGTTTATCTTTCCCACCT | | |
| | | 1217 | ATCTTTCCCACCTACTCAAAGTAGT | | |
| | | 1218 | GTAGTTCCAAGATTAGGGCTAGTTT | | |
| | | 1219 | GTGGACCACTGTAAACGAGGGCCTA | | |
| | | 1220 | CGAGGGCCTAGTTCAGTGTCTGCCT | | |
| | | 1221 | GGGAAGCTTCCAATAAATACCTTTG | | |
| SLC35E2 | 217122_s_at | 1222 | GTCTCTGAAGTATTTCCTCCAGTTT | gtctctgaagtatttcctccagtttccctgcgggcccctatgtttgagtttga | AL031282 |
| | | | | tggctgctggatcctcactcaacgaaaactcggttggaaactgttccgcctgg | |
| | | | | cagtccttttttgttgttttccatctcatttcccttccatctgaaagtggcat | |
| | | | | tcagctgacttgctcatttagactgttcacggagtctgaatctgccaacgtgg | |
| | | | | tgttggaggctccaccttgaaaagggccacagtcagggcaactttccccatac | |
| | | | | aggaaaacttgaaaattacatcaacagtctacgtcacagccaaattatatttc | |
| | | | | ctttataccaaacaaaactatggagaactaaaagtacatcacacaaaacgttt | |
| | | | | atagtgttttgcatgtgacctatttcagtatttatataactagattagtgctt | |
| | | | | tctagcaaacggttctgttaattagcgagtcactgttgattctgctgtggtggtaag | |
| | | | | ttgataccgtgtaactaatcccgtggat | |
| | | 1223 | GGGCCCCTATGTTTGAGTTTGATGG | | |
| | | 1224 | GGATCCTCACTCAACGAAAACTCGG | | |
| | | 1225 | CTCGGTTGGAAACTGTTCCGCCTGG | | |
| | | 1226 | GACTTGCTCATTTAGACTGTTCACG | | |
| | | 1227 | GAGTCTGAATCTGCCAACGTGGTGT | | |
| | | 1228 | TCAGGGCAACTTTCCCCATACAGGA | | |
| | | 1229 | TACATCAACAGTCTACGTCACAGCC | | |
| | | 1230 | GTGCTTTCTAGCAAACGGTTCTGTT | | |
| | | 1231 | TAGCGAGTCACTGTTGATTCTGCTG | | |
| | | 1232 | ATACCGTGTAACTAATCCCGTGGAT | | |
| TRA@ // | 217143_s_at | 1233 | GTTGACCTGTCATAGCCTTGTTAAA | gaaggtgaacatgatgtccctcacagtgcttgggctacgaatgctgtttgcaa | X06557.1 |
| TRD@ | | | | agactgttgccgtcaattttctcttgactgccaagttatttttcttgtaaggc | |
| | | | | tgactggcatgaggaagctacactcctgaagaaaccaaaggcttacaaaaatg | |
| | | | | catctccttggcttctgacttctttgtgattcaagttgacctgtcatagcctt | |
| | | | | gttaaaatggctgctagccaaaccactttttcttcaaagacaacaaacccagc | |
| | | | | tcatcctccagcttgatgggaagacaaaagtcctggggaaggggggtttatgtcctaa | |
| | | | | ctgctttgta | |
| | | 1234 | TCAAAGACAACAAACCCAGCTCATC | | |
| | | 1235 | GCTCATCCTCCAGCTTGATGGGAAG | | |
| | | 1236 | GGGTTTATGTCCTAACTGCTTTGTA | | |
| | | 1237 | GAAGGTGAACATGATGTCCCTCACA | | |
| | | 1238 | GCTTGGGCTACGAATGCTGTTTGCA | | |
| | | 1239 | AGACTGTTGCCGTCAATTTTCTCTT | | |
| | | 1240 | AATTTTCTCTTGACTGCCAAGTTAT | | |
| | | 1241 | TTTTCTTGTAAGGCTGACTGGCATG | | |
| | | 1242 | GAAGCTACACTCCTGAAGAAACCAA | | |
| | | 1243 | AAAAATGCATCTCCTTGGCTTCTGA | | |
| TRATRD | 217147_s_at | 1244 | TCTCCTTTCTCACCAATGGGCAATA | tctcctttctcaccaatgggcaatagcccataattgaaataaatttctgattg | AJ240085.1 |
| | | | | aaaggtataggaaacattaaaatgcattactaagagaagtaatataattttct | |
| | | | | tacaaagtatttttcccaaagatagctttactatttcaaaaattgtcaaatta | |
| | | | | atgcatgctccttacaacaaacaaatatcaaaaagagtttaggaattctacta | |
| | | | | gccagagatagtcacttggagaaactttctatatatccttctaaatatttttc | |
| | | | | tgggcatgctcatgtatgtacatcagttgtttctttttattttgaaccaaaaa | |
| | | | | tgtggtttcttttgtacacattacttaaactttctttccagtcaacaatatat | |
| | | | | tgtggatttattttcactgttatatttaactatatataaatacgcatatattgtaat | |
| | | | | tttaatgtctgcttagcaccccactgataaccaaatcacag | |
| | | 1245 | TCCTTTCTCACCAATGGGCAATAGC | | |
| | | 1246 | TATTTTTCCCAAAGATAGCTTTACT | | |
| | | 1247 | GTCAAATTAATGCATGCTCCTTACA | | |
| | | 1248 | AATGCATGCTCCTTACAACAAACAA | | |
| | | 1249 | ATGCTCCTTACAACAAACAAATATC | | |
| | | 1250 | GAGTTTAGGAATTCTACTAGCCAGA | | |
| | | 1251 | ACTAGCCAGAGATAGTCACTTGGAG | | |
| | | 1252 | GATAGTCACTTGGAGAAACTTTCTA | | |
| | | 1253 | GAAACTTTCTATATATCCTTCTAAA | | |
| | | 1254 | CACCCCACTGATAACCAAATCACAG | | |
| S100A6 | 217728_at | 1255 | GGGACCGCTATAAGGCCAGTCGGAC | gggaccgctataaggccagtcggactgcgacatagcccatcccctcgaccgct | NM_014624.2 |
| | | | | cgcgtcgcatttggccgcctccctaccgctccaagcccagccctcagccatgg | |
| | | | | catgccccctggatcaggccattggcctcctcgtggccatcttccacaagtac | |
| | | | | tccggcagggagggtgacaagcacaccctgagcaagaaggagctgaaggagctgatc | |
| | | | | cagaaggagctcaccattggctcgaagctgcagg | |
| | | 1256 | TCGTGGCCATCTTCCACAAGTACTC | | |
| | | 1257 | TTCCACAAGTACTCCGGCAGGGAGG | | |
| | | 1258 | CCGCTATAAGGCCAGTCGGACTGCG | | |
| | | 1259 | TCCGGCAGGGAGGGTGACAAGCACA | | |
| | | 1260 | GACAAGCACACCCTGAGCAAGAAGG | | |
| | | 1261 | GCTGATCCAGAAGGAGCTCACCATT | | |
| | | 1262 | GAAGGAGCTCACCATTGGCTCGAAG | | |
| | | 1263 | AGCTCACCATTGGCTCGAAGCTGCA | | |
| | | 1264 | CTCACCATTGGCTCGAAGCTGCAGG | | |
| | | 1265 | GCCAGTCGGACTGCGACATAGCCCA | | |
| RAB31 | 217763_s_at | 1266 | AACATTGTAATGGCCATCGCTGGAA | aacattgtaatggccatcgctggaaacaagtgcgacctctcagatattaggga | NM_006868.1 |
| | | | | ggttcccctgaaggatgctaaggaatacgctgaatccataggtgccatcgtgg | |
| | | | | ttgagacaagtgcaaaaaatgctattaatatcgaagagctctttcaaggaatc | |
| | | | | agccgccagatcccacccttggacccccatgaaaatggaaacaatggaacaat | |
| | | | | caaagttgagaagccaaccatgcaagccagccgccggtgctgttgacccaagg | |
| | | | | gcgtggtccacggtacttgaagaagccagagcccacatcctgtgcactgctga | |
| | | | | aggaccctacgctcggtggcctggcacctcactttgagaagagtgagcacact | |
| | | | | ggctttgcatcctggaaggcctgcagggggcggggcaggaaatgtacctgaaa | |
| | | | | aggattttagaaaaccctgggaaacccaccacaccaccacaaaatggcctttagtgt | |
| | | 1267 | GAAACAAGTGCGACCTCTCAGATAT | | |
| | | 1268 | GGAGGTTCCCCTGAAGGATGCTAAG | | |
| | | 1269 | TACGCTGAATCCATAGGTGCCATCG | | |
| | | 1270 | GTGCCATCGTGGTTGAGACAAGTGC | | |
| | | 1271 | TTCAAGGAATCAGCCGCCAGATCCC | | |
| | | 1272 | TGAGAAGCCAACCATGCAAGCCAGC | | |
| | | 1273 | CGTGGTCCACGGTACTTGAAGAAGC | | |
| | | 1274 | ATCCTGTGCACTGCTGAAGGACCCT | | |
| | | 1275 | GAGTGAGCACACTGGCTTTGCATCC | | |
| | | 1276 | ACCACCACAAAATGGCCTTTAGTGT | | |
| EVL | 217838_s_at | 1277 | GATCATCGACGCCATCAGGCAGGAG | gatcatcgacgccatcaggcaggagctgagtgggatcagcaccacgtaagggg | NM_016337.1 |
| | | | | ccggcctcgctgcgctgattcgtcgagcccatccggcgacagaggacagccag | |
| | | | | aagcccagccagccccagactccagtgcaccagagcacgcacaggagcctggg | |
| | | | | cgcgctgctgtgaaacgtcctgacctgtgatcacacatgacagtgaggaaacc | |
| | | | | aagtgcaactcctgggtttttttagattctgcctgacacggaacaccaggtct | |
| | | | | gctcgtcttttttgtgttttatatttgcttatttaaggtacatttctttgggtttcta | |
| | | | | gagacgcccctaagtcacctgcttcattagacggtttccaggttttct | |
| | | 1278 | TGGGATCAGCACCACGTAAGGGGCC | | |
| | | 1279 | CCCATCCGGCGACAGAGGACAGCCA | | |
| | | 1280 | GTGCACCAGAGCACGCACAGGAGCC | | |
| | | 1281 | TGAAACGTCCTGACCTGTGATCACA | | |
| | | 1282 | GGAAACCAAGTGCAACTCCTGGGTT | | |
| | | 1283 | TCCTGGGTTTTTTTAGATTCTGCCT | | |
| | | 1284 | TAGATTCTGCCTGACACGGAACACC | | |
| | | 1285 | CTGACACGGAACACCAGGTCTGCTC | | |
| | | 1286 | GGTACATTTCTTTGGGTTTCTAGAG | | |
| | | 1287 | TCATTAGACGGTTTCCAGGTTTTCT | | |
| SMAD3 | 218284_at | 1288 | GGTGTAGTGGCTTTTTGGCTCAGCA | ggtgtagtggctttttggctcagcatccagaaacaccaaaccaggctggctaa | NM_015400.1 |
| | | | | acaagtggccgcgtgtaaaaacagacagctctgagtcaaatctgggcccttcc | |
| | | | | acaagggtcctctgaaccaagccccactcccttgctaggggtgaaagcattac | |
| | | | | agagagatggagccatctatccaagaagccttcactcaccttcactgctgctg | |
| | | | | ttgcaactcggctgttctggactctgatgtgtgtggagggatggggaatagaa | |
| | | | | cattgactgtgttgattaccttcactattcggccagcctgaccttttaataac | |
| | | | | tttgtaaaaagcatgtatgtatttatagtgttttagatttttctaacttttat | |
| | | | | atcttaaaagcagagcacctgtttaagcattgtacccctattgttaaagatttgtgt | |
| | | | | cctctcattccctctcttcctcttgtaagtgcccttctaata | |
| | | 1289 | GGCTCAGCATCCAGAAACACCAAAC | | |
| | | 1290 | GGCTGGCTAAACAAGTGGCCGCGTG | | |
| | | 1291 | CAGCTCTGAGTCAAATCTGGGCCCT | | |
| | | 1292 | CCCACTCCCTTGCTAGGGGTGAAAG | | |
| | | 1293 | GAGCCATCTATCCAAGAAGCCTTCA | | |
| | | 1294 | CTGTTCTGGACTCTGATGTGTGTGG | | |
| | | 1295 | GCCAGCCTGACCTTTTAATAACTTT | | |
| | | 1296 | GCACCTGTTTAAGCATTGTACCCCT | | |
| | | 1297 | GTTAAAGATTTGTGTCCTCTCATTC | | |
| | | 1298 | TCCTCTTGTAAGTGCCCTTCTAATA | | |
| MAPBPIP | 218291_at | 1299 | AGCCAAGCCAACACTGGAGGCGTCC | gagaggcacctcggagatctgggtgcaaaagcccagggttaggaaccgtagca | NM_014017.1 |
| | | | | tgctgcgccccaaggctttgacccaggtgctaagccaagccaacactggaggc | |
| | | | | gtccagagcaccctgctgctgaataacgagggatcactgctggcctactctgg | |
| | | | | ttacggggacactgacgcccgggtcaccgctgccatagccagtaacatctggg | |
| | | | | ccgcctacgaccggaacgggaaccaagcgtttaatgaagacaatctcaaattc | |
| | | | | atcctcatggactgcatggagggccgtgtagccatcacccgagtggccaacct | |
| | | | | tctgctgtgtatgtatgccaaggagaccgtgggctttggaatgctcaaggcca | |
| | | | | aggcccaggctttggtgcagtacctggaggagcccctcacccaagtggcggcatctt | |
| | | | | aacggcattg | |
| | | 1300 | ATAACGAGGGATCACTGCTGGCCTA | | |
| | | 1301 | CTACTCTGGTTACGGGGACACTGAC | | |
| | | 1302 | TAGCCAGTAACATCTGGGCCGCCTA | | |
| | | 1303 | AATCTCAAATTCATCCTCATGGACT | | |
| | | 1304 | TGGACTGCATGGAGGGCCGTGTAGC | | |
| | | 1305 | GAGAGGCACCTCGGAGATCTGGGTG | | |
| | | 1306 | GACCGTGGGCTTTGGAATGCTCAAG | | |
| | | 1307 | AAGTGGCGGCATCTTAACGGCATTG | | |
| | | 1308 | GGTTAGGAACCGTAGCATGCTGCGC | | |
| | | 1309 | CCAAGGCTTTGACCCAGGTGCTAAG | | |
| PGLS | 218388_at | 1310 | CCTACAGGAGCGGGAGAAGATTGTG | cctacaggagcgggagaagattgtggctcccatcagtgactccccgaagccac | NM_012088.1 |
| | | | | cgccacagcgtgtgaccctcacactacctgtcctgaatgcagcacgaactgtc | |
| | | | | atctttgtggcaactggagaaggcaaggcagctgttctgaagcgcattttgga | |
| | | | | ggaccaggaggaaaacccgctgcccgccgccctggtccagccccacaccgggaaact | |
| | | | | gtgctggttcttggacgag | |
| | | 1311 | GAAGATTGTGGCTCCCATCAGTGAC | | |
| | | 1312 | CTCACACTACCTGTCCTGAATGCAG | | |
| | | 1313 | ACCTGTCCTGAATGCAGCACGAACT | | |
| | | 1314 | CAGCACGAACTGTCATCTTTGTGGC | | |
| | | 1315 | GTGGCAACTGGAGAAGGCAAGGCAG | | |
| | | 1316 | GGCAAGGCAGCTGTTCTGAAGCGCA | | |
| | | 1317 | GGCAGCTGTTCTGAAGCGCATTTTG | | |
| | | 1318 | AAGCGCATTTTGGAGGACCAGGAGG | | |
| | | 1319 | CCCCACACCGGGAAACTGTGCTGGT | | |
| | | 1320 | GAAACTGTGCTGGTTCTTGGACGAG | | |
| SPON2 | 218638_s_at | 1321 | CTGCCCCGAGCTCGAAGAAGAGGCT | ctgccccgagctcgaagaagaggctgagtgcgtccctgataactgcgtctaag | NM_012445.1 |
| | | | | accagagccccgcagcccctggggcccccggagccatggggtgtcgggggctc | |
| | | | | ctgtgcaggctcatgctgcaggcggccgaggcacagggggtttcgcgctgctc | |
| | | | | ctgaccgcggtgaggccgcgccgaccatctctgcactgaagggccctctggtg | |
| | | | | gccggcacgggcattgggaaacagcctcctcctttcccaaccttgcttcttag | |
| | | | | gggcccccgtgtcccgtctgctctcagcctcctcctcctgcaggataaagtca | |
| | | | | tccccaaggctccagctactctaaattatggtctccttataagttattgctgc | |
| | | | | tccaggagattgtccttcatcgtccaggggcctggctcccacgtggttgcaga | |
| | | | | tacctcagacctggtgctctaggctgtgctgagcccactctcccgagggcgca | |
| | | | | tccaagcgggggccacttgagaagtgaataaatggggcggtttcggaagcgtcagtg | |
| | | | | tttccatgttatgg | |
| | | 1322 | AAGAAGAGGCTGAGTGCGTCCCTGA | | |
| | | 1323 | GTCCCTGATAACTGCGTCTAAGACC | | |
| | | 1324 | GCCGGCACGGGCATTGGGAAACAGC | | |
| | | 1325 | AGGATAAAGTCATCCCCAAGGCTCC | | |
| | | 1326 | AAGGCTCCAGCTACTCTAAATTATG | | |
| | | 1327 | CCAGGAGATTGTCCTTCATCGTCCA | | |
| | | 1328 | CTCCCACGTGGTTGCAGATACCTCA | | |
| | | 1329 | TGCAGATACCTCAGACCTGGTGCTC | | |
| | | 1330 | CATCCAAGCGGGGGCCACTTGAGAA | | |
| | | 1331 | AAGCGTCAGTGTTTCCATGTTATGG | | |
| CRTC3 | 218648_at | 1332 | CCAGTTGTGGTCCTCAGCATTTGAA | ccagttgtggtcctcagcatttgaagcagctgcatacttcagagtaaactatt | NM_022769.1 |
| | | | | tttcattatttagttttgtcacaagaaatcgaccattgtactactctcactta | |
| | | | | cagcagttaaacagcatagaactaaaaacctgtctgcatttccattttttctt | |
| | | | | tctgtatggttgtgggttttaggacatagggggttaggagaaggggtttcttg | |
| | | | | atcatgtcatgaattctcctttgtcctgtttctcctgtttcatttctcctccg | |
| | | | | cctgctgtatattacctgagctggtgttgtatcttcaagtccatatgcgtatt | |
| | | | | tgcagacctttcctgttcccactcttgttggctcttctgatttatgcacagat | |
| | | | | ggttcccagcatgtgtccagtgcttcatggatgggaccatcccagcaactaat | |
| | | | | cagacttcctgccagtgtcctaacccccagggcaccctgttcaaccatatttaaa | |
| | | 1333 | GAAATCGACCATTGTACTACTCTCA | | |
| | | 1334 | GTACTACTCTCACTTACAGCAGTTA | | |
| | | 1335 | AAAACCTGTCTGCATTTCCATTTTT | | |
| | | 1336 | GAGCTGGTGTTGTATCTTCAAGTCC | | |
| | | 1337 | TATGCGTATTTGCAGACCTTTCCTG | | |
| | | 1338 | CTCTTGTTGGCTCTTCTGATTTATG | | |
| | | 1339 | GATTTATGCACAGATGGTTCCCAGC | | |
| | | 1340 | CCCAGCATGTGTCCAGTGCTTCATG | | |
| | | 1341 | ACTAATCAGACTTCCTGCCAGTGTC | | |
| | | 1342 | GGCACCCTGTTCAACCATATTTAAA | | |
| PRKCH | 218764_at | 1343 | CACCAAGACGACTGCTTCAGCTTCT | caccaagacgactgcttcagcttcttctcttatccttactttctttaatagat | NM_024064.1 |
| | | | | atttattaaactgtccagtgaaaaggtgccacaatgcccagtattgtaaacaa | |
| | | | | caggtttgcattcatgaagctttcattcattctggagtctactaatttacctg | |
| | | | | aatggtgtttgcattctgtgaaatgcctctccacgttgcatatgtcacacttt | |
| | | | | tgtctgcacataactcttttttcacaagaagggtcactgccacaacagcacag | |
| | | | | tcagcgggtgaattacaggtgcctgctgcctgcctacctgggtaatctgatct | |
| | | | | tgtctgtatcgccgtgtgctcatcactgaagaattgcaggccactcatgtcagt | |
| | | 1344 | TCTCTTATCCTTACTTTCTTTAATA | | |
| | | 1345 | AAAGGTGCCACAATGCCCAGTATTG | | |
| | | 1346 | AGCTTTCATTCATTCTGGAGTCTAC | | |
| | | 1347 | ATTCTGTGAAATGCCTCTCCACGTT | | |
| | | 1348 | TCTCCACGTTGCATATGTCACACTT | | |
| | | 1349 | GTCTGCACATAACTCTTTTTTCACA | | |
| | | 1350 | GCCACAACAGCACAGTCAGCGGGTG | | |
| | | 1351 | GTCAGCGGGTGAATTACAGGTGCCT | | |
| | | 1352 | GTAATCTGATCTTGTCTGTATCGCC | | |
| | | 1353 | AGAATTGCAGGCCACTCATGTCAGT | | |
| CHST12 | 218927_s_at | 1354 | GACCCGCACACGGAGAAGCTGGCGC | gacccgcacacggagaagctggcgcccttcaacgagcactggcggcaggtgta | NM_018641.1 |
| | | | | ccgcctctgccacccgtgccagatcgactacgacttcgtggggaagctggaga | |
| | | | | ctctggacgaggacgccgcgcagctgctgcagctactccaggtggaccggcag | |
| | | | | ctccgcttccccccgagctaccggaacaggaccgccagcagctgggaggagga | |
| | | | | ctggttcgccaagatccccctggcctggaggcagcagctgtataaactctacg | |
| | | | | aggccgactttgttctcttcggctaccccaagcccgaaaacctcctccgagac | |
| | | | | tgaaagctttcgcgttgctttttctcgcgtgcctggaacctgacgcacgcgca | |
| | | | | ctccagtttttttatgacctacgattttgcaatctgggcttcttgttcactccactg | |
| | | | | cctctatccattgagtac | |
| | | 1355 | CACTGGCGGCAGGTGTACCGCCTCT | | |
| | | 1356 | GCCAGATCGACTACGACTTCGTGGG | | |
| | | 1357 | GCTGGAGACTCTGGACGAGGACGCC | | |
| | | 1358 | GGAGGAGGACTGGTTCGCCAAGATC | | |
| | | 1359 | TAAACTCTACGAGGCCGACTTTGTT | | |
| | | 1360 | GAAAACCTCCTCCGAGACTGAAAGC | | |
| | | 1361 | AAAGCTTTCGCGTTGCTTTTTCTCG | | |
| | | 1362 | GCGTGCCTGGAACCTGACGCACGCG | | |
| | | 1363 | TTTGCAATCTGGGCTTCTTGTTCAC | | |
| | | 1364 | TCCACTGCCTCTATCCATTGAGTAC | | |
| C16ORF68 | 218945_at | 1365 | ACTGGACTGGCTGAAGGACGACCTC | actggactggctgaaggacgacctctgcacagatcccaaggtccccttcagtt | NM_024109.1 |
| | | | | ggtcacaagaggaaatttctgacctgtacgatcacaccaccatcctgtttgca | |
| | | | | gccgaagtgttttacgacgacgacttgactgatgctgtgtttaaaacgctctc | |
| | | | | ccgactcgcccacagattgaaaaatgcctgcacagccatactgtcggtggaga | |
| | | | | agaggctcaacttcacactgagacacttggacgtcacatgtgaagcctacgat | |
| | | | | cacttccgctcctgcctgcacgcgctggagcagctcacagatggcaagctgcg | |
| | | | | cttcgtggtggagcccgtggaggcctccttcccacagctcctggtttacgagc | |
| | | | | gcctccagcagctggagctctggaagatcatcgcagaaccagtaacatgacccatcg | |
| | | | | cctccaccaggcgcggcgtctcgactgttcttagagtg | |
| | | 1366 | AATTTCTGACCTGTACGATCACACC | | |
| | | 1367 | ACCATCCTGTTTGCAGCCGAAGTGT | | |
| | | 1368 | TACGACGACGACTTGACTGATGCTG | | |
| | | 1369 | TGCTGTGTTTAAAACGCTCTCCCGA | | |
| | | 1370 | CCTGCACAGCCATACTGTCGGTGGA | | |
| | | 1371 | ATGTGAAGCCTACGATCACTTCCGC | | |
| | | 1372 | GCTCACAGATGGCAAGCTGCGCTTC | | |
| | | 1373 | TCCCACAGCTCCTGGTTTACGAGCG | | |
| | | 1374 | CAGAACCAGTAACATGACCCATCGC | | |
| | | 1375 | CGGCGTCTCGACTGTTCTTAGAGTG | | |
| TTC17 | 218972_at | 1376 | CTCCTGGGCCACAAGGGCTACTAGA | ctcctgggccacaagggctactagactggaagaccaggaaagtgccatagaca | NM_018259.1 |
| | | | | taatgtaactggatttcagcaaggcatttaacagagcctcttatgatatcctt | |
| | | | | gtgaaccagatggagagatgtgggcttgaagccttcccattgcctacaggata | |
| | | | | aaattcaaacttcctagtgtggtgtacaagaccctttacagcccgcctctgtg | |
| | | | | tacccttcaacaccattctctgaaccaaccatgctcatgtttttacctcagtg | |
| | | | | cctttgcacatgctattccctctgcctggaatgccctgtgccccctctgccct | |
| | | | | ctgccgtgctaaaatatcactcatccttaaacttcaaaatcaagtgccatctc | |
| | | | | ttccttgttaccttcaggcagaattagttactctttcctctgtgcaattgttc | |
| | | | | tatatcttcgctctagctcttttcctgttgtattgtaatgatttgtttatgtt | |
| | | | | taccttccttactagactgtgagctcaagagcaggccgtcttaattattcctttctg | |
| | | | | tacccctagtgtcttttatggttctcagccc | |
| | | 1377 | CAGAGCCTCTTATGATATCCTTGTG | | |
| | | 1378 | GATGTGGGCTTGAAGCCTTCCCATT | | |
| | | 1379 | TGGTGTACAAGACCCTTTACAGCCC | | |
| | | 1380 | TGCCCTCTGCCGTGCTAAAATATCA | | |
| | | 1381 | CTCTTCCTTGTTACCTTCAGGCAGA | | |
| | | 1382 | AGAATTAGTTACTCTTTCCTCTGTG | | |
| | | 1383 | GTGCAATTGTTCTATATCTTCGCTC | | |
| | | 1384 | TTATGTTTACCTTCCTTACTAGACT | | |
| | | 1385 | GCAGGCCGTCTTAATTATTCCTTTC | | |
| | | 1386 | TAGTGTCTTTTATGGTTCTCAGCCC | | |
| PLEKHA1 | 219024_at | 1387 | ACTCTTTGGTCTCAACCTTTACCAT | acaacgtctcgaactttctatgtgcaggctgatagccctgaagagatgcacag | NM_021622.1 |
| | | | | ttggattaaagcagtctctggcgccattgtagcacagcggggtcccggcagat | |
| | | | | ctgcgtcttctgagcatccccccggtccttcagaatccaaacacgctttccgt | |
| | | | | cctaccaacgcagccgccgccacctcacattccacagcctctcgcagcaactc | |
| | | | | tttggtctcaacctttaccatggagaagcgaggattttacgagtctcttgcca | |
| | | | | aggtcaagccagggaacttcaaggtccagactgtctctccaagagaaccagct | |
| | | | | tccaaagtgactgaacaagctctgttaagacctcaaagtaaaaatggccctca | |
| | | | | ggaaaaagattgtgacctagtagacttggacgatgcgagccttccggtcagtg | |
| | | | | acgtgtgaggcagaagcgcacggagcctgcctgcctctgccgtcctcagttacctttc | |
| | | | | atgaggcttctagcc | |
| | | 1388 | GGATTTTACGAGTCTCTTGCCAAGG | | |
| | | 1389 | TTCAAGGTCCAGACTGTCTCTCCAA | | |
| | | 1390 | TCTCTCCAAGAGAACCAGCTTCCAA | | |
| | | 1391 | AGTAGACTTGGACGATGCGAGCCTT | | |
| | | 1392 | AGCCTTCCGGTCAGTGACGTGTGAG | | |
| | | 1393 | GAGGCAGAAGCGCACGGAGCCTGCC | | |
| | | 1394 | GTTACCTTTCATGAGGCTTCTAGCC | | |
| | | 1395 | ACAACGTCTCGAACTTTCTATGTGC | | |
| | | 1396 | CTCTGGCGCCATTGTAGCACAGCGG | | |
| | | 1397 | TCAGAATCCAAACACGCTTTCCGTC | | |
| GIMAP4 | 219243_at | 1398 | TCTTCTAGATTCTCTCTATGTTGGC | tcttctagattctctctatgttggcagataatctccccttgtagcttccactc | NM_018326.1 |
| | | | | acttattcttgcattcagagtcacaatgatcatcttacccatgtggtttttga | |
| | | | | gaaagaaagatcaattctttgtttgcagtgggtaatcttagagatggagatga | |
| | | | | ttgtagaattattcctagatgagtgtcaatttatttaattccattgtcatata | |
| | | | | aggagtcaaattgtttcttatcatttgttcattgaagaacagagacctgtctg | |
| | | | | gaaaatcgatctctacaaattcaattaaataatgatccccaaatgctgaaaaa | |
| | | | | gtgaaatacagcaattcaacagataatagagcaatgtttagtatattcagctg | |
| | | | | tatctgtagaaactctttgacgaacctcaatttaaccaatttgatgaataccc | |
| | | | | agttctcttcttttctagagaaagatagttgcaacctcacctccctcactcaacactt | |
| | | | | tgaatacttattgtttggcaggtcatccacacact | |
| | | 1399 | TGTTGGCAGATAATCTCCCCTTGTA | | |
| | | 1400 | TTCCACTCACTTATTCTTGCATTCA | | |
| | | 1401 | GAGTCACAATGATCATCTTACCCAT | | |
| | | 1402 | GATCAATTCTTTGTTTGCAGTGGGT | | |
| | | 1403 | AATTGTTTCTTATCATTTGTTCATT | | |
| | | 1404 | TGTAGAAACTCTTTGACGAACCTCA | | |
| | | 1405 | TGATGAATACCCAGTTCTCTTCTTT | | |
| | | 1406 | GAAAGATAGTTGCAACCTCACCTCC | | |
| | | 1407 | CTCCCTCACTCAACACTTTGAATAC | | |
| | | 1408 | TTGTTTGGCAGGTCATCCACACACT | | |
| CENTA2 | 219358_s_at | 1409 | CCAGCTACTCCGGACACTGATGTGA | ccagctactccggacactgatgtgagaggatcacttgagccagggaggtcatg | NM_018404.1 |
| | | | | gctacagtgacccctcattgcaccactttacttagcctgggtgacagagtgag | |
| | | | | accctatctcaaaaaaaaaaaaaatctatgcattgtatgggactttcctttgg | |
| | | | | atcccccaatcaaaggataagcaatgcgtaagcctgtgtccttcctgaagctt | |
| | | | | ctcgactgcccagatagggaggtgagtcctctctatctcctctggctctggaa | |
| | | | | gcaccttgaaaatgtgcattttcaaggacacttgctgggttgtgcattaaggg | |
| | | | | ccagtttacttgtctgcctctttgaccacctgtgaactctgttgggtgtactctgcta | |
| | | | | agt | |
| | | 1410 | GGGAGGTCATGGCTACAGTGACCCC | | |
| | | 1411 | TTGCACCACTTTACTTAGCCTGGGT | | |
| | | 1412 | TATGCATTGTATGGGACTTTCCTTT | | |
| | | 1413 | AAGCAATGCGTAAGCCTGTGTCCTT | | |
| | | 1414 | TTCTCGACTGCCCAGATAGGGAGGT | | |
| | | 1415 | TAGGGAGGTGAGTCCTCTCTATCTC | | |
| | | 1416 | AAGGACACTTGCTGGGTTGTGCATT | | |
| | | 1417 | GGTTGTGCATTAAGGGCCAGTTTAC | | |
| | | 1418 | TTGACCACCTGTGAACTCTGTTGGG | | |
| | | 1419 | CTCTGTTGGGTGTACTCTGCTAAGT | | |
| SERTAD3 | 219382_at | 1420 | TTTGTTCCCATTTCAGGGTTCCACA | tgtgtttttgtgggggctcgaagcagtgactatggcctcctttgttcccattt | NM_013368.1 |
| | | | | cagggttccacaaactgtcttgcatgtgtgtgtgtgtctggttaccccgacct | |
| | | | | tctgtgaaggtgggtcttcctgaattaatttatctattccaaatgccttaacg | |
| | | | | agactctgtttctgggagtctgattttccacttacacatttcttccacctttc | |
| | | | | ctgctagttcccactcccctgtgaccactggggcctcagggaagataaagaaa | |
| | | | | gctgggcctgtcgaaggatgacagggatgtgctgccaggttgctatagaaacc | |
| | | | | caggctctgcctcttgcaccttgagggggtgggaggggctggtgtcctccctc | |
| | | | | caggctgaaccccacttcctcggcaggaccccagtctcagcagcctcctgatt | |
| | | | | tcataaccaggccggaccacgtgcaatagggtggaaaccaaactgctccatgccggg | |
| | | | | ttatttaaaagaaaggcagagtttgtggtggcttttttt | |
| | | 1421 | TCAGGGTTCCACAAACTGTCTTGCA | | |
| | | 1422 | GCCTTAACGAGACTCTGTTTCTGGG | | |
| | | 1423 | TGATTTTCCACTTACACATTTCTTC | | |
| | | 1424 | GGATGTGCTGCCAGGTTGCTATAGA | | |
| | | 1425 | GCCTCTTGCACCTTGAGGGGGTGGG | | |
| | | 1426 | TGATTTCATAACCAGGCCGGACCAC | | |
| | | 1427 | ACTGCTCCATGCCGGGTTATTTAAA | | |
| | | 1428 | GGCAGAGTTTGTGGTGGCTTTTTTT | | |
| | | 1429 | TGTGTTTTTGTGGGGGCTCGAAGCA | | |
| | | 1430 | GCTCGAAGCAGTGACTATGGCCTCC | | |
| HPSE | 219403_s_at | 1431 | ATTGGGCCTGTCAGCCCGAATGGGA | attgggcctgtcagcccgaatgggaatagaagtggtgatgaggcaagtattct | NM_006665.1 |
| | | | | ttggagcaggaaactaccatttagtggatgaaaacttcgatcctttacctgat | |
| | | | | tattggctatctcttctgttcaagaaattggtgggcaccaaggtgttaatggc | |
| | | | | aagcgtgcaaggttcaaagagaaggaagcttcgagtataccttcattgcacaa | |
| | | | | acactgacaatccaaggtataaagaaggagatttaactctgtatgccataaac | |
| | | | | ctccataatgtcaccaagtacttgcggttaccctatcctttttctaacaagca | |
| | | | | agtggataaataccttctaagacctttgggacctcatggattactttccaaat | |
| | | | | ctgtccaactcaatggtctaactctaaagatggtggatgatcaaaccttgcca | |
| | | | | cctttaatggaaaaacctctccggccaggaagttcactgggcttgccagctttctca | |
| | | | | tatagtttttttgtgataagaaatgccaaagttgctgcttgcatctga | |
| | | 1432 | GAAAACTTCGATCCTTTACCTGATT | | |
| | | 1433 | GATTATTGGCTATCTCTTCTGTTCA | | |
| | | 1434 | AGCTTCGAGTATACCTTCATTGCAC | | |
| | | 1435 | AACTCTGTATGCCATAAACCTCCAT | | |
| | | 1436 | CAAGTACTTGCGGTTACCCTATCCT | | |
| | | 1437 | GACCTTTGGGACCTCATGGATTACT | | |
| | | 1438 | GATTACTTTCCAAATCTGTCCAACT | | |
| | | 1439 | GAAGTTCACTGGGCTTGCCAGCTTT | | |
| | | 1440 | GCCAGCTTTCTCATATAGTTTTTTT | | |
| | | 1441 | TGCCAAAGTTGCTGCTTGCATCTGA | | |
| CLIC3 | 219529_at | 1442 | ACGCCAAGACAGACACGCTGCAGAT | acgccaagacagacacgctgcagatcgaggactttctggaggagacgctgggg | NM_004669.1 |
| | | | | ccgcccgacttccccagcctggcgcctcgttacagggagtccaacaccgccgg | |
| | | | | caacgacgttttccacaagttctccgcgttcatcaagaacccggtgc | |
| | | 1443 | AGACACGCTGCAGATCGAGGACTTT | | |
| | | 1444 | ACACGCTGCAGATCGAGGACTTTCT | | |
| | | 1445 | GCTGCAGATCGAGGACTTTCTGGAG | | |
| | | 1446 | CGAGGACTTTCTGGAGGAGACGCTG | | |
| | | 1447 | GCCTCGTTACAGGGAGTCCAACACC | | |
| | | 1448 | TCGTTACAGGGAGTCCAACACCGCC | | |
| | | 1449 | GCAACGACGTTTTCCACAAGTTCTC | | |
| | | 1450 | CAAGTTCTCCGCGTTCATCAAGAAC | | |
| | | 1451 | TTCTCCGCGTTCATCAAGAACCCGG | | |
| | | 1452 | TCCGCGTTCATCAAGAACCCGGTGC | | |
| PLEKHF1 | 219566_at | 1453 | TTGGTAACAAACGCCACCTTACACT | ttggtaacaaacgccaccttacactctgcaggctgcagcggcagctccagatg | NM_024310.1 |
| | | | | gcctcctgagctggacgaccccaggtctccagacatctagggaccagagcagg | |
| | | | | tttgggaacacagagggaagacaggatgggagtgtagccacagaacccacctg | |
| | | | | caccctgacaggcacaccccactgaagagcctgagtcccaggaggcctcctgg | |
| | | | | aagcccaggactgcccacccaccacgctggtgcccaccgcctggccagccaag | |
| | | | | ccctgccgatcagacatgtgggctccccgaagcccagccagagactgccgtgc | |
| | | | | tgtgggtgccaccaggcccagggactgcagcctgagctccccgaggcccaggg | |
| | | | | cagccgggtgaggactctgtcctgtgtcacctctctccaggtgtccagctgtc | |
| | | | | tcatgcctttttgtcctgtcctcagctctccgtgtggtcagcgaaaccattgttttct | |
| | | | | gttaggactcagttgcaa | |
| | | 1454 | CCCAGGTCTCCAGACATCTAGGGAC | | |
| | | 1455 | ATCTAGGGACCAGAGCAGGTTTGGG | | |
| | | 1456 | TGGGAGTGTAGCCACAGAACCCACC | | |
| | | 1457 | CAGGCACACCCCACTGAAGAGCCTG | | |
| | | 1458 | CCCACTGAAGAGCCTGAGTCCCAGG | | |
| | | 1459 | AAGCCCTGCCGATCAGACATGTGGG | | |
| | | 1460 | CAGCCAGAGACTGCCGTGCTGTGGG | | |
| | | 1461 | CTCTCCGTGTGGTCAGCGAAACCAT | | |
| | | 1462 | GTCAGCGAAACCATTGTTTTCTGTT | | |
| | | 1463 | GTTTTCTGTTAGGACTCAGTTGCAA | | |
| CLDN15 | 219640_at | 1464 | CCTCCAGGCCAAGAACTGCTCTTGG | taccccggaaccaagtacgagctgggccccgccctctacctggggtggagcgc | NM_014343.1 |
| | | | | ctcactgatctccatcctgggtggcctctgcctctgctccgcctgctgctgcg | |
| | | | | gctctgacgaggacccagccgccagcgcccggcggccctaccaggctcccgtg | |
| | | | | tccgtgatgcccgtcgccacctcggaccaagaaggcgacagcagctttggcaa | |
| | | | | atacggcagaaacgcctacgtgtagcagctctggcccgtgggccccgctgtct | |
| | | | | tcccactgccccaaggagaggggacctggccggggcccattcccctatagtaa | |
| | | | | cctcaggggccggccacgccccgctcccgtagccccgccccggccacggcccc | |
| | | | | gtgtcttgcactctcatggcccctccaggccaagaactgctcttgggaagtcg | |
| | | | | catatctcccctctgaggctggatccctcatcttctgaccctgggttctgggctgtg | |
| | | | | aaggggacggtgtccccgcacgtttgtattgtgtat | |
| | | 1465 | TCTTGGGAAGTCGCATATCTCCCCT | | |
| | | 1466 | TCATCTTCTGACCCTGGGTTCTGGG | | |
| | | 1467 | TGACCCTGGGTTCTGGGCTGTGAAG | | |
| | | 1468 | GTGAAGGGGACGGTGTCCCCGCACG | | |
| | | 1469 | GTCCCCGCACGTTTGTATTGTGTAT | | |
| | | 1470 | TACCCCGGAACCAAGTACGAGCTGG | | |
| | | 1471 | GTCGCCACCTCGGACCAAGAAGGCG | | |
| | | 1472 | GACCAAGAAGGCGACAGCAGCTTTG | | |
| | | 1473 | GAAACGCCTACGTGTAGCAGCTCTG | | |
| | | 1474 | CTTCCCACTGCCCCAAGGAGAGGGG | | |
| SIDT1 | 219734_at | 1475 | GAGAAGTTCTACATTGACCAGGCCC | gagaagttctacattgaccaggcccccttgttgcctggagtatgacgtaatca | NM_017699.1 |
| | | | | gaaaatagacgtataaatgtgcacatgcgtatgtatttgcttgtgaaattaaa | |
| | | | | gtcacctcttgcctctgctttcctgatcattcgttagagaaatggatcaggca | |
| | | | | tttttttaaattattattctttctctaaactatttgcattgtgttcaaaaacc | |
| | | | | cattttagaagtttgaacagcaagcttttcctgattttaaaaacacaaagttg | |
| | | | | ctttcaatgaaatattttgtgatttttttaaagtccccaaatgtgtacttagc | |
| | | | | cttctgttattccttattctttaagcagtgttggcttccattgaccatatgaaggcc | |
| | | | | accaattaaatggttgtg | |
| | | 1476 | CCCCTTGTTGCCTGGAGTATGACGT | | |
| | | 1477 | GTGCACATGCGTATGTATTTGCTTG | | |
| | | 1478 | CTGCTTTCCTGATCATTCGTTAGAG | | |
| | | 1479 | TGCATTGTGTTCAAAAACCCATTTT | | |
| | | 1480 | GAACAGCAAGCTTTTCCTGATTTTA | | |
| | | 1481 | GTCCCCAAATGTGTACTTAGCCTTC | | |
| | | 1482 | TACTTAGCCTTCTGTTATTCCTTAT | | |
| | | 1483 | GTTATTCCTTATTCTTTAAGCAGTG | | |
| | | 1484 | CAGTGTTGGCTTCCATTGACCATAT | | |
| | | 1485 | GAAGGCCACCAATTAAATGGTTGTG | | |
| PVRIG | 219812_at | 1486 | GCCCAGGGCCATGGAAGGACCCTTA | gctttgtctctgttgagaatggactctacgctcaggcaggggagaggcctcct | NM_024070.1 |
| | | | | cacactggtcccggcctcactcttttccctgaccctcgggggcccagggccat | |
| | | | | ggaaggacccttaggagttcgatgagagagaccatgaggccactgggctttcc | |
| | | | | ccctcccaggcctcctgggtgtcatccccttactttaattcttgggcctccaa | |
| | | | | taagtgtcccataggtgtctggccaggcccacctgctgcggatgtggtctgtg | |
| | | | | tgcgtgtgtgggcacaggtgtgagtgtgtgagtgacagttaccccatttcagtcattt | |
| | | | | cctgctgcaac | |
| | | 1487 | AGGACCCTTAGGAGTTCGATGAGAG | | |
| | | 1488 | TCATCCCCTTACTTTAATTCTTGGG | | |
| | | 1489 | TTCTTGGGCCTCCAATAAGTGTCCC | | |
| | | 1490 | TAAGTGTCCCATAGGTGTCTGGCCA | | |
| | | 1491 | GTGCGTGTGTGGGCACAGGTGTGAG | | |
| | | 1492 | TGTGAGTGACAGTTACCCCATTTCA | | |
| | | 1493 | GACAGTTACCCCATTTCAGTCATTT | | |
| | | 1494 | CATTTCAGTCATTTCCTGCTGCAAC | | |
| | | 1495 | GCTTTGTCTCTGTTGAGAATGGACT | | |
| | | 1496 | TGAGAATGGACTCTACGCTCAGGCA | | |
| GFOD1 | 219821_s_at | 1497 | GATTGATTGGGCTTCCTCATAGGAA | gattgattgggcttcctcataggaagcactgagggtgtgtctttgtacttggt | NM_018988.1 |
| | | | | tcattgcccttcacctggtagagaaagagaggtcagaaatagcaagcaaaaag | |
| | | | | caggactcccaggagccacaagaaaagagcacaggctgcaccaaagcaggggc | |
| | | | | agcagagaataaaatatccctttgaacttgtcaacaattaaaaaactgcaagg | |
| | | | | agtcaccttataacactatttccagtaaaggtggaattgagtatcagagggat | |
| | | | | tactgcggtgttaaggtagccctgccacgtggctctccaggcagggccaagaa | |
| | | | | gacagcacaaagtatgggtttggccataagctcatatgctgcccccaaagact | |
| | | | | ggggagagctgtgtgcctcagtgttgcagtgtgaattcctaaatagagggtaa | |
| | | | | agtgagcctagccaggaggtgtttggggctctatcgcgcatctctcctaccaa | |
| | | | | gctgggcaagagcttttaggagattcatccagctttgtggatttagaaaggaagcctt | |
| | | | | cagttccaatcagaatc | |
| | | 1498 | GTGTCTTTGTACTTGGTTCATTGCC | | |
| | | 1499 | TCATTGCCCTTCACCTGGTAGAGAA | | |
| | | 1500 | GTCACCTTATAACACTATTTCCAGT | | |
| | | 1501 | CTGCGGTGTTAAGGTAGCCCTGCCA | | |
| | | 1502 | TGGCTCTCCAGGCAGGGCCAAGAAG | | |
| | | 1503 | TTGGCCATAAGCTCATATGCTGCCC | | |
| | | 1504 | AGACTGGGGAGAGCTGTGTGCCTCA | | |
| | | 1505 | GTGTGCCTCAGTGTTGCAGTGTGAA | | |
| | | 1506 | CAGGAGGTGTTTGGGGCTCTATCGC | | |
| | | 1507 | GAAGCCTTCAGTTCCAATCAGAATC | | |
| LUC7L2 | 220099_s_at | 1508 | GATGCTGATCTCTTTATTCTTTCAA | gatgctgatctctttattctttcaagtaagagtgctagtgaacaaattgtgtt | NM_016007.1 |
| | | | | acttggccttgggattttttgaacgtttgtaaaatgctgtcttcctagtccaa | |
| | | | | acagctgcagctttgggcatttttctttttaattattcttcctctgactttgt | |
| | | | | atcccttaatacctacactctccaattgtaagagaaagggggcagggaagcaa | |
| | | | | tatagcttccattctaaggctgtattcccgttatgaattactagctgattaca | |
| | | | | gttcagagcattgatcctggaatgtgtgctggagaaatttaaaatactggggt | |
| | | | | tttttgtttaatggtgcctatttagagttggaagttgaacagctgttgcatta | |
| | | | | catacttttgcttttttattgaaattttgaaatcaaacgtcttgatttttctg | |
| | | | | ttctgttgaattgctatgttcaggatgttctagggggtgggggcagggactcttttcg | |
| | | | | taataag | |
| | | 1509 | AATGCTGTCTTCCTAGTCCAAACAG | | |
| | | 1510 | AGCTGCAGCTTTGGGCATTTTTCTT | | |
| | | 1511 | AATTATTCTTCCTCTGACTTTGTAT | | |
| | | 1512 | TCCTCTGACTTTGTATCCCTTAATA | | |
| | | 1513 | CTTAATACCTACACTCTCCAATTGT | | |
| | | 1514 | AAGGCTGTATTCCCGTTATGAATTA | | |
| | | 1515 | GAACAGCTGTTGCATTACATACTTT | | |
| | | 1516 | GATTTTTCTGTTCTGTTGAATTGCT | | |
| | | 1517 | GTTCAGGATGTTCTAGGGGGTGGGG | | |
| | | 1518 | GGGCAGGGACTCTTTTCGTAATAAG | | |
| MNAB | 220202_s_at | 1519 | TGGGGTGCGATTTCCAGATCTTCCC | tggggtgcgatttccagatcttcccgtacaggttaccataccacagatcctgt | NM_018835.1 |
| | | | | ccaggccactgcttcccaaggaagtgcgactaagcccatcagtgtatcagatt | |
| | | | | atgtcccttatgtcaatgctgttgattcaaggtggagttcatatggcaacgag | |
| | | | | gccacatcatcagcacactatgttgaaagggacagattcattgttactgattt | |
| | | | | atctggtcatagaaagcattccagtactggggaccttttgagccttgaacttc | |
| | | | | agcaggccaagagcaactcattacttcttcagagagaggccaatgctttggccatgc | |
| | | | | aacagaagtggaattccctggatgaaggccgtcac | |
| | | 1520 | TTCCCGTACAGGTTACCATACCACA | | |
| | | 1521 | GTGCGACTAAGCCCATCAGTGTATC | | |
| | | 1522 | TATGTCCCTTATGTCAATGCTGTTG | | |
| | | 1523 | GTGGAGTTCATATGGCAACGAGGCC | | |
| | | 1524 | GCCACATCATCAGCACACTATGTTG | | |
| | | 1525 | TACTGGGGACCTTTTGAGCCTTGAA | | |
| | | 1526 | GAGCCTTGAACTTCAGCAGGCCAAG | | |
| | | 1527 | AGAGCAACTCATTACTTCTTCAGAG | | |
| | | 1528 | CAATGCTTTGGCCATGCAACAGAAG | | |
| | | 1529 | GAATTCCCTGGATGAAGGCCGTCAC | | |
| CECR7 | 220452_x_at | 1530 | GATGAGAAAGACCTGACTGTGCCCC | gatgagaaagacctgactgtgccccagcccgacacccataaagggtctgtgct | NM_021031.1 |
| | | | | gaggtggattagtaaaagaggaaagcctcttgcagttgagatagaggaaggcc | |
| | | | | actgtctctgcctgcccctgggaactgaatgtctcggtataaaaccgattgta | |
| | | | | catttgttcaattctgagataggagaaaaccgccctatggtgggagcgagaca | |
| | | | | tgtttcgagcaatgctgccttgttattctttactccgctgagatgtttgggtg | |
| | | | | gagagaaacataaatctggcctacatgcacatccgggcatagtaccttccctt | |
| | | | | gaacttaatcatgacacagattcttttgctcacatgttttttgctgaccttct | |
| | | | | ccttattatcaccctgctgtcctactacattcctttttgctgaaataatgaaa | |
| | | | | ataatagtcaataaaaactgagggaactcaaaggccggtgccagtgcaggtcc | |
| | | | | ttggtgtgtcgaatactggtcccc | |
| | | 1531 | TAGAGGAAGGCCACTGTCTCTGCCT | | |
| | | 1532 | GCCCCTGGGAACTGAATGTCTCGGT | | |
| | | 1533 | GTGGGAGCGAGACATGTTTCGAGCA | | |
| | | 1534 | AGCAATGCTGCCTTGTTATTCTTTA | | |
| | | 1535 | ATAAATCTGGCCTACATGCACATCC | | |
| | | 1536 | AGTACCTTCCCTTGAACTTAATCAT | | |
| | | 1537 | GACACAGATTCTTTTGCTCACATGT | | |
| | | 1538 | GCTCACATGTTTTTTGCTGACCTTC | | |
| | | 1539 | TCAAAGGCCGGTGCCAGTGCAGGTC | | |
| | | 1540 | CTTGGTGTGTCGAATACTGGTCCCC | | |
| TH1L | 220607_x_at | 1541 | ACTTCCTGTTGTCAGTTACATCCGA | acttcctgttgtcagttacatccgaaagtgtctggagaagctggacactgaca | NM_016397.1 |
| | | | | tttcactcattcgctattttgtcactgaggtgctggacgtcattgctcctcct | |
| | | | | tatacctctgacttcgtgcaacttttcctccccatcctggagaatgacagcat | |
| | | | | cgcaggtaccatcaaaacggaaggcgagcatgaccctgtgacggagtttatag | |
| | | | | ctcactgcaaatctaacttcatcatggtgaactaatttagagcatcctccaga | |
| | | | | gctgaagcagaacattccagaacccgttgtggaaaaaccctttcaagaagctg | |
| | | | | ttttaagaggctcgggcagcgtcttgaaaatgggcaccgctgggaggaggtgg | |
| | | | | atgacttctttacaaaggaaaatggcaggcgctgggctcccacgacccctcag | |
| | | | | gacagatctggccgtcagccgcgggccgctgggaactccactcggggaactcctttcc | |
| | | | | aagctgacctcagttttctcac | |
| | | 1542 | GGACACTGACATTTCACTCATTCGC | | |
| | | 1543 | TCACTCATTCGCTATTTTGTCACTG | | |
| | | 1544 | GTCACTGAGGTGCTGGACGTCATTG | | |
| | | 1545 | TTTTCCTCCCCATCCTGGAGAATGA | | |
| | | 1546 | GAATGACAGCATCGCAGGTACCATC | | |
| | | 1547 | CGAGCATGACCCTGTGACGGAGTTT | | |
| | | 1548 | GAGCATCCTCCAGAGCTGAAGCAGA | | |
| | | 1549 | GCAGAACATTCCAGAACCCGTTGTG | | |
| | | 1550 | TTAAGAGGCTCGGGCAGCGTCTTGA | | |
| | | 1551 | TCCAAGCTGACCTCAGTTTTCTCAC | | |
| KLRF1 | 220646_s_at | 1552 | ATCCAGGATTTTTATTCGTCGCTTA | atattcttcataaagggaccagctaaagaaaacagctgtgctgccattaagga | NM_016523.1 |
| | | | | aagcaaaattttctctgaaacctgcagcagtgttttcaaatggatttgtcagt | |
| | | | | attagagtttgacaaaattcacagtgaaataatcaatgatcactatttttggc | |
| | | | | ctattagtttctaatattaatctccaggtgtaagattttaaagtgcaattaaa | |
| | | | | tgccaaaatctcttctcccttctccctccatcatcgacactggtctagcctca | |
| | | | | gagtaacccctgttaacaaactaaaatgtacacttcaaaatttttacgtgata | |
| | | | | gtataaaccaatgtgacttcatgtgatcatatccaggatttttattcgtcgct | |
| | | | | tattttatgccaaatgtgatcaaattatgcctgtttttctgtatcttgcgttt | |
| | | | | taaattcttaataaggtcctaaacaaaatttcttatatttctaatggttgaat | |
| | | | | tataatgtgggtttatacattttttacccttttgtcaaagagaattaactttgtttcc | |
| | | | | aggcttttgctact | |
| | | 1553 | TTATTCGTCGCTTATTTTATGCCAA | | |
| | | 1554 | AATGTGATCAAATTATGCCTGTTTT | | |
| | | 1555 | ACTTTGTTTCCAGGCTTTTGCTACT | | |
| | | 1556 | ATATTCTTCATAAAGGGACCAGCTA | | |
| | | 1557 | ACAGCTGTGCTGCCATTAAGGAAAG | | |
| | | 1558 | AAATTTTCTCTGAAACCTGCAGCAG | | |
| | | 1559 | CAATGATCACTATTTTTGGCCTATT | | |
| | | 1560 | CTCCATCATCGACACTGGTCTAGCC | | |
| | | 1561 | TAGCCTCAGAGTAACCCCTGTTAAC | | |
| | | 1562 | GTGACTTCATGTGATCATATCCAGG | | |
| TBX21 | 220684_at | 1563 | TCCTGGCCCACGATGAAACCTGAGA | tcctggcccacgatgaaacctgagaggggtgtccccttgccccatcctctgcc | NM_013351.1 |
| | | | | ctaactacagtcgtttacctggtgctgcgtcttgcttttggtttccagctgga | |
| | | | | gaaaagaagacaagaaagtcttgggcatgaaggagctttttgcatctagtggg | |
| | | | | tgggaggggtcaggtgtgggacatgggagcaggagactccactttcttccttt | |
| | | | | gtacagtaactttcaaccttttcgttggcatgtgtgttaatccctgatccaaa | |
| | | | | aagaacaaatacacgtatgttataaccatcagcccgccagggtcagggaaagg | |
| | | | | actcacctgactttggacagctggcctgggctccccctgctcaaacacagtgg | |
| | | | | ggatcagagaaaaggggctggaaaggggggaatggcccacatctcaagaagcaa | |
| | | 1564 | CTCTGCCCTAACTACAGTCGTTTAC | | |
| | | 1565 | TACAGTCGTTTACCTGGTGCTGCGT | | |
| | | 1566 | GGAGCTTTTTGCATCTAGTGGGTGG | | |
| | | 1567 | GGGGTCAGGTGTGGGACATGGGAGC | | |
| | | 1568 | AACTTTCAACCTTTTCGTTGGCATG | | |
| | | 1569 | GGCATGTGTGTTAATCCCTGATCCA | | |
| | | 1570 | CGTATGTTATAACCATCAGCCCGCC | | |
| | | 1571 | GCCCGCCAGGGTCAGGGAAAGGACT | | |
| | | 1572 | GAAAGGACTCACCTGACTTTGGACA | | |
| | | 1573 | GAATGGCCCACATCTCAAGAAGCAA | | |
| DDX47 | 220890_s_at | 1574 | AGCCCAAAGGTTTGCCCGAATGGAG | agcccaaaggtttgcccgaatggagttaagggagcatggagaaaagaagaaac | NM_016355.1 |
| | | | | gctcgcgagaggatgctggagataatgatgacacagagggtgctattggtgtc | |
| | | | | aggaacaaggtggctggaggaaaaatgaagaagcggaaaggccgttaatcact | |
| | | | | tttatgaaggctcgagttctgctgttctgtaaaagagaattggagaatgaaac | |
| | | | | ctgctccaacagagatcatgagactgaaattggtcagaattgtgtccagaatg | |
| | | | | tgctcagctaattcagtattcttccccattctgggttggagtttactgcagag | |
| | | | | taattcttacagtgctgatgtcaagactgttactgttcttcgactttgattcc | |
| | | | | ttgctcatgacatgagtagggtgtgctcttctgtcacttcacacagacctttt | |
| | | | | gccttttttagctgcaagtcaaggactaggttgatgatgcccatgacctgtaa | |
| | | | | ttgtaaagaagcttggacatctgcaaatgatatttaaaccatcttggcttgtg | |
| | | | | ctt | |
| | | 1575 | GAAACGCTCGCGAGAGGATGCTGGA | | |
| | | 1576 | GAGGGTGCTATTGGTGTCAGGAACA | | |
| | | 1577 | AATTGTGTCCAGAATGTGCTCAGCT | | |
| | | 1578 | TCAGCTAATTCAGTATTCTTCCCCA | | |
| | | 1579 | GTTACTGTTCTTCGACTTTGATTCC | | |
| | | 1580 | GACTTTGATTCCTTGCTCATGACAT | | |
| | | 1581 | CATGAGTAGGGTGTGCTCTTCTGTC | | |
| | | 1582 | CTGTCACTTCACACAGACCTTTTGC | | |
| | | 1583 | GATGATGCCCATGACCTGTAATTGT | | |
| | | 1584 | ATTTAAACCATCTTGGCTTGTGCTT | | |
| DENND2D | 221081_s_at | 1585 | TTCTCACTTTTCATCCAGGAAGCCG | ttctcacttttcatccaggaagccgagaagagcaagaatcctcctgcaggcta | NM_024901.1 |
| | | | | tttccaacagaaaatacttgaatatgaggaacagaagaaacagaagaaaccaa | |
| | | | | gggaaaaaactgtgaaataagagctgtggtgaataagaatgactagagctaca | |
| | | | | caccatttctggacttcagcccctgccagtgtggcaggatcagcaaaactgtc | |
| | | | | agctcccaaaatccatatcctcactctgagtcttggtatccaggtattgcttc | |
| | | | | aaactggtgtctgagatttggatccctggtattgatttctcaggactttggag | |
| | | | | ggctctgacaccatgctcacagaactgggctcagagctccattttttgcagag | |
| | | | | gtgacacaggtaggaaacagtagtacatgtgttgtagacacttggttagaagc | |
| | | | | tgctgcaactgccctctcccatcattataacatcttcaacacagaacacactt | |
| | | | | tgtggtcgaaaggctcagcctctctacatgaagtctg | |
| | | 1586 | AAGAGCAAGAATCCTCCTGCAGGCT | | |
| | | 1587 | GAGCTACACACCATTTCTGGACTTC | | |
| | | 1588 | GGATCAGCAAAACTGTCAGCTCCCA | | |
| | | 1589 | ATCCTCACTCTGAGTCTTGGTATCC | | |
| | | 1590 | GGTGTCTGAGATTTGGATCCCTGGT | | |
| | | 1591 | GACACCATGCTCACAGAACTGGGCT | | |
| | | 1592 | GGCTCAGAGCTCCATTTTTTGCAGA | | |
| | | 1593 | TGGTTAGAAGCTGCTGCAACTGCCC | | |
| | | 1594 | TTTGTGGTCGAAAGGCTCAGCCTCT | | |
| | | 1595 | GCTCAGCCTCTCTACATGAAGTCTG | | |
| LOC339047 | 221501_x_at | 1596 | TGATAACTCCCTGAGCCTCAAGACA | gaatggcggcagtggagcatcgtcattcttcaggattgccctactggccctac | AF229069.1 |
| | | | | ctcacagctgaaactttaaaaaacaggatgggccaccagccacctcctccaac | |
| | | | | tcaacaacattctataattgataactccctgagcctcaagacaccttccgagt | |
| | | | | gtgtgctctatccccttccaccctcagcggatgataatctcaagacacctccc | |
| | | | | gagtgtctgctcactccccttccaccctcagctctaccctcagcggatgataa | |
| | | | | tctcaagacacctgccgagtgcctgctctatccccttccaccctcagcggatg | |
| | | | | ataatctcaagacacctcccgagtgtctgctcactccccttccaccctcagctccac | |
| | | | | cctcagcggatgataatctcaagacacctcctgagtgtgtctgctca | |
| | | 1597 | AGACACCTTCCGAGTGTGTGCTCTA | | |
| | | 1598 | GAATGGCGGCAGTGGAGCATCGTCA | | |
| | | 1599 | CCTTCCACCCTCAGCGGATGATAAT | | |
| | | 1600 | TGATAATCTCAAGACACCTCCCGAG | | |
| | | 1601 | AGCTCTACCCTCAGCGGATGATAAT | | |
| | | 1602 | TAATCTCAAGACACCTGCCGAGTGC | | |
| | | 1603 | GATGATAATCTCAAGACACCTCCCG | | |
| | | 1604 | CATCGTCATTCTTCAGGATTGCCCT | | |
| | | 1605 | GCTCCACCCTCAGCGGATGATAATC | | |
| | | 1606 | GACACCTCCTGAGTGTGTCTGCTCA | | |
| PYCARD | 221666_s_at | 1607 | CTGGATGCGCTGGAGAACCTGACCG | ctggatgcgctggagaacctgaccgccgaggagctcaagaagttcaagctgca | BC004470.1 |
| | | | | ggcggccacgcaccagggctctggagccgcgccagctgggatccaggcccctc | |
| | | | | ctcagtcggcagccaagccaggcctgcactttatagaccagcaccgggctgcg | |
| | | | | cttatcgcgagggtcacaaacgttgagtggctgctggatgctctgtacgggaa | |
| | | | | ggtcctgacggatgagcagtaccaggcagtgcgggccgagcccaccaacccaa | |
| | | | | gcaagatgcggaagctcttcagtttcacaccagcctggaactggacctgcaaggact | |
| | | | | tgctcctccaggccctaagggagtcccagtcctacctggtggaggac | |
| | | 1608 | GGCCTGCACTTTATAGACCAGCACC | | |
| | | 1609 | GCGCTTATCGCGAGGGTCACAAACG | | |
| | | 1610 | GTCACAAACGTTGAGTGGCTGCTGG | | |
| | | 1611 | CTGCTGGATGCTCTGTACGGGAAGG | | |
| | | 1612 | GTACGGGAAGGTCCTGACGGATGAG | | |
| | | 1613 | TGAGCAGTACCAGGCAGTGCGGGCC | | |
| | | 1614 | TGCGGAAGCTCTTCAGTTTCACACC | | |
| | | 1615 | GGAACTGGACCTGCAAGGACTTGCT | | |
| | | 1616 | CTCCAGGCCCTAAGGGAGTCCCAGT | | |
| | | 1617 | GTCCCAGTCCTACCTGGTGGAGGAC | | |
| IMP3 | 221688_s_at | 1618 | TCAATAAATGCCCCAACTGCTTTGT | gcgcagcatggaggactttgtcacttgggtggactcgtccaagatcaagcggc | AL136913.1 |
| | | | | acgtgctagagtacaatgaggagcgcgatgacttcgatctggaagcctagcgg | |
| | | | | atctcccactttgcatggctgtcttttacagatgggaaaactgaggcctgatg | |
| | | | | ctggagattctatgagggtgctctcctcaagggtatcagacggtcgtaggttc | |
| | | | | ttaagaatttgattcatcagtggcaggccatgcatagagccacgggaggtgcg | |
| | | | | tccttgttttccaggaaatgttcttagaacttggactactgattattaattga | |
| | | | | ctgtgccttgggaaacagtgggaagtaacttggtgcagcactggggtattgtt | |
| | | | | ggactggttcaattcgtttaactcgaattcttgctcctggccgtggttaagct | |
| | | | | gtgtacagatgatggagagtttggcctcaagtttttataaactgagcgagact | |
| | | | | agtgttcaggatctcctcccttgtttaaatgtcaataaatgccccaactgctttgt | |
| | | 1619 | GCGCAGCATGGAGGACTTTGTCACT | | |
| | | 1620 | TTTGTCACTTGGGTGGACTCGTCCA | | |
| | | 1621 | GACTCGTCCAAGATCAAGCGGCACG | | |
| | | 1622 | GGAGCGCGATGACTTCGATCTGGAA | | |
| | | 1623 | CCCACTTTGCATGGCTGTCTTTTAC | | |
| | | 1624 | GAGGCCTGATGCTGGAGATTCTATG | | |
| | | 1625 | GGTGCTCTCCTCAAGGGTATCAGAC | | |
| | | 1626 | GCATAGAGCCACGGGAGGTGCGTCC | | |
| | | 1627 | CTCCTGGCCGTGGTTAAGCTGTGTA | | |
| | | 1628 | ATCTCCTCCCTTGTTTAAATGTCAA | | |
| CSPG2 | 221731_x_at | 1629 | TTTCAGCACCGATGGCCATGTAAAT | tttcagcaccgatggccatgtaaataagatgatttaatgttgattttaatcct | J02814.1 |
| | | | | gtatataaantaaaaagtncncaatgagtttngggcatatttaatgatgatta | |
| | | | | tggagccttagaggtctttaatcattggttcnggctgcttttatgtagtttag | |
| | | | | gctggaaatggtttcacttgctctttgactgtcagcaagactgaagatggctt | |
| | | | | ttcctggacagctagaaaacacaaaatcttgtaggtcattgcacctatctcag | |
| | | | | ccataggtgcagtttgcttctacatgatgctaaaggctgcgaatgggatcctg | |
| | | | | atggaactaaggactccaatgtcgaactcttctttgctgcattcctttttctt | |
| | | | | cacttacaagaaaggcctgaatggaggacttttctgtaaccaggaacattttt | |
| | | | | taggggtcaaagtgctaataattaactcaaccaggtctactttttaatggctt | |
| | | | | tcataacactaactcataaggttaccgatcaatgcatttcatacggatatagacctag | |
| | | | | ggctctggagggtgggg | |
| | | 1630 | GAAATGGTTTCACTTGCTCTTTGAC | | |
| | | 1631 | GAAGATGGCTTTTCCTGGACAGCTA | | |
| | | 1632 | TGTAGGTCATTGCACCTATCTCAGC | | |
| | | 1633 | GGTGCAGTTTGCTTCTACATGATGC | | |
| | | 1634 | GGCTGCGAATGGGATCCTGATGGAA | | |
| | | 1635 | CCAATGTCGAACTCTTCTTTGCTGC | | |
| | | 1636 | CATTCCTTTTTCTTCACTTACAAGA | | |
| | | 1637 | GGTCTACTTTTTAATGGCTTTCATA | | |
| | | 1638 | AAGGTTACCGATCAATGCATTTCAT | | |
| | | 1639 | AGACCTAGGGCTCTGGAGGGTGGGG | | |
| GNLY | 37145_at | 1640 | TCCTTGCAGCCATGCTCCTGGGCAA | tccttgcagccatgctcctgggcaacccaggtctggtcttntctcgtctgagc | M85276 |
| | | | | ccnnngtacnacgancnngcaagancccacctnnntgntgaggagaaatcctn | |
| | | | | gcccgtgncnngnccaggaggnnccnnnnnnnnnnnnnnngaccaaaacacag | |
| | | | | gnnnnnnnnnnnnnnnnnnnnnnnnnnnnnnnnnnnnnnnnnnnnnnnnnnnn | |
| | | | | nnnnnnggataagcccacccagagaagtgtttccaatgctgcgacccgggtgt | |
| | | | | gtaggacggggaggtcacgatggcgcgacgtctgcagaaatttcatgaggagg | |
| | | | | tatcagtctagagttacccagggcctcgtggccggagaaactgcccagcagat | |
| | | | | ctgtgaggacctcaggttgtgtataccttctacaggtcccctctgagccctctcacc | |
| | | | | ttgtcctgtggaagaagcacag | |
| | | 1641 | ATGCTCCTGGGCAACCCAGGTCTGG | | |
| | | 1642 | TGCTCCTGGGCAACCCAGGTCTGGT | | |
| | | 1643 | CCACCCAGAGAAGTGTTTCCAATGC | | |
| | | 1644 | CACCCAGAGAAGTGTTTCCAATGCT | | |
| | | 1645 | GAGAAGTGTTTCCAATGCTGCGACC | | |
| | | 1646 | TGTTTCCAATGCTGCGACCCGGGTG | | |
| | | 1647 | TCCAATGCTGCGACCCGGGTGTGTA | | |
| | | 1648 | CACGATGGCGCGACGTCTGCAGAAA | | |
| | | 1649 | GCGCGACGTCTGCAGAAATTTCATG | | |
| | | 1650 | GACGTCTGCAGAAATTTCATGAGGA | | |
| | | 1651 | GTATCAGTCTAGAGTTACCCAGGGC | | |
| | | 1652 | TGCCCAGCAGATCTGTGAGGACCTC | | |
| | | 1653 | ATACCTTCTACAGGTCCCCTCTGAG | | |
| | | 1654 | GCCCTCTCACCTTGTCCTGTGGAAG | | |
| | | 1655 | ACCTTGTCCTGTGGAAGAAGCACAG | | |
| TMEM161A | 43977_at | 1656 | CCTCATCTGGTGGACGGCTGCCTGC | cctcatctggtggacggctgcctgccagctgctcgccagccttttcggcctct | AI660497 |
| | | | | acttccaccagcacttggcaggctcctagctgcctgcagaccctcctggggcc | |
| | | | | ctgaggtctgttcctggggcagcgggacactagcctgccccctctgtttgcgc | |
| | | | | ccccgtgtccccagctgcaaggtggggccggactccccggcgttcccttcacc | |
| | | | | acagtgcctgacccgcggccccccttggacgccgagtttctgcctcagaactg | |
| | | | | tctctcctgggcccagcagcatgagggtcccgaggccattgtctccgaagcgt | |
| | | | | atgtgccaggtttgagtggcgagggtgatgctggctgctcttctgaacaaataaag | |
| | | 1657 | CTCATCTGGTGGACGGCTGCCTGCC | | |
| | | 1658 | TACTTCCACCAGCACTTGGCAGGCT | | |
| | | 1659 | CCAGCACTTGGCAGGCTCCTAGCTG | | |
| | | 1660 | CTTGGCAGGCTCCTAGCTGCCTGCA | | |
| | | 1661 | TCCTGGGGCCCTGAGGTCTGTTCCT | | |
| | | 1662 | CCCTGAGGTCTGTTCCTGGGGCAGC | | |
| | | 1663 | CCCGTGTCCCCAGCTGCAAGGTGGG | | |
| | | 1664 | TGGACGCCGAGTTTCTGCCTCAGAA | | |
| | | 1665 | GTTTCTGCCTCAGAACTGTCTCTCC | | |
| | | 1666 | CATTGTCTCCGAAGCGTATGTGCCA | | |
| | | 1667 | CTCCGAAGCGTATGTGCCAGGTTTG | | |
| | | 1668 | CCGAAGCGTATGTGCCAGGTTTGAG | | |
| | | 1669 | CGAGGGTGATGCTGGCTGCTCTTCT | | |
| | | 1670 | TGATGCTGGCTGCTCTTCTGAACAA | | |
| | | 1671 | TGGCTGCTCTTCTGAACAAATAAAG | | |

What is claimed is:

1. A method for treating a subject having cancer with an immunotherapeutic agent, comprising
determining expression level of at least one gene in a blood sample obtained from the subject,
wherein the at least one gene is selected from a first group of genes as listed in Table 2 and a second group of genes as listed in Table 3;
determining likelihood of clinical response of the subject to the treatment based on the expression level of the at least one gene in the blood sample,
wherein the expression level of the at least one gene selected from the first group of genes is positively correlated with the likelihood of clinical response, and
wherein the expression level of the at least one gene selected from the second group of genes is negatively correlated with the likelihood of clinical response; and
administering to the subject a therapeutically effective amount of the immunotherapeutic agent for treating the cancer.

2. The method claim 1, wherein the at least one gene is selected from IL2RB, KLRK1, G3BP, PPP1R16B, CLIC3, PRF1, SPON2, HOP, GNLY, TMEM161A, PRKCH, RUNX3, EOMES, SLC25A5, GZMB, IMP3, and ZAP70, wherein the expression level of the at least one gene is positively correlated with the likelihood of clinical response.

3. The method claim 1, wherein the at least one gene is selected from ASGR1, ASGR2, CENTA2, PGLS, MAPBPIP, STX10, C16ORF68, and RAB31, wherein the expression level of the at least one gene is negatively correlated with the likelihood of clinical response.

4. The method claim 1, wherein the expression level of at least two genes in the blood sample is determined, and wherein determining the likelihood of clinical response is based on the expression level of the at least two genes in the blood sample.

5. The method of claim 4, wherein a first gene of the at least two genes is selected from the first group of genes as listed in Table 2, and a second gene of the at least two genes is selected from the second group of genes as listed in Table 3.

6. The method of claim 5, wherein the first gene is selected from IL2RB, KLRK1, G3BP, PPP1R16B, CLIC3, PRF1, SPON2, HOP, GNLY, TMEM161A, PRKCH, RUNX3, EOMES, SLC25A5, GZMB, IMP3, and ZAP70.

7. The method of claim 5, wherein the second gene is selected from ASGR1, ASGR2, CENTA2, PGLS, MAPBPIP, STX10, C16ORF68, and RAB31.

8. The method of claim 5, wherein the first gene is IL2RB and the second gene is selected from ASGR1 and ASGR2.

9. The method of claim 8, wherein the first gene is IL2RB and the second gene is ASGR2.

10. The method of claims 4, wherein determining the likelihood of clinical response comprises
subjecting the expression level of the at least two genes to a formula to calculate a score,
wherein the formula is pre-determined by statistical analysis of (a) clinical response of a plurality of patients having the cancer to treatment with the immunotherapeutic agent and (b) the expression level of the at least two genes in pre-treatment blood samples from the plurality of patients.

11. The method of claim 10, wherein a first gene of the at least two genes is selected from the first group of genes as listed in Table 2, and a second gene of the at least two genes is selected from the second group of genes as listed in Table 3,
wherein the formula for calculating the score is
Score=−C₁*X_{first gene}+C₂*X_{second gene},
wherein X_{first gene} and X_{second gene} are normalized mRNA expression level of the first and the second gene, respectively, and C1 and C2 are each, independently, a number ranging from 0.01 to 3, and
wherein the score is negatively correlated with the likelihood of clinical response.

12. The method of claim 11, wherein C₁ ranges from 0.1 to 2, and C₂ ranges from 0.1 to 1.5.

13. The method of claim 11, wherein the first gene is IL2RB, and the second gene is ASGR2, and wherein C₁ ranges from 0.2 to 1.5, and C₂ ranges from 0.1 to 1.

14. The method of claim 11, wherein the score is compared to a predetermined threshold, wherein a score that is lower than the threshold is indicative of high likelihood of clinical response, and a score that is higher than the threshold is indicative of low likelihood of clinical response.

15. The method of any of claims 1-14, wherein the expression level of the at least one gene is measured by at least one method selected from microarray, quantitative polymerase chain reaction (qPCR), and flow cytometry.

16. The method of any one of claims 1-15, wherein the immunotherapeutic agent is an anti-CTLA4 antibody.

17. The method of claim 16, wherein the anti-CTLA4 antibody is ipilimumab.

18. The method of any one of claims 1-17, wherein the cancer is selected from melanoma, prostate cancer, lung cancer, ovarian cancer, gastric cancer, and glioblastoma.

19. The method of claim 18, wherein the cancer is advanced melanoma.

20. The method of claim 18, wherein the cancer is metastatic melanoma.

21. The method of claim 18, wherein the cancer is stage III or IV melanoma.

22. The method of claim 21, wherein the cancer is unresectable stage III or IV melanoma.

23. The method of claim 1-22, wherein determining the likelihood of clinical response is based on the gene expression level and at least one additional factor.

24. The method claim 23, wherein the at least one additional factor is selected from baseline serum LDH level and disease stage.

25. The method claim 24, wherein the at least one additional factor is baseline serum LDH level.

26. The method of any one of claims 1-25, wherein the subject is not being treated with the immunotherapeutic agent at the time the likelihood of clinical response of the subject is determined.

27. A method of predicting likelihood of clinical response of a subject having cancer o treatment with an immunotherapeutic agent, comprising:
obtaining a blood sample from the subject before the treatment,
determining expression level of at least one gene in the blood sample,
wherein the at least one gene is selected from a first group of genes as listed in Table 2 and a second group of genes as listed in Table 3;
determining likelihood of clinical response to the treatment based on the expression level of the at least one gene in the blood sample,
wherein the expression level of the at least one gene selected from the first group of genes is positively correlated with the likelihood of clinical response, and
wherein the expression level of the at least one gene selected from the second group of genes is negatively correlated with the likelihood of clinical response.

28. The method claim 27, wherein the at least one gene is selected from IL2RB, KLRK1, G3BP, PPP1R16B, CLIC3, PRF1, SPON2, HOP, GNLY, TMEM161A, PRKCH, RUNX3, EOMES, SLC25A5, GZMB, IMP3, and ZAP70, wherein the expression level of the at least one gene is positively correlated with the likelihood of clinical response.

29. The method claim 27, wherein the at least one gene is selected from ASGR1, ASGR2, CENTA2, PGLS, MAPBPIP, STX10, C16ORF68, and RAB31, wherein the expression level of the at least one gene is negatively correlated with the likelihood of clinical response.

30. The method claim 27, wherein the expression level of at least two genes in the blood sample is determined, and wherein determining the likelihood of clinical response is based on the expression level of the at least two genes in the blood sample.

31. The method of claim 30, wherein a first gene of the at least two genes is selected from the first group of genes as listed in Table 2, and a second gene of the at least two genes is selected from the second group of genes as listed in Table 3.

32. The method of claim 31, wherein the first gene is selected from IL2RB, KLRK1, G3BP, PPP1R16B, CLIC3, PRF1, SPON2, HOP, GNLY, TMEM161A, PRKCH, RUNX3, EOMES, SLC25A5, GZMB, IMP3, and ZAP70.

33. The method of claim 31, wherein the second gene is selected from ASGR1, ASGR2, CENTA2, PGLS, MAPBPIP, STX10, C16ORF68, and RAB31.

34. The method of claim 31, wherein the first gene is IL2RB and the second gene is selected from ASGR1 and ASGR2.

35. The method of claim 34, wherein the first gene is IL2RB and the second gene is ASGR2.

36. The method of claims 30, wherein determining the likelihood of clinical response comprises
subjecting the expression level of the at least two genes to a formula to calculate a score,
wherein the formula is pre-determined by statistical analysis of (a) clinical response of a plurality of patients having the cancer to treatment with the immunotherapeutic agent and (b) the expression level of the at least two genes in pre-treatment blood samples from the plurality of patients.

37. The method of claim 36, wherein a first gene of the at least two genes is selected from the first group of genes as listed in Table 2, and a second gene of the at least two genes is selected from the second group of genes as listed in Table 3,
wherein the formula for calculating the score is
Score=−C₁*X_{first gene}+C₂*X_{second gene},
wherein X_{first gene} and X_{second gene} are normalized mRNA expression level of the first and the second gene, respectively, and C1 and C2 are each, independently, a number ranging from 0.01 to 3, and
wherein the score is negatively correlated with the likelihood of clinical response.

38. The method of claim 37, wherein C₁ ranges from 0.1 to 2, and C₂ ranges from 0.1 to 1.5.

39. The method of claim 38, wherein the first gene is IL2RB, and the second gene is ASGR2, and wherein C₁ ranges from 0.2 to 1.5, and C₂ ranges from 0.1 to 1.

40. The method of claim 32, wherein the score is compared to a predetermined threshold, wherein a score that is lesser than the threshold is indicative of high likelihood of clinical response, and a score that is greater than the threshold is indicative of low likelihood of clinical response.

41. The method of any of claims 27-40, wherein the expression level of the at least one gene is measured by at least one method selected from microarray, quantitative polymerase chain reaction (qPCR), and flow cytometry.

42. The method of any one of claims 27-41, wherein the immunotherapeutic agent is an anti-CTLA4 antibody.

43. The method of claim 42, wherein the anti-CTLA4 antibody is ipilimumab.

44. The method of any one of claims 27-43, wherein the cancer is selected from melanoma, prostate cancer, lung cancer, ovarian cancer, gastric cancer, and glioblastoma.

45. The method of claim 44, wherein the cancer is advanced melanoma.

46. The method of claim 44, wherein the cancer is metastatic melanoma.

47. The method of claim 44, wherein the cancer is stage III or IV melanoma.

48. The method of claim 47, wherein the cancer is unresectable stage III or IV melanoma.

49. The method of claim 27-48, wherein determining the likelihood of clinical response is based on the gene expression level and at least one additional factor.

50. The method claim 49, wherein the at least one additional factor is selected from baseline serum LDH level and disease stage.

51. The method claim 50, wherein the at least one additional factor is baseline serum LDH level.

52. The method of any one of claims 27-51, wherein the subject is not being treated with the immunotherapeutic agent at the time the likelihood of clinical response of the subject is determined.

53. A method for treating a subject having melanoma with an ipilimumab, comprising
determining expression level of at least one gene in a blood sample obtained from the subject,
wherein the at least one gene is selected from a first group of genes as listed in Table 2 and a second group of genes as listed in Table 3;
determining likelihood of clinical response to the treatment based on the expression level of the at least one gene in the blood sample,
wherein the expression level of the at least one gene selected from the first group of genes is positively correlated with the likelihood of clinical response, and
wherein the expression level of the at least one gene selected from the second group of genes is negatively correlated with the likelihood of clinical response; and
administering to the subject a therapeutically effective amount of the ipilimumab for treating melanoma.

54. A method for treating a subject having melanoma with an ipilimumab, comprising
determining expression level of at least one gene in a blood sample obtained from the subject,
wherein the at least one gene is selected from a first group of genes as listed in Table 2 and a second group of genes as listed in Table 3;
determining likelihood of clinical response to the treatment based on the expression level of the at least one gene in the blood sample,
wherein the expression level of the at least one gene selected from the first group of genes is positively correlated with the likelihood of clinical response, and
wherein the expression level of the at least one gene selected from the second group of genes is negatively correlated with the likelihood of clinical response; and
administering to the subject a therapeutically effective amount of the ipilimumab for treating melanoma if the likelihood of clinical response is higher than a predetermined value.

55. A method for determining whether to treat a subject having cancer with a immunotherapeutic agent, comprising
obtaining a blood sample from the subject,
determining expression level of at least one gene in a blood sample obtained from the subject,
wherein the at least one gene is selected from a first group of genes as listed in Table 2 and a second group of genes as listed in Table 3;
determining likelihood of clinical response to the treatment based on the expression level of the at least one gene in the blood sample,
wherein the expression level of the at least one gene selected from the first group of genes is positively correlated with the likelihood of clinical response, and
wherein the expression level of the at least one gene selected from the second group of genes is negatively correlated with the likelihood of clinical response; and
determining whether to treat the subject having cancer with the immunotherapeutic agent based on the likelihood of clinical response.

56. A method for determining whether to treat a subject having melanoma with ipilimumab, comprising
obtaining a blood sample from the subject,
determining expression level of at least one gene in a blood sample obtained from the subject,
wherein the at least one gene is selected from a first group of genes as listed in Table 2 and a second group of genes as listed in Table 3;
determining likelihood of clinical response to the treatment based on the expression level of the at least one gene in the blood sample,
wherein the expression level of the at least one gene selected from the first group of genes is positively correlated with the likelihood of clinical response, and
wherein the expression level of the at least one gene selected from the second group of genes is negatively correlated with the likelihood of clinical response; and
determining whether to treat the subject having cancer with ipilimumab based on the likelihood of clinical response.

57. A kit comprising one or more reagents for determining expression level of at least one gene in a blood sample, wherein the at least one gene is selected from a first group of genes as listed in Table 2 and a second group of genes as listed in Table 3.

58. The kit of claim 57, wherein the one or more reagents are used to determine mRNA expression level of the at least one gene.

59. The kit of claim 57, comprising at least one polynucleotide capable of specifically hybridizing to the at least one gene.

60. The method claim 57, wherein the at least one gene is selected from IL2RB, KLRK1, G3BP, PPP1R16B, CLIC3, PRF1, SPON2, HOP, GNLY, TMEM161A, PRKCH, RUNX3, EOMES, SLC25A5, GZMB, IMP3, and ZAP70.

61. The method claim 57, wherein the at least one gene is selected from ASGR1, ASGR2, CENTA2, PGLS, MAPBPIP, STX10, C16ORF68, and RAB31.

62. The method claim 57, wherein the kit comprises one or more reagents for determining expression level of at least two genes in the blood sample.

63. The method of claim 62, wherein a first gene of the at least two genes is selected from the first group of genes as listed in Table 2, and a second gene of the at least two genes is selected from the second group of genes as listed in Table 3.

64. The method of claim 63, wherein the first gene is selected from IL2RB, KLRK1, G3BP, PPP1R16B, CLIC3, PRF1, SPON2, HOP, GNLY, TMEM161A, PRKCH, RUNX3, EOMES, SLC25A5, GZMB, IMP3, and ZAP70.

65. The method of claim 63, wherein the second gene is selected from ASGR1, ASGR2, CENTA2, PGLS, MAPBPIP, STX10, C16ORF68, and RAB31.

66. The method of claim 63, wherein the first gene is IL2RB and the second gene is selected from ASGR1 and ASGR2.

67. The method of claim 66, wherein the first gene is IL2RB and the second gene is ASGR2.

68. A method for treating a subject having cancer with an immunotherapeutic agent, comprising
determining expression levels of a first gene and a second gene in a blood sample obtained from the subject,
wherein the first gene is IL2RB and a second gene is selected from ASGR1 and ASGR2;
determining likelihood of longer overall survival of the subject following the treatment based on the expression levels of the first gene and the second gene in the blood sample,
wherein the expression levels of the first gene and the second gene are used to calculate a score according to formula:
Score=−C₁*X_{first gene}+C₂*X_{second gene},
wherein X_{first gene} and X_{second gene} are normalized mRNA expression levels of the first and the second gene, respectively, and C1 and C2 are each, independently, a number ranging from 0.01 to 3,
wherein the score is negatively correlated with the likelihood of longer overall survival;
administering to the subject a therapeutically effective amount of the immunotherapeutic agent for treating the cancer.

69. The method of claim 68, wherein C₁ ranges from 0.1 to 2, and C₂ ranges from 0.1 to 1.5.

70. The method of claim 68, wherein the first gene is IL2RB, and the second gene is ASGR2, and wherein C₁ ranges from 0.2 to 1.5, and C₂ ranges from 0.1 to 1.

71. The method of claim 68, wherein the score is compared to a predetermined threshold, wherein a score that is lower than the threshold is indicative of high likelihood of longer overall survival, and a score that is higher than the threshold is indicative of low likelihood of longer overall survival.

72. The method of any of claims 68-71, wherein the expression level of the at least one gene is measured by at least one method selected from microarray and quantitative polymerase chain reaction (qPCR).

73. The method of any one of claims 68-72, wherein the immunotherapeutic agent is an anti-CTLA4 antibody.

74. The method of claim 73, wherein the anti-CTLA4 antibody is ipilimumab.

75. The method of any one of claims 68-74, wherein the cancer is selected from melanoma, prostate cancer, lung cancer, ovarian cancer, gastric cancer, and glioblastoma.

76. The method of claim 75, wherein the cancer is advanced melanoma.

77. The method of claim 75, wherein the cancer is metastatic melanoma.

78. The method of claim 75, wherein the cancer is stage III or IV melanoma.

79. The method of claim 78, wherein the cancer is unresectable stage III or IV melanoma.

80. The method of claim 68-79, wherein determining the likelihood of clinical response is based on the gene expression level and at least one additional factor.

81. The method claim 80, wherein the at least one additional factor is selected from baseline serum LDH level and disease stage.

82. The method claim 80, wherein the at least one additional factor is baseline serum LDH level.

83. The method of any one of claims 68-82, wherein the subject is not being treated with the immunotherapeutic agent at the time the likelihood of clinical response of the subject is determined.

84. A method of predicting likelihood of longer overall survival of a subject having cancer to treatment with an immunotherapeutic agent, comprising:
obtaining a blood sample from the subject before the treatment,
determining expression levels of a first gene and a second gene in the blood sample obtained from the subject,
wherein the first gene is IL2RB and a second gene is selected from ASGR1 and ASGR2;
determining likelihood of longer overall survival of the subject following the treatment based on the expression levels of the first gene and the second gene in the blood sample,
wherein the expression levels of the first gene and the second gene are used to calculate a score according to formula:
Score=−C₁*X_{first gene}+C₂*X_{second gene},
wherein X_{first gene} and X_{second gene} are normalized mRNA expression levels of the first and the second gene, respectively, and C1 and C2 are each, independently, a number ranging from 0.01 to 3,
wherein the score is negatively correlated with the likelihood of longer overall survival.

85. The method of claim 84, wherein C₁ ranges from 0.1 to 2, and C₂ ranges from 0.1 to 1.5.

86. The method of claim 84, wherein the first gene is IL2RB, and the second gene is ASGR2, and wherein C₁ ranges from 0.2 to 1.5, and C₂ ranges from 0.1 to 1.

87. The method of claim 84, wherein the score is compared to a predetermined threshold, wherein a score that is lower than the threshold is indicative of high likelihood of longer overall survival, and a score that is higher than the threshold is indicative of low likelihood of longer overall survival.

88. The method of any of claims 84-87, wherein the expression level of the at least one gene is measured by at least one method selected from microarray and quantitative polymerase chain reaction (qPCR).

89. The method of any one of claims 84-88, wherein the immunotherapeutic agent is an anti-CTLA4 antibody.

90. The method of claim 89, wherein the anti-CTLA4 antibody is ipilimumab.

91. The method of any one of claims 84-90, wherein the cancer is selected from melanoma, prostate cancer, lung cancer, ovarian cancer, gastric cancer, and glioblastoma.

92. The method of claim 91, wherein the cancer is advanced melanoma.

93. The method of claim 91, wherein the cancer is metastatic melanoma.

94. The method of claim 91, wherein the cancer is stage III or IV melanoma.

95. The method of claim 94, wherein the cancer is unresectable stage III or IV melanoma.

96. The method of claim 84-95, wherein determining the likelihood of clinical response is based on the gene expression level and at least one additional factor.

97. The method claim 96, wherein the at least one additional factor is selected from baseline serum LDH level and disease stage.

98. The method claim 96, wherein the at least one additional factor is baseline serum LDH level.

99. The method of any one of claims 84-98, wherein the subject is not being treated with the immunotherapeutic agent at the time the likelihood of clinical response of the subject is determined.
